(19) United States
(12) Patent Application Publication
(10) Pub. No.: US 2015/0094294 A1
(43) Pub. Date: Apr. 2, 2015

(54) COMPOUNDS AND METHODS FOR TREATING MALARIA

(71) Applicants: Cellceutix Corporation, Beverly, MA (US); The Trustees of the University of Pennsylvania, Philadelphia, PA (US)

(72) Inventors: Richard W. Scott, Radnor, PA (US); Robert W. Kavash, Radnor, PA (US); Wenxi Pan, Radnor, PA (US); Katie Freeman, Radnor, PA (US); Michael J. Costanzo, Radnor, PA (US); Doron Greenbaum, Philadeiphia, PA (US); Yongjiang Xu, Radnor, PA (US); Trevor Young, Radnor, PA (US)

(21) Appl. No.: 14/382,104

(22) PCT Filed: Feb. 28, 2013
(86) PCT No.: PCT/US13/28221

§ 371 Date: Aug. 29, 2014

Related U.S. Application Data
(60) Provisional application No. 61/604,566, filed on Feb. 29, 2012.

(51) Int. Cl. C07D 409/14; A01N 41/10; C07C 279/14; A01N 47/44; C07D 295/096; A01N 43/60; C07D 333/76; C07D 239/28; C07D 249/06; A01N 43/647; C07C 317/40; C07D 403/14; C07D 213/81; A01N 43/40; A01N 47/30; A01N 43/78; C07C 317/22

(52) U.S. Cl. CPC C07D 409/14; C07C 317/22; A01N 41/10; C07C 279/14; A01N 47/44; C07D 295/096; A01N 43/60; C07D 333/76; C07D 239/28; C07D 249/06; A01N 43/647; C07C 317/40; C07D 403/14; C07D 213/81; A01N 43/40; A01N 47/30; A01N 43/78
USPC 514/210.2; 564/389; 514/655; 564/155; 514/616; 544/357; 514/252.11; 544/335; 514/256; 548/255; 514/359; 564/154; 546/323; 514/354; 544/316; 514/274

(57) ABSTRACT

The present disclosure provides compounds, specifically pyrimidin-4,6-dicarboxylic acid amide derivatives, or pharmaceutically acceptable salts thereof and pharmaceutical compositions containing said compounds. Also provided are methods for treating a mammal having malaria, or killing or inhibiting the growth of a Plasmodium species, comprising administering to said mammal or contacting said Plasmodium species with, an effective amount of a pyrimidin-4,6-dicarboxylic acid amide derivative.

### REFERENCE TO GOVERNMENT GRANTS

The present disclosure was supported by funds from the U.S. Government (NIH/NIAID Grant No. 1R44AI090762-01) and the U.S. Government may therefore have certain rights in the disclosure.

### FIELD

The present disclosure is directed, in part, to compounds, and pharmaceutically acceptable salts thereof, and compositions thereof for treating a mammal having malaria, or killing or inhibiting the growth of a Plasmodium species.

### BACKGROUND

World-wide, 41% of the population live in areas where malaria is transmitted, such as parts of Africa, Asia, Middle East, Central and South America, Hispaniola, and Oceania. Each year between 350 and 500 million cases of malaria occur worldwide, and over one million people die, most of them young children in sub-Saharan Africa. In areas of Africa with high malaria transmission, an estimated 990,000 people died of malaria in 1995. In 2002, malaria was the fourth cause of death in children in developing countries. In addition, malaria caused 10.7% of all children's deaths in developing countries.

Antimicrobial peptides (AMPs) represent a component of the innate immune system that provides resistance to a variety of pathogenic bacteria. AMPs have provided new leads for developing antibiotics, because they play a central role in the innate immune system. Some AMPs display very broad spectrum action against bacteria, yeast, fungus, and even viruses. Anti-parasitic activities have also been reported for a number of host defense peptides. The best studied organisms include Plasmodium, Leishmania, and Trypanosoma (Vizioli et al., Trends in Pharmacol., 2002, 18, 475-476; Jacobs et al., Antimicrob. Agents Chemother., 2003, 47, 607-613; and Brand et al., J. Biol. Chem., 2002, 277, 49332-49340), the parasitic agents of malaria, leishmaniasis and Chagas' disease, respectively. Additional protozoan parasites reported to be killed by the host defense peptides are Cryptosporidium (Giacometti et al., Antimicrob. Agents Chemother., 2000, 44, 3473-3475) and Giardia (Aley et al., Infect. Immun., 1994, 62, 5397-5403), human pathogens transmitted in contaminated drinking water. The peptides appear to kill protozoa by interacting with the cytoplasmic membrane causing excessive permeability, lysis and death; a mechanism which is similar to their mechanism of action against bacteria. Specificity for the parasite versus the host cell can be attributed to differences in phospholipid content and the lack of cholesterol in the protozoan membrane. Because the site of action is at the membrane and not to any specific receptor or intracellular target, the development of resistance to the cytotoxic properties of the antimicrobial peptides is highly unlikely.

With regard to anti-malarial activities, natural host defense proteins and their analogs have been shown to inhibit oocyst development of several Plasmodium species in various mosquito hosts (Gwadz et al., Infect. Immun., 1989, 57, 2628-2633; and Possani et al., Toxicon, 1998, 36, 1683-1692) and are directly cytotoxic against early sporogonic stages of Plasmodium in cell culture (Arrighi et al., Antimicrob. Agents Chemother., 2002, 46, 2104-2110). Furthermore, several antimicrobial peptides have been identified which selectively kill intraerthrocytic parasites (plasmodia life forms growing in red blood cells) by either attacking the infected erythrocyte while sparring normal erthrocytes (Feder et al., J. Biol. Chem., 2000, 275, 4230-4238; and Krugliak et al., Antimicrob. Agents Chemother., 2000, 44, 2442-2451) or interacting with and killing the intracellular parasite without harming the infected red blood cell (Dagan et al., Antimicrob. Agents Chemother., 2002, 46, 1059-1066; and Efron et al., J. Biol. Chem., 2002, 277, 24067-24072). Recognizing the significant therapeutic limitations of peptides, the development of nonpeptidic mimics of these anti-plasmodia peptides would represent a novel and powerful therapy to combat malaria.

### SUMMARY

The present disclosure provides compounds of Formula I:

R¹-A₁-X—Y-A₂-Y—X-A₁-R² (I)

or a pharmaceutically acceptable salt thereof, wherein: each X is, independently, —NR⁸, —N(R⁸)N(R⁸)—, O, or S, wherein each R⁸ is, independently, hydrogen or alkyl; each Y is, independently, C═O, C═S, or O═S═O; each A₁ and A₂ is, independently, aryl optionally substituted with one or more W, or heteroaryl optionally substituted with one or more W; each W is, independently, halo, —CF₃, cyano, C₁₋₄alkoxy, —S—(CH₂)₁₋₅NHC(═NH)NH₂, —S—(CH₂)₁₋₅NH₂, —S—(CH₂)₁₋₅N(CH₃)₂, —S—(CH₂)₁₋₅NHS(═O)₂—C₁₋₄alkyl, —C₁₋₄alkyl, —O—(CH₂)₁₋₅NHC(═NH)NH₂, or O-heterocycle (wherein the heterocycle is optionally substituted with one or more cyano, amino, C₁₋₄alkyl, guanidino, hydroxyl, amidino, C₁₋₄alkoxy, CF₃, or halo); and R¹ and R² are, independently, —(CH₂)₁₋₄NH₂, —(CH₂)₁₋₅NHC(═NH)NH₂, —S—(CH₂)₁₋₅N(CH₃)₂, —O—(CH₂)₁₋₅NHC(═NH)NH₂, —S—(CH₂)₁₋₅NHC(═NH)NH₂, —O—(CH₂)₁₋₅N(CH₃)₂, amino, —O-heterocycle or —O-cycloalkyl (wherein the heterocycle and cycloalkyl are optionally substituted), —S-heterocycle (wherein the heterocycle is optionally substituted), —NHC(═O)—(CH₂)₁₋₅NHC(═NH)NH₂, —NHC(═O)—C₁₋₄alkyl, heterocycle (wherein the heterocycle is optionally substituted), —NHC(═O)—(CH₂)₁₋₅NHS(═O)₂—C₁₋₄alkyl, —NHC(═O)—(CH₂)₁₋₅-aryl (wherein either or both —(CH₂)₁₋₅ and aryl is optionally substituted), —NHC(═O)-aryl (wherein the aryl is optionally substituted), or —NHC(═O)—(CH₂)₁₋₅NH-aryl (wherein either or both —NH and aryl is optionally substituted), wherein the optional substituents are chosen from one or more cyano, amino, C₁₋₄alkyl, guanidino, hydroxyl, amidino, C₁₋₄alkoxy, CF₃, halo, and —N(CH₃)₂; provided that the compound of Formula I is not one of the following:

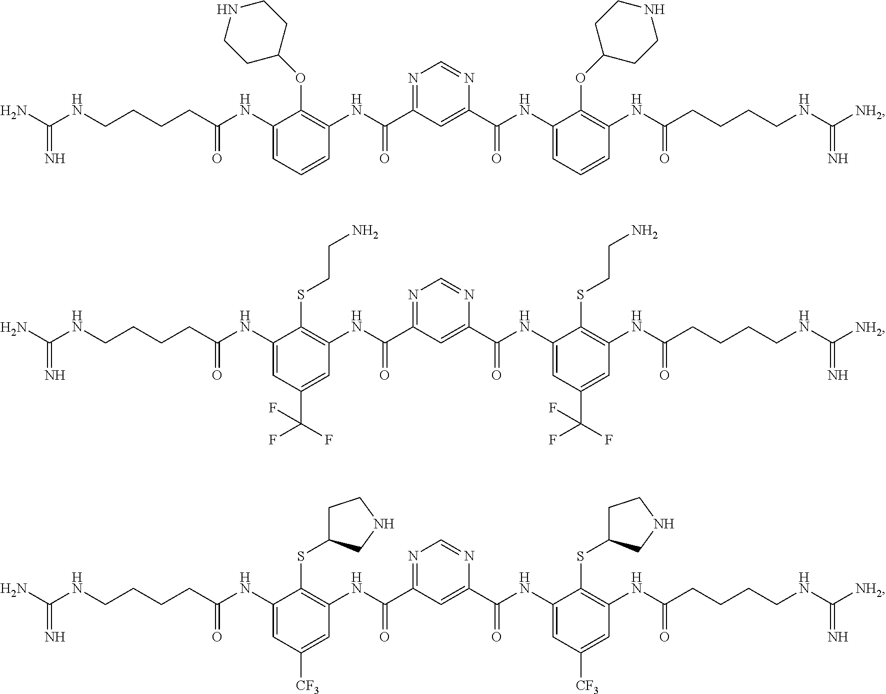 [SMILES: N=C(N)NCCCCC(=O)Nc1cccc(NC(=O)c2cc(C(=O)Nc3cccc(NC(=O)CCCCNC(=N)N)c3OC3CCNCC3)ncn2)c1OC1CCNCC1 ; N=C(N)NCCCCC(=O)Nc1cc(C(F)(F)F)cc(NC(=O)c2cc(C(=O)Nc3cc(C(F)(F)F)cc(NC(=O)CCCCNC(=N)N)c3SCCN)ncn2)c1SCCN ; N=C(N)NCCCCC(=O)Nc1cc(C(F)(F)F)cc(NC(=O)c2cc(C(=O)Nc3cc(C(F)(F)F)cc(NC(=O)CCCCNC(=N)N)c3S[C@H]3CCNC3)ncn2)c1S[C@H]1CCNC1]

or its enantiomer,

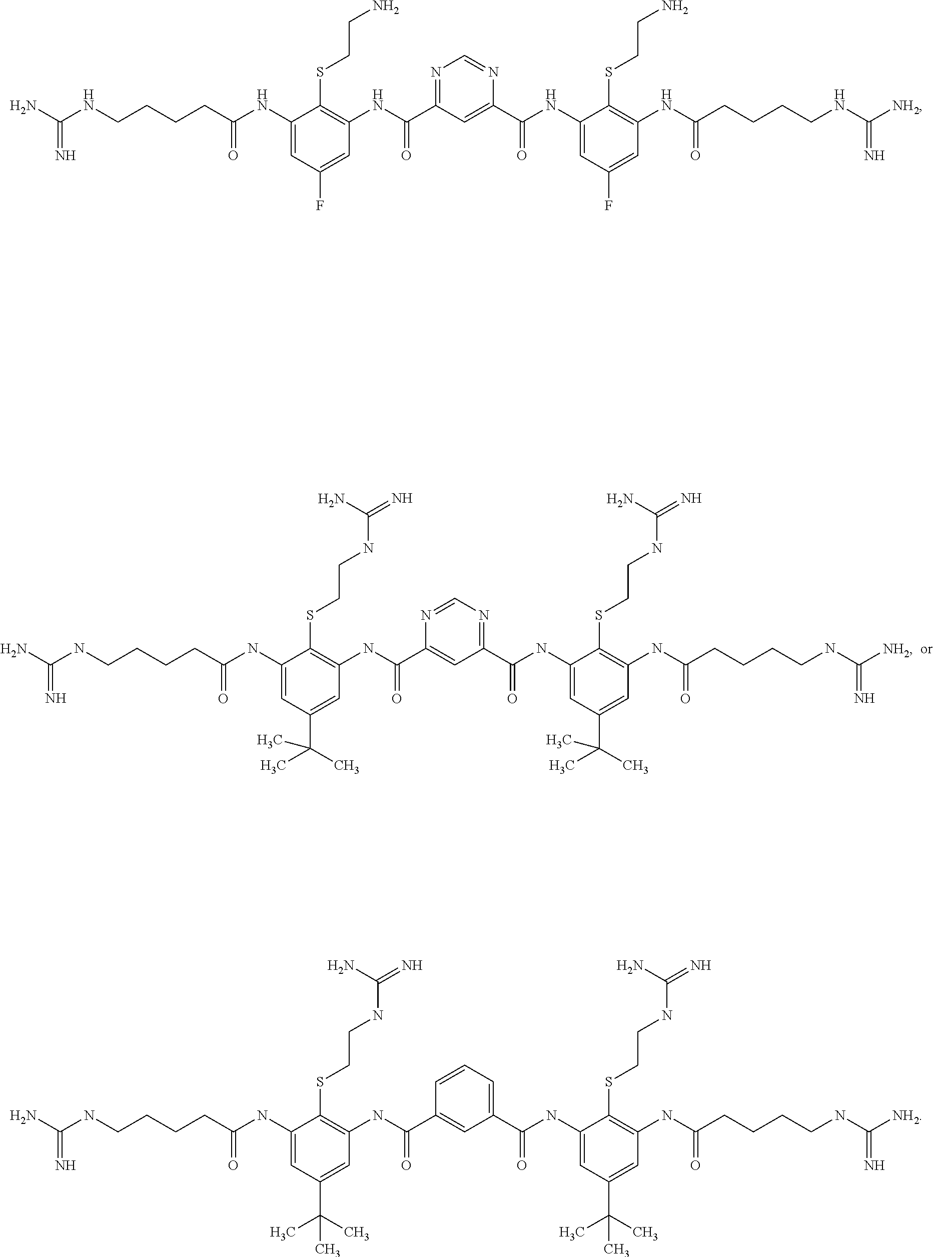 [SMILES: N=C(N)NCCCCC(=O)Nc1cc(F)cc(NC(=O)c2cc(C(=O)Nc3cc(F)cc(NC(=O)CCCCNC(=N)N)c3SCCN)ncn2)c1SCCN ; CC(C)(C)c1cc(NC(=O)CCCCNC(=N)N)c(SCCNC(=N)N)c(NC(=O)c2cc(C(=O)Nc3cc(C(C)(C)C)cc(NC(=O)CCCCNC(=N)N)c3SCCNC(=N)N)ncn2)c1 ; "or" ; CC(C)(C)c1cc(NC(=O)CCCCNC(=N)N)c(SCCNC(=N)N)c(NC(=O)c2cccc(C(=O)Nc3cc(C(C)(C)C)cc(NC(=O)CCCCNC(=N)N)c3SCCNC(=N)N)c2)c1]

The present invention also provides compounds of Formula IIa:

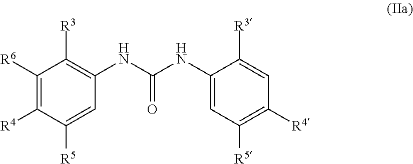

or a pharmaceutically acceptable salt thereof, wherein: R³ and R^{3′} are, independently, H or —O—C₁₋₄alkyl; R⁶ is H, —(CH₂)₁₋₄NHC(═NH)NH₂, or —(CH₂)₁₋₄NH₂; R⁵ and R^{5′} are, independently, H, —CF₃, —(CH₂)₁₋₄NHC(═NH)NH₂, or —(CH₂)₁₋₄NH₂; and R⁴ and R^{4′} are, independently, H, —S—(CH₂)₁₋₄NHC(═NH)NH₂, —S—(CH₂)₁₋₄NH₂, NH₂,

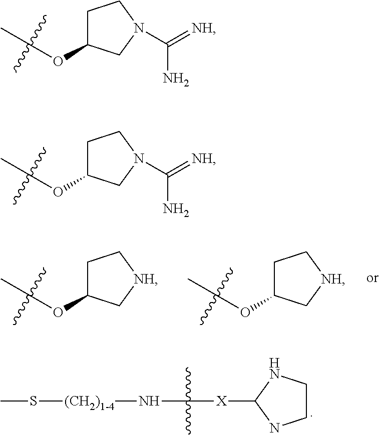

The present invention also provides compounds of Formula III:

R¹—X-A₁-A₂-A₁-X—R (III)

or a pharmaceutically acceptable salt thereof, wherein: A₂ is aryl optionally substituted with one or more W, or heteroaryl optionally substituted with one or more W; each A₁ is, independently, aryl optionally substituted with one or more W, or heteroaryl optionally substituted with one or more W; each X is, independently, absent, —NR⁸, —N(R⁸)N(R⁸)—, O, or S, wherein each R⁸ is, independently, hydrogen or alkyl; R¹ and R² are, independently, absent, —C(═O)—(CH₂)₁₋₅NHC(═NH)NH₂, hydrogen, —(CH₂)₁₋₅NHC(═NH)NH₂, —C(═O)—CH(NH₂)C₁₋₄alkyl, halo, —NO₂, —CF₃, —N(═O)O—, amino, —S—(CH₂)₁₋₅NHC(═NH)NH₂, —O—(CH₂)₁₋₅NHC(═NH)NH₂, —(CH₂)₁₋₅NHC(═NH)NH₂, —S—(CH₂)₁₋₅N(CH₃)₂, —O—(CH₂)₁₋₅N(CH₃)₂, —NHC(═O)—(CH₂)₁₋₅NHC(═NH)NH₂, —NHC(═O)—C₁₋₄alkyl, or —NHC(═O)—(CH₂)₁₋₅NHS(═O)₂—C₁₋₄-alkyl; and each W is, independently, —O—(CH₂)₁₋₅NHC(═NH)NH₂, —O—(CH₂)₁₋₅NH₂, —CF₃, halo, —C₁₋₄alkyl, —N((CH₂)₁₋₅—NH₂)((CH₂)₁₋₅N(C₁₋₄alkyl)₂), —N((CH₂)₁₋₅N(C₁₋₄alkyl)₂)₂, —C₁₋₄alkylamino, —(CH₂)₁₋₅NHC(═NH)NH₂, —S—(CH₂)₁₋₅NHC(═NH)NH₂, —S—(CH₂)₁₋₅NH₂, —S—(CH₂)₁₋₅N(CH₃)₂, —S—(CH₂)₁₋₅NHS(═O)₂—C₁₋₄alkyl, —C≡C—(CH₂)₁₋₅—NHC(═NH)NH₂, —C≡C—(CH₂)₁₋₅—NH₂, or aryl or heterocycle each optionally substituted with one or more —CF₃, cyano, amino, C₁₋₄alkyl, C₁₋₄alkoxy, guanidino, hydroxyl, amidino, halo, —(CH₂)₁₋₄NH₂, —O—(CH₂)₁₋₄NH₂, —(CH₂)₁₋₄NHC(═NH)NH₂, or —O—(CH₂)₁₋₄NHC(═NH)NH₂.

The present invention also provides compounds of Formula IV:

Het¹-X—Y-Het²-Y—X-Het¹ (IV)

or a pharmaceutically acceptable salt thereof, wherein: each X is, independently, —NR⁸, —N(R⁸)N(R⁸)—, O, or S, wherein each R⁸ is, independently, hydrogen or alkyl; each Y is, independently, C═O, C═S, or O═S═O; each Het¹ is, independently, a fused bicyclic ring chosen from naphthalene, isobenzofuran, indolizine, isoindole, indole, purine, isoquinoline, quinolone, phthalazine, naphthyridine, quinoxaline, quinazoline, pteridine, chroman, isochroman, indoline, isoindoline, and

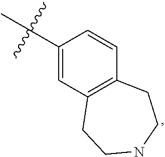 [SMILES: *c1ccc2c(c1)CCNCC2]

each of which is optionally substituted with one or more W; Het² is an aryl or heteroaryl chosen from thiophene, furan, pyran, pyrrole, imidazole, pyrazole, isothiaole, isoxazole, pyridine, pyrazine, pyrimidine, and pyridazine; and each W is, independently, —(CH₂)₁₋₅NH₂, —C₁₋₄alkyl, —(CH₂)₁₋₅NHC(═NH)NH₂, —O—(CH₂)₁₋₅NHC(═NH)NH₂, —O—(CH₂)₁₋₅NH₂, —CF₃, halo, C₁₋₄alkyl, —S—(CH₂)₁₋₅NH₂, —N((CH₂)₁₋₅N(C₁₋₄alkyl)₂)₂, —N((CH₂)₁₋₅—NH₂)((CH₂)₁₋₅N(C₁₋₄alkyl)₂), —S—(CH₂)₁₋₅N(CH₃)₂, —(CH₂)₁₋₅NHC(═NH)NH₂, —S—(CH₂)₁₋₅NHC(═NH)NH₂, —S—(CH₂)₁₋₅NHS(═O)₂—C₁₋₄alkyl, or cycloalkyl optionally substituted with one or more —CF₃, cyano, amino, nitro, C₁₋₄alkyl, C₁₋₄alkoxy, guanidino, hydroxyl, amidino, or halo.

The present invention also provides compounds of Formula VII:

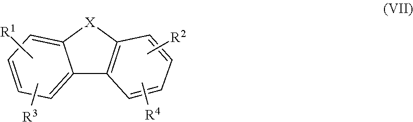

or a pharmaceutically acceptable salt thereof, wherein: X is C(R⁷)C(R⁸), C(R⁷)(R⁸), S, or —N(R⁹); R⁷, R⁸, and R⁹ are, independently, C₁-C₄alkyl, —(CH₂)₀₋₄NH₂, or —(CH₂)₀₋₄NHC(═NH)NH₂; R¹ and R² are, independently, H, C₁-C₈alkyl, C₁-C₈alkoxy, halo, OH, CN, CF₃, or haloC₁-C₈alkyl; R³ and R⁴ are, independently, H, carbocycle(R⁵)(R⁶), —NH-carbocycle(R⁵)(R⁶); each R⁵ and each R⁶ are, independently, H, C₁-C₈alkyl, C₁-C₈alkoxy, halo, amino, amidino, OH, CF₃, —N—(CH₃)₂, —O—(CH₂)₁₋₅—NH₂, —O—(CH₂)₁₋₅NHC(═NH)NH₂, —S—(CH₂)₁₋₈—NH₂, —N((CH₂)₁₋₅NH₂)₂, —S—(CH₂)₁₋₅NHC(═NH)NH₂, —C(═O)NH(CH₂)₁₋₅NH₂, —(CH₂)₁₋₈—NH₂, —(CH₂)₁₋₅N((CH₂)₁₋₅NH₂)₂, aromatic group, heterocycle, —(CH₂)₁₋₈—NH—(CH₂)₁₋₈—NH₂, or —(CH₂)₁₋₈—NH—C(═NH)NH₂.

The present invention also provides compounds of Formula VIII:

Q-X—Z—X-Q (VIII)

or a pharmaceutically acceptable salt thereof, wherein: Z is

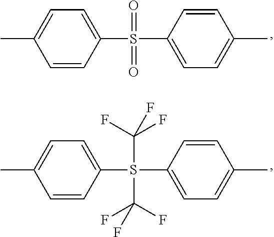

or phenyl; each Q is

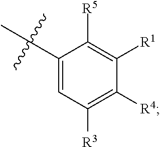

each X is, independently, O, S, or N; each R¹ is, independently, H, CF₃, C(CH₃)₃, halo, or OH; each R³ is, independently, H, —NHR², —(CH₂)₁₋₂NH₂, —NH₂, —NH(CH₂)₁₋₃NH₂,

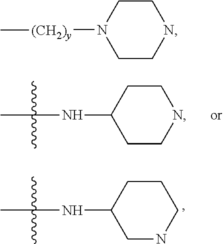

wherein each y is, independently, 1 or 2; each R² is, independently, H, or the free base or salt form of —(CH₂)₁₋₄NH₂ or —(CH₂)₁₋₄NHC(═NH)NH₂; each R⁴ is, independently, H, —CF₃, —NHC(═O)(CH₂)₁₋₆NHC(═NH)NH₂,

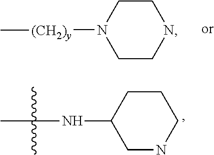

wherein each q is, independently, 1 or 2; and each R⁵ is, independently, H, —(CH₂)₁₋₄NH₂, or CF₃; wherein the compound comprises: a)

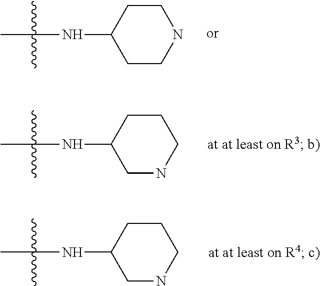

—(CH₂)₁₋₄NH₂ at at least one R⁵.

The present invention also provides compounds of Formula IX:

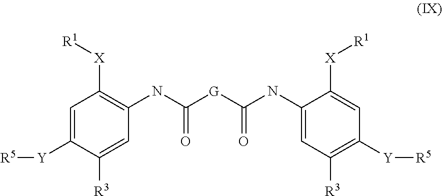

or a pharmaceutically acceptable salt thereof, wherein: G is

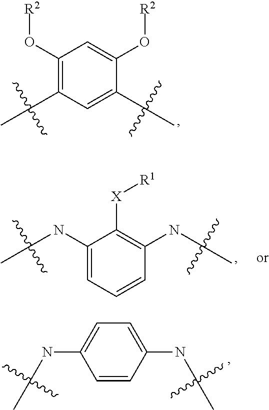

each X is, independently, O or S; each Y is, independently, O or S; each R¹ is, independently,

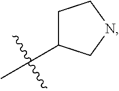

—(CH₂)₁₋₄NH₂, or —(CH₂)₁₋₄NHC(═NH)NH₂; each R² is, independently, H, C₁-C₈alkyl, —(CH₂)₁₋₄NH₂, or —(CH₂)₁₋₄NHC(═NH)NH₂; each R³ is, independently, H, —CF₃, —C(CH₃)₃, halo, or OH; and each R⁵ is, independently, —(CH₂)₁₋₄NH₂ or —(CH₂)₁₋₄NHC(═NH)NH₂.

The present invention also provides compounds of Formula Xa:

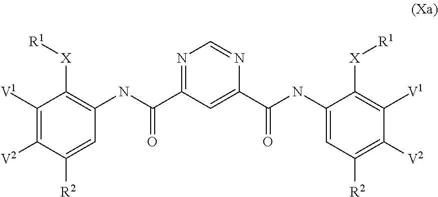

or a pharmaceutically acceptable salt thereof, wherein: each X is, independently, O, S, or S(═O)₂; each R¹ is, independently, —(CH₂)₁₋₄C(═O)OH, —(CH₂)₁₋₄NH₂, —(CH₂)₁₋₄OH, —(CH₂)₁₋₄NHC(═O)C₁₋₄alkyl, or —(CH₂)₁₋₄C(═O)OC₁₋₄alkyl; each R² is, independently, H, halo, —CF₃, or —C(CH₃)₃; each V² is H; and each V¹ is, independently, amino or —NC(═O)—R³, where each R³ is, independently, —C₁₋₄alkyl, —(CH₂)₁₋₄—NH₂, —(CH₂)₁₋₄—NH—C(═NH)NH₂, or aryl (optionally substituted with one or more, independently, halo, cyano, or —C₁₋₄alkoxy).

The present invention also provides compounds of Formula Xb:

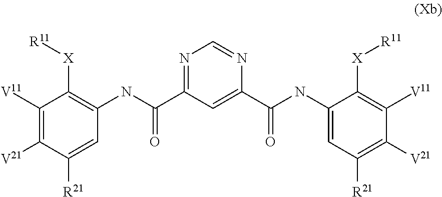

or a pharmaceutically acceptable salt thereof, wherein: each X is, independently, O or S; each R¹¹ is, independently, —(CH₂)₁₋₄NH₂ or —C₁₋₄alkyl; each R²¹ is, independently, H, halo, —CF₃, or —C(CH₃)₃; each V¹¹ is H; and each V² is, independently, —S—(CH₂)₁₋₄—NH₂ or —S—(CH₂)₁₋₄—NHC(═NH)NH₂.

The present invention also provides compoundc of Formula XI:

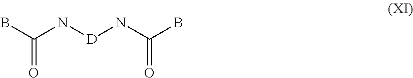

or a pharmaceutically acceptable salt thereof, wherein: D is

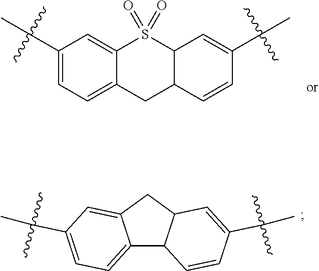 [SMILES: *C1=CC2C(C=C1)Cc1ccc(*)cc1S2(=O)=O ; *C1=CC2Cc3cc(*)ccc3C2C=C1]

each B is, independently,

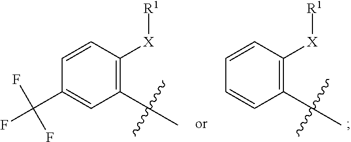

each X is, independently, O or S; and each R¹ is, independently, —(CH₂)₁₋₄NHC(═NH)NH₂.

The present invention also provides compounds of Formula XII:

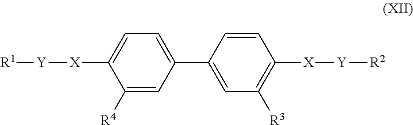

or a pharmaceutically acceptable salt thereof, wherein: each X is, independently, —NH—, O, S, or absent; each Y is, independently, —C(═O), —C(═S), or absent; R¹ is —(CH₂)ₙNHC(═NH)NH₂, —NH(CH₂)ₙNH₂, —NH(CH₂)ₙNC(═N)NH₂, —(CH₂)ₙNH₂, —O—(CH₂)ₙNH₂, —O—(CH₂)ₙNC(═N)NH₂, —CH═CH—CH₂NH₂, —CH═CH—CH₂NC(═N)NH₂, —CH═CH—(CH₂)₂NH₂, —CH═CH—(CH₂)₂NC(═N)NH₂, —C≡C—CH₂NH₂, —C≡C—(CH₂)₂NH₂, —CH≡CH—CH₂NC(═N)NH₂, or —C≡C—(CH₂)₂—NC(═N)NH₂, where n is 2, 3, or 4; or an aryl, heteroaryl, or heterocycle (each of which is optionally substituted with one or more W); R² is —(CH₂)ₙNHC(═NH)NH₂, —NH(CH₂)ₙNH₂, —NH(CH₂)ₙNC(═N)NH₂, —(CH₂)ₙNH₂, —O—(CH₂)ₙNH₂, —O—(CH₂)ₙNC(═N)NH₂, —CH═CH—CH₂NH₂, —CH═CH—CH₂NC(═N)NH₂, —CH═CH—(CH₂)₂NH₂, —CH═CH—(CH₂)₂NC(═N)NH₂, —C≡C—CH₂NH₂, —C≡C—(CH₂)₂NH₂, —CH≡CH—CH₂NC(═N)NH₂, or —C≡C—(CH₂)₂—NC(═N)NH₂, where n is 2, 3, or 4; or an aryl, heteroaryl, or heterocycle (each of which is optionally substituted with one or more W); R³ is H, —CF₃, —NH(CH₂)ₙNH₂, —NH(CH₂)ₙNC(═N)NH₂, —(CH₂)ₙNH₂, —(CH₂)ₙNC(═N)NH₂, —O—(CH₂)ₙNH₂, —O—(CH₂)ₙNC(═N)NH₂, —CH═CH—CH₂NH₂, —CH═CH—CH₂NC(═N)NH₂, —CH═CH—(CH₂)₂NH₂, —CH═CH—(CH₂)₂NC(═N)NH₂, —C≡C—CH₂NH₂, —C≡C—(CH₂)₂NH₂, —CH≡CH—CH₂NC(═N)NH₂, or —C≡C—(CH₂)₂—NC(═N)NH₂, where n is 2, 3, or 4; R⁴ is H, —CF₃, —NH(CH₂)ₙNH₂, —NH(CH₂)ₙNC(═N)NH₂, —(CH₂)ₙNH₂, —(CH₂)ₙNC(═N)NH₂, —O—(CH₂)ₙNH₂, —O—(CH₂)ₙNC(═N)NH₂, —CH═CH—CH₂NH₂, —CH═CH—CH₂NC(═N)NH₂, —CH═CH—(CH₂)₂NH₂, —CH═CH—(CH₂)₂NC(═N)NH₂, —C≡C—CH₂NH₂, —C≡C—(CH₂)₂NH₂, —CH≡CH—CH₂NC(═N)NH₂, or —C≡C—(CH₂)₂—NC(═N)NH₂, where n is 2, 3, or 4; and each W is, independently, halo, —CF₃, —S—(CH₂)₁₋₅NHC(═NH)NH₂, —S—(CH₂)₁₋₅NH₂, —S—(CH₂)₁₋₅N(CH₃)₂, —S—(CH₂)₁₋₅NHS(═O)₂—C₁₋₄alkyl, —C₁₋₄alkyl, —O—(CH₂)₁₋₅NHC(═NH)NH₂; or —O-heterocycle or heterocycle wherein the heterocycle is optionally substituted with one or more cyano, amino, guanyl, C₁₋₄alkyl, guanidino, hydroxyl, amidino, C₁₋₄alkoxy, CF₃, or halo.

The present invention also provides compounds of Formula XIII:

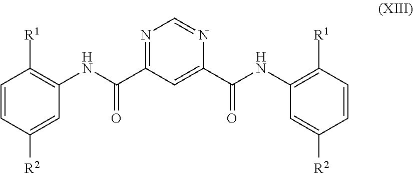

or pharmaceutically acceptable salt thereof, wherein: each R¹ is, independently, —S—(CH₂)₁₋₅NH₂, —S—(CH₂)₁₋₅NHC(═NH)NH₂, —C₁₋₄alkoxy, —S—(CH₂)₁₋₅N(C₁₋₄alkyl)₂, —O—(CH₂)₁₋₅NH₂, —O—(CH₂)₁₋₅NHC(═NH)NH₂, —(CH₂)₁₋₅NH₂, —O—(CH₂)₁₋₅N(C₁₋₄alkyl)₂, —C(═O)—(CH₂)₁₋₅NH₂, —C(═O)—(CH₂)₁₋₅NHC(═NH)NH₂, —C(═O)—(CH₂)₁₋₅N(C₁₋₄alkyl)₂, —(CH₂)₁₋₅NHC(═NH)NH₂, —(CH₂)₁₋₅N(C₁₋₄alkyl)₂, —NHC(═O)—(CH₂)₁₋₅NHC(═NH)NH₂, —NHC(═O)—C₁₋₄alkyl, or —NHC(═O)—(CH₂)₁₋₅NHS(═O)₂—C₁₋₄alkyl; and each R² is, independently, —CF₃, —(CH₂)₁₋₅NH₂, —C₁₋₄alkyl, or halo.

The present invention also provides compounds of Formula XIV:

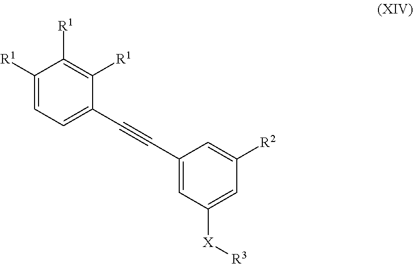

or pharmaceutically acceptable salt thereof, wherein: each R¹ is, independently, absent or halo, CF₃, cyano, amino, nitro, amidino, C₁₋₄alkyl, guanidino, hydroxyl, or C₁₋₄alkoxy; each R² is halo, cyano, amino, nitro, amidino, C₁₋₄alkyl, guanidino, hydroxyl, CF₃, or C₁₋₄alkoxy; and each R³ is, independently, —S—(CH₂)₁₋₅NH₂, —S—(CH₂)₁₋₅NHC(═NH)NH₂, —C₁₋₄alkoxy, —S—(CH₂)₁₋₅N(C₁₋₄alkyl)₂, —O—(CH₂)₁₋₅NH₂, —O—(CH₂)₁₋₅NHC(═NH)NH₂, —(CH₂)₁₋₅NH₂, —O—(CH₂)₁₋₅N(C₁₋₄alkyl)₂, —C(═O)—(CH₂)₁₋₅NH₂, —C(═O)—(CH₂)₁₋₅NHC(═NH)NH₂, —C(═O)—(CH₂)₁₋₅N(C₁₋₄alkyl)₂, —(CH₂)₁₋₅NHC(═NH)NH₂, —(CH₂)₁₋₅N(C₁₋₄alkyl)₂, —NHC(═O)—(CH₂)₁₋₅NHC(═NH)NH₂, —NHC(═O)—C₁₋₄alkyl, or —NHC(═O)—(CH₂)₁₋₅NHS(═O)₂—C₁₋₄alkyl.

The present invention also provides compounds of Formula XV:

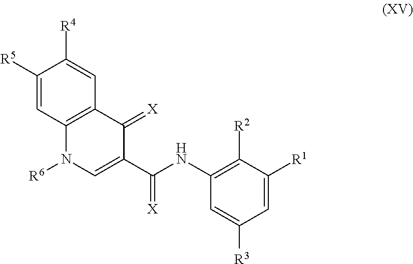

or a pharmaceutically acceptable salt thereof, wherein: each X is, independently, O or S; R¹ is amino, cyano, C₁₋₄alkyl, guanidino, hydroxyl, amidino, C₁₋₄alkoxy, CF₃, or halo; R² is —S—(CH₂)₁₋₅NH₂, —S—(CH₂)₁₋₅NHC(═NH)NH₂, —S—(CH₂)₁₋₅N(C₁₋₄alkyl)₂, —O—(CH₂)₁₋₅NH₂, —O—(CH₂)₁₋₅NHC(═NH)NH₂, —(CH₂)₁₋₅NH₂, —O—(CH₂)₁₋₅N(C₁₋₄alkyl)₂, —C(═O)—(CH₂)₁₋₅NH₂, —C(═O)—(CH₂)₁₋₅NHC(═NH)NH₂, —C(═O)—(CH₂)₁₋₅N(C₁₋₄alkyl)₂, —(CH₂)₁₋₅NHC(═NH)NH₂, (CH₂)₁₋₅N(C₁₋₄alkyl)₂, —NHC(═O)—(CH₂)₁₋₅NHC(═NH)NH₂, —NHC(═O)—C₁₋₄alkyl, or —NHC(═O)—(CH₂)₁₋₅NHS(═O)₂—C₁₋₄alkyl; R³ is CF₃, amino, cyano, C₁₋₄alkyl, guanidino, hydroxyl, amidino, C₁₋₄alkoxy, or halo; R⁴ is halo, amino, cyano, C₁₋₄alkyl, guanidino, hydroxyl, amidino, C₁₋₄alkoxy, or CF₃; R⁵ is a heterocycloalkyl; and R⁶ is a cycloalkyl.

The present invention also provides compounds of Formula XVI:

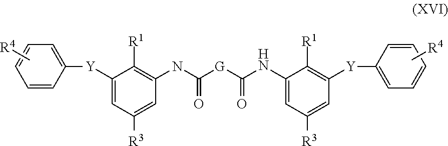

or a pharmaceutically acceptable salt thereof, wherein: G is

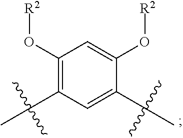

each Y is —NH—C(═O)—; each R¹ is, independently, H or —S—(CH₂)₁₋₄NH₂, or —S—(CH₂)₁₋₄NHC(═NH)NH₂; each R² is, independently, H, C₁-C₄alkyl, —(CH₂)₁₋₄NH₂, or —(CH₂)₁₋₄NHC(═NH)NH₂; each R³ is, independently, H, —CF₃, —C(CH₃)₃, halo, or OH; and each R⁴ is, independently, H, —CF₃, —C(CH₃)₃, halo, cyano, or OH.

The present invention also provides methods of treating malaria in a mammal, or killing or inhibiting the growth of a Plasmodium species, comprising administering to the mammal, optionally in need thereof, or contacting the species with, an effective amount of a compound of Formula II:

R¹-A₁-X—Z—X-A₁-R² (II)

or a pharmaceutically acceptable salt thereof, wherein: each X is, independently, —NR⁸, —N(R⁸)N(R⁸)—, O, or S, wherein each R⁸ is, independently, hydrogen or alkyl; each Z is, independently, C═O, C═S, or O═S═O; each A₁ is, independently, aryl optionally substituted with one or more W, or heteroaryl optionally substituted with one or more W; each W is, independently, —CF₃, halo, C₁₋₄alkyl, —O—(CH₂)₁₋₅NHC(═NH)NH₂, —N((CH₂)₁₋₅—NH₂)((CH₂)₁₋₅N(C₁₋₄alkyl)₂), —N((CH₂)₁₋₅N(C₁₋₄alkyl)₂)₂, heterocycle, —S—(CH₂)₁₋₅NHC(═NH)NH₂, —S—(CH₂)₁₋₅NH₂, —S—(CH₂)₁₋₅N(CH₃)₂, —C₁₋₄alkyl, —S—(CH₂)₁₋₅NHS(═O)₂—C₁₋₄alkyl, O-heterocycle (wherein the heterocycle is optionally substituted with one or more cyano, amino, C₁₋₄alkyl, C₁₋₄alkoxy, guanidino, hydroxyl, amidino, or halo); and R¹ and R² are, independently, hydrogen, halo, —NO₂, —S—(CH₂)₁₋₅NHC(═NH)NH₂, —O—(CH₂)₁₋₅NHC(═NH)NH₂, —CF₃, —N⁺(═O)O⁻, —(CH₂)₁₋₅NHC(═NH)NH₂, amino, —S—(CH₂)₁₋₅N(CH₃)₂, —O—(CH₂)₁₋₅N(CH₃)₂, —O-heterocycle (wherein the heterocycle is optionally substituted), —S-heterocycle (wherein the heterocycle is optionally substituted), —NHC(═O)—C₁₋₄alkyl, —NHC(═O)—(CH₂)₁₋₅NHC(═NH)NH₂, —NHC(═O)—(CH₂)₁₋₅-aryl (wherein either or both the —(CH₂)₁₋₅ or phenyl is optionally substituted), —NHC(═O)—(CH₂)₁₋₅NH-aryl (wherein either or both —NH and/or aryl is optionally substituted), —NHC(═O)-aryl (wherein the aryl is optionally substituted), or —NHC(═O)—(CH₂)₁₋₅NHS(═O)₂—C₁₋₄alkyl, wherein the optional substituents are chosen from one or more cyano, amino, C₁₋₄alkyl, guanidino, hydroxyl, amidino, C₁₋₄alkoxy, CF₃, and halo.

The present invention also provides methods of treating malaria in a mammal, or killing or inhibiting the growth of a Plasmodium species, comprising administering to the mammal, optionally in need thereof, or contacting the species with, an effective amount of a compound of Formula V:

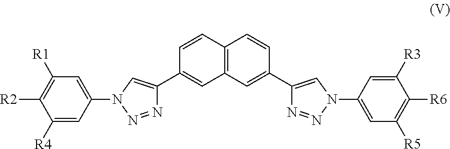

or a pharmaceutically acceptable salt thereof, wherein: R¹ is H, —NH(CH₂)ₙNH₂, —NH(CH₂)ₙNC(═N)NH₂, —(CH₂)ₙNH₂, —O—(CH₂)ₙNH₂, —(CH₂)ₙNC(═N)NH₂, —O—(CH₂)ₙNC(═N)NH₂, —CH═CH—CH₂NH₂, —CH═CH—CH₂NC(═N)NH₂, —CH═CH—(CH₂)₂NH₂, —CH═CH—(CH₂)₂NC(═N)NH₂, —C≡C—CH₂NH₂, —C≡C—(CH₂)₂NH₂, —CH≡CH—CH₂NC(═N)NH₂, or —C≡C—(CH₂)₂—NC(═N)NH₂, where n is 2, 3, or 4; R² is H, —NH(CH₂)ₙNH₂, —NH(CH₂)ₙNC(═N)NH₂, —(CH₂)ₙNH₂, —O—(CH₂)ₙNH₂, —(CH₂)ₙNC(═N)NH₂, —O—(CH₂)ₙNC(═N)NH₂, —CH═CH—CH₂NH₂, —CH═CH—CH₂NC(═N)NH₂, —CH═CH—(CH₂)₂NH₂, —CH≡CH—(CH₂)₂NC(═N)NH₂, —C≡C—CH₂NH₂, —C≡C—(CH₂)₂NH₂, —CH≡CH—CH₂NC(═N)NH₂, or —C≡C—(CH₂)₂—NC(═N)NH₂, where n is 2, 3, or 4; R³ is H, —NH(CH₂)ₙNH₂, —NH(CH₂)ₙNC(═N)NH₂, —(CH₂)ₙNH₂, —O—(CH₂)ₙNH₂, —(CH₂)ₙNC(═N)NH₂, —O—(CH₂)ₙNC(═N)NH₂, —CH═CH—CH₂NH₂, —CH═CH—CH₂NC(═N)NH₂, —CH═CH—(CH₂)₂NH₂, —CH≡CH—(CH₂)₂NC(═N)NH₂, —C≡C—CH₂NH₂, —C≡C—(CH₂)₂NH₂, —CH≡CH—CH₂NC(═N)NH₂, or —C≡C—(CH₂)₂—NC(═N)NH₂, where n is 2, 3, or 4; R⁴ is H, —NH(CH₂)ₙNH₂, —NH(CH₂)ₙNC(═N)NH₂, —(CH₂)ₙNH₂, —O—(CH₂)ₙNH₂, —(CH₂)ₙNC(═N)NH₂, —O—(CH₂)ₙNC(═N)NH₂, —CH═CH—CH₂NH₂, —CH═CH—CH₂NC(═N)NH₂, —CH═CH—(CH₂)₂NH₂, —CH═CH—(CH₂)₂NC(═N)NH₂, —C≡C—CH₂NH₂, —C≡C—(CH₂)₂NH₂, —CH≡CH—CH₂NC(═N)NH₂, or —C≡C—(CH₂)₂—NC(═N)NH₂, where n is 2, 3, or 4; R⁵ is H, —NH(CH₂)ₙNH₂, —NH(CH₂)ₙNC(═N)NH₂, —(CH₂)ₙNH₂, —O—(CH₂)ₙNH₂, —(CH₂)ₙNC(═N)NH₂, —O—(CH₂)ₙNC(═N)NH₂, —CH═CH—CH₂NH₂, —CH═CH—CH₂NC(═N)NH₂, —CH═CH—(CH₂)₂NH₂, —CH═CH—(CH₂)₂NC(═N)NH₂, —C≡C—CH₂NH₂, —C≡C—(CH₂)₂NH₂, —CH≡CH—CH₂NC(═N)NH₂, or —C≡C—(CH₂)₂—NC(═N)NH₂, where n is 2, 3, or 4; and R⁶ is H, —NH(CH₂)ₙNH₂, —NH(CH₂)ₙNC(═N)NH₂, —(CH₂)ₙNH₂, —O—(CH₂)ₙNH₂, —(CH₂)ₙNC(═N)NH₂, —O—(CH₂)ₙNC(═N)NH₂, —CH═CH—CH₂NH₂, —CH═CH—CH₂NC(═N)NH₂, —CH═CH—(CH₂)₂NH₂, —CH═CH—(CH₂)₂NC(═N)NH₂, —C≡C—CH₂NH₂, —C≡C—(CH₂)₂NH₂, —CH≡CH—CH₂NC(═N)NH₂, or —C≡C—(CH₂)₂—NC(═N)NH₂, where n is 2, 3, or 4; or a pharmaceutically acceptable salt thereof.

The present invention also provides methods of treating malaria in a mammal, or killing or inhibiting the growth of a Plasmodium species, comprising administering to the mammal, optionally in need thereof, or contacting the species with, an effective amount of a compound of Formula VI:

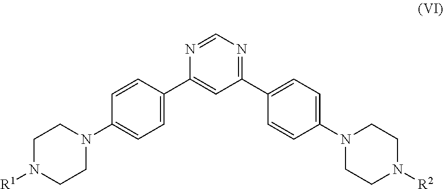

or a pharmaceutically acceptable salt thereof, wherein R¹ and R² are, independently, hydrogen, —C₁₋₄alkyl, —C(═NH)NH₂, —(CH₂)ₙNH₂, or —(CH₂)ₙNC(═NH)NH₂, where n is 2, 3, or 4.

The present invention also provides methods of treating malaria in a mammal, or killing or inhibiting the growth of a Plasmodium species, comprising administering to the mammal, optionally in need thereof, or contacting the species with, an effective amount of a compound, or a pharmaceutically acceptable salt thereof, chosen from:

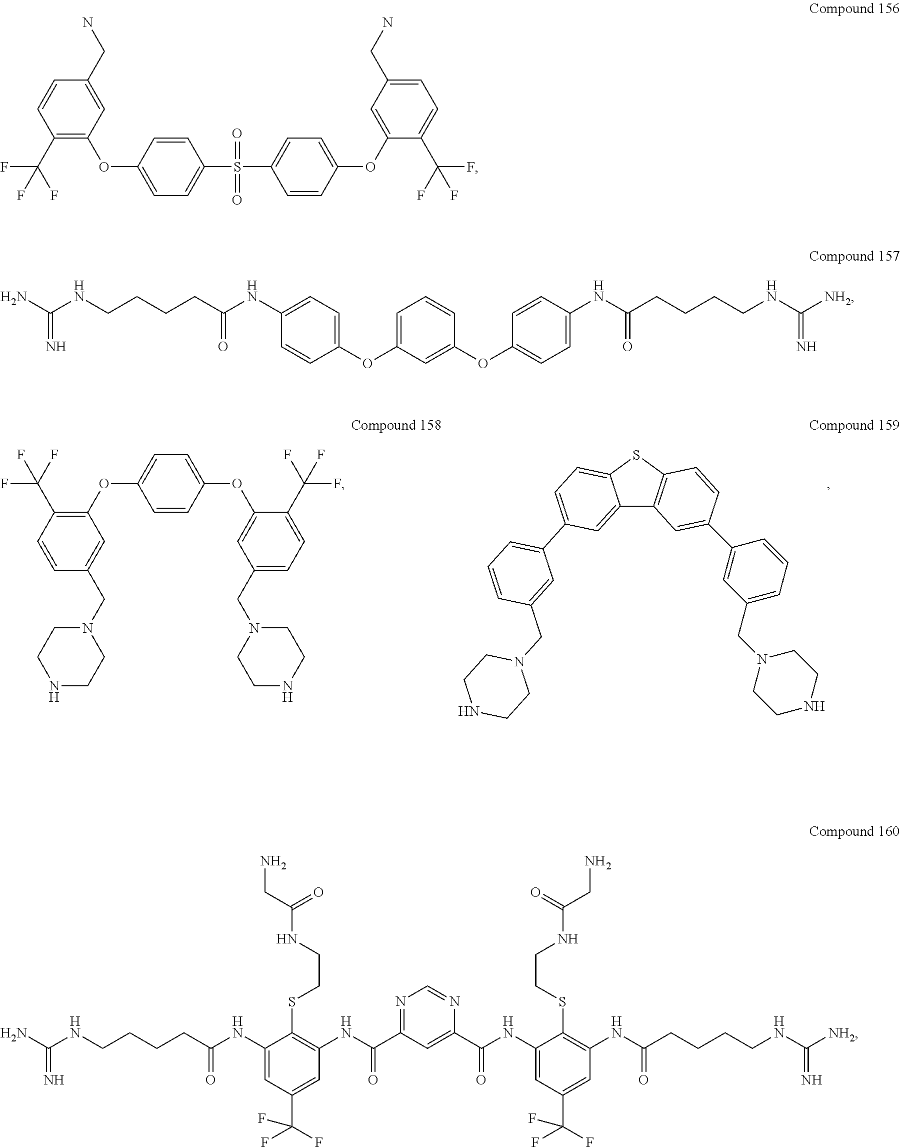 [SMILES: NCc1ccc(C(F)(F)F)c(Oc2ccc(S(=O)(=O)c3ccc(Oc4cc(CN)ccc4C(F)(F)F)cc3)cc2)c1 ; N=C(N)NCCCCC(=O)Nc1ccc(Oc2cccc(Oc3ccc(NC(=O)CCCCNC(=N)N)cc3)c2)cc1 ; FC(F)(F)c1ccc(CN2CCNCC2)cc1Oc1ccc(Oc2cc(CN3CCNCC3)ccc2C(F)(F)F)cc1 ; c1cc(CN2CCNCC2)cc(-c2ccc3sc4ccc(-c5cccc(CN6CCNCC6)c5)cc4c3c2)c1 ; N=C(N)NCCCCC(=O)Nc1cc(C(F)(F)F)cc(NC(=O)c2cc(C(=O)Nc3cc(C(F)(F)F)cc(NC(=O)CCCCNC(=N)N)c3SCCNC(=O)CN)ncn2)c1SCCNC(=O)CN]

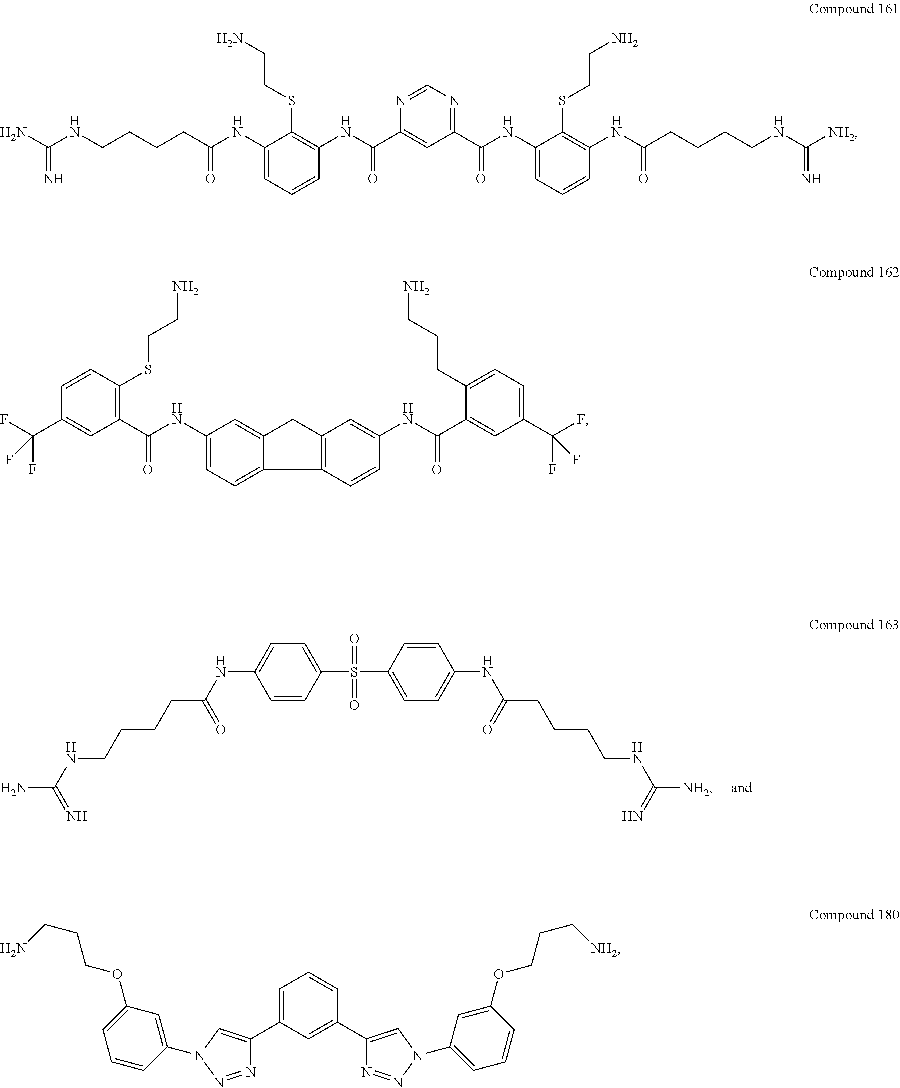 [SMILES: N=C(N)NCCCCC(=O)Nc1cccc(NC(=O)c2cc(C(=O)Nc3cccc(NC(=O)CCCCNC(=N)N)c3SCCN)ncn2)c1SCCN ; NCCCc1ccc(C(F)(F)F)cc1C(=O)Nc1ccc2c(c1)Cc1cc(NC(=O)c3cc(C(F)(F)F)ccc3SCCN)ccc1-2 ; N=C(N)NCCCCC(=O)Nc1ccc(S(=O)(=O)c2ccc(NC(=O)CCCCNC(=N)N)cc2)cc1 ; "and" ; NCCCOc1cccc(-n2cc(-c3cccc(-c4cn(-c5cccc(OCCCN)c5)nn4)c3)nn2)c1]

or a pharmaceutically acceptable salt thereof.

The present disclosure also provides pharmaceutical compositions comprising any one or more of the foregoing compounds, or a pharmaceutically acceptable salt thereof, and a pharmaceutically acceptable carrier.

The present disclosure also provides methods of treating malaria in a mammal comprising administering to the mammal, optionally in need thereof, a therapeutically effective amount of any one or more of the foregoing compounds, or a pharmaceutically acceptable salt thereof, or compositions comprising the same.

The present disclosure also provides methods of killing or inhibiting the growth of a Plasmodium species comprising contacting the species with an effective amount of any one or more of the foregoing compounds, or a pharmaceutically acceptable salt thereof, or compositions comprising the same.

The present disclosure also provides any one or more of the foregoing compounds, or compositions comprising the same, for treating malaria in a mammal, or killing or inhibiting the growth of a Plasmodium species.

The present disclosure also provides any one or more of the foregoing compounds, or compositions comprising the same, for use in the manufacture of a medicament for treating malaria in a mammal, or killing or inhibiting the growth of a Plasmodium species.

The present disclosure also provides uses of any one or more of the foregoing compounds, or compositions comprising the same, for treating malaria in a mammal, or killing or inhibiting the growth of a Plasmodium species.

The present disclosure also provides uses of any one or more of the foregoing compounds, or compositions comprising the same, in the manufacture of a medicament for treating malaria in a mammal, or killing or inhibiting the growth of a Plasmodium species.

### DESCRIPTION OF EMBODIMENTS

Unless defined otherwise, all technical and scientific terms have the same meaning as is commonly understood by one of ordinary skill in the art to which the embodiments disclosed belongs.

As used herein, the terms “a” or “an” means that “at least one” or “one or more” unless the context clearly indicates otherwise.

As used herein, the term “about” means that the numerical value is approximate and small variations would not significantly affect the practice of the disclosed embodiments. Where a numerical limitation is used, unless indicated otherwise by the context, “about” means the numerical value can vary by ±10% and remain within the scope of the disclosed embodiments.

As used herein, the term “alkenyl” means a straight or branched alkyl group having one or more double carbon-carbon bonds and 2-20 carbon atoms, including, but not limited to, ethenyl, 1-propenyl, 2-propenyl, 2-methyl-1-propenyl, 1-butenyl, 2-butenyl, and the like. In some embodiments, the alkenyl chain is from 2 to 10 carbon atoms in length, from 2 to 8 carbon atoms in length, from 2 to 6 carbon atoms in length, or from 2 to 4 carbon atoms in length.

As used herein, the term “alkoxy” means a straight or branched —O-alkyl group of 1 to 20 carbon atoms, including, but not limited to, methoxy, ethoxy, n-propoxy, isopropoxy, t-butoxy, and the like. In some embodiments, the alkoxy chain is from 1 to 10 carbon atoms in length, from 1 to 8 carbon atoms in length, from 1 to 6 carbon atoms in length, from 1 to 4 carbon atoms in length, from 2 to 10 carbon atoms in length, from 2 to 8 carbon atoms in length, from 2 to 6 carbon atoms in length, or from 2 to 4 carbon atoms in length.

As used herein, the term “alkyl” means a saturated hydrocarbon group which is straight-chained or branched. An alkyl group can contain from 1 to 20, from 2 to 20, from 1 to 10, from 2 to 10, from 1 to 8, from 2 to 8, from 1 to 6, from 2 to 6, from 1 to 4, from 2 to 4, from 1 to 3, or 2 or 3 carbon atoms. Examples of alkyl groups include, but are not limited to, methyl (Me), ethyl (Et), propyl (e.g., n-propyl and isopropyl), butyl (e.g., n-butyl, t-butyl, isobutyl), pentyl (e.g., n-pentyl, isopentyl, neopentyl), hexyl, isohexyl, heptyl, 4,4-dimethylpentyl, octyl, 2,2,4-trimethylpentyl, nonyl, decyl, undecyl, dodecyl, 2-methyl-1-propyl, 2-methyl-2-propyl, 2-methyl-1-butyl, 3-methyl-1-butyl, 2-methyl-3-butyl, 2-methyl-1-pentyl, 2,2-dimethyl-1-propyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, and the like.

As used herein, the term “alkylene” or “alkylenyl” means a divalent alkyl linking group. An example of an alkylene (or alkylenyl) is methylene or methylenyl (—CH₂—).

As used herein, the term “alkynyl” means a straight or branched alkyl group having one or more triple carbon-carbon bonds and 2-20 carbon atoms, including, but not limited to, acetylene, 1-propylene, 2-propylene, and the like. In some embodiments, the alkynyl chain is 2 to 10 carbon atoms in length, from 2 to 8 carbon atoms in length, from 2 to 6 carbon atoms in length, or from 2 to 4 carbon atoms in length.

As used herein, the term “amidino” means —C(═NH)NH₂.

As used herein, the term “amino” means —NH₂.

As used herein, the term “animal” includes, but is not limited to, humans and non-human vertebrates such as wild, domestic, and farm animals.

As used herein, the phrase “anti-malarial effective amount” of a compound can be measured by the anti-malarial effectiveness of the compound. In some embodiments, an anti-malarial effective amount inhibits growth of a Plasmodium species by at least 10%, by at least 20%, by at least 30%, by at least 40%, by at least 50%, by at least 60%, by at least 70%, by at least 80%, by at least 90%, or by at least 95%. In some embodiments, an “anti-malarial effective amount” is also a “therapeutically effective amount” whereby the compound reduces or eliminates at least one harmful effect of malaria in a mammal.

As used herein, the term “aryl” means a monocyclic, bicyclic, or polycyclic (e.g., having 2, 3 or 4 fused rings) aromatic hydrocarbons. In some embodiments, aryl groups have from 6 to 20 carbon atoms or from 6 to 10 carbon atoms. Examples of aryl groups include, but are not limited to, phenyl, naphthyl, anthracenyl, phenanthrenyl, indanyl, indenyl, tetrahydronaphthyl, and the like.

As used herein, the term “arylene” means an aryl linking group, i.e., an aryl group that links one group to another group in a molecule.

As used herein, the term “carbocycle” means a 5- or 6-membered, saturated or unsaturated cyclic ring, optionally containing O, S, or N atoms as part of the ring. Examples of carbocycles include, but are not limited to, cyclopentyl, cyclohexyl, cyclopenta-1,3-diene, phenyl, and any of the heterocycles recited above.

As used herein, the term “carrier” means a diluent, adjuvant, or excipient with which a compound is administered. Pharmaceutical carriers can be liquids, such as water and oils, including those of petroleum, animal, vegetable or synthetic origin, such as peanut oil, soybean oil, mineral oil, sesame oil and the like. The pharmaceutical carriers can also be saline, gum acacia, gelatin, starch paste, talc, keratin, colloidal silica, urea, and the like. In addition, auxiliary, stabilizing, thickening, lubricating and coloring agents can be used.

As used herein, the term “chemically nonequivalent termini” means a functional group such as an ester, amide, sulfonamide, or N-hydroxyoxime that, when reversing the orientation of the functional group (e.g., —(C═O)O—) produces different chemical entities (e.g., —R¹C(═O)OR²— vs. —R¹OC(═O)R²—).

As used herein, the term, “compound” means all stereoisomers, tautomers, and isotopes of the compounds described herein.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

As used herein, the term “cyano” means —CN.

As used herein, the term “cycloalkyl” means non-aromatic cyclic hydrocarbons including cyclized alkyl, alkenyl, and alkynyl groups that contain up to 20 ring-forming carbon atoms. Cycloalkyl groups can include mono- or polycyclic ring systems such as fused ring systems, bridged ring systems, and spiro ring systems. In some embodiments, polycyclic ring systems include 2, 3, or 4 fused rings. A cycloalkyl group can contain from 3 to 15, from 3 to 10, from 3 to 8, from 3 to 6, from 4 to 6, from 3 to 5, or 5 or 6 ring-forming carbon atoms. Ring-forming carbon atoms of a cycloalkyl group can be optionally substituted by oxo or sulfido.

Examples of cycloalkyl groups include, but are not limited to, cyclopropyl, cyclobutyl, cyclopentyl, cyclohexyl, cycloheptyl, cyclooctyl, cyclononyl, cyclopentenyl, cyclohexenyl, cyclohexadienyl, cycloheptatrienyl, norbornyl, norpinyl, norcarnyl, adamantyl, and the like. Also included in the definition of cycloalkyl are moieties that have one or more aromatic rings fused (having a bond in common with) to the cycloalkyl ring, for example, benzo or thienyl derivatives of pentane, pentene, hexane, and the like (e.g., 2,3-dihydro-1H-indene-1-yl, or 1H-inden-2(3H)-one-1-yl).

As used herein, the phrase “groups with chemically nonequivalent termini” means functional groups such as esters amides, sulfonamides and N-hydroxyoximes where reversing the orientation of the substituents, e.g. R¹C(═O)OR² vs. R¹⁰(O═)CR², produces unique chemical entities.

As used herein, the term “guanidino” means —NHC(═NH)NH₂.

As used herein, the term “halo” means halogen groups including, but not limited to fluoro, chloro, bromo, and iodo.

As used herein, the term “haloalkoxy” means an —O-haloalkyl group. An example of an haloalkoxy group is OCF₃.

As used herein, the term “haloalkyl” means a C₁₋₆alkyl group having one or more halogen substituents. Examples of haloalkyl groups include, but are not limited to, CF₃, C₂F₅, CHF₂, CCl₃, CHCl₂, C₂Cl₅, CH₂CF₃, and the like.

As used herein, the term “heteroaryl” means an aromatic heterocycle having up to 20 ring-forming atoms (e.g., C) and having at least one heteroatom ring member (ring-forming atom) such as sulfur, oxygen, or nitrogen. In some embodiments, the heteroaryl group has at least one or more heteroatom ring-forming atoms, each of which are, independently, sulfur, oxygen, or nitrogen. In some embodiments, the heteroaryl group has from 3 to 20 ring-forming atoms, from 3 to 10 ring-forming atoms, from 3 to 6 ring-forming atoms, or from 3 to 5 ring-forming atoms. In some embodiments, the heteroaryl group contains 2 to 14 carbon atoms, from 2 to 7 carbon atoms, or 5 or 6 carbon atoms. In some embodiments, the heteroaryl group has 1 to 4 heteroatoms, 1 to 3 heteroatoms, or 1 or 2 heteroatoms. Heteroaryl groups include monocyclic and polycyclic (e.g., having 2, 3 or 4 fused rings) systems. Examples of heteroaryl groups include, but are not limited to, pyridyl, pyrimidinyl, pyrazinyl, pyridazinyl, triazinyl, furyl, quinolyl, isoquinolyl, thienyl, imidazolyl, thiazolyl, indolyl (such as indol-3-yl), pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, indolinyl, pyranyl, oxadiazolyl, isoxazolyl, triazolyl, thianthrenyl, pyrazolyl, indolizinyl, isoindolyl, isobenzofuranyl, benzoxazolyl, xanthenyl, 2H-pyrrolyl, pyrrolyl, 3H-indolyl, 4H-quinolizinyl, phthalazinyl, naphthyridinyl, quinazolinyl, phenanthridinyl, acridinyl, perimidinyl, phenanthrolinyl, phenazinyl, isothiazolyl, phenothiazinyl, isoxazolyl, furazanyl, phenoxazinyl groups, and the like. Suitable heteroaryl groups include 1,2,3-triazole, 1,2,4-triazole, 5-amino-1,2,4-triazole, imidazole, oxazole, isoxazole, 1,2,3-oxadiazole, 1,2,4-oxadiazole, 3-amino-1,2,4-oxadiazole, 1,2,5-oxadiazole, 1,3,4-oxadiazole, pyridine, and 2-aminopyridine.

As used herein, the term “heteroarylene” means a heteroaryl linking group, i.e., a heteroaryl group that links one group to another group in a molecule.

As used herein, the term “heterocycle” or “heterocyclic ring” means a 5- to 7-membered mono- or bicyclic or 7- to 10-membered bicyclic heterocyclic ring system any ring of which may be saturated or unsaturated, and which consists of carbon atoms and from one to three heteroatoms chosen from N, O and S, and wherein the N and S heteroatoms may optionally be oxidized, and the N heteroatom may optionally be quaternized, and including any bicyclic group in which any of the above-defined heterocyclic rings is fused to a benzene ring. Particularly useful are rings containing one oxygen or sulfur, one to three nitrogen atoms, or one oxygen or sulfur combined with one or two nitrogen atoms. The heterocyclic ring may be attached at any heteroatom or carbon atom which results in the creation of a stable structure. Examples of heterocyclic groups include, but are not limited to, piperidinyl, piperazinyl, 2-oxopiperazinyl, 2-oxopiperidinyl, 2-oxopyrrolodinyl, 2-oxoazepinyl, azepinyl, pyrrolyl, 4-piperidonyl, pyrrolidinyl, pyrazolyl, pyrazolidinyl, imidazolyl, imidazolinyl, imidazolidinyl, pyridyl, pyrazinyl, pyrimidinyl, pyridazinyl, oxazolyl, oxazolidinyl, isoxazolyl, isoxazolidinyl, morpholinyl, thiazolyl, thiazolidinyl, isothiazolyl, quinuclidinyl, isothiazolidinyl, indolyl, quinolinyl, isoquinolinyl, benzimidazolyl, thiadiazoyl, benzopyranyl, benzothiazolyl, benzoxazolyl, furyl, tetrahydrofuryl, tetrahydropyranyl, thienyl, benzothienyl, thiamorpholinyl, thiamorpholinyl sulfoxide, thiamorpholinyl sulfone, and oxadiazolyl. Morpholino is the same as morpholinyl.

As used herein, the term “heterocycloalkyl” means non-aromatic heterocycles having up to 20 ring-forming atoms including cyclized alkyl, alkenyl, and alkynyl groups, where one or more of the ring-forming carbon atoms is replaced by a heteroatom such as an O, N, or S atom. Heterocycloalkyl groups can be mono or polycyclic (e.g., fused, bridged, or spiro systems). In some embodiments, the heterocycloalkyl group has from 1 to 20 carbon atoms, or from 3 to 20 carbon atoms. In some embodiments, the heterocycloalkyl group contains 3 to 14 ring-forming atoms, 3 to 7 ring-forming atoms, or 5 or 6 ring-forming atoms. In some embodiments, the heterocycloalkyl group has 1 to 4 heteroatoms, 1 to 3 heteroatoms, or 1 or 2 heteroatoms. In some embodiments, the heterocycloalkyl group contains 0 to 3 double bonds. In some embodiments, the heterocycloalkyl group contains 0 to 2 triple bonds. Examples of heterocycloalkyl groups include, but are not limited to, morpholino, thiomorpholino, piperazinyl, tetrahydrofuranyl, tetrahydrothienyl, 2,3-dihydrobenzofuryl, 1,3-benzodioxole, benzo-1,4-dioxane, piperidinyl, pyrrolidinyl, isoxazolidinyl, oxazolidinyl, isothiazolidinyl, pyrazolidinyl, thiazolidinyl, imidazolidinyl, pyrrolidin-2-one-3-yl, and the like. In addition, ring-forming carbon atoms and heteroatoms of a heterocycloalkyl group can be optionally substituted by oxo or sulfido. For example, a ring-forming S atom can be substituted by 1 or 2 oxo (form a S(O) or S(O)₂). For another example, a ring-forming C atom can be substituted by oxo (form carbonyl). Also included in the definition of heterocycloalkyl are moieties that have one or more aromatic rings fused (having a bond in common with) to the nonaromatic heterocyclic ring including, but not limited to, pyridinyl, thiophenyl, phthalimidyl, naphthalimidyl, and benzo derivatives of heterocycles such as indolene, isoindolene, 4,5,6,7-tetrahydrothieno[2,3-c]pyridine-5-yl, 5,6-dihydrothieno[2,3-c]pyridin-7(4H)-one-5-yl, isoindolin-1-one-3-yl, and 3,4-dihydroisoquinolin-1(2H)-one-3yl groups. Ring-forming carbon atoms and heteroatoms of the heterocycloalkyl group can be optionally substituted by oxo or sulfido.

As used herein, the term “hydroxy” or “hydroxyl” means an —OH group.

As used herein, the term “hydroxyalkyl” or “hydroxylalkyl” means an alkyl group substituted by a hydroxyl group. Examples of a hydroxylalkyl include, but are not limited to, —CH₂OH and —CH₂CH₂OH.

As used herein, the term “individual” or “patient,” used interchangeably, means any animal, including mammals, such as mice, rats, other rodents, rabbits, dogs, cats, swine, cattle, sheep, horses, or primates, such as humans.

As used herein, the phrase “inhibiting the growth” means reducing by any measurable amount the growth of one or more Plasmodium species. In some embodiments, the inhibition of growth may result in cell death of the Plasmodium species.

As used herein, the phrase “in need thereof” means that the animal or mammal has been identified as having a need for the particular method or treatment. In some embodiments, the identification can be by any means of diagnosis. In any of the methods and treatments described herein, the animal or mammal can be in need thereof. In some embodiments, the animal or mammal is in an environment or will be traveling to an environment in which malaria is prevelant.

As used herein, the phrase “in situ gellable” means embracing not only liquids of low viscosity that form gels upon contact with the eye or with lacrimal fluid in the exterior of the eye, but also more viscous liquids such as semi-fluid and thixotropic gels that exhibit substantially increased viscosity or gel stiffness upon administration to the eye.

As used herein, the phrase “integer from 1 to 5” means 1, 2, 3, 4, or 5.

As used herein, the term “isolated” means that the compounds described herein are separated from other components of either (a) a natural source, such as a plant or cell, such as a bacterial culture, or (b) a synthetic organic chemical reaction mixture, such as by conventional techniques.

As used herein, the term “malarialcidal” means that the compound inhibits, prevents, or destroys the growth or proliferation of a Plasmodium species.

As used herein, the term “mammal” means a rodent (i.e., a mouse, a rat, or a guinea pig), a monkey, a cat, a dog, a cow, a horse, a pig, or a human. In some embodiments, the mammal is a human.

As used herein, the term “nitro” means —NO₂.

As used herein, the term “n-membered”, where n is an integer, typically describes the number of ring-forming atoms in a moiety, where the number of ring-forming atoms is n. For example, pyridine is an example of a 6-membered heteroaryl ring and thiophene is an example of a 5-membered heteroaryl ring.

As used used herein, the phrase “optionally substituted” means that substitution is optional and therefore includes both unsubstituted and substituted atoms and moieties. A “substituted” atom or moiety indicates that any hydrogen on the designated atom or moiety can be replaced with a selection from the indicated substituent groups, provided that the normal valency of the designated atom or moiety is not exceeded, and that the substitution results in a stable compound. For example, if a methyl group is optionally substituted, then 3 hydrogen atoms on the carbon atom can be replaced with substituent groups.

As used herein, the phrase “pharmaceutically acceptable” means those compounds, materials, compositions, and/or dosage forms which are, within the scope of sound medical judgment, suitable for use in contact with tissues of humans and animals. In some embodiments, “pharmaceutically acceptable” means approved by a regulatory agency of the Federal or a state government or listed in the U.S. Pharmacopeia or other generally recognized pharmacopeia for use in animals, and more particularly in humans.

As used herein, the phrase “pharmaceutically acceptable salt(s),” includes, but is not limited to, salts of acidic or basic groups. Compounds that are basic in nature are capable of forming a wide variety of salts with various inorganic and organic acids. Acids that may be used to prepare pharmaceutically acceptable acid addition salts of such basic compounds are those that form non-toxic acid addition salts, i.e., salts containing pharmacologically acceptable anions including, but not limited to, sulfuric, thiosulfuric, citric, maleic, acetic, oxalic, hydrochloride, hydrobromide, hydroiodide, nitrate, sulfate, bisulfate, bisulfite, phosphate, acid phosphate, isonicotinate, borate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, bicarbonate, malonate, mesylate, esylate, napsydisylate, tosylate, besylate, orthophoshate, trifluoroacetate, and pamoate (i.e., 1,1′-methylene-bis-(2-hydroxy-3-naphthoate)) salts. Compounds that include an amino moiety may form pharmaceutically acceptable salts with various amino acids, in addition to the acids mentioned above. Compounds that are acidic in nature are capable of forming base salts with various pharmacologically acceptable cations. Examples of such salts include, but are not limited to, alkali metal or alkaline earth metal salts and, particularly, calcium, magnesium, ammonium, sodium, lithium, zinc, potassium, and iron salts. The present disclosure also includes quaternary ammonium salts of the compounds described herein, where the compounds have one or more tertiary amine moiety.

As used herein, the term “phenyl” means —C₆H₅. A phenyl group can be unsubstituted or substituted with one, two, or three suitable substituents.

As used herein, the terms “prevention” or “preventing” mean a reduction of the risk of acquiring a particular disease, condition, or disorder.

As used herein, the term “prodrug” means a derivative of a known direct acting drug, which derivative has enhanced delivery characteristics and therapeutic value as compared to the drug, and is transformed into the active drug by an enzymatic or chemical process.

As used herein, the term “purified” means that when isolated, the isolate contains at least 90%, at least 95%, at least 98%, or at least 99% of a compound described herein by weight of the isolate.

As used herein, the phrase “quaternary ammonium salts” means derivatives of the disclosed compounds with one or more tertiary amine moieties wherein at least one of the tertiary amine moieties in the parent compound is modified by converting the tertiary amine moiety to a quaternary ammonium cation via alkylation (and the cations are balanced by anions such as Cl⁻, CH₃COO⁻, and CF₃COO⁻), for example methylation or ethylation.

As used herein, the phrase “solubilizing agent” means agents that result in formation of a micellar solution or a true solution of the drug.

As used herein, the term “solution/suspension” means a liquid composition wherein a first portion of the active agent is present in solution and a second portion of the active agent is present in particulate form, in suspension in a liquid matrix.

As used herein, the phrase “substantially isolated” means a compound that is at least partially or substantially separated from the environment in which it is formed or detected.

As used herein, the phrase “suitable substituent” or “substituent” means a group that does not nullify the synthetic or pharmaceutical utility of the compounds described herein or the intermediates useful for preparing them. Examples of suitable substituents include, but are not limited to: C₁-C₆alkyl, C₁-C₆alkenyl, C₁-C₆alkynyl, C₅-C₆aryl, C₁-C₆alkoxy, C₃-C₅heteroaryl, C₃-C₆cycloalkyl, C₅-C₆aryloxy, —CN, —OH, oxo, halo, haloalkyl, —NO₂, —CO₂H, —NH₂, —NH(C₁-C₈alkyl), —N(C₁-C₈alkyl)₂, —NH(C₆aryl), —N(C₅-C₆aryl)₂, —CHO, —CO(C₁-C₆alkyl), —CO((C₅-C₆)aryl), —CO₂((C₁-C₆)alkyl), and —CO₂((C₅-C₆)aryl). One of skill in art can readily choose a suitable substituent based on the stability and pharmacological and synthetic activity of the compounds described herein.

As used herein, the phrase “therapeutically effective amount” means the amount of active compound or pharmaceutical agent that elicits the biological or medicinal response that is being sought in a tissue, system, animal, individual or human by a researcher, veterinarian, medical doctor or other clinician. The therapeutic effect is dependent upon the disorder being treated or the biological effect desired. As such, the therapeutic effect can be a decrease in the severity of symptoms associated with the disorder and/or inhibition (partial or complete) of progression of the disorder, or improved treatment, healing, prevention or elimination of a disorder, or side-effects. The amount needed to elicit the therapeutic response can be determined based on the age, health, size and sex of the subject. Optimal amounts can also be determined based on monitoring of the subject's response to treatment.

As used herein, the terms “treat,” “treated,” or “treating” mean both therapeutic treatment and prophylactic or preventative measures wherein the object is to prevent or slow down (lessen) an undesired physiological condition, disorder or disease, or obtain beneficial or desired clinical results. For purposes of this disclosure, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment includes eliciting a clinically significant response without excessive levels of side effects. Treatment also includes prolonging survival as compared to expected survival if not receiving treatment. Thus, “treatment of malaria” or “treating malaria” means an activity that prevents, alleviates or ameliorates any of the primary phenomena or secondary symptoms associated with the disease.

At various places in the present specification, substituents of compounds may be disclosed in groups or in ranges. It is specifically intended that the disclosure include each and every individual subcombination of the members of such groups and ranges. For example, the term “C₁₋₆alkyl” is specifically intended to individually disclose methyl, ethyl, propyl, C₄alkyl, C₅alkyl, and C₆alkyl.

For compounds in which a variable appears more than once, each variable can be a different moiety selected from the Markush group defining the variable. For example, where a structure is described having two R groups that are simultaneously present on the same compound, the two R groups can represent different moieties selected from the Markush groups defined for R. In another example, when an optionally multiple substituent is designated in the form, for example,

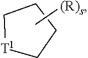

then it is understood that substituent R can occur s number of times on the ring, and R can be a different moiety at each occurrence. Further, in the above example, where the variable T¹ is defined to include hydrogens, such as when T¹ is CH₂, NH, etc., any H can be replaced with a substituent.

It is further appreciated that certain features of the disclosure, which are, for clarity, described in the context of separate embodiments, can also be provided in combination in a single embodiment. Conversely, various features of the disclosure which are, for brevity, described in the context of a single embodiment, can also be provided separately or in any suitable subcombination.

It is understood that the present disclosure encompasses the use, where applicable, of stereoisomers, diastereomers and optical stereoisomers of the compounds of the disclosure, as well as mixtures thereof. Additionally, it is understood that stereoisomers, diastereomers, and optical stereoisomers of the compounds of the disclosure, and mixtures thereof, are within the scope of the disclosure. By way of non-limiting example, the mixture may be a racemate or the mixture may comprise unequal proportions of one particular stereoisomer over the other.

Additionally, the compounds can be provided as a substantially pure stereoisomers, diastereomers and optical stereoisomers (such as epimers).

The compounds described herein can be asymmetric (e.g., having one or more stereocenters). All stereoisomers, such as enantiomers and diastereomers, are intended to be included within the scope of the disclosure unless otherwise indicated. Compounds that contain asymmetrically substituted carbon atoms can be isolated in optically active or racemic forms.

Methods of preparation of optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds are also included within the scope of the disclosure and can be isolated as a mixture of isomers or as separated isomeric forms. Where a compound capable of stereoisomerism or geometric isomerism is designated in its structure or name without reference to specific R/S or cis/trans configurations, it is intended that all such isomers are contemplated.

Resolution of racemic mixtures of compounds can be carried out by any of numerous methods known in the art, including, for example, fractional recrystallizaion using a chiral resolving acid which is an optically active, salt-forming organic acid. Suitable resolving agents for fractional recrystallization methods include, but are not limited to, optically active acids, such as the D and L forms of tartaric acid, diacetyltartaric acid, dibenzoyltartaric acid, mandelic acid, malic acid, lactic acid, and the various optically active camphorsulfonic acids such as β-camphorsulfonic acid. Other resolving agents suitable for fractional crystallization methods include, but are not limited to, stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like. Resolution of racemic mixtures can also be carried out by elution on a column packed with an optically active resolving agent (e.g., dinitrobenzoylphenylglycine). Suitable elution solvent compositions can be determined by one skilled in the art.

Compounds may also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Examples of prototropic tautomers include, but are not limited to, ketone-enol pairs, amide-imidic acid pairs, lactam-lactim pairs, amide-imidic acid pairs, enamine-imine pairs, and annular forms where a proton can occupy two or more positions of a heterocyclic system including, but not limited to, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds also include hydrates and solvates, as well as anhydrous and non-solvated forms.

Compounds can also include all isotopes of atoms occurring in the intermediates or final compounds. Isotopes include those atoms having the same atomic number but different mass numbers. For example, isotopes of hydrogen include tritium and deuterium.

In some embodiments, the compounds, or salts thereof, are substantially isolated. Partial separation can include, for example, a composition enriched in the compound of the disclosure.

Substantial separation can include compositions containing at least about 50%, at least about 60%, at least about 70%, at least about 80%, at least about 90%, at least about 95%, at least about 97%, or at least about 99% by weight of the compound of the disclosure, or salt thereof. Methods for isolating compounds and their salts are routine in the art.

Although the disclosed compounds are suitable, other functional groups can be incorporated into the compound with an expectation of similar results. In particular, thioamides and thioesters are anticipated to have very similar properties. The distance between aromatic rings can impact the geometrical pattern of the compound and this distance can be altered by incorporating aliphatic chains of varying length, which can be optionally substituted or can comprise an amino acid, a dicarboxylic acid or a diamine. The distance between and the relative orientation of monomers within the compounds can also be altered by replacing the amide bond with a surrogate having additional atoms. Thus, replacing a carbonyl group with a dicarbonyl alters the distance between the monomers and the propensity of dicarbonyl unit to adopt an anti arrangement of the two carbonyl moiety and alter the periodicity of the compound. Pyromellitic anhydride represents still another alternative to simple amide linkages which can alter the conformation and physical properties of the compound. Modern methods of solid phase organic chemistry (E. Atherton and R. C. Sheppard, Solid Phase Peptide Synthesis A Practical Approach IRL Press Oxford 1989) now allow the synthesis of homodisperse compounds with molecular weights approaching 5,000 Daltons. Other substitution patterns are equally effective.

The compounds also include derivatives referred to as prodrugs, which can be prepared by modifying functional groups present in the compounds in such a way that the modifications are cleaved, either in routine manipulation or in vivo, to the parent compounds. Examples of prodrugs include compounds of the disclosure as described herein that contain one or more molecular moieties appended to a hydroxyl, amino, sulfhydryl, or carboxyl group of the compound, and that when administered to a patient, cleaves in vivo to form the free hydroxyl, amino, sulfhydryl, or carboxyl group, respectively. Examples of prodrugs include, but are not limited to, acetate, formate and benzoate derivatives of alcohol and amine functional groups in the compounds of the disclosure. Preparation and use of prodrugs is discussed in T. Higuchi et al., “Pro-drugs as Novel Delivery Systems,” Vol. 14 of the A.C.S. Symposium Series, and in Bioreversible Carriers in Drug Designs, ed. Edward B. Roche, American Pharmaceutical Association and Pergamon Press, 1987, both of which are incorporated herein by reference in their entireties.

Compounds containing an amine function can also form N-oxides. A reference herein to a compound that contains an amine function also includes the N-oxide. Where a compound contains several amine functions, one or more than one nitrogen atom can be oxidized to form an N-oxide. Examples of N-oxides include N-oxides of a tertiary amine or a nitrogen atom of a nitrogen-containing heterocycle. N-Oxides can be formed by treatment of the corresponding amine with an oxidizing agent such as hydrogen peroxide or a per-acid (e.g., a peroxycarboxylic acid) (see, Advanced Organic Chemistry, by Jerry March, 4th Edition, Wiley Interscience).

The structures depicted herein may omit necessary hydrogen atoms to complete the appropriate valency. Thus, in some instances a carbon atom or nitrogen atom may appear to have an open valency (i.e., a carbon atom with only two bonds showing would implicitly also be bonded to two hydrogen atoms; in addition, a nitrogen atom with a single bond depicted would implicitly also be bonded to two hydrogen atoms). For example, “—N” would be considered by one skilled in the art to be “—NH₂.” Thus, in any structure depicted herein wherein a valency is open, a hydrogen atom is implicit, and is only omitted for brevity.

The present disclosure provides anti-malarial compounds of Formula I:

R¹-A₁-X—Y-A₂-Y—X-A₁-R² (I)

or a pharmaceutically acceptable salt thereof,
wherein:

each X is, independently, —NR⁸, —N(R⁸)N(R⁸)—, O, or S, wherein each R⁸ is, independently, hydrogen or alkyl;

each Y is, independently, C═O, C═S, or O═S═O;

each A₁ and A₂ is, independently, aryl optionally substituted with one or more W, or heteroaryl optionally substituted with one or more W;

each W is, independently, halo, —CF₃, cyano, C₁₋₄alkoxy, —S—(CH₂)₁₋₅NHC(═NH)NH₂, —S—(CH₂)₁₋₅NH₂, —S—(CH₂)₁₋₅N(CH₃)₂, —S—(CH₂)₁₋₅NHS(═O)₂—C₁₋₄alkyl, —C₁₋₄alkyl, —O—(CH₂)₁₋₅NHC(═NH)NH₂, or O-heterocycle (wherein the heterocycle is optionally substituted with one or more cyano, amino, C₁₋₄alkyl, guanidino, hydroxyl, amidino, C₁₋₄alkoxy, CF₃, or halo); and

R¹ and R² are, independently, —(CH₂)₁₋₄NH₂, —(CH₂)₁₋₅NHC(═NH)NH₂, —S—(CH₂)₁₋₅N(CH₃)₂, —O—(CH₂)₁₋₅NHC(═NH)NH₂, —S—(CH₂)₁₋₅NHC(═NH)NH₂, —O—(CH₂)₁₋₅N(CH₃)₂, amino, —O-heterocycle or —O-cycloalkyl (wherein the heterocycle and cycloalkyl are optionally substituted), —S-heterocycle (wherein the heterocycle is optionally substituted), —NHC(═O)—(CH₂)₁₋₅NHC(═NH)NH₂, —NHC(═O)—C₁₋₄alkyl, heterocycle (wherein the heterocycle is optionally substituted), —NHC(═O)—(CH₂)₁₋₅NHS(═O)₂—C₁₋₄alkyl, —NHC(═O)—(CH₂)₁₋₅-aryl (wherein either or both —(CH₂)₁₋₅ and aryl is optionally substituted), —NHC(═O)-aryl (wherein the aryl is optionally substituted), or —NHC(═O)—(CH₂)₁₋₅NH-aryl (wherein either or both —NH and aryl is optionally substituted), wherein the optional substituents are chosen from one or more cyano, amino, C₁₋₄alkyl, guanidino, hydroxyl, amidino, C₁₋₄alkoxy, CF₃, halo, and —N(CH₃)₂.

In some embodiments, each X is, independently, NR⁸, O, or S, wherein each R⁸ is, independently, hydrogen or alkyl. In some embodiments, each X is, independently, NR⁸, wherein each R⁸ is, independently, hydrogen or C₁₋₄alkyl. In some embodiments, each X is NH.

In some embodiments, each Y is C═O.

In some embodiments, A₂ is optionally substituted phenyl, pyridine, pyrimidine, pyrazine, or pyrazole. In some embodiments, A₂ is unsubstituted o-, m-, orp-phenyl, o-, m-, or p-pyridine, o-, m-, orp-pyrimidine, o-, m-, orp-pyrazine, or o-, m-, orp-pyrazole. In some embodiments, A₂ is unsubstituted m-phenyl, m-pyridine, m-pyrimidine, m-pyrazine, or m-pyrazole. In some embodiments, A₂ is unsubstituted m-phenyl, m-pyridine, m-pyrimidine, or m-pyrazole.

In some embodiments, each A₁ is, independently, optionally substituted o-, m-, or p-phenyl. In some embodiments, each A₁ is, independently, substituted o-, m-, orp-phenyl.

In some embodiments, each W is, independently, F, —CF₃, —S—(CH₂)₂NH₂, —S—(CH₂)₂NHC(═NH)NH₂, —S—(CH₂)₂N(CH₃)₂, —S—(CH₂)₂NHS(═O)₂CH₃, —C(CH₃)₃, —O—(CH₂)₄NHC(═NH)NH₂, or —O-pyrrolidine (wherein the pyrrolidine is optionally substituted with amidino).

In some embodiments, R¹ and R² are, independently, —(CH₂)₂₋₃NHC(═NH)NH₂, —O—(CH₂)₄NHC(═NH)NH₂, —S—(CH₂)₂NHC(═NH)NH₂, —S—(CH₂)₂N(CH₃)₂, —O-pyrrolidine (wherein the pyrrolidine is optionally substituted with amidino), amino, —NHC(═O)—CH₃, —NHC(═O)—(CH₂)₄₋₅NHC(═NH)NH₂, —NHC(═O)—(CH₂)₄NHS(═O)₂—CH₃, —NHC(═O)CH(NH₂)CH₂-phenyl, —NHC(═O)—(CH₂)₄N(CH₃)-pyridine, or —NHC(═O)-phenyl (wherein the phenyl is substituted with cyano).

In some embodiments, each X is, independently, —NR⁸, —N(R⁸)N(R⁸)—, O, or S, wherein each R⁸ is, independently, hydrogen or alkyl; each Y is, independently, C═O, C═S, or O═S═O; each A₁ and A₂ is, independently, aryl optionally substituted with one or more W, or heteroaryl optionally substituted with one or more W; each W is, independently, halo, —CF₃, —S—(CH₂)₁₋₅NHC(═NH)NH₂, —S—(CH₂)₁₋₅NH₂, —S—(CH₂)₁₋₅N(CH₃)₂, —S—(CH₂)₁₋₅NHS(═O)₂—C₁₋₄alkyl, —C₁₋₄alkyl, —O—(CH₂)₁₋₅NHC(═NH)NH₂, or O-heterocycle (wherein the heterocycle is optionally substituted with one or more cyano, amino, C₁₋₄alkyl, guanidino, hydroxyl, amidino, C₁₋₄alkoxy, CF₃, or halo); and R¹ and R² are, independently, —(CH₂)₁₋₅NHC(═NH)NH₂, —S—(CH₂)₁₋₅N(CH₃)₂, —O—(CH₂)₁₋₅NHC(═NH)NH₂, —S—(CH₂)₁₋₅NHC(═NH)NH₂, —O—(CH₂)₁₋₅N(CH₃)₂, amino, —O-heterocycle (wherein the heterocycle is optionally substituted), —S-heterocycle (wherein the heterocycle is optionally substituted), —NHC(═O)—(CH₂)₁₋₅NHC(═NH)NH₂, —NHC(═O)—C₁₋₄alkyl, —NHC(═O)—(CH₂)₁₋₅NHS(═O)₂—C₁₋₄alkyl, —NHC(═O)—(CH₂)₁₋₅-aryl (wherein either or both —(CH₂)₁₋₅ and aryl is optionally substituted), —NHC(═O)-aryl (wherein the aryl is optionally substituted), or —NHC(═O)—(CH₂)₁₋₅NH-aryl (wherein either or both —NH and aryl is optionally substituted), wherein the optional substituents are chosen from one or more cyano, amino, C₁₋₄alkyl, guanidino, hydroxyl, amidino, C₁₋₄alkoxy, CF₃, and halo.

In some embodiments: each X is NH; each Y is C═O; A₂ is unsubstituted pyrimidine; each A₁ is, independently, phenyl optionally substituted with one or more W; R¹ and R² are, independently, —(CH₂)₂₋₃NHC(═NH)NH₂, —NHC(═O)-phenyl (wherein the phenyl is substituted with cyano), —S—(CH₂)₂N(CH₃)₂, —O-pyrrolidine (wherein the pyrrolidine is optionally substituted with amidino), —O—(CH₂)₄NHC(═NH)NH₂, —S—(CH₂)₂NHC(═NH)NH₂, or —NHC(═O)—(CH₂)₄₋₅NHC(═NH)NH₂; and each W is, independently, F, —CF₃, —S—(CH₂)₂NH₂, —S—(CH₂)₂N(CH₃)₂, —O-pyrrolidine (wherein the pyrrolidine is optionally substituted with amidino), —O—(CH₂)₄NHC(═NH)NH₂, or —S—(CH₂)₂NHC(═NH)NH₂.

In some embodiments: each X is NH; each Y is C═O; A₂ is unsubstituted phenyl; each A₁ is, independently, phenyl substituted with one or more W; R¹ and R² are, independently, amino, —NHC(═O)—(CH₂)₄NHS(═O)₂—CH₃, —NHC(═O)—CH₃, —NHC(═O)—(CH₂)₄N(CH₃)-pyridine, or —NHC(═O)CH(NH₂)CH₂-phenyl; and each W is, independently, —C(CH₃)₃, —CF₃, —S—(CH₂)₂NH₂, —S—(CH₂)₂NHS(═O)₂CH₃, or —S—(CH₂)₂NHC(═NH)NH₂.

In some embodiments: each X is NH; each Y is C═O; A₂ is unsubstituted pyridine; each A₁ is, independently, phenyl substituted with one or more W; R¹ and R² are amino; and each W is, independently, —C(CH₃)₃ or —S—(CH₂)₂NH₂.

In some embodiments: each X is NH; each Y is C═O; A₂ is unsubstituted pyrazole; each A₁ is, independently, phenyl substituted with one or more W; R¹ and R² are —NHC(═O)—(CH₂)₄₋₅NHC(═NH)NH₂; and each W is, independently, —CF₃ or —S—(CH₂)₂NH₂.

In some embodiments, the compound of Formula I is not one of the following:

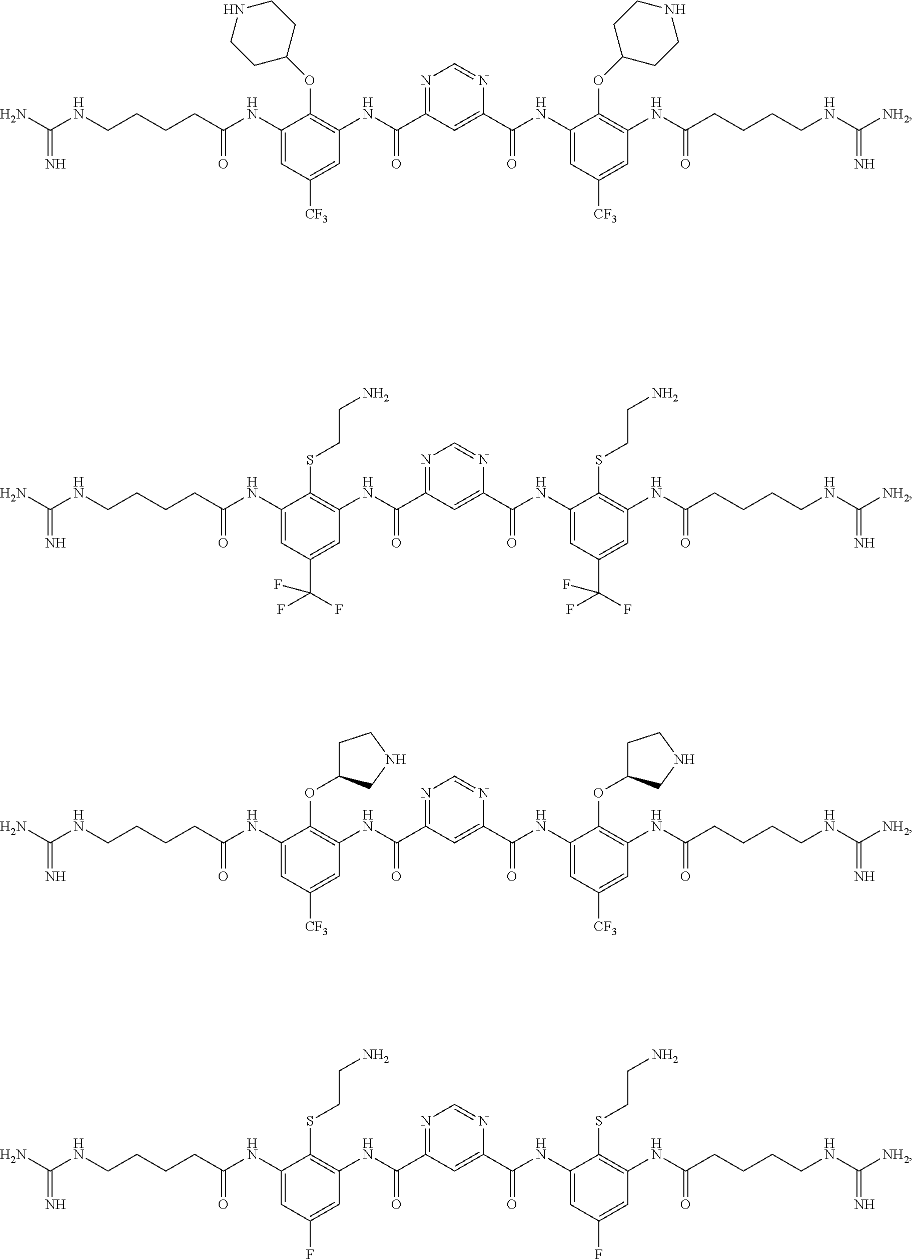 [SMILES: N=C(N)NCCCCC(=O)Nc1cc(C(F)(F)F)cc(NC(=O)c2cc(C(=O)Nc3cc(C(F)(F)F)cc(NC(=O)CCCCNC(=N)N)c3OC3CCNCC3)ncn2)c1OC1CCNCC1 ; N=C(N)NCCCCC(=O)Nc1cc(C(F)(F)F)cc(NC(=O)c2cc(C(=O)Nc3cc(C(F)(F)F)cc(NC(=O)CCCCNC(=N)N)c3SCCN)ncn2)c1SCCN ; N=C(N)NCCCCC(=O)Nc1cc(C(F)(F)F)cc(NC(=O)c2cc(C(=O)Nc3cc(C(F)(F)F)cc(NC(=O)CCCCNC(=N)N)c3O[C@H]3CCNC3)ncn2)c1O[C@H]1CCNC1 ; N=C(N)NCCCCC(=O)Nc1cc(F)cc(NC(=O)c2cc(C(=O)Nc3cc(F)cc(NC(=O)CCCCNC(=N)N)c3SCCN)ncn2)c1SCCN]

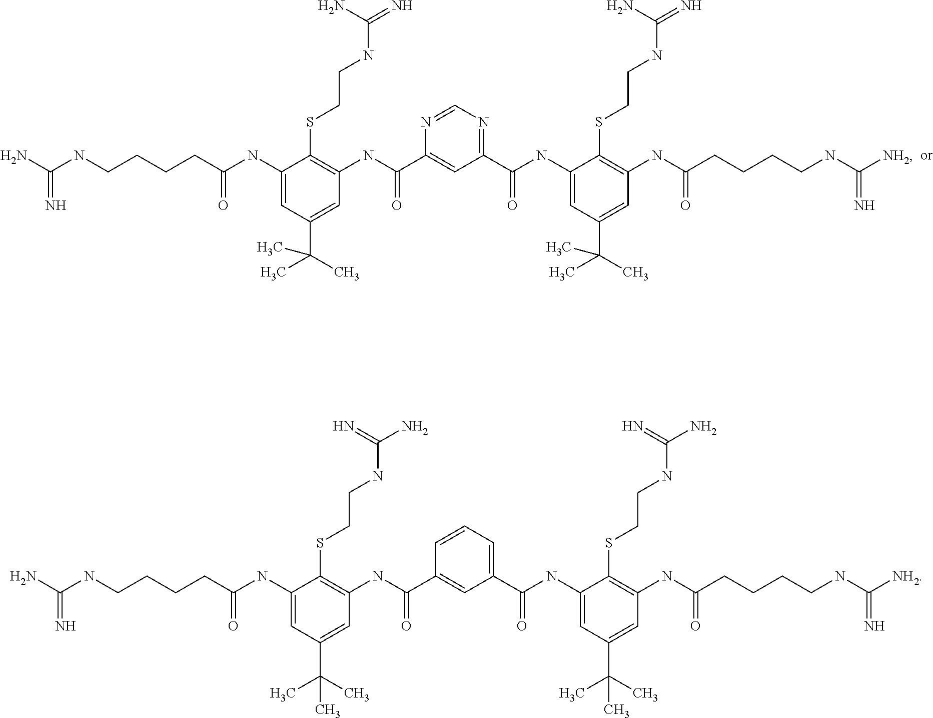 [SMILES: CC(C)(C)c1cc(NC(=O)CCCCNC(=N)N)c(SCCNC(=N)N)c(NC(=O)c2cc(C(=O)Nc3cc(C(C)(C)C)cc(NC(=O)CCCCNC(=N)N)c3SCCNC(=N)N)ncn2)c1 ; CC(C)(C)c1cc(NC(=O)CCCCNC(=N)N)c(SCCNC(=N)N)c(NC(=O)c2cccc(C(=O)Nc3cc(C(C)(C)C)cc(NC(=O)CCCCNC(=N)N)c3SCCNC(=N)N)c2)c1]

In some embodiments, the compound of Formula I is chosen from:

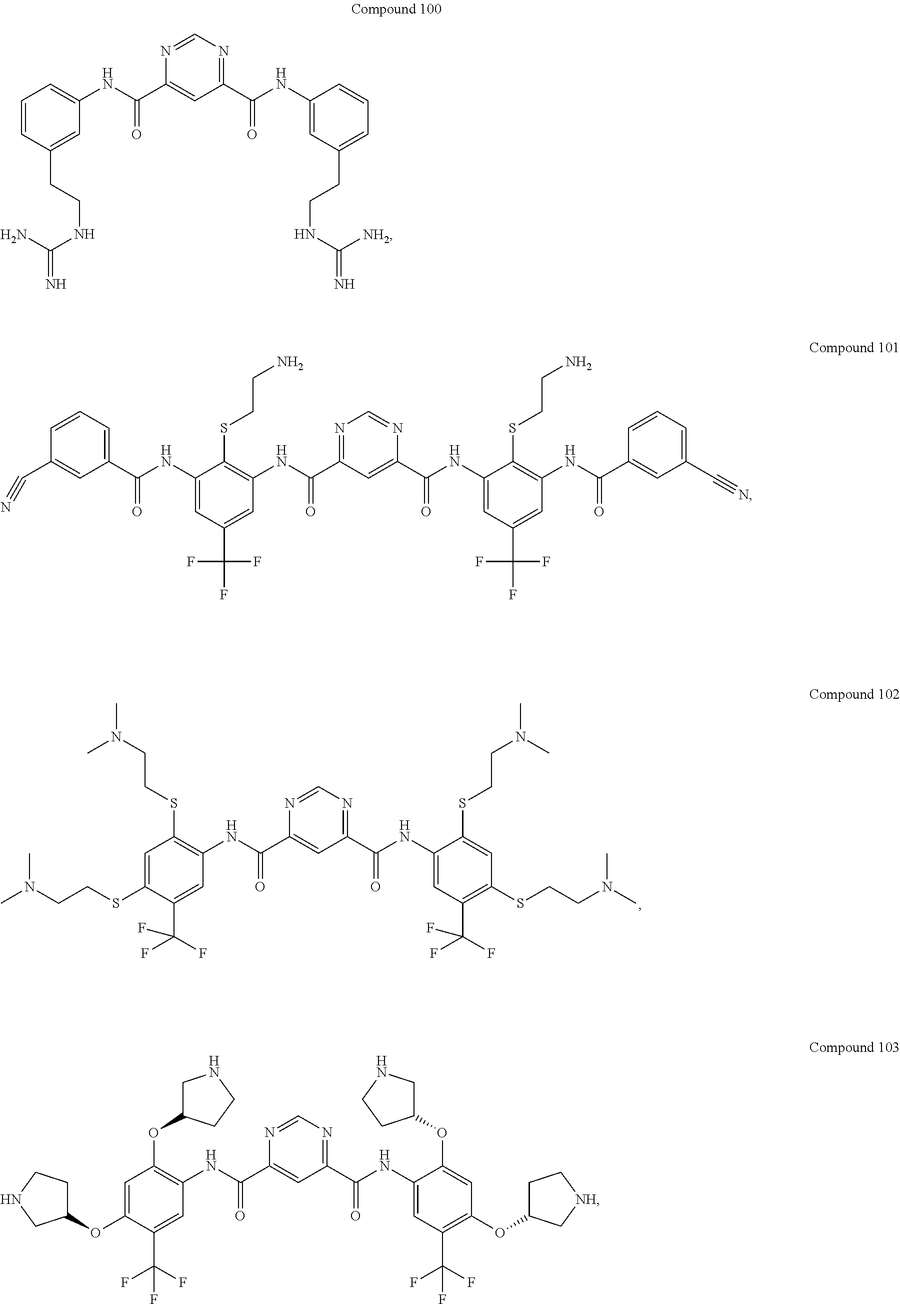

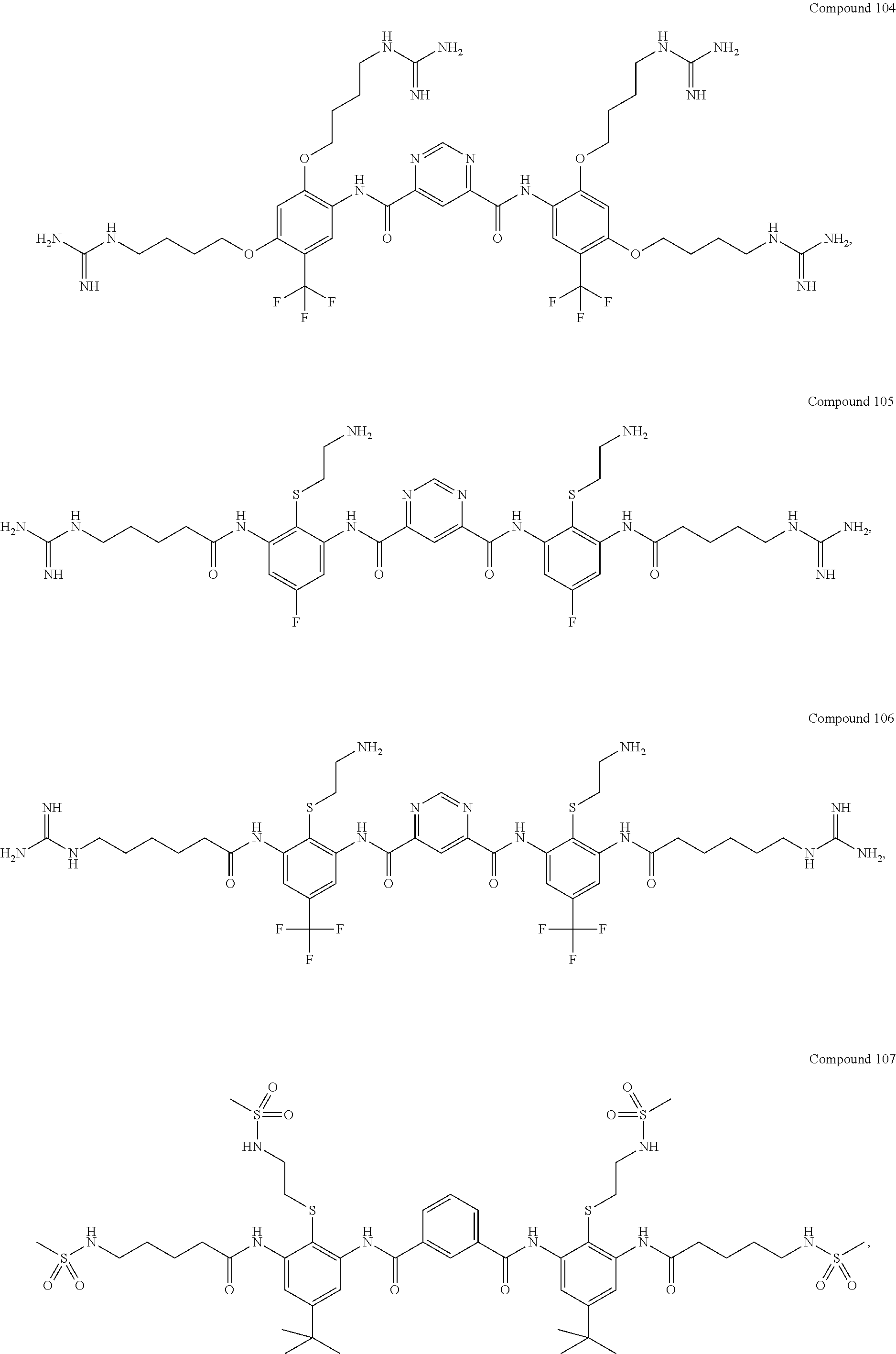

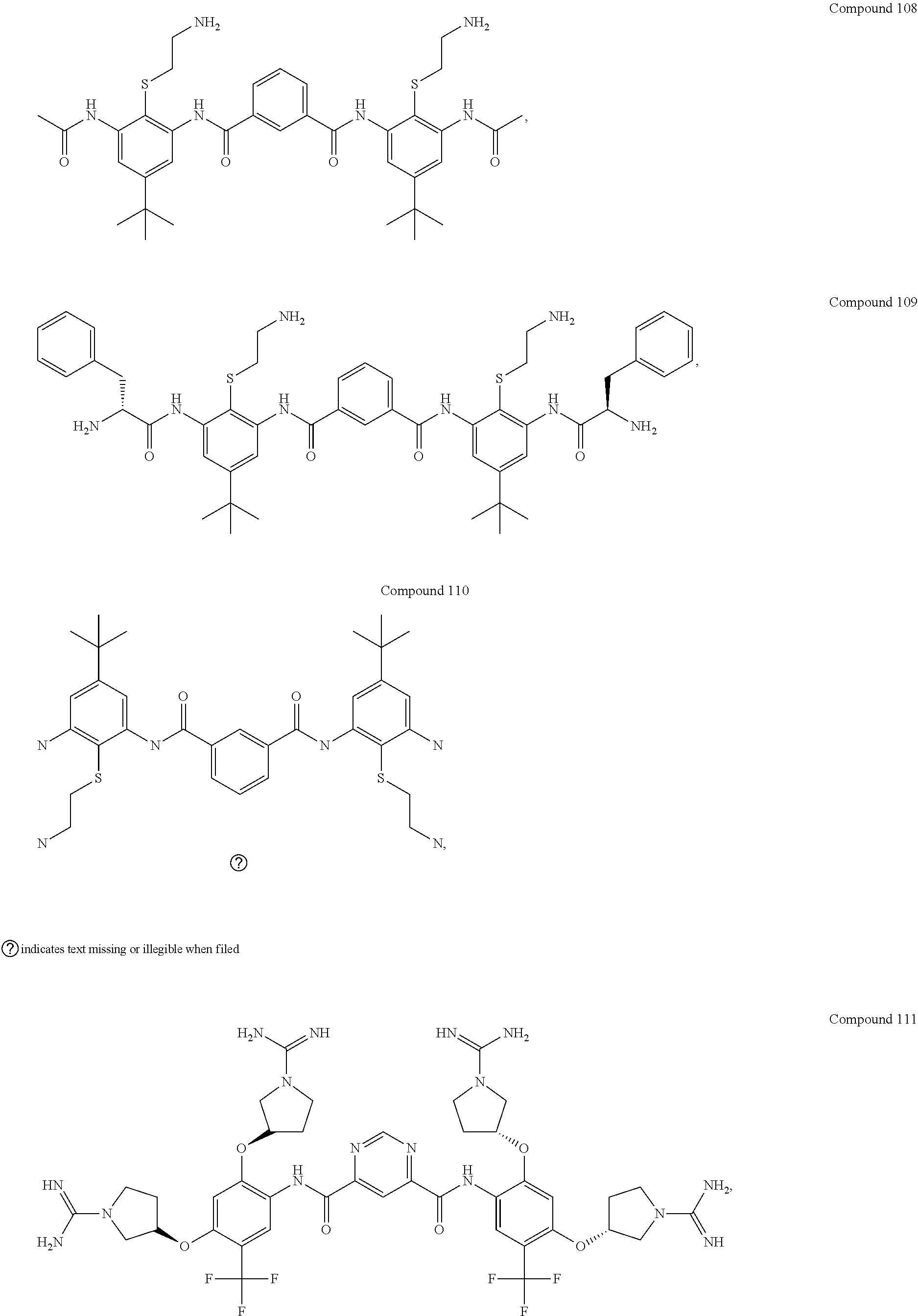

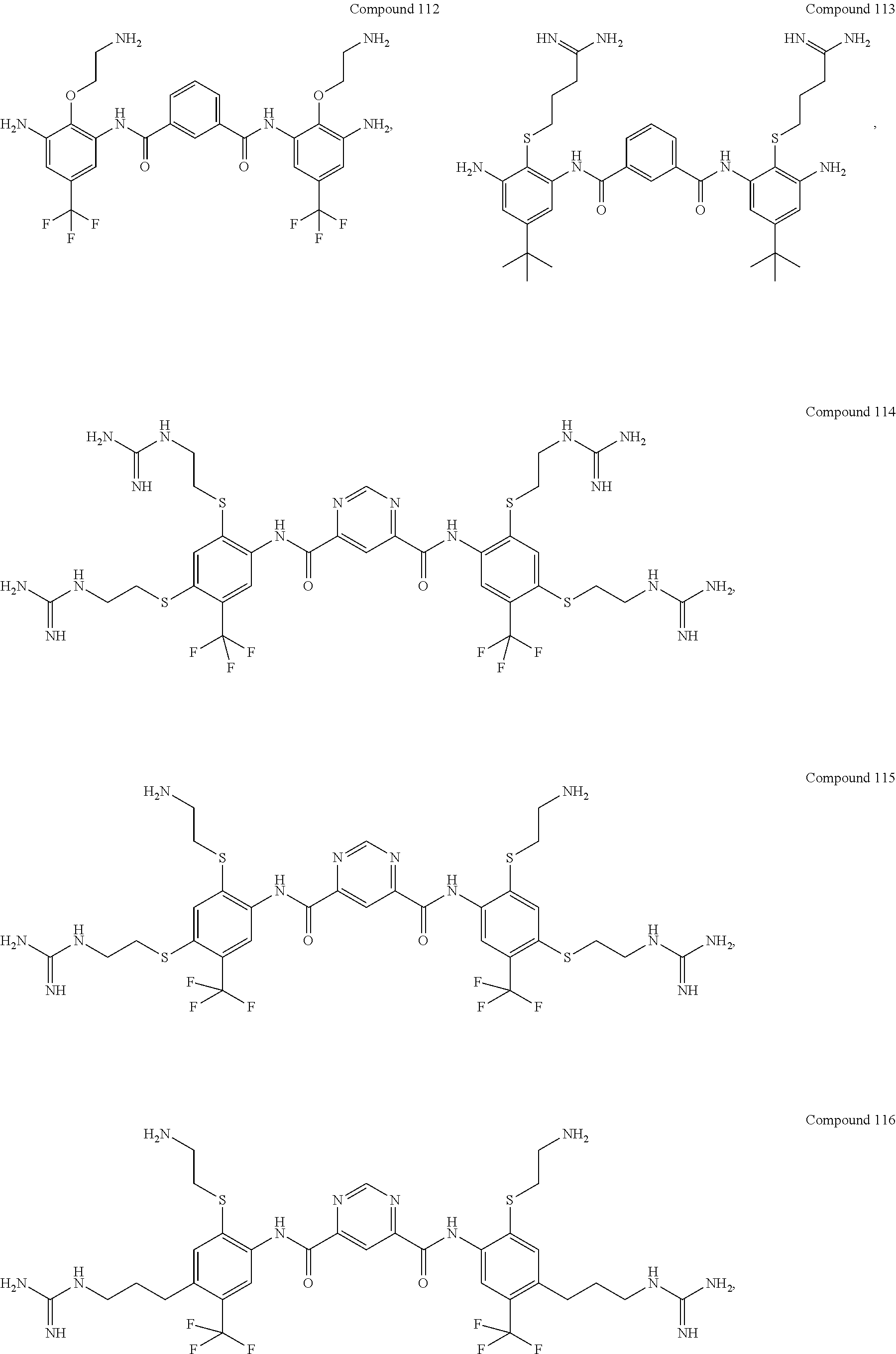 [SMILES: NCCOc1c(N)cc(C(F)(F)F)cc1NC(=O)c1cccc(C(=O)Nc2cc(C(F)(F)F)cc(N)c2OCCN)c1 ; CC(C)(C)c1cc(N)c(SCCCC(=N)N)c(NC(=O)c2cccc(C(=O)Nc3cc(C(C)(C)C)cc(N)c3SCCCC(=N)N)c2)c1 ; N=C(N)NCCSc1cc(SCCNC(=N)N)c(C(F)(F)F)cc1NC(=O)c1cc(C(=O)Nc2cc(C(F)(F)F)c(SCCNC(=N)N)cc2SCCNC(=N)N)ncn1 ; N=C(N)NCCSc1cc(SCCN)c(NC(=O)c2cc(C(=O)Nc3cc(C(F)(F)F)c(SCCNC(=N)N)cc3SCCN)ncn2)cc1C(F)(F)F ; N=C(N)NCCCc1cc(SCCN)c(NC(=O)c2cc(C(=O)Nc3cc(C(F)(F)F)c(CCCNC(=N)N)cc3SCCN)ncn2)cc1C(F)(F)F]

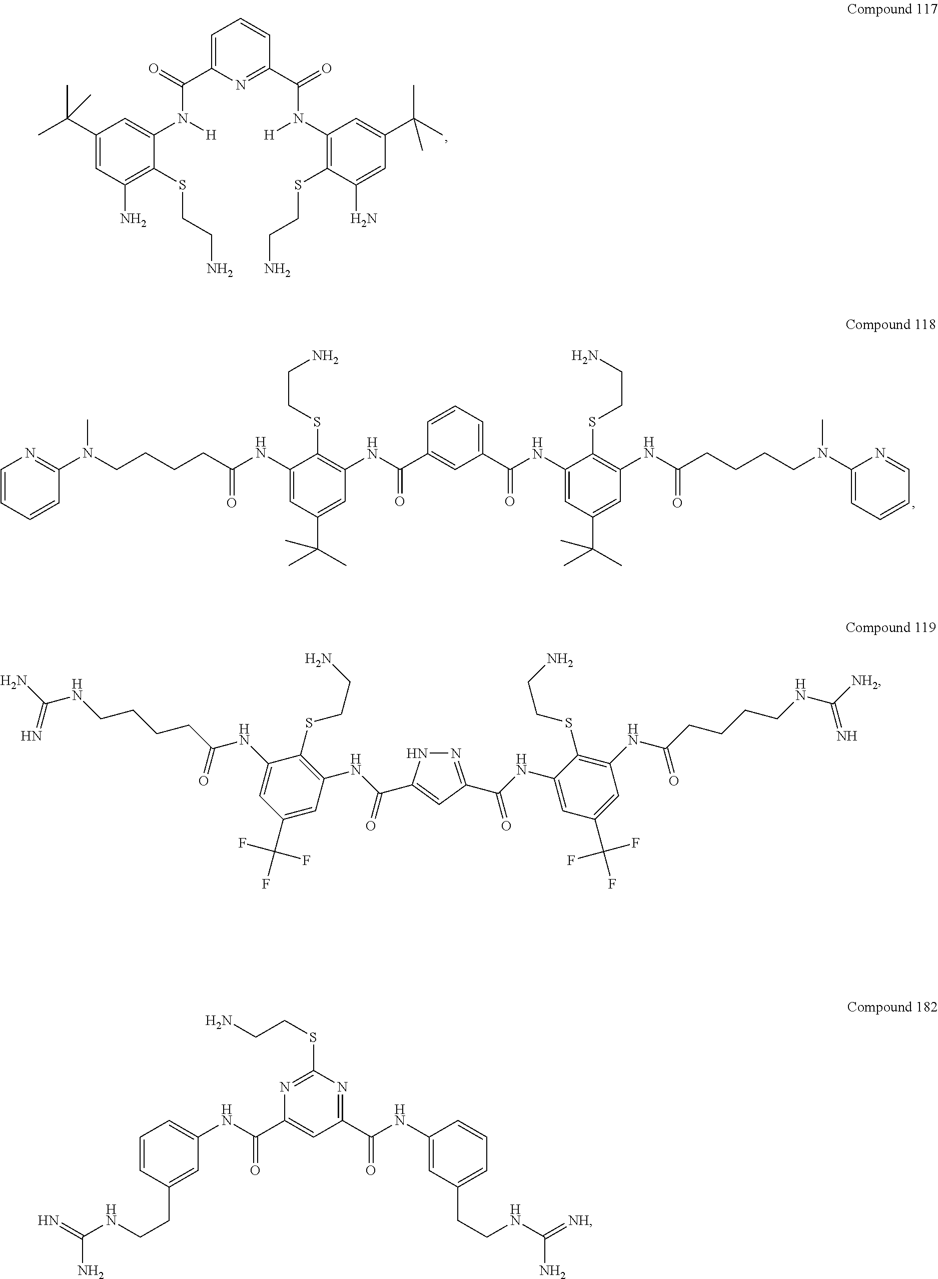 [SMILES: CC(C)(C)c1cc(N)c(SCCN)c(NC(=O)c2cccc(C(=O)Nc3cc(C(C)(C)C)cc(N)c3SCCN)n2)c1 ; CN(CCCCC(=O)Nc1cc(C(C)(C)C)cc(NC(=O)c2cccc(C(=O)Nc3cc(C(C)(C)C)cc(NC(=O)CCCCN(C)c4ccccn4)c3SCCN)c2)c1SCCN)c1ccccn1 ; N=C(N)NCCCCC(=O)Nc1cc(C(F)(F)F)cc(NC(=O)c2cc(C(=O)Nc3cc(C(F)(F)F)cc(NC(=O)CCCCNC(=N)N)c3SCCN)[nH]n2)c1SCCN ; N=C(N)NCCc1cccc(NC(=O)c2cc(C(=O)Nc3cccc(CCNC(=N)N)c3)nc(SCCN)n2)c1]

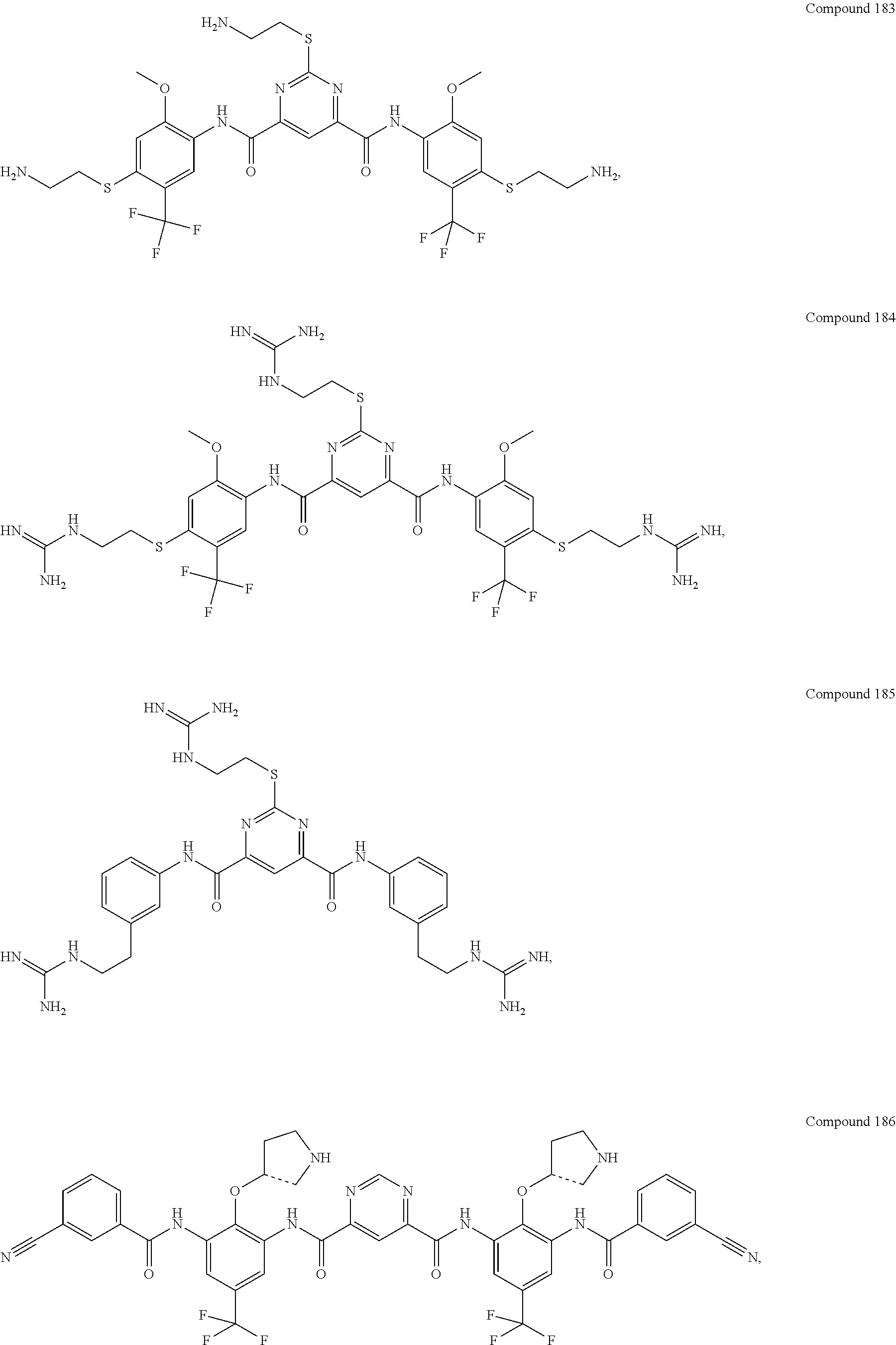

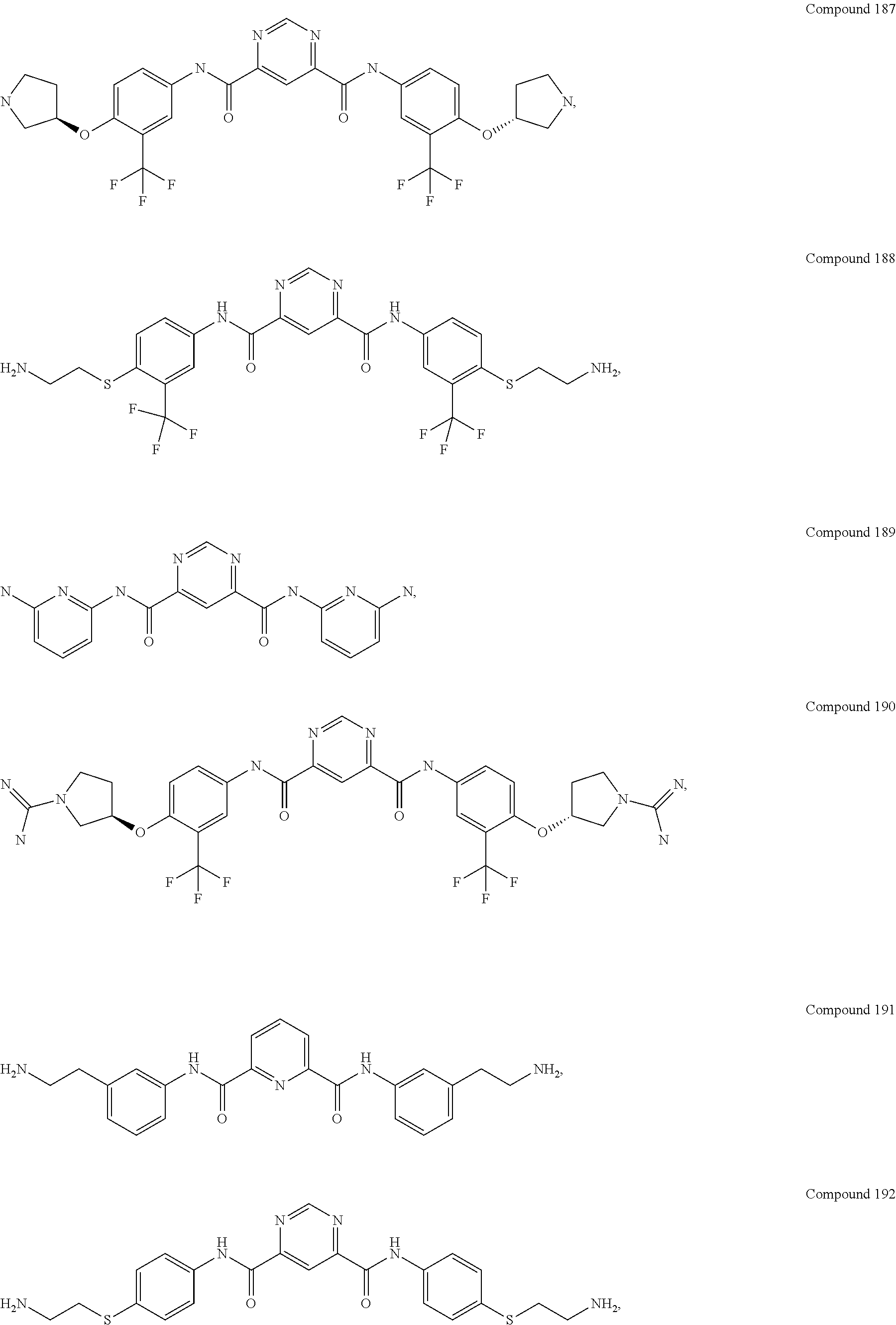

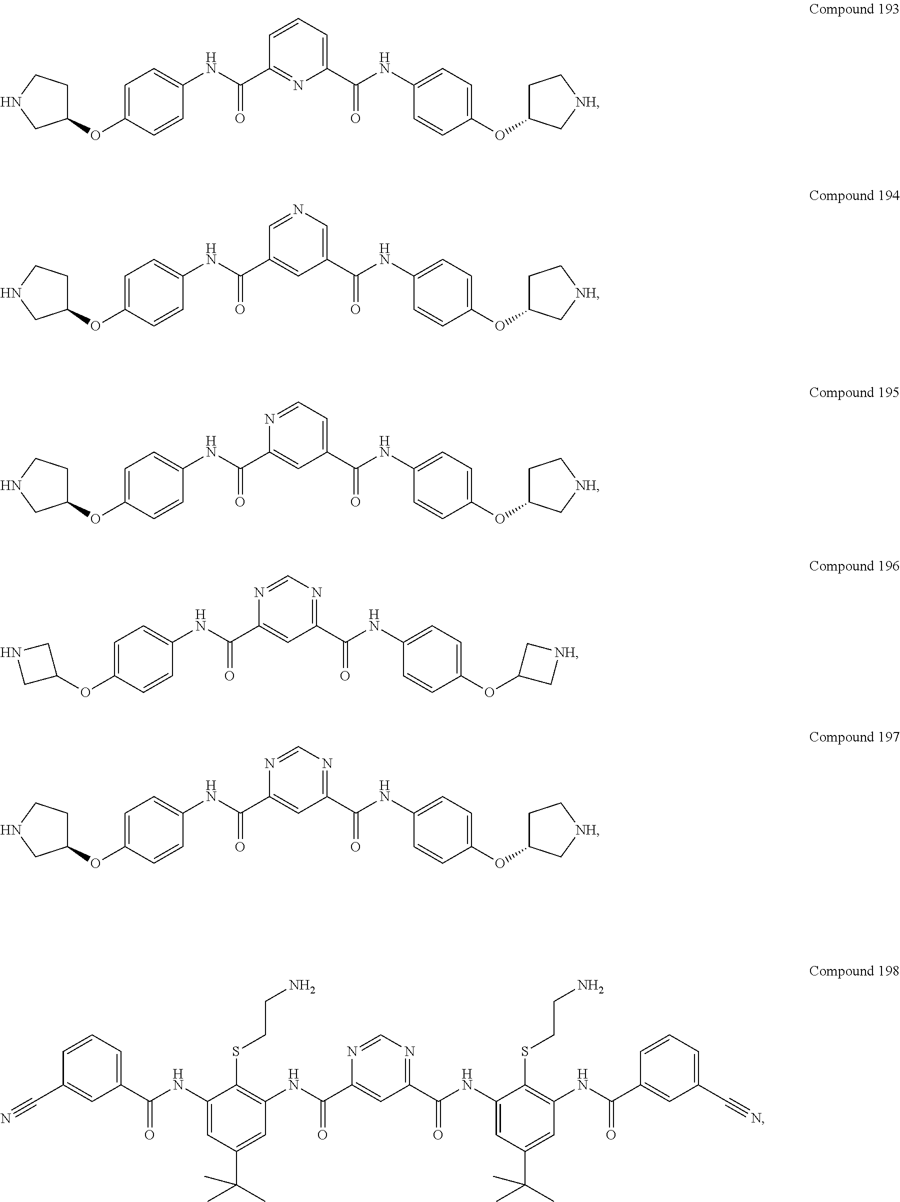 [SMILES: O=C(Nc1ccc(O[C@@H]2CCNC2)cc1)c1cccc(C(=O)Nc2ccc(O[C@@H]3CCNC3)cc2)n1 ; O=C(Nc1ccc(O[C@@H]2CCNC2)cc1)c1cncc(C(=O)Nc2ccc(O[C@@H]3CCNC3)cc2)c1 ; O=C(Nc1ccc(O[C@@H]2CCNC2)cc1)c1ccnc(C(=O)Nc2ccc(O[C@@H]3CCNC3)cc2)c1 ; O=C(Nc1ccc(OC2CNC2)cc1)c1cc(C(=O)Nc2ccc(OC3CNC3)cc2)ncn1 ; O=C(Nc1ccc(O[C@@H]2CCNC2)cc1)c1cc(C(=O)Nc2ccc(O[C@@H]3CCNC3)cc2)ncn1 ; CC(C)(C)c1cc(NC(=O)c2cccc(C#N)c2)c(SCCN)c(NC(=O)c2cc(C(=O)Nc3cc(C(C)(C)C)cc(NC(=O)c4cccc(C#N)c4)c3SCCN)ncn2)c1]

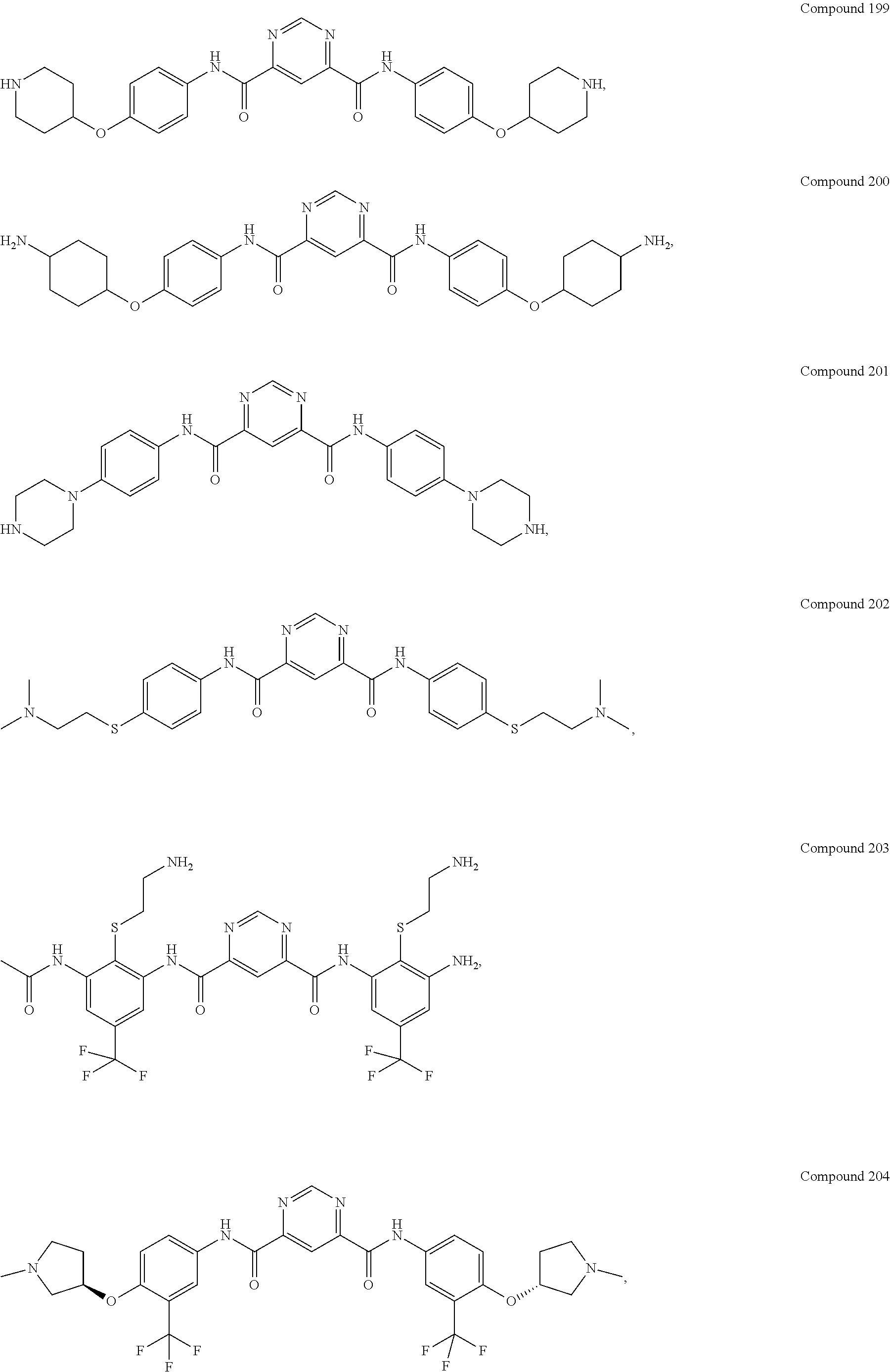

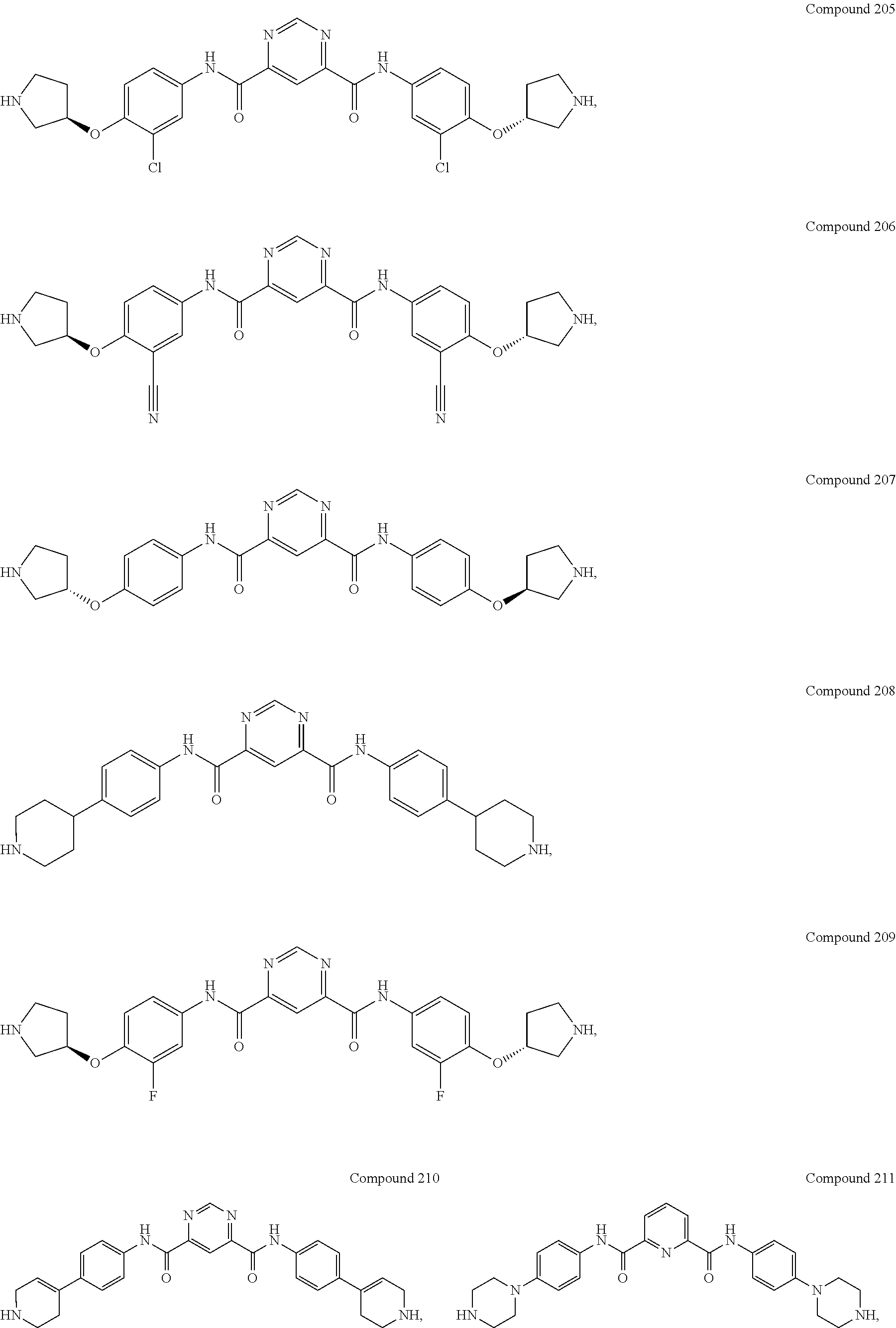

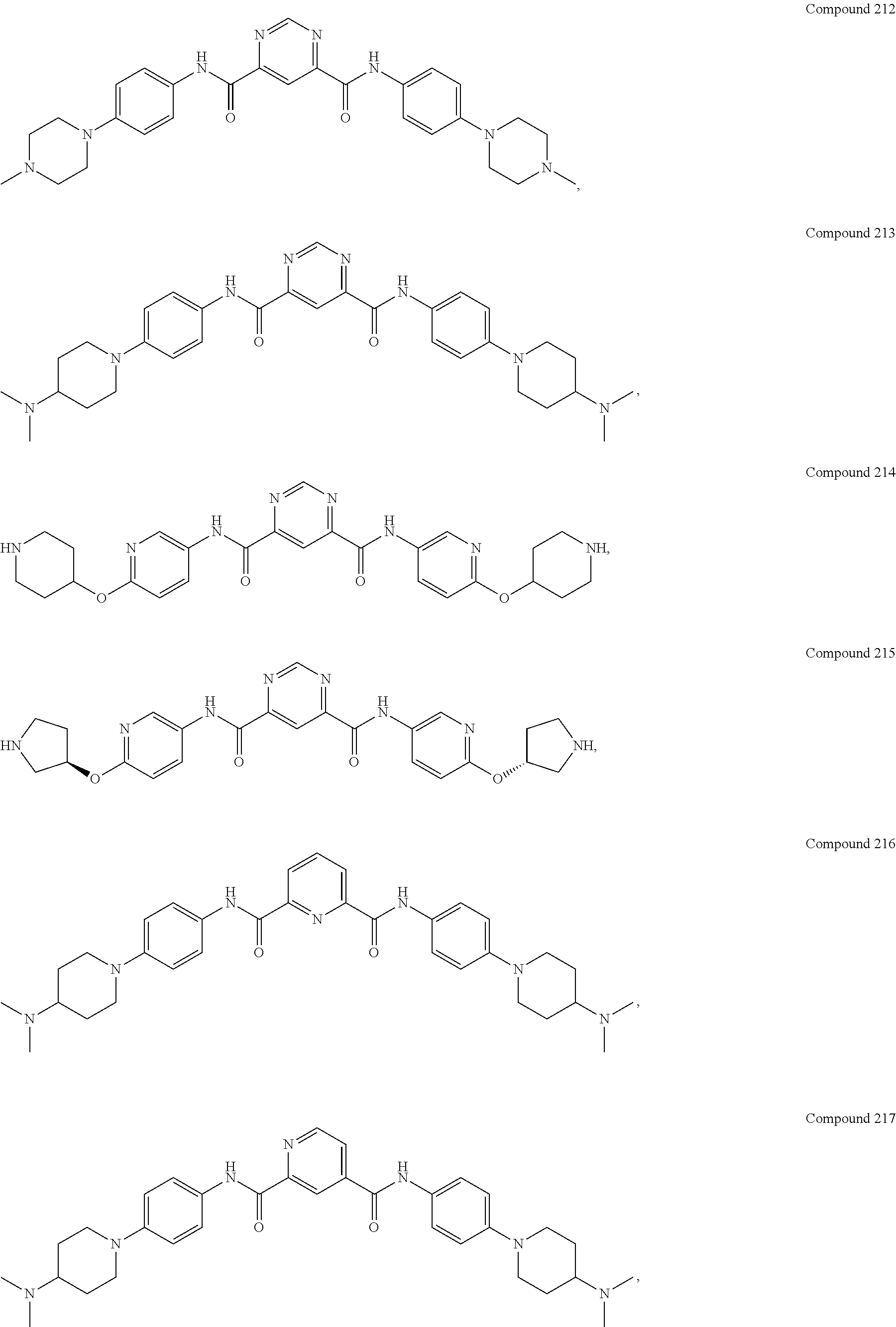

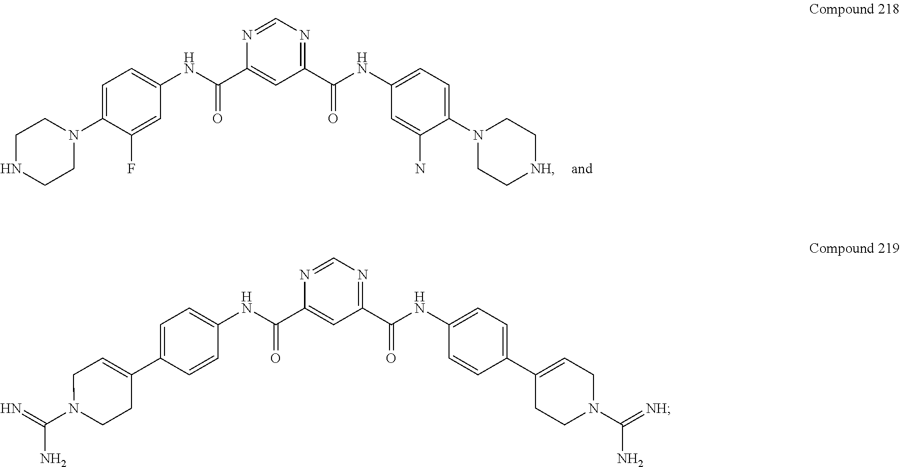 [SMILES: Nc1cc(NC(=O)c2cc(C(=O)Nc3ccc(N4CCNCC4)c(F)c3)ncn2)ccc1N1CCNCC1 ; "and" ; N=C(N)N1CC=C(c2ccc(NC(=O)c3cc(C(=O)Nc4ccc(C5=CCN(C(=N)N)CC5)cc4)ncn3)cc2)CC1]

or a pharmaceutically acceptable salt thereof.

In some embodiments, the compound is not Compound 105. In some embodiments, the compound is not any one or more of Compounds 182 to 219.

The present disclosure provides anti-malarial compounds of Formula II:

R¹-A₁-X—Z—X-A₁-R² (II)

or a pharmaceutically acceptable salt thereof,
wherein:
each X is, independently, —NR⁸, —N(R⁸)N(R⁸)—, O, or S, wherein each R⁸ is, independently, hydrogen or alkyl;

each Z is, independently, C═O, C═S, or O═S═O;

each A₁ is, independently, aryl optionally substituted with one or more W, or heteroaryl optionally substituted with one or more W;

each W is, independently, —CF₃, halo, C₁₋₄alkyl, —O—(CH₂)₁₋₅NHC(═NH)NH₂, —N((CH₂)₁₋₅—NH₂)((CH₂)₁₋₅N(C₄alkyl)₂), —N((CH₂)₁₋₅N(C₁₋₄alkyl)₂)₂, heterocycle, —S—(CH₂)₁₋₅NHC(═NH)NH₂, —S—(CH₂)₁₋₅NH₂, —S—(CH₂)₁₋₅N(CH₃)₂, —C₁₋₄alkyl, —S—(CH₂)₁₋₅NHS(═O)₂—C₁₋₄alkyl, O-heterocycle (wherein the heterocycle is optionally substituted with one or more cyano, amino, C₁₋₄alkyl, C₁₋₄alkoxy, guanidino, hydroxyl, amidino, or halo); and

R¹ and R² are, independently, hydrogen, halo, —NO₂, —S—(CH₂)₁₋₅NHC(═NH)NH₂, —O—(CH₂)₁₋₅NHC(═NH)NH₂, —CF₃, —N⁺(═O)O⁻, —(CH₂)₁₋₅NHC(═NH)NH₂, amino, —S—(CH₂)₁₋₅N(CH₃)₂, —O—(CH₂)₁₋₅N(CH₃)₂, —O-heterocycle (wherein the heterocycle is optionally substituted), —S-heterocycle (wherein the heterocycle is optionally substituted), —NHC(═O)—C₁₋₄alkyl, —NHC(═O)—(CH₂)₁₋₅NHC(═NH)NH₂, —NHC(═O)—(CH₂)₁₋₅-aryl (wherein either or both the —(CH₂)₁₋₅ or phenyl is optionally substituted), —NHC(═O)—(CH₂)₁₋₅NH-aryl (wherein either or both —NH and/or aryl is optionally substituted), —NHC(═O)-aryl (wherein the aryl is optionally substituted), or —NHC(═O)—(CH₂)₁₋₅NHS(═O)₂—C₁₋₄alkyl, wherein the optional substituents are chosen from one or more cyano, amino, C₁₋₄alkyl, guanidino, hydroxyl, amidino, C₁₋₄alkoxy, CF₃, and halo.

In some embodiments, each X is, independently, —NR⁸, O, or S, wherein each R⁸ is, independently, hydrogen or alkyl. In some embodiments, each X is, independently, —NR⁸, wherein each R⁸ is, independently, hydrogen or C₁₋₄alkyl. In some embodiments, each X is NH.

In some embodiments, each Z is C═O.

In some embodiments, each A₁ is, independently, phenyl, pyridine, pyrimidine, pyrazine, or pyrazole, each optionally substituted. In some embodiments, each A₁ is, independently, phenyl, pyridine, pyrimidine, pyrazine, or pyrazole, each of which is substituted. In some embodiments, each A₁ is, independently, phenyl, pyridine, pyrimidine, or pyrazole, each of which is substituted. In some embodiments, both A₁ are substituted phenyl.

In some embodiments, each W is, independently, —CF₃, halo, C₁₋₄alkyl, —O—(CH₂)₄NHC(═NH)NH₂, —N((CH₂)₂—NH₂)((CH₂)₂N(Et)₂), —N((CH₂)₂N(Et)₂)₂, or piperazine. In some embodiments, each W is, independently, —CF₃, Cl, BR, F, —C(CH₃)₃, —O—(CH₂)₄NHC(═NH)NH₂, —N((CH₂)₂—NH₂)((CH₂)₂N(Et)₂), —N((CH₂)₂N(Et)₂)₂, or piperazine.

In some embodiments, R¹ and R² are, independently, hydrogen, Cl, F, —NO₂, —S—(CH₂)₂NHC(═NH)NH₂, —O—(CH₂)₄NHC(═NH)NH₂, —CF₃, or —N⁺(═O)O⁻.

In some embodiments: each X is NH; each Z is C═O; each A₁ is, independently, substituted phenyl; R¹ and R² are, independently, hydrogen, halo, —NO₂, —N⁺(═O)O⁻, —CF₃, —S—(CH₂)₁₋₅NHC(═NH)NH₂, or —O—(CH₂)₁₋₅NHC(═NH)NH₂; and each W is, independently, —CF₃, halo, C₁₋₄alkyl, —O—(CH₂)₁₋₅NHC(═NH)NH₂, —N((CH₂)₁₋₅—NH₂)((CH₂)₁₋₅N(C₁₋₄alkyl)₂), —N((CH₂)₁₋₅N(C₁₋₄alkyl)₂)₂, or heterocycle.

In some embodiments: each X is NH; each Z is C═O; each A₁ is substituted phenyl; R¹ and R² are, independently, hydrogen, Cl, F, —NO₂, —S—(CH₂)₂NHC(═NH)NH₂, —CF₃, —N⁺(═O)O⁻, or —O—(CH₂)₄NHC(═NH)NH₂; and each W is, independently, —CF₃, Cl, —C(CH₃)₃, —O—(CH₂)₄NHC(═NH)NH₂, —N((CH₂)₂—NH₂)((CH₂)₂N(Et)₂), —N((CH₂)₂N(Et)₂)₂, or piperazine.

In some embodiments, the compound of Formula II is chosen from:

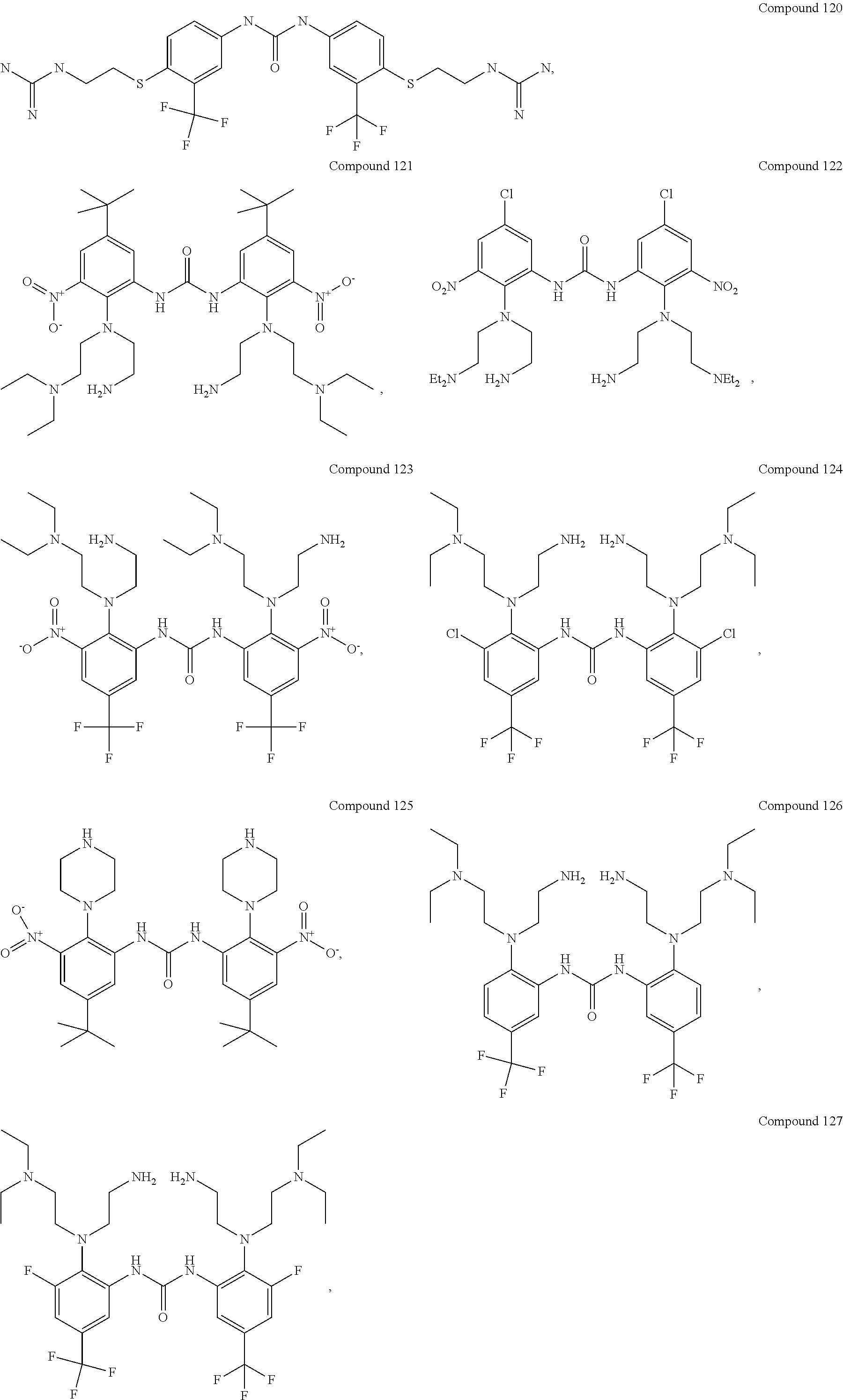

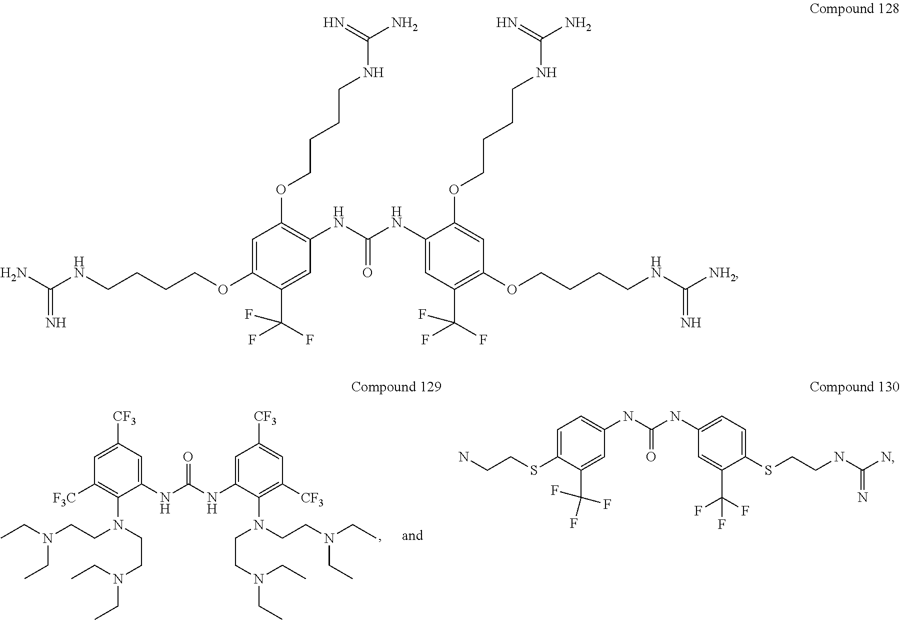

or a pharmaceutically acceptable salt thereof.

The present disclosure provides anti-malarial compounds of Formula IIa:

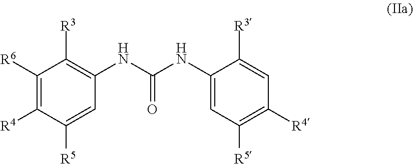

or a pharmaceutically acceptable salt thereof,
wherein:

R³ and R^{3′} are, independently, H or —O—C₁₋₄alkyl;

R⁶ is H, —(CH₂)₁₋₄NHC(═NH)NH₂, or —(CH₂)₁₋₄NH₂;

R⁵ and R^{5′} are, independently, H, —CF₃, —(CH₂)₁₋₄NHC(═NH)NH₂, or —(CH₂)₁₋₄NH₂; and

R⁴ and R^{4′}are, independently, H, —S—(CH₂)₁₋₄NHC(═NH)NH₂, —S—(CH₂)₁₋₄NH₂,

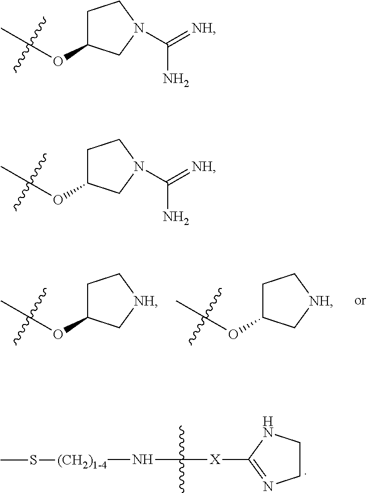

In some embodiments, R³ and R^{3′}are both H. In some embodiments, R³ is —O—CH₃ and R^{3′} is H. In some embodiments, R³ and R^{3′}are both —O—CH₃.

In some embodiments, R⁶ is H or —(CH₂)₂NHC(═NH)NH₂.

In some embodiments, both R⁵ and R^{5′} are —CF₃. In some embodiments, both R⁵ and R^{5′} are —(CH₂)₂NHC(═NH)NH₂. In some embodiments, R⁵ is H and R^{5′}is —CF₃.

In some embodiments, both R⁴ and R^{4′} are H, —S—(CH₂)₂₋₃NH₂, or

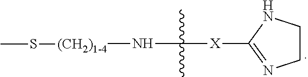

In some embodiments, R⁴ is H and R^{4′} is —S—(CH₂)₂NHC(═NH)NH₂. In some embodiments, R⁴ is

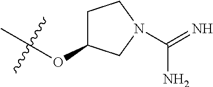 [SMILES: *O[C@H]1CCN(C(=N)N)C1]

### and R^{4′} is

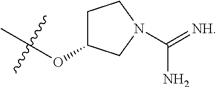 [SMILES: *O[C@@H]1CCN(C(=N)N)C1]

In some embodiments, R⁴ is

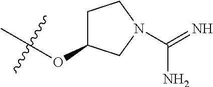 [SMILES: *O[C@H]1CCN(C(=N)N)C1]

and R^{4′} is —S—(CH₂)₂NHC(═NH)NH₂. In some embodiments, R⁴ is

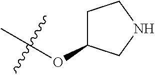 [SMILES: *O[C@H]1CCNC1]

### and R^{4′} is

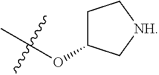 [SMILES: *O[C@@H]1CCNC1]

In some embodiments, R⁴ is —S—(CH₂)₂NHC(═NH)NH₂ and R^{4′} is —S—(CH₂)₃NH₂. In some embodiments, R⁴ is —S—(CH₂)₂NH₂ and R^{4′} is —S—(CH₂)₂NHC(═NH)NH₂.

In some embodiments, the compound of Formula IIa is chosen from:

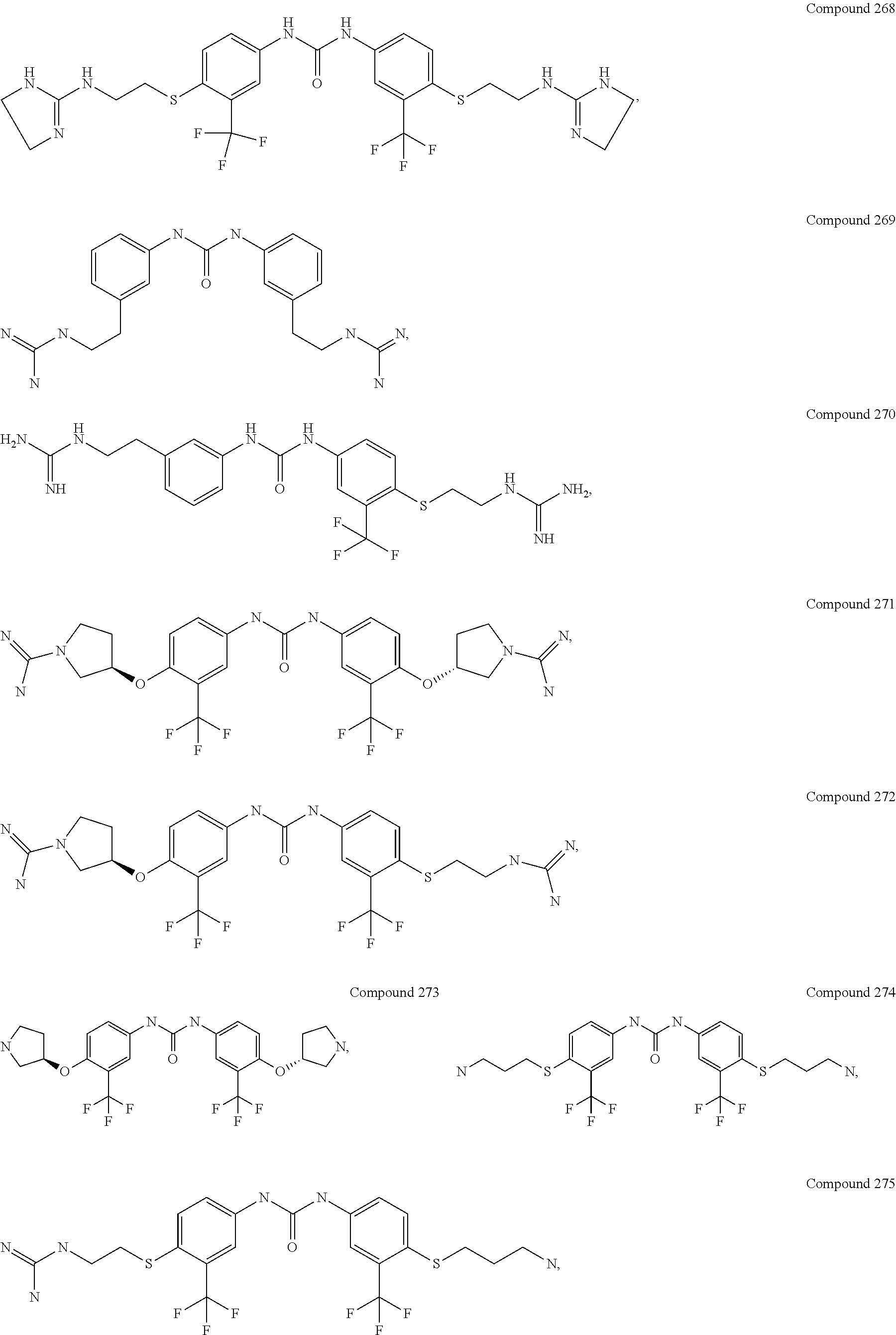

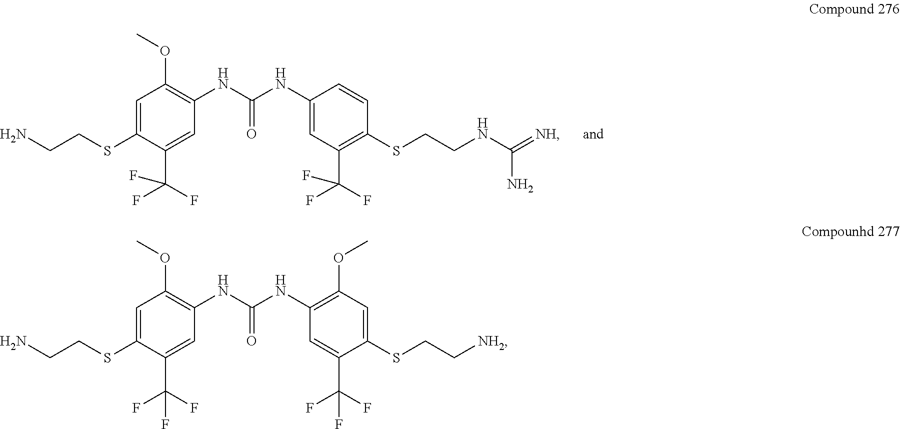

or a pharmaceutically acceptable salt thereof.

In some embodiments, the compound is not any one or more of Compounds 268 to 277.

The present disclosure also provides anti-malarial compounds of Formula III:

—R¹—X-A₁-A₂-A₁-X—R² (III)

or a pharmaceutically acceptable salt thereof,
wherein:

A₂ is aryl optionally substituted with one or more W, or heteroaryl optionally substituted with one or more W;

each A₁ is, independently, aryl optionally substituted with one or more W, or heteroaryl optionally substituted with one or more W;

each X is, independently, absent, —NR⁸, —N(R⁸)N(R⁸)—, O, or S, wherein each R⁸ is, independently, hydrogen or alkyl;

R¹ and R² are, independently, absent, —C(═O)—(CH₂)₁₋₅NHC(═NH)NH₂, hydrogen, —(CH₂)₁₋₅NHC(═NH)NH₂, —C(═O)—CH(NH₂)C₁₋₄alkyl, halo, —NO₂, —CF₃, —N⁺(═O)O⁻, amino, —S—(CH₂)₁₋₅NHC(═NH)NH₂, —O—(CH₂)₁₋₅NHC(═NH)NH₂, —(CH₂)₁₋₅NHC(═NH)NH₂, —S—(CH₂)₁₋₅N(CH₃)₂, —O—(CH₂)₁₋₅N(CH₃)₂, —NHC(═O)—(CH₂)₁₋₅NHC(═NH)NH₂, —NHC(═O)—C₁₋₄alkyl, or —NHC(═O)—(CH₂)₁₋₅NHS(═O)₂—C₁₋₄alkyl; and

each W is, independently, —O—(CH₂)₁₋₅NHC(═NH)NH₂, —O—(CH₂)₁₋₅NH₂, —CF₃, halo, —C₁₋₄alkyl, —N((CH₂)₁₋₅—NH₂)((CH₂)₁₋₅N(C₁₋₄alkyl)₂), —N((CH₂)₁₋₅N(C₁₋₄alkyl)₂)₂, —C₁₋₄alkylamino, —(CH₂)₁₋₅NHC(═NH)NH₂, —S—(CH₂)₁₋₅NHC(═NH)NH₂, —S—(CH₂)₁₋₅NH₂, —S—(CH₂)₁₋₅N(CH₃)₂, —S—(CH₂)₁₋₅NHS(═O)₂—C₁₋₄alkyl, —C≡C—(CH₂)₁₋₅—NHC(═NH)NH₂, —C≡C—(CH₂)₁₋₅—NH₂, or aryl or heterocycle each optionally substituted with one or more —CF₃, cyano, amino, C₁₋₄alkyl, C₁₋₄alkoxy, guanidino, hydroxyl, amidino, halo, —(CH₂)₁₋₄NH₂, —O—(CH₂)₁₋₄NH₂, —(CH₂)₁₋₄NHC(═NH)NH₂, or —O—(CH₂)₁₋₄NHC(═NH)NH₂.

In some embodiments, A₂ is oxadiazole, pyridine, pyrimidine, thienyl, furyl, thiazolyl, imidazolyl, triazolyl, or phenyl, each of which is optionally substituted. In some embodiments, A₂ is oxadiazole, pyridine, pyrimidine, or phenyl, each of which is optionally substituted.

In some embodiments, each A₁ is, independently, phenyl or pyridine. In some embodiments, each A₁ is, independently, phenyl, pyrimidine, or pyridine.

In some embodiments, each X is, independently, absent, —NH or O.

In some embodiments, R¹ and R² are, independently, absent, —(CH₂)₃NHC(═NH)NH₂, —C(═O)—(CH₂)₄NHC(═NH)NH₂, or —C(═O)—CH(NH₂)CH₃.

In some embodiments, each W is, independently, —O—(CH₂)₁₋₅NHC(═NH)NH₂, —CF₃, —O—(CH₂)₁₋₅NH₂, —(CH₂)₁₋₅NHC(═NH)NH₂, —C≡C—(CH₂)₁₋₅—NHC(═NH)NH₂, halo, —C₁₋₄alkyl, —C₁₋₄alkylamino, —C≡C—(CH₂)₁₋₅—NH₂, or phenyl or piperazine each optionally substituted with one or more —CF₃, cyano, amino, guanyl, C₁₋₄alkyl, C₁₋₄alkoxy, guanidino, hydroxyl, amidino, halo, —(CH₂)₁₋₄NH₂, —O—(CH₂)₁₋₄NH₂, —(CH₂)₁₋₄NHC(═NH)NH₂, or —O—(CH₂)₁₋₄NHC(═NH)NH₂.

In some embodiments, each W is, independently, —O—(CH₂)₁₋₅NHC(═NH)NH₂, —CF₃, —O—(CH₂)₁₋₅NH₂, —(CH₂)₁₋₅NHC(═NH)NH₂, —C≡C—(CH₂)₁₋₅—NHC(═NH)NH₂, halo, —C₁₋₄alkyl, —C≡C—(CH₂)₁₋₅—NH₂, or phenyl optionally substituted with one or more —CF₃, cyano, amino, guanyl, C₁₋₄alkyl, C₁₋₄alkoxy, guanidino, hydroxyl, amidino, or halo. In some embodiments, each W is, independently, —O—(CH₂)₃NHC(═NH)NH₂, —O—(CH₂)₃NH₂, —C(CH₃)₃, F, —CF₃, —(CH₂)₂₋₃NHC(═NH)NH₂, —C≡C—(CH₂)₁₋₅—NHC(═NH)NH₂, —C≡C—(CH₂)₂—NH₂, or phenyl optionally substituted with one or more —CF₃.

In some embodiments, A₂ is aryl optionally substituted with one or more W, or heteroaryl optionally substituted with one or more W; each A₁ is, independently, aryl optionally substituted with one or more W, or heteroaryl optionally substituted with one or more W; each X is, independently, absent, —NR⁸, —N(R⁸)N(R⁸)—, O, or S, wherein each R⁸ is, independently, hydrogen or alkyl; R¹ and R² are, independently, absent, —C(═O)—(CH₂)₁₋₅NHC(═NH)NH₂, hydrogen, —(CH₂)₁₋₅NHC(═NH)NH₂, —C(═O)—CH(NH₂)C₁₋₄alkyl, halo, —NO₂, —CF₃, —N⁺(═O)O⁻, amino, —S—(CH₂)₁₋₅NHC(═NH)NH₂, —O—(CH₂)₁₋₅NHC(═NH)NH₂, —(CH₂)₁₋₅NHC(═NH)NH₂, —S—(CH₂)₁₋₅N(CH₃)₂, —O—(CH₂)₁₋₅N(CH₃)₂, —NHC(═O)—(CH₂)₁₋₅NHC(═NH)NH₂, —NHC(═O)—C₁₋₄alkyl, or —NHC(═O)—(CH₂)₁₋₅NHS(═O)₂—C₁₋₁₄alkyl; and each W is, independently, —O—(CH₂)₁₋₅NHC(═NH)NH₂, —O—(CH₂)₁₋₅NH₂, —CF₃, halo, C₁₋₄alkyl, —C₁₋₄alkyl, —N((CH₂)₁₋₅—NH₂)((CH₂)₁₋₅N(C₁₋₄alkyl)₂), —N((CH₂)₁₋₅N(C₁₋₄alkyl)₂)₂, —(CH₂)₁₋₅NHC(═NH)NH₂, —S—(CH₂)₁₋₅NHC(═NH)NH₂, —S—(CH₂)₁₋₅NH₂, —S—(CH₂)₁₋₅N(CH₃)₂, —S—(CH₂)₁₋₅NHS(═O)₂—C₁₋₄alkyl, —C≡C—(CH₂)₁₋₅—NHC(═NH)NH₂, —C≡C—(CH₂)₁₋₅—NH₂, or aryl optionally substituted with one or more —CF₃, cyano, amino, C₁₋₄alkyl, C₁₋₄alkoxy, guanidino, hydroxyl, amidino, or halo.

In some embodiments, the compound of Formula III is chosen from:

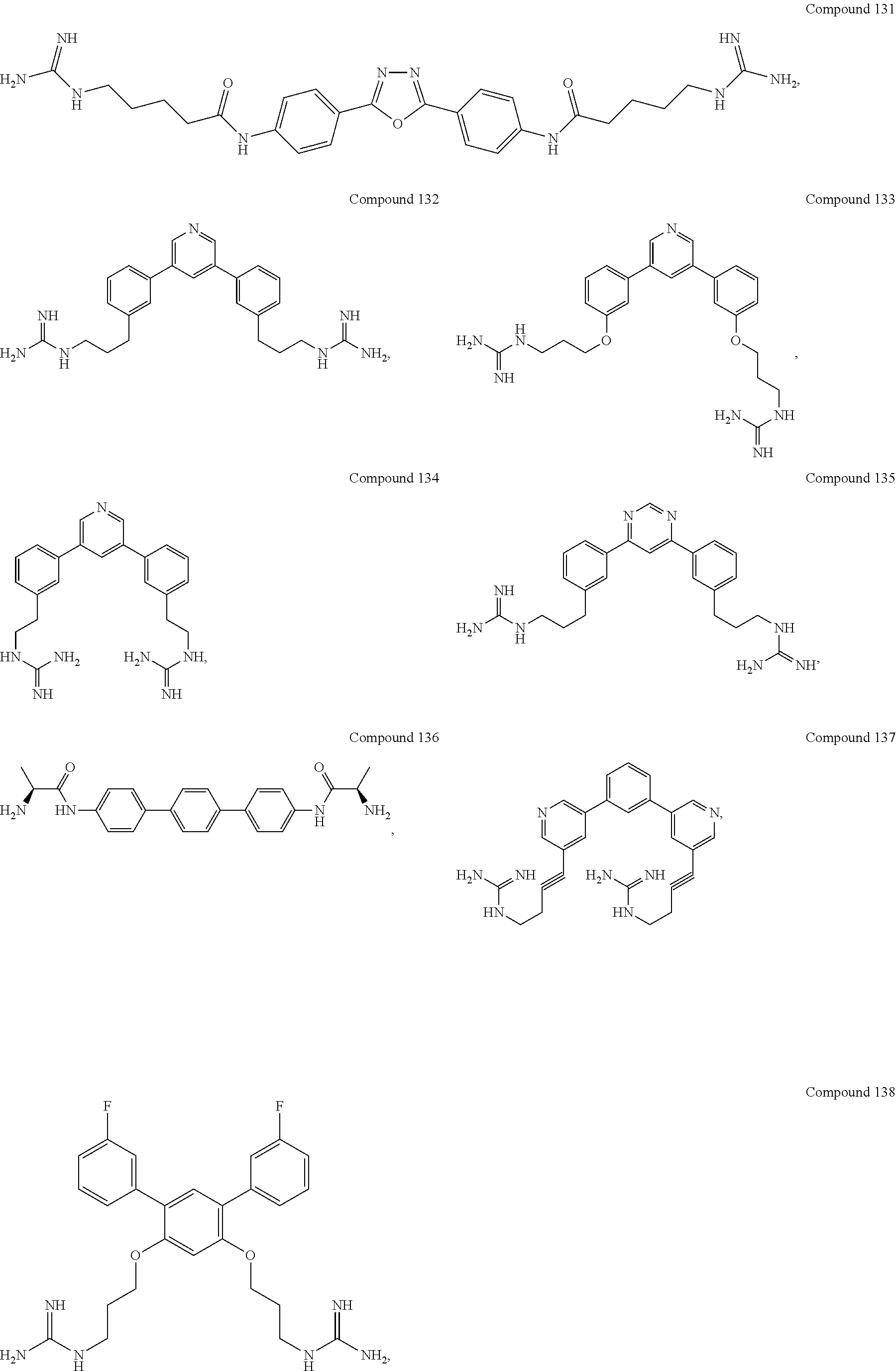

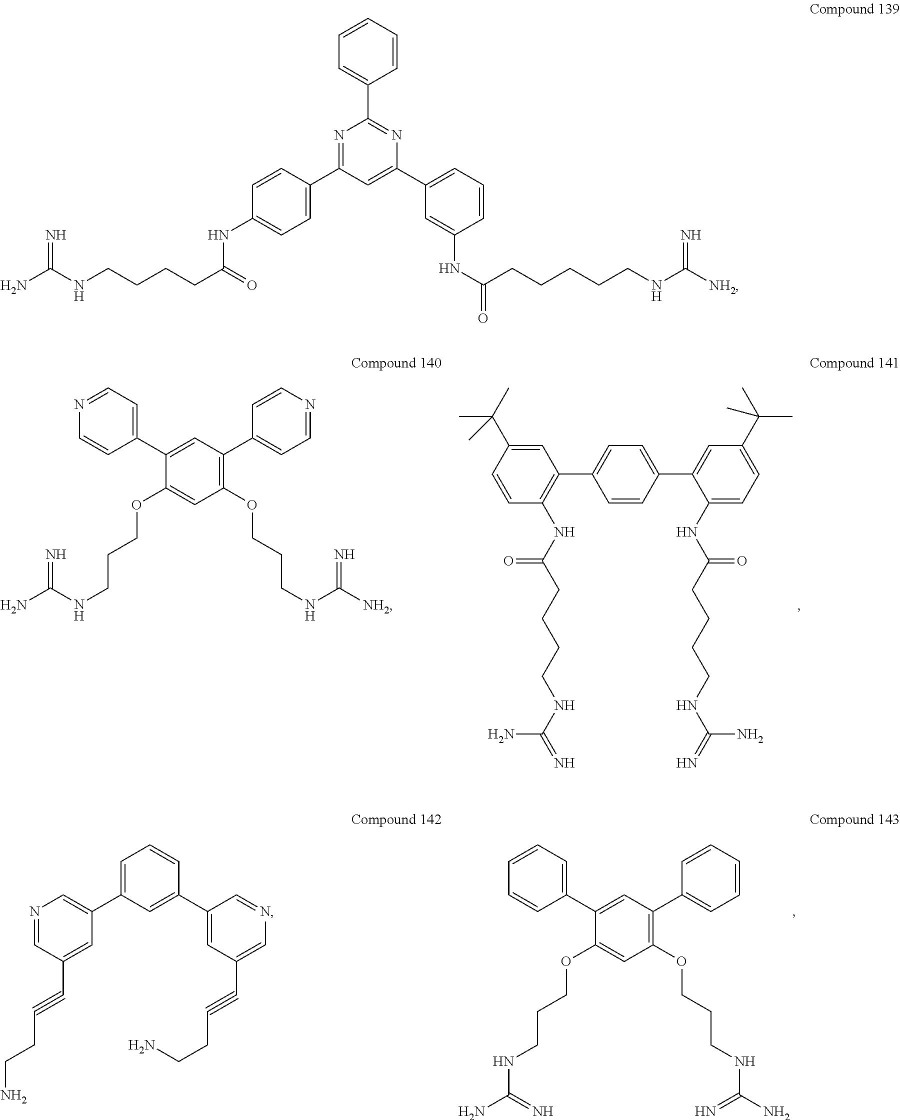 [SMILES: N=C(N)NCCCCCC(=O)Nc1cccc(-c2cc(-c3ccc(NC(=O)CCCCNC(=N)N)cc3)nc(-c3ccccc3)n2)c1 ; N=C(N)NCCCOc1cc(OCCCNC(=N)N)c(-c2ccncc2)cc1-c1ccncc1 ; CC(C)(C)c1ccc(NC(=O)CCCCNC(=N)N)c(-c2ccc(-c3cc(C(C)(C)C)ccc3NC(=O)CCCCNC(=N)N)cc2)c1 ; NCCC#Cc1cncc(-c2cccc(-c3cncc(C#CCCN)c3)c2)c1 ; N=C(N)NCCCOc1cc(OCCCNC(=N)N)c(-c2ccccc2)cc1-c1ccccc1]

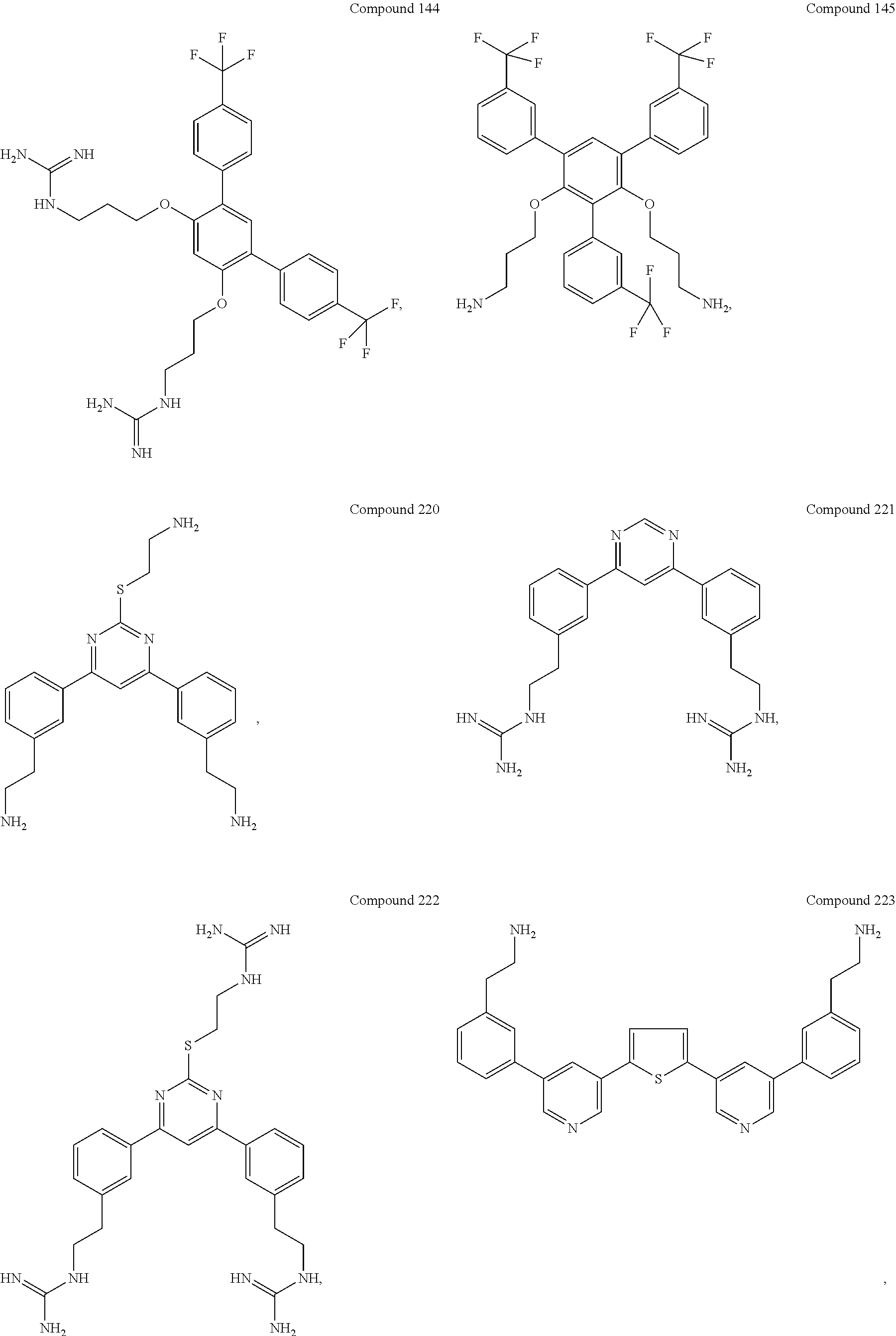 [SMILES: N=C(N)NCCCOc1cc(OCCCNC(=N)N)c(-c2ccc(C(F)(F)F)cc2)cc1-c1ccc(C(F)(F)F)cc1 ; NCCCOc1c(-c2cccc(C(F)(F)F)c2)cc(-c2cccc(C(F)(F)F)c2)c(OCCCN)c1-c1cccc(C(F)(F)F)c1 ; NCCSc1nc(-c2cccc(CCN)c2)cc(-c2cccc(CCN)c2)n1 ; N=C(N)NCCc1cccc(-c2cc(-c3cccc(CCNC(=N)N)c3)ncn2)c1 ; N=C(N)NCCSc1nc(-c2cccc(CCNC(=N)N)c2)cc(-c2cccc(CCNC(=N)N)c2)n1 ; NCCc1cccc(-c2cncc(-c3ccc(-c4cncc(-c5cccc(CCN)c5)c4)s3)c2)c1]

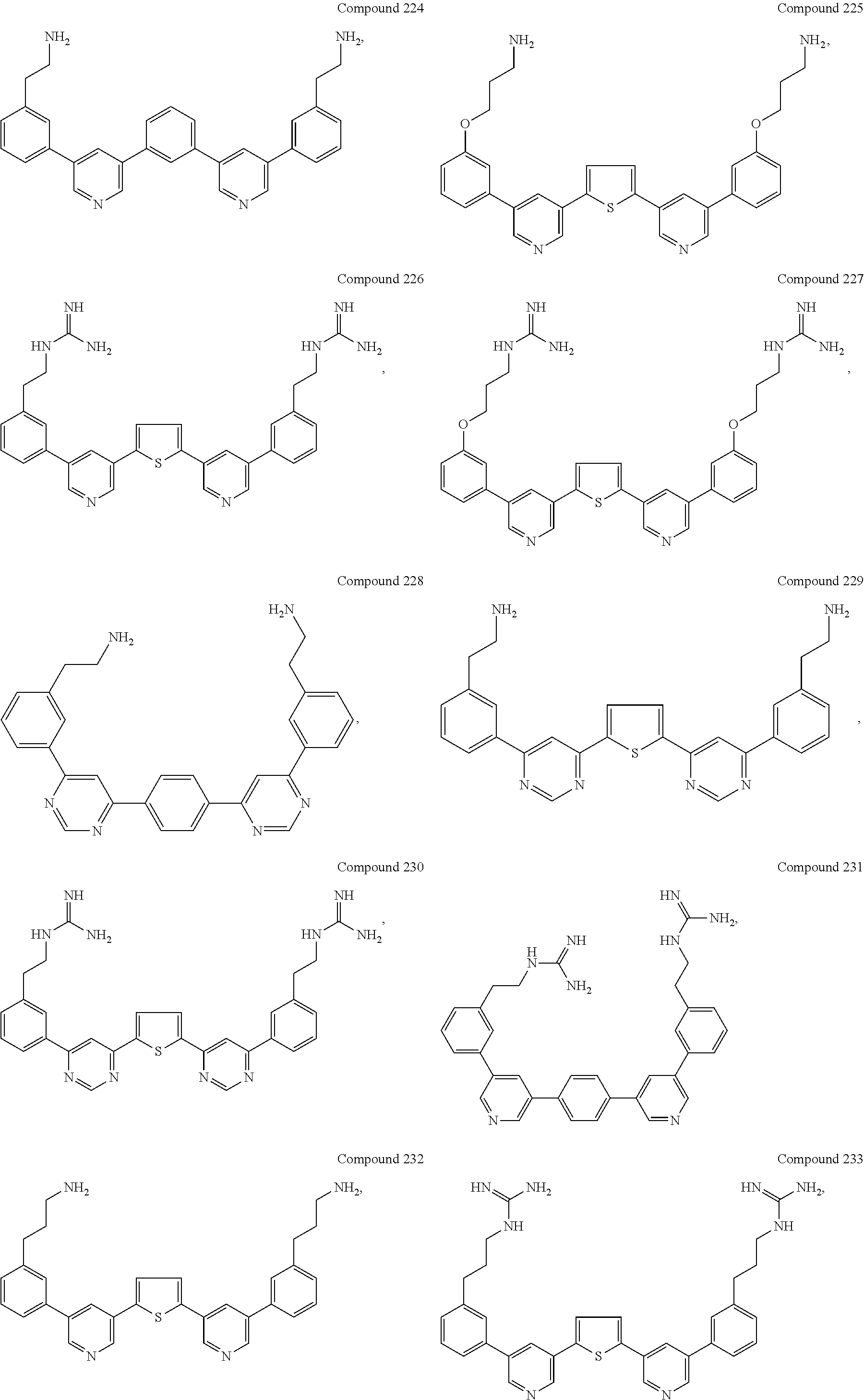

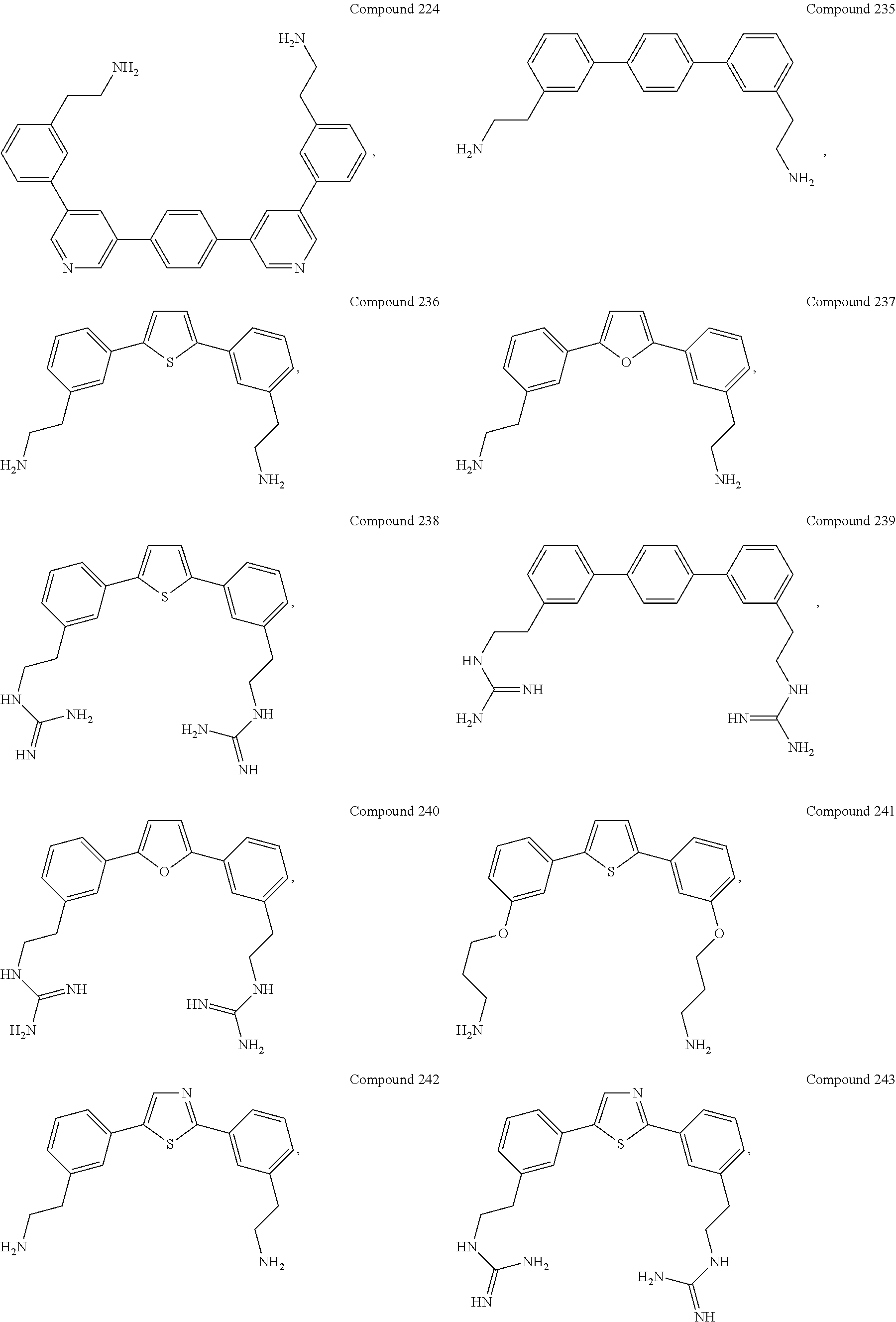

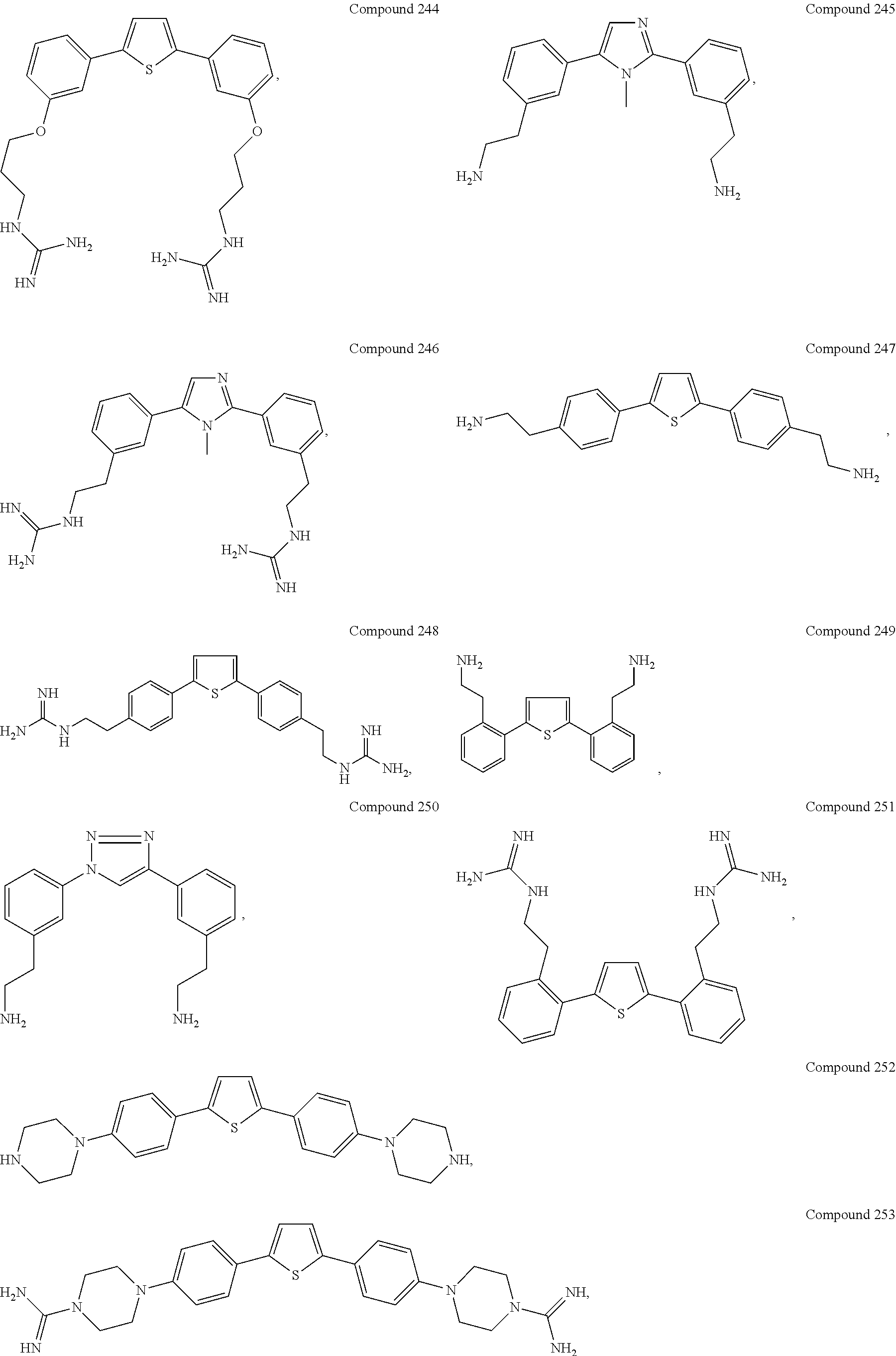

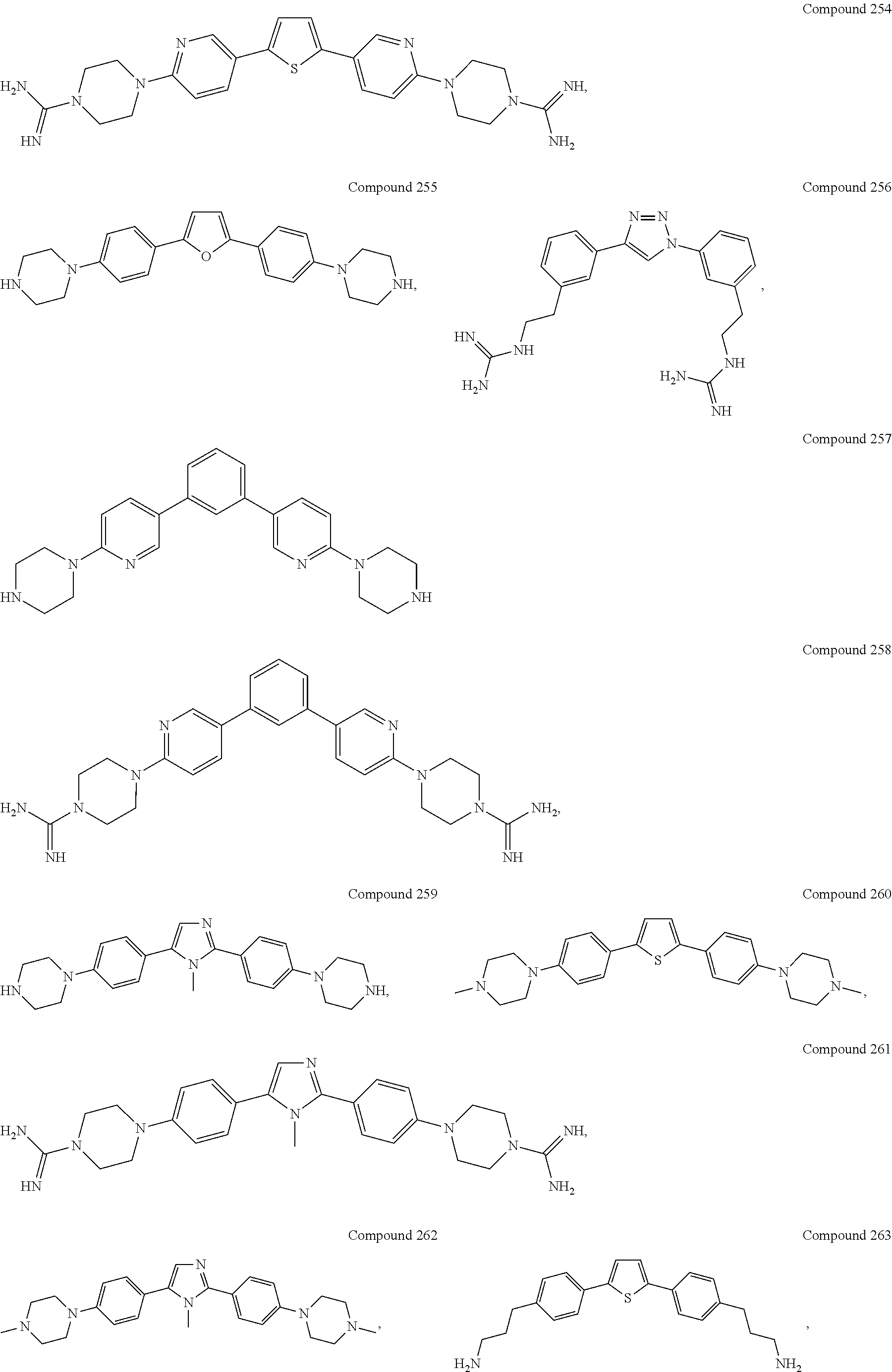

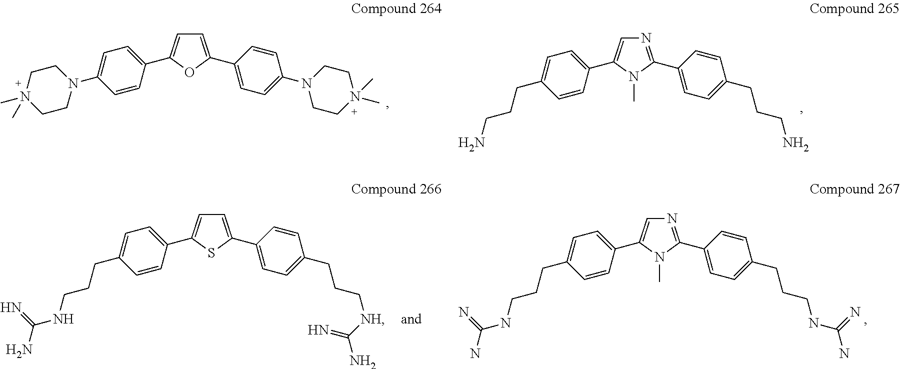

or a pharmaceutically acceptable salt thereof.

In some embodiments, the compound is not any one or more of Compounds 220 to 267.

The present disclosure also provides anti-malarial compounds of Formula IV:

Het¹-X—Y-Het²-Y—X-Het¹ (IV)

or a pharmaceutically acceptable salt thereof,
wherein:

each X is, independently, —NR⁸, —N(R⁸)N(R⁸)—, O, or S, wherein each R⁸ is, independently, hydrogen or alkyl;

each Y is, independently, C═O, C═S, or O═S═O;

each Het¹ is, independently, a fused bicyclic ring chosen from naphthalene, isobenzofuran, indolizine, isoindole, indole, purine, isoquinoline, quinolone, phthalazine, naphthyridine, quinoxaline, quinazoline, pteridine, chroman, isochroman, indoline, isoindoline, and

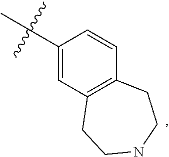 [SMILES: *c1ccc2c(c1)CCNCC2]

each of which is optionally substituted with one or more W;

Het² is an aryl or heteroaryl chosen from thiophene, furan, pyran, pyrrole, imidazole, pyrazole, isothiaole, isoxazole, pyridine, pyrazine, pyrimidine, and pyridazine; and each W is, independently, —(CH₂)₁₋₅NH₂, —C₁₋₄alkyl, —(CH₂)₁₋₅NHC(═NH)NH₂, —O—(CH₂)₁₋₅NHC(═NH)NH₂, —O—(CH₂)₁₋₅NH₂, —CF₃, halo, C₁₋₄alkyl, —S—(CH₂)₁₋₅NH₂, —N((CH₂)₁₋₅N(C₁₋₄alkyl)₂)₂, —N((CH₂)₁₋₅—NH₂)((CH₂)₁₋₅N(C₁₋₄alkyl)₂), —S—(CH₂)₁₋₅N(CH₃)₂, —(CH₂)₁₋₅NHC(═NH)NH₂, —S—(CH₂)₁₋₅NHC(═NH)NH₂, —S—(CH₂)₁₋₅NHS(═O)₂—C₁₋₄alkyl, or cycloalkyl optionally substituted with one or more —CF₃, cyano, amino, nitro, C₁₋₄alkyl, C₁₋₄alkoxy, guanidino, hydroxyl, amidino, or halo.

In some embodiments, each X is, independently, —NR⁸, wherein each R⁸ is, independently, hydrogen or alkyl. In some embodiments, each X is NH.

In some embodiments, each Y is C═O.

In some embodiments, each Het¹ is, independently, a fused bicyclic ring chosen from naphthalene, chroman, isochroman, indoline, isoindoline, and

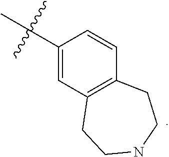 [SMILES: *c1ccc2c(c1)CCNCC2]

In some embodiments, each Het¹ is

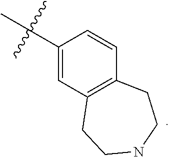 [SMILES: *c1ccc2c(c1)CCNCC2]

In some embodiments, Het² is a heteroaryl chosen from pyrazine, pyrimidine, and pyridazine. In some embodiments, Het² is pyrimidine.

In some embodiments, each W is, independently, —(CH₂)₁₋₅NH₂, —C₁₋₄alkyl, —(CH₂)₁₋₅NHC(═NH)NH₂, or cycloalkyl optionally substituted with one or more —CF₃, cyano, amino, nitro, C₁₋₄alkyl, hydroxyl, amidino, or halo. In some embodiments, each W is, independently, —(CH₂)₂NH₂, —CH(CH₃)₂, —(CH₂)₂₋₄NHC(═NH)NH₂, or cycloalkyl optionally substituted with one or more —CF₃, amino, or halo.

In some embodiments: each X is, independently, —NR⁸, wherein each R⁸ is, independently, hydrogen or alkyl; each Y is C═O; each Het¹ is, independently, a fused bicyclic ring chosen from naphthalene, chroman, isochroman, indoline, isoindoline, and

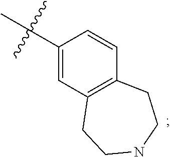 [SMILES: *c1ccc2c(c1)CCNCC2]

Het² is a heteroaryl chosen from pyrazine, pyrimidine, and pyridazine; and each W is, independently, —(CH₂)₁₋₅NH₂, —C₁₋₄alkyl, —(CH₂)₁₋₅NHC(═NH)NH₂, or cycloalkyl optionally substituted with one or more —CF₃, cyano, amino, nitro, C₁₋₄alkyl, hydroxyl, amidino, or halo.

In some embodiments: each X is NH; each Y is C═O; each Het¹ is

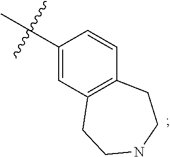 [SMILES: *c1ccc2c(c1)CCNCC2]

Het² is pyrimidine; and each W is, independently, —(CH₂)₂NH₂, —CH(CH₃)₂, —(CH₂)₂₋₄NHC(═NH)NH₂, or cycloalkyl optionally substituted with one or more —CF₃, amino, or halo.

In some embodiments, the compound of Formula IV is chosen from:

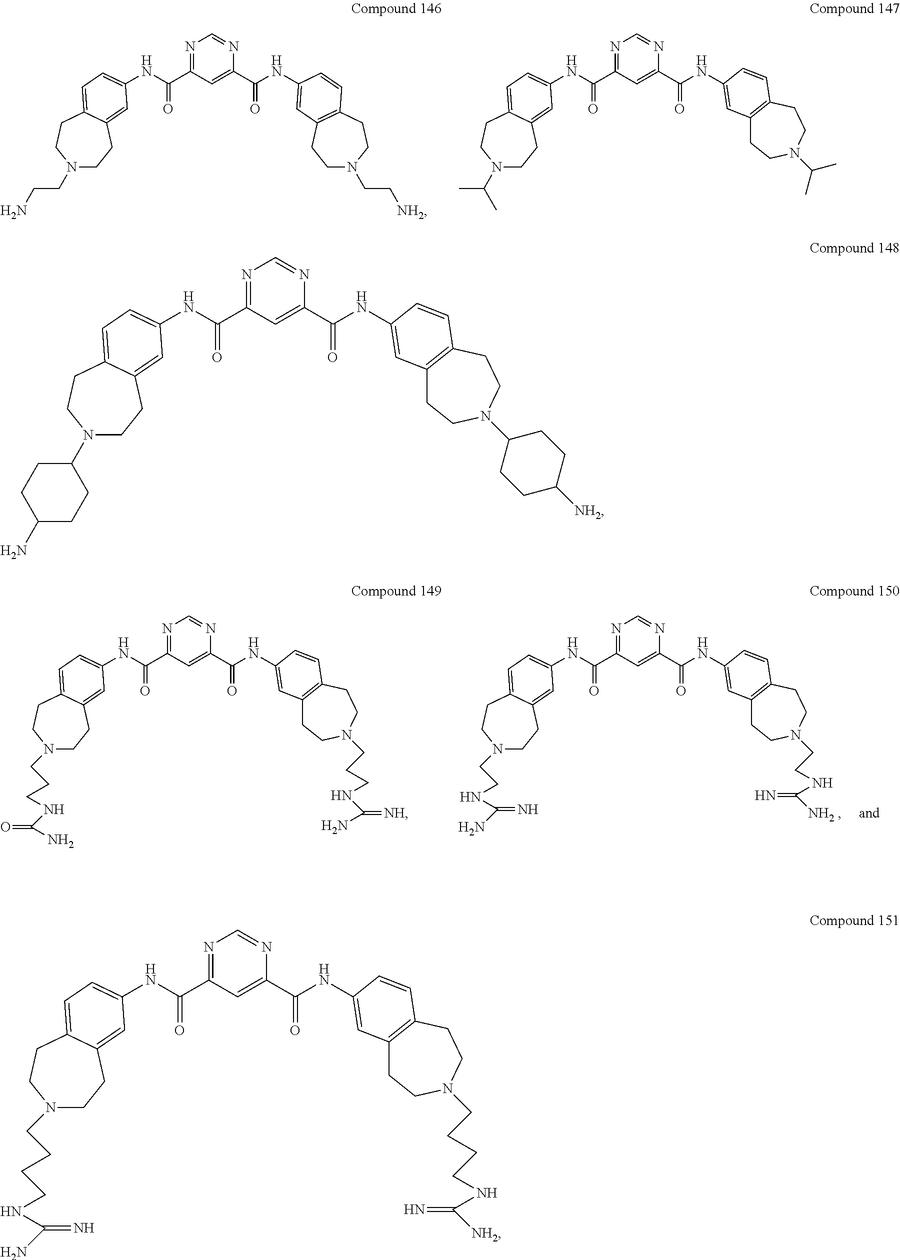 [SMILES: NCCN1CCc2ccc(NC(=O)c3cc(C(=O)Nc4ccc5c(c4)CCN(CCN)CC5)ncn3)cc2CC1 ; CC(C)N1CCc2ccc(NC(=O)c3cc(C(=O)Nc4ccc5c(c4)CCN(C(C)C)CC5)ncn3)cc2CC1 ; NC1CCC(N2CCc3ccc(NC(=O)c4cc(C(=O)Nc5ccc6c(c5)CCN(C5CCC(N)CC5)CC6)ncn4)cc3CC2)CC1 ; N=C(N)NCCCN1CCc2ccc(NC(=O)c3cc(C(=O)Nc4ccc5c(c4)CCN(CCCNC(N)=O)CC5)ncn3)cc2CC1 ; N=C(N)NCCN1CCc2ccc(NC(=O)c3cc(C(=O)Nc4ccc5c(c4)CCN(CCNC(=N)N)CC5)ncn3)cc2CC1 ; N=C(N)NCCCCN1CCc2ccc(NC(=O)c3cc(C(=O)Nc4ccc5c(c4)CCN(CCCCNC(=N)N)CC5)ncn3)cc2CC1]

or a pharmaceutically acceptable salt thereof.

In some embodiments, the compound is not any one or more of Compounds 146 to 151.

The present disclosure also provides anti-malarial compounds of Formula V:

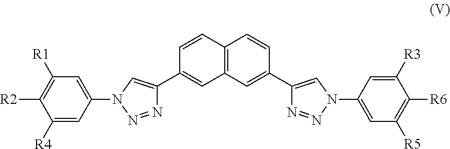

or a pharmaceutically acceptable salt thereof,
wherein:

R¹ is H, —NH(CH₂)ₙNH₂, —NH(CH₂)ₙNC(═N)NH₂, —(CH₂)ₙNH₂, —O—(CH₂)ₙNH₂, —(CH₂)ₙNC(═N)NH₂, —O—(CH₂)ₙNC(═N)NH₂, —CH═CH—CH₂NH₂, —CH═CH—CH₂NC(═N)NH₂, —CH≡CH—(CH₂)₂NH₂, —CH≡CH—(CH₂)₂NC(═N)NH₂, —C≡C—CH₂NH₂, —C≡C—(CH₂)₂NH₂, —CH≡CH—CH₂NC(═N)NH₂, or —C≡C—(CH₂)₂—NC(═N)NH₂, where n is 2, 3, or 4;

R² is H, —NH(CH₂)ₙNH₂, —NH(CH₂)ₙNC(═N)NH₂, —(CH₂)ₙNH₂, —O—(CH₂)ₙNH₂, —(CH₂)ₙNC(═N)NH₂, —O—(CH₂)ₙNC(═N)NH₂, —CH═CH—CH₂NH₂, —CH═CH—CH₂NC(═N)NH₂, —CH═CH—(CH₂)₂NH₂, —CH═CH—(CH₂)₂NC(═N)NH₂, —C≡C—CH₂NH₂, —C≡C—(CH₂)₂NH₂, —CH≡CH—CH₂NC(═N)NH₂, or —C≡C—(CH₂)₂—NC(═N)NH₂, where n is 2, 3, or 4;

R³ is H, —NH(CH₂)ₙNH₂, —NH(CH₂)ₙNC(═N)NH₂, —(CH₂)ₙNH₂, —O—(CH₂)ₙNH₂, —(CH₂)ₙNC(═N)NH₂, —O—(CH₂)ₙNC(═N)NH₂, —CH═CH—CH₂NH₂, —CH═CH—CH₂NC(═N)NH₂, —CH═CH—(CH₂)₂NH₂, —CH═CH—(CH₂)₂NC(═N)NH₂, —C≡C—CH₂NH₂, —C≡C—(CH₂)₂NH₂, —CH≡CH—CH₂NC(═N)NH₂, or —C≡C—(CH₂)₂—NC(═N)NH₂, where n is 2, 3, or 4;

R⁴ is H, —NH(CH₂)ₙNH₂, —NH(CH₂)ₙNC(═N)NH₂, —(CH₂)ₙNH₂, —O—(CH₂)ₙNH₂, —(CH₂)ₙNC(═N)NH₂, —O—(CH₂)ₙNC(═N)NH₂, —CH═CH—CH₂NH₂, —CH═CH—CH₂NC(═N)NH₂, —CH═CH—(CH₂)₂NH₂, —CH═CH—(CH₂)₂NC(═N)NH₂, —C≡C—CH₂NH₂, —C≡C—(CH₂)₂NH₂, —CH≡CH—CH₂NC(═N)NH₂, or —C≡C—(CH₂)₂—NC(═N)NH₂, where n is 2, 3, or 4;

R⁵ is H, —NH(CH₂)ₙNH₂, —NH(CH₂)ₙNC(═N)NH₂, —(CH₂)ₙNH₂, —O—(CH₂)ₙNH₂, —(CH₂)ₙNC(═N)NH₂, —O—(CH₂)ₙNC(═N)NH₂, —CH═CH—CH₂NH₂, —CH═CH—CH₂NC(═N)NH₂, —CH═CH—(CH₂)₂NH₂, —CH═CH—(CH₂)₂NC(═N)NH₂, —C≡C—CH₂NH₂, —C≡C—(CH₂)₂NH₂, —CH≡CH—CH₂NC(═N)NH₂, or —C≡C—(CH₂)₂—NC(═N)NH₂, where n is 2, 3, or 4; and

R⁶ is H, —NH(CH₂)ₙNH₂, —NH(CH₂)ₙNC(═N)NH₂, —(CH₂)ₙNH₂, —O—(CH₂)ₙNH₂, —(CH₂)ₙNC(═N)NH₂, —O—(CH₂)ₙNC(═N)NH₂, —CH═CH—CH₂NH₂, —CH═CH—CH₂NC(═N)NH₂, —CH═CH—(CH₂)₂NH₂, —CH═CH—(CH₂)₂NC(═N)NH₂, —C≡C—CH₂NH₂, —C≡C—(CH₂)₂NH₂, —CH≡CH—CH₂NC(═N)NH₂, or —C≡C—(CH₂)₂—NC(═N)NH₂, where n is 2, 3, or 4.

In some embodiments, R² is H, —NH(CH₂)ₙNH₂, —NH(CH₂)ₙNC(═N)NH₂, —(CH₂)ₙNH₂, —O—(CH₂)ₙNH₂, —(CH₂)ₙNC(═N)NH₂, or —O—(CH₂)ₙNC(═N)NH₂, where n is 2, 3, or 4. In some embodiments, R² is H, —NH(CH₂)ₙNH₂, —(CH₂)ₙNH₂, —O—(CH₂)ₙNH₂, or —(CH₂)ₙNC(═N)NH₂, where n is 2, 3, or 4. In some embodiments, R² is H, —(CH₂)ₙNH₂, or —O—(CH₂)ₙNH₂, where n is 2, 3, or 4. In some embodiments, R² is H or —(CH₂)ₙNH₂, where n is 2, 3, or 4. In some embodiments, R² is H.

In some embodiments, R⁴ is H, —NH(CH₂)ₙNH₂, —NH(CH₂)ₙNC(═N)NH₂, —(CH₂)ₙNH₂, —O—(CH₂)ₙNH₂, —(CH₂)ₙNC(═N)NH₂, or —O—(CH₂)ₙNC(═N)NH₂, where n is 2, 3, or 4. In some embodiments, R⁴ is H, —NH(CH₂)ₙNH₂, —(CH₂)ₙNH₂, —O—(CH₂)ₙNH₂, or —(CH₂)ₙNC(═N)NH₂, where n is 2, 3, or 4. In some embodiments, R⁴ is H, —(CH₂)ₙNH₂, or —O—(CH₂)ₙNH₂, where n is 2, 3, or 4. In some embodiments, R⁴ is H or —(CH₂)ₙNH₂, where n is 2, 3, or 4. In some embodiments, R⁴ is H.

In some embodiments, R⁵ is H, —NH(CH₂)ₙNH₂, —NH(CH₂)ₙNC(═N)NH₂, —(CH₂)ₙNH₂, —O—(CH₂)ₙNH₂, —(CH₂)ₙNC(═N)NH₂, or —O—(CH₂)ₙNC(═N)NH₂, where n is 2, 3, or 4. In some embodiments, R⁵ is H, —NH(CH₂)ₙNH₂, —(CH₂)ₙNH₂, —O—(CH₂)ₙNH₂, or —(CH₂)ₙNC(═N)NH₂, where n is 2, 3, or 4. In some embodiments, R⁵ is H, —(CH₂)ₙNH₂, or —O—(CH₂)ₙNH₂, where n is 2, 3, or 4. In some embodiments, R⁵ is H or —(CH₂)ₙNH₂, where n is 2, 3, or 4. In some embodiments, R⁵ is H.

In some embodiments, R⁶ is H, —NH(CH₂)ₙNH₂, —NH(CH₂)ₙNC(═N)NH₂, —(CH₂)ₙNH₂, —O—(CH₂)ₙNH₂, —(CH₂)ₙNC(═N)NH₂, or —O—(CH₂)ₙNC(═N)NH₂, where n is 2, 3, or 4. In some embodiments, R⁶ is H, —NH(CH₂)ₙNH₂, —(CH₂)ₙNH₂, —O—(CH₂)ₙNH₂, or —(CH₂)ₙNC(═N)NH₂, where n is 2, 3, or 4. In some embodiments, R⁶ is H, —(CH₂)ₙNH₂, or —O—(CH₂)ₙNH₂, where n is 2, 3, or 4. In some embodiments, R⁶ is H or —(CH₂)ₙNH₂, where n is 2, 3, or 4. In some embodiments, R⁶ is H.

In some embodiments, R¹ is H, —NH(CH₂)ₙNH₂, —NH(CH₂)ₙNC(═N)NH₂, —(CH₂)ₙNH₂, —O—(CH₂)ₙNH₂, —(CH₂)ₙNC(═N)NH₂, or —O—(CH₂)ₙNC(═N)NH₂, where n is 2, 3, or 4. In some embodiments, R¹ is H, —NH(CH₂)ₙNH₂, —(CH₂)ₙNH₂, —O—(CH₂)ₙNH₂, or —(CH₂)ₙNC(═N)NH₂, where n is 2, 3, or 4. In some embodiments, R¹ is —NH(CH₂)ₙNH₂, —(CH₂)ₙNH₂, or —O—(CH₂)ₙNH₂, where n is 2, 3, or 4. In some embodiments, R¹ is —(CH₂)ₙNH₂ or —O—(CH₂)ₙNH₂, where n is 2, 3, or 4. In some embodiments, R¹ is —O—(CH₂)ₙNH₂, where n is 2, 3, or 4.

In some embodiments, R³ is H, —NH(CH₂)ₙNH₂, —NH(CH₂)ₙNC(═N)NH₂, —(CH₂)ₙNH₂, —O—(CH₂)ₙNH₂, —(CH₂)ₙNC(═N)NH₂, or —O—(CH₂)ₙNC(═N)NH₂, where n is 2, 3, or 4. In some embodiments, R³ is H, —NH(CH₂)ₙNH₂, —(CH₂)ₙNH₂, —O—(CH₂)ₙNH₂, or —(CH₂)ₙNC(═N)NH₂, where n is 2, 3, or 4. In some embodiments, R³ is —NH(CH₂)ₙNH₂, —(CH₂)ₙNH₂, or —O—(CH₂)ₙNH₂, where n is 2, 3, or 4. In some embodiments, R³ is —(CH₂)ₙNH₂ or —O—(CH₂)ₙNH₂, where n is 2, 3, or 4. In some embodiments, R³ is —O—(CH₂)ₙNH₂, where n is 2, 3, or 4. In some embodiments: R² is H, —NH(CH₂)ₙNH₂, —NH(CH₂)ₙNC(═N)NH₂, —(CH₂)ₙNH₂, —O—(CH₂)ₙNH₂, —(CH₂)ₙNC(═N)NH₂, or —O—(CH₂)ₙNC(═N)NH₂, where n is 2, 3, or 4; R⁴ is H, —NH(CH₂)ₙNH₂, —NH(CH₂)ₙNC(═N)NH₂, —(CH₂)ₙNH₂, —O—(CH₂)ₙNH₂, —(CH₂)ₙNC(═N)NH₂, or —O—(CH₂)ₙNC(═N)NH₂, where n is 2, 3, or 4; R⁵ is H, —(CH₂)ₙNH₂, —NH(CH₂)ₙNH₂, —NH(CH₂)ₙNC(═N)NH₂, —O—(CH₂)ₙNH₂, —(CH₂)ₙNC(═N)NH₂, or —O—(CH₂)ₙNC(═N)NH₂, where n is 2, 3, or 4; R⁶ is H, —NH(CH₂)ₙNH₂, —(CH₂)ₙNH₂, —NH(CH₂)ₙNC(═N)NH₂, —O—(CH₂)ₙNH₂, —(CH₂)ₙNC(═N)NH₂, or —O—(CH₂)ₙNC(═N)NH₂, where n is 2, 3, or 4; R¹ is H, —NH(CH₂)ₙNH₂, —NH(CH₂)ₙNC(═N)NH₂, —(CH₂)ₙNH₂, —O—(CH₂)ₙNH₂, —(CH₂)ₙNC(═N)NH₂, or —O—(CH₂)ₙNC(═N)NH₂, where n is 2, 3, or 4; and R³ is H, —NH(CH₂)ₙNH₂, —NH(CH₂)ₙNC(═N)NH₂, —(CH₂)ₙNH₂, —O—(CH₂)ₙNH₂, —(CH₂)ₙNC(═N)NH₂, or —O—(CH₂)ₙNC(═N)NH₂, where n is 2, 3, or 4.

In some embodiments: R² is H, —NH(CH₂)ₙNH₂, —(CH₂)ₙNH₂, —O—(CH₂)ₙNH₂, or —(CH₂)ₙNC(═N)NH₂, where n is 2, 3, or 4; R⁴ is H, —NH(CH₂)ₙNH₂, —(CH₂)ₙNH₂, —O—(CH₂)ₙNH₂, or —(CH₂)ₙNC(═N)NH₂, where n is 2, 3, or 4; R⁵ is H, —NH(CH₂)ₙNH₂, —(CH₂)ₙNH₂, —O—(CH₂)ₙNH₂, or —(CH₂)ₙNC(═N)NH₂, where n is 2, 3, or 4; R⁶ is H, —NH(CH₂)ₙNH₂, —(CH₂)ₙNH₂, —O—(CH₂)ₙNH₂, or —(CH₂)ₙNC(═N)NH₂, where n is 2, 3, or 4; R¹ is H, —NH(CH₂)ₙNH₂, —(CH₂)ₙNH₂, —O—(CH₂)ₙNH₂, or —(CH₂)ₙNC(═N)NH₂, where n is 2, 3, or 4; and R³ is H, —NH(CH₂)ₙNH₂, —(CH₂)ₙNH₂, —O—(CH₂)ₙNH₂, or —(CH₂)ₙNC(═N)NH₂, where n is 2, 3, or 4.

In some embodiments: R² is H, —(CH₂)ₙNH₂, or —O—(CH₂)ₙNH₂, where n is 2, 3, or 4; R⁴ is H, —(CH₂)ₙNH₂, or —O—(CH₂)ₙNH₂, where n is 2, 3, or 4; R⁵ is H, —(CH₂)ₙNH₂, or —O—(CH₂)ₙNH₂, where n is 2, 3, or 4; R⁶ is H, —(CH₂)ₙNH₂, or —O—(CH₂)ₙNH₂, where n is 2, 3, or 4; R¹ is —NH(CH₂)ₙNH₂, —(CH₂)ₙNH₂, or —O—(CH₂)ₙNH₂, where n is 2, 3, or 4; and R³ is —NH(CH₂)ₙNH₂, —(CH₂)ₙNH₂, or —O—(CH₂)ₙNH₂, where n is 2, 3, or 4.

In some embodiments: R² is H or —(CH₂)ₙNH₂, where n is 2, 3, or 4; R⁴ is H or —(CH₂)ₙNH₂, where n is 2, 3, or 4; R⁵ is H or —(CH₂)ₙNH₂, where n is 2, 3, or 4; R⁶ is H or —(CH₂)ₙNH₂, where n is 2, 3, or 4; R¹ is —(CH₂)ₙNH₂ or —O—(CH₂)ₙNH₂, where n is 2, 3, or 4; and R³ is —(CH₂)ₙNH₂ or —O—(CH₂)ₙNH₂, where n is 2, 3, or 4.

In some embodiments: R², R⁴, R⁵, and R⁶ are H; R¹ is —O—(CH₂)ₙNH₂, where n is 2, 3, or 4; and R³ is —O—(CH₂)ₙNH₂, where n is 2, 3, or 4.

In some embodiments, the compound of Formula V is

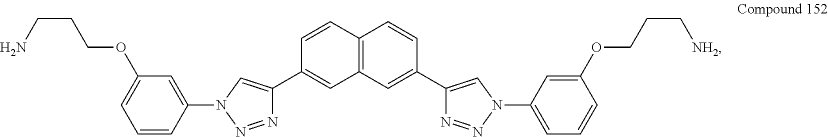 [SMILES: NCCCOc1cccc(-n2cc(-c3ccc4ccc(-c5cn(-c6cccc(OCCCN)c6)nn5)cc4c3)nn2)c1]

or a pharmaceutically acceptable salt thereof.

In some embodiments, the compound is not Compound 152.

The present disclosure also provides anti-malarial compounds of Formula VI:

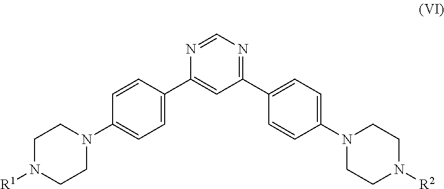

or a pharmaceutically acceptable salt thereof,
wherein:

R¹ and R² are, independently, hydrogen, —C₁₋₄alkyl, —C(═NH)NH₂, —(CH₂)ₙNH₂, or —(CH₂)ₙNC(═NH)NH₂, where n is 2, 3, or 4.

In some embodiments, R¹ and R² are, independently, hydrogen, —C(═NH)NH₂, —(CH₂)ₙNH₂, or —(CH₂)ₙNC(═NH)NH₂, where n is 2, 3, or 4. In some embodiments, R¹ and R² are, independently, —C(═NH)NH₂, —(CH₂)ₙNH₂, or —(CH₂)ₙNC(═NH)NH₂, where n is 2 or 3. In some embodiments, R¹ and R² are, independently, —C(═NH)NH₂ or —(CH₂)ₙNH₂, where n is 2 or 3.

In some embodiments, the compound of Formula VI is:

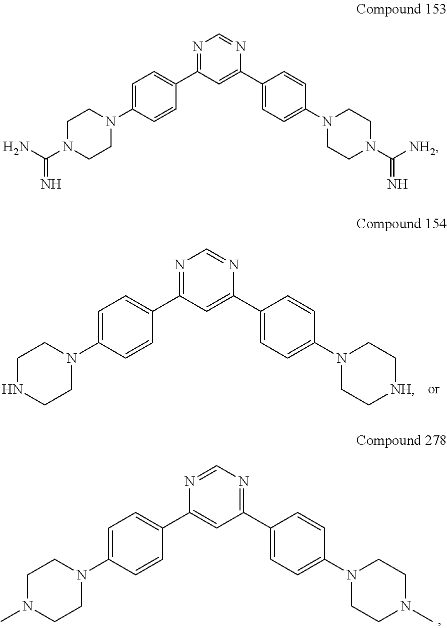

or a pharmaceutically acceptable salt thereof.

In some embodiments, the compound is not Compound 153, 154, or 278.

The present disclosure also provides anti-malarial compounds of Formula VII:

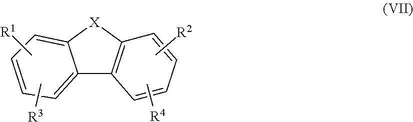

or a pharmaceutically acceptable salt thereof,
wherein:

X is C(R⁷)C(R⁸), C(R⁷)(R⁸), S, or —N(R⁹);

R⁷, R⁸, and R⁹ are, independently, C₁-C₄alkyl, —(CH₂)₀₋₄NH₂, or —(CH₂)₀₋₄NHC(═NH)NH₂;

R¹ and R² are, independently, H, C₁-C₈alkyl, C₁-C₈alkoxy, halo, OH, CN, CF₃, or haloC₁-C₈alkyl;

R³ and R⁴ are, independently, H, carbocycle(R⁵)(R⁶), —NH-carbocycle(R⁵)(R⁶);

each R⁵ and each R⁶ are, independently, H, C₁-C₈alkyl, C₁-C₈alkoxy, halo, amino, amidino, OH, CF₃, —N—(CH₃)₂, —O—(CH₂)₁₋₅—NH₂, —O—(CH₂)₁₋₅NHC(═NH)NH₂, —S—(CH₂)₁₋₅—NH₂, —N((CH₂)₁₋₅NH₂)₂, —S—(CH₂)₁₋₅NHC(═NH)NH₂, —C(═O)NH(CH₂)₁₋₅NH₂, —(CH₂)₁₋₈—NH₂, —(CH₂)₁₋₅N((CH₂)₁₋₅NH₂)₂, aromatic group, heterocycle, —(CH₂)₁₋₈—NH—(CH₂)₁₋₈—NH₂, or —(CH₂)₁₋₈—NH—C(═NH)NH₂.

In some embodiments, X is C(R⁷)C(R⁸) or —N(R⁹). In some embodiments, X is C(R⁷)(R⁸) or S. In some embodiments, X is —N(R⁹). In some embodiments, X is N(CH₂)₂₋₃NH₂ or —(CH₂)₂₋₃NHC(═NH)NH₂. In some embodiments, X is N(CH₂)₃NH₂ or —(CH₂)₃NHC(═NH)NH₂.

In some embodiments, R⁷, R⁸, and R⁹ are, independently, —(CH₂)₀₋₄NH₂ or —(CH₂)₀₋₄NHC(═NH)NH₂.

In some embodiments, R¹ and R² are, independently, H, C₁-C₃alkyl, C₁-C₃alkoxy, halo, OH, haloC₁-C₃alkyl, or CN. In some embodiments, R¹ and R² are, independently, H, C₁-C₃alkyl, C₁-C₃alkoxy, halo, or OH. In some embodiments, R¹ and R² are, independently, H, C₁-C₃alkyl, or halo. In some embodiments, R¹ and R² are H.

In some embodiments, R³ and R⁴ are, independently, H or carbocycle(R⁵)(R⁶). In some embodiments, R³ and R⁴ are, independently, H or carbocycle(R⁵)(R⁶), where R⁵ and R⁶ can be positioned anywhere on the carbocycle. In some embodiments, R³ and R⁴ are, independently,

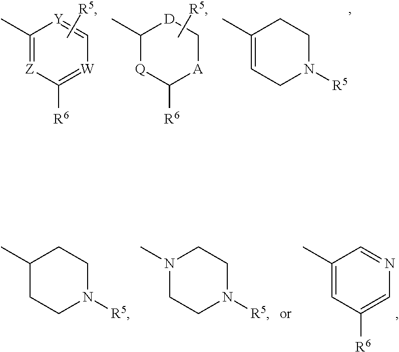

wherein each W, Y, and Z are, independently, C or N, each A, D, and Q are, independently, C(R¹⁰)C(R¹¹), C(═O), N(R¹²), O, or S, and each R¹⁰, R¹¹, and R¹² are, independently, H, C₁-C₈alkyl, C₁-C₈alkoxy, halo, OH, CF₃, or aromatic group. In some embodiments, R³ and R⁴ are, independently,

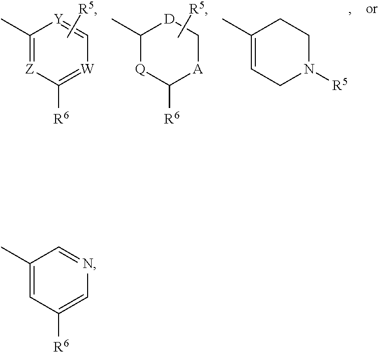

wherein each W, Y, and Z are, independently, C or N, each A, D, and Q are, independently, C(R¹⁰)C(R¹¹), C(═O), N(R¹²), O, or S, and each R¹⁰, R¹¹, and R¹² are, independently, H, C₁-C₈alkyl, C₁-C₈alkoxy, halo, OH, CF₃, or aromatic group. In some embodiments, R³ and R⁴ are, independently,

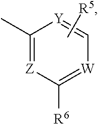

wherein each W, Y, and Z are, independently, C or N. In some embodiments, R³ and R⁴ are, independently,

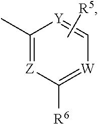

wherein each W, Y, and Z are C, or each Y and Z are C and each W is N.

In some embodiments, each R⁵ and each R⁶ are, independently, H, C₁-C₈alkyl, C₁-C₈alkoxy, halo, amino, OH, CF₃, —O—(CH₂)₁₋₅—NH₂, —O—(CH₂)₁₋₅NHC(═NH)NH₂, —S—(CH₂)₁₋₅—NH₂, —N((CH₂)₁₋₅NH₂)₂, —S—(CH₂)₁₋₅NHC(═NH)NH₂, —C(═O)NH(CH₂)₁₋₅NH₂, —(CH₂)₁₋₅N((CH₂)₁₋₅NH₂)₂, aromatic group, heterocycle, —(CH₂)₁₋₈—NH₂, —(CH₂)₁₋₈—NH—(CH₂)₁₋₈—NH₂, or —(CH₂)₁₋₈—NH—C(═NH)NH₂.

In some embodiments, each R⁵ is, independently, H, C₁-C₈alkyl, C₁-C₈alkoxy, halo, amino, OH, CF₃, —O—(CH₂)₁₋₅—NH₂, —O—(CH₂)₁₋₅NHC(═NH)NH₂, —S—(CH₂)₁₋₅—NH₂, —N((CH₂)₁₋₅NH₂)₂, —S—(CH₂)₁₋₅NHC(═NH)NH₂, —C(═O)NH(CH₂)₁₋₅NH₂, —(CH₂)₁₋₈—NH₂, —(CH₂)₁₋₅N((CH₂)₁₋₅NH₂)₂, —(CH₂)₁₋₈—NH—(CH₂)₁₋₈—NH₂, or —(CH₂)₁₋₈—NH—C(═NH)NH₂, and each R⁶ is, independently, amino, heterocycle, —O—(CH₂)₁₋₅—NH₂, —O—(CH₂)₁₋₅NHC(═NH)NH₂, —S—(CH₂)₁₋₅—NH₂, —S—(CH₂)₁₋₅NHC(═NH)NH₂, —N((CH₂)₁₋₅NH₂)₂, —C(═O)NH(CH₂)₁₋₅NH₂, —(CH₂)₁₋₅N((CH₂)₁₋₅NH₂)₂, —(CH₂)₁₋₈—NH₂, —(CH₂)₁₋₈—NH—(CH₂)₁₋₈—NH₂, or —(CH₂)₁₋₈—NH—C(═NH)NH₂.

In some embodiments, each R⁵ is, independently, H, C₁-C₃alkyl, C₁-C₃alkoxy, halo, OH, CF₃, or —O—(CH₂)₁₋₅—NH₂, and each R⁶ is, independently, heterocycle, —O—(CH₂)₁₋₅—NH₂, or —(CH₂)₁₋₈—NH₂.

In some embodiments, each R⁵ is, independently, H, C₁-C₃alkyl, halo, OH, or —O—(CH₂)₂₋₃—NH₂, and each R⁶ is, independently, heterocycle, —O—(CH₂)₂₋₃—NH₂, or —(CH₂)₁₋₄—NH₂.

In some embodiments, each R⁵ is, independently, H, C₁-C₃alkyl, halo, OH, or —O—(CH₂)₃—NH₂, and each R⁶ is, independently, 6-membered heterocycle, —O—(CH₂)₃—NH₂, or —(CH₂)₁₋₃—NH₂.

In some embodiments, each R⁵ is, independently, H, halo, or —O—(CH₂)₃—NH₂, and each R⁶ is piperazinyl, —O—(CH₂)₃—NH₂, or —(CH₂)₁₋₃—NH₂.

In some embodiments, each R⁵ is —O—(CH₂)₃—NH₂ or piperazinyl, and each R⁶ is, independently, H, C₁-C₃alkyl, C₁-C₃alkoxy, halo, OH, CF₃, or —O—(CH₂)₃—NH₂.

In some embodiments, each R⁵ is piperazinyl or —O—(CH₂)₃—NH₂, and each R⁶ is H, C₁-C₃alkyl, halo, OH, CF₃, or —O—(CH₂)₃—NH₂.

In some embodiments, X is C(R⁷)C(R⁸) or —N(R⁹); R⁷, R⁸, and R⁹ are, independently, —(CH₂)₀₋₄NH₂ or —(CH₂)₀₋₄NHC(═NH)NH₂; R¹ and R² are, independently, H, C₁-C₈alkyl, C₁-C₈alkoxy, halo, OH, CN, CF₃, or haloC₁-C₈alkyl; R³ and R⁴ are, independently, H or carbocycle(R⁵)(R⁶); and each R⁵ and each R⁶ are, independently, H, C₁-C₈alkyl, C₁-C₈alkoxy, halo, amino, OH, CF₃, —O—(CH₂)₁₋₅—NH₂, —O—(CH₂)₁₋₅NHC(═NH)NH₂, —S—(CH₂)₁₋₅—NH₂, —N((CH₂)₁₋₅NH₂)₂, —S—(CH₂)₁₋₅NHC(═NH)NH₂, —C(═O)NH(CH₂)₁₋₅NH₂, —(CH₂)₁₋₅N((CH₂)₁₋₅NH₂)₂, aromatic group, heterocycle, —(CH₂)₁₋₈—NH₂, —(CH₂)₁₋₈—NH—(CH₂)₁₋₈—NH₂, or —(CH₂)₁₋₈—NH—C(═NH)NH₂.

In some embodiments, X is N(CH₂)₂₋₃NH₂ or —(CH₂)₂₋₃NHC(═NH)NH₂; R¹ and R² are H; R³ and R⁴ are, independently,

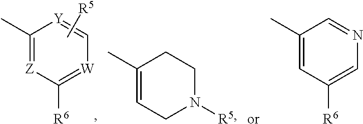

wherein: each W, Y, and Z are, independently, C or N; each R⁵ and each R⁶ are, independently, H, heterocycle, —O—(CH₂)₁₋₃—NH₂, or —(CH₂)₁₋₃—NH₂.

In some embodiments, X is N(CH₂)₂₋₃NH₂ or —(CH₂)₂₋₃NHC(═NH)NH₂; R¹ and R² are H; R³ and R⁴ are

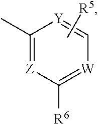

wherein: each Z and Y are C, and each W is N; or each W, Y, and Z are C; each R⁵ is, independently, H, amino, halo, —O—(CH₂)₂₋₃—NH₂, —C(═O)NH(CH₂)₂₋₃NH₂, —N((CH₂)₂₋₃NH₂)₂, or —(CH₂)₂₋₃N((CH₂)₂₋₃NH₂)₂, and each R⁶ is piperazinyl, amino, —O—(CH₂)₂₋₃NH₂, —N((CH₂)₂₋₃NH₂)₂, —C(═O)NH(CH₂)₂₋₃NH₂, —(CH₂)₂₋₃N((CH₂)₂₋₃NH₂)₂, or —(CH₂)₁₋₃—NH₂.

In some embodiments, each R⁵ is piperazinyl or —O—(CH₂)₃—NH₂, and each R⁶ is, independently, H, C₁-C₃alkyl, C₁-C₃alkoxy, halo, OH, CF₃, or —O—(CH₂)₃—NH₂.

In some embodiments, X is N(CH₂)₂₋₃NH₂ or —(CH₂)₂₋₃NHC(═NH)NH₂; R¹ and R² are H; R³ and R⁴ are

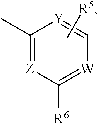

wherein: each Z and Y are C, and each W is N; or each W, Y, and Z are C; each R⁵ is H or —O—(CH₂)₃—NH₂, and each R⁶ is piperazinyl, —O—(CH₂)₃—NH₂, —(CH₂)₁₋₃NH₂, or each R⁵ is piperazinyl or —O—(CH₂)₃NH₂, and each R⁶ is H or —O—(CH₂)₃—NH₂.

In some embodiments, the compound of Formula VII is:

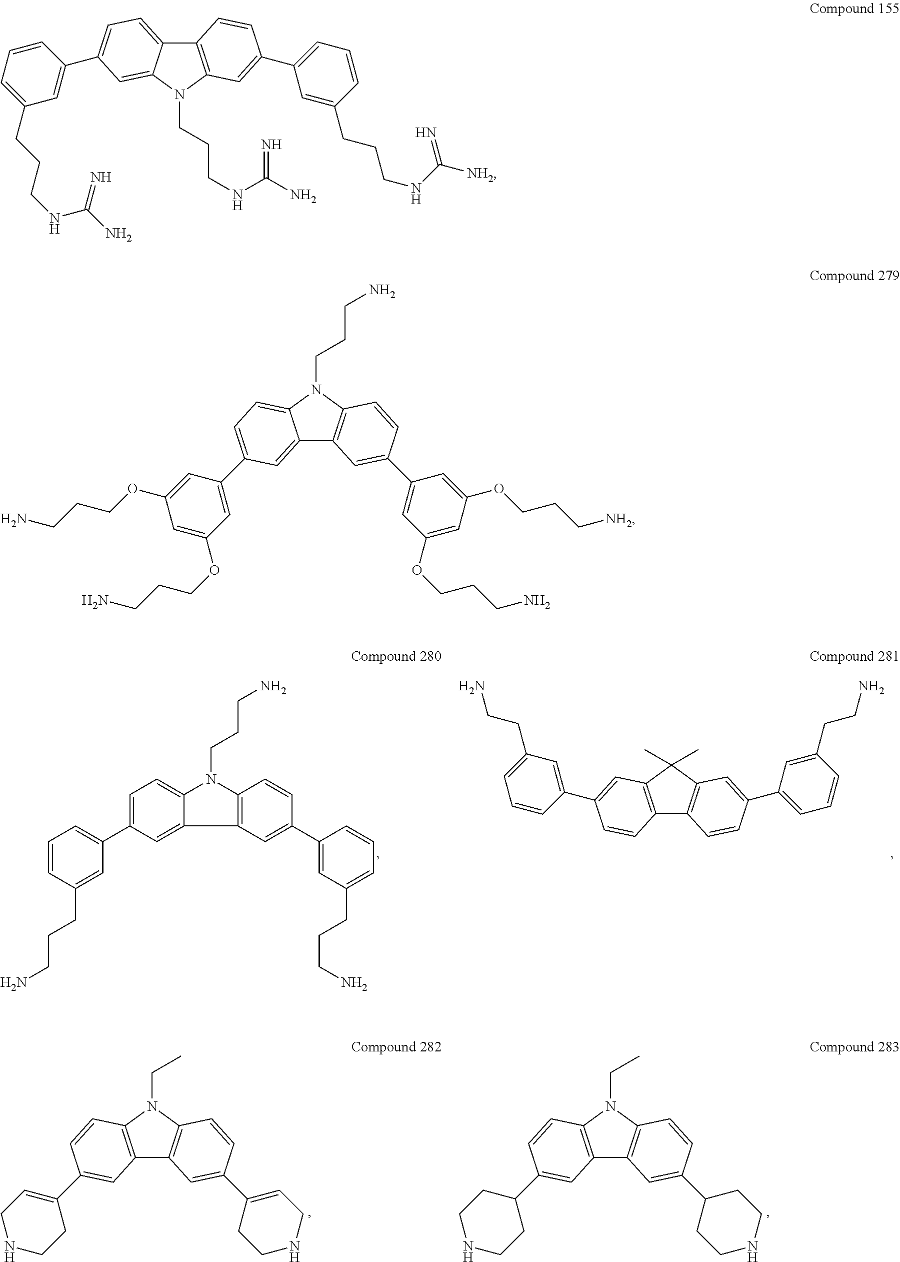 [SMILES: N=C(N)NCCCc1cccc(-c2ccc3c4ccc(-c5cccc(CCCNC(=N)N)c5)cc4n(CCCNC(=N)N)c3c2)c1 ; NCCCOc1cc(OCCCN)cc(-c2ccc3c(c2)c2cc(-c4cc(OCCCN)cc(OCCCN)c4)ccc2n3CCCN)c1 ; NCCCc1cccc(-c2ccc3c(c2)c2cc(-c4cccc(CCCN)c4)ccc2n3CCCN)c1 ; CC1(C)c2cc(-c3cccc(CCN)c3)ccc2-c2ccc(-c3cccc(CCN)c3)cc21 ; CCn1c2ccc(C3=CCNCC3)cc2c2cc(C3=CCNCC3)ccc21 ; CCn1c2ccc(C3CCNCC3)cc2c2cc(C3CCNCC3)ccc21]

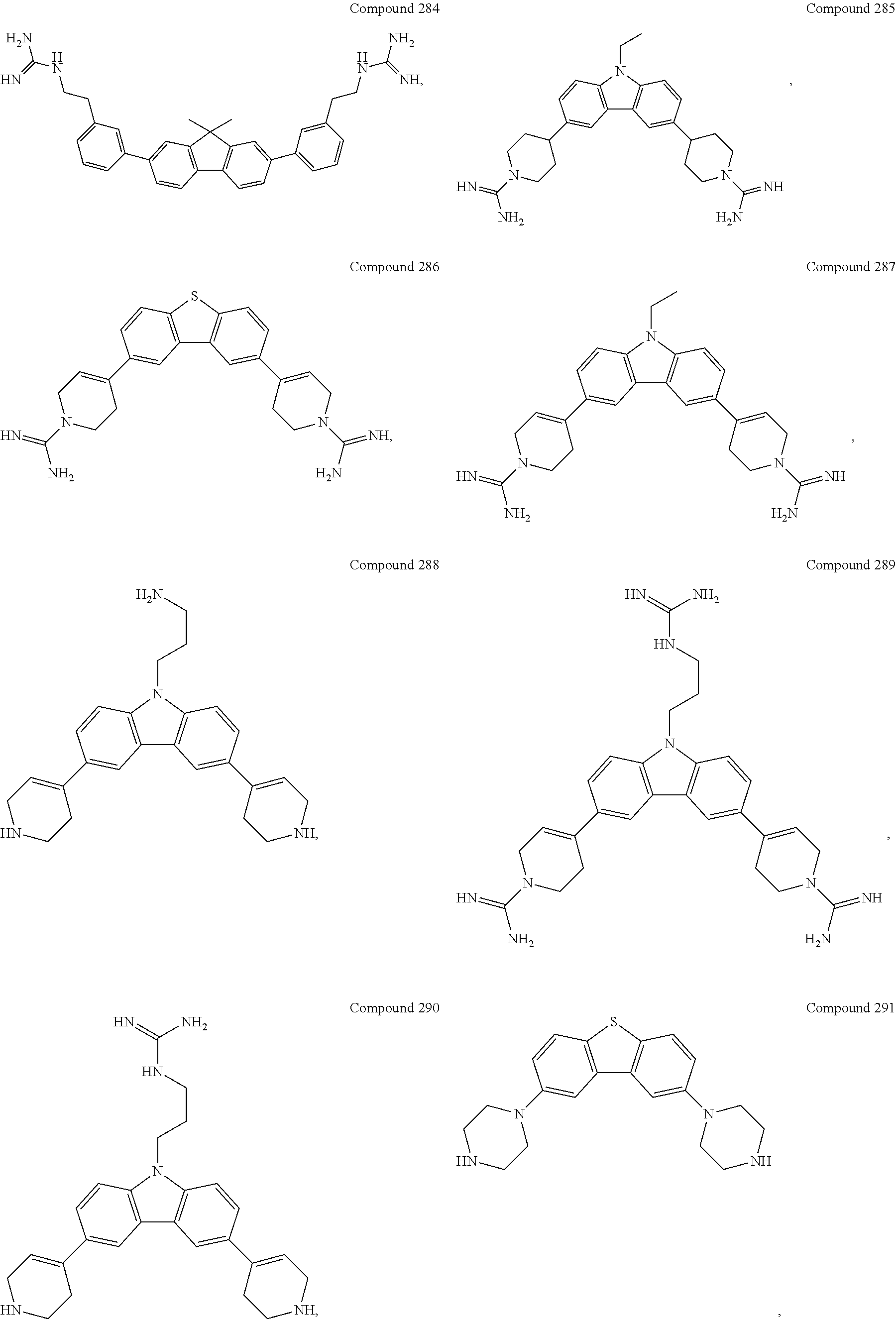

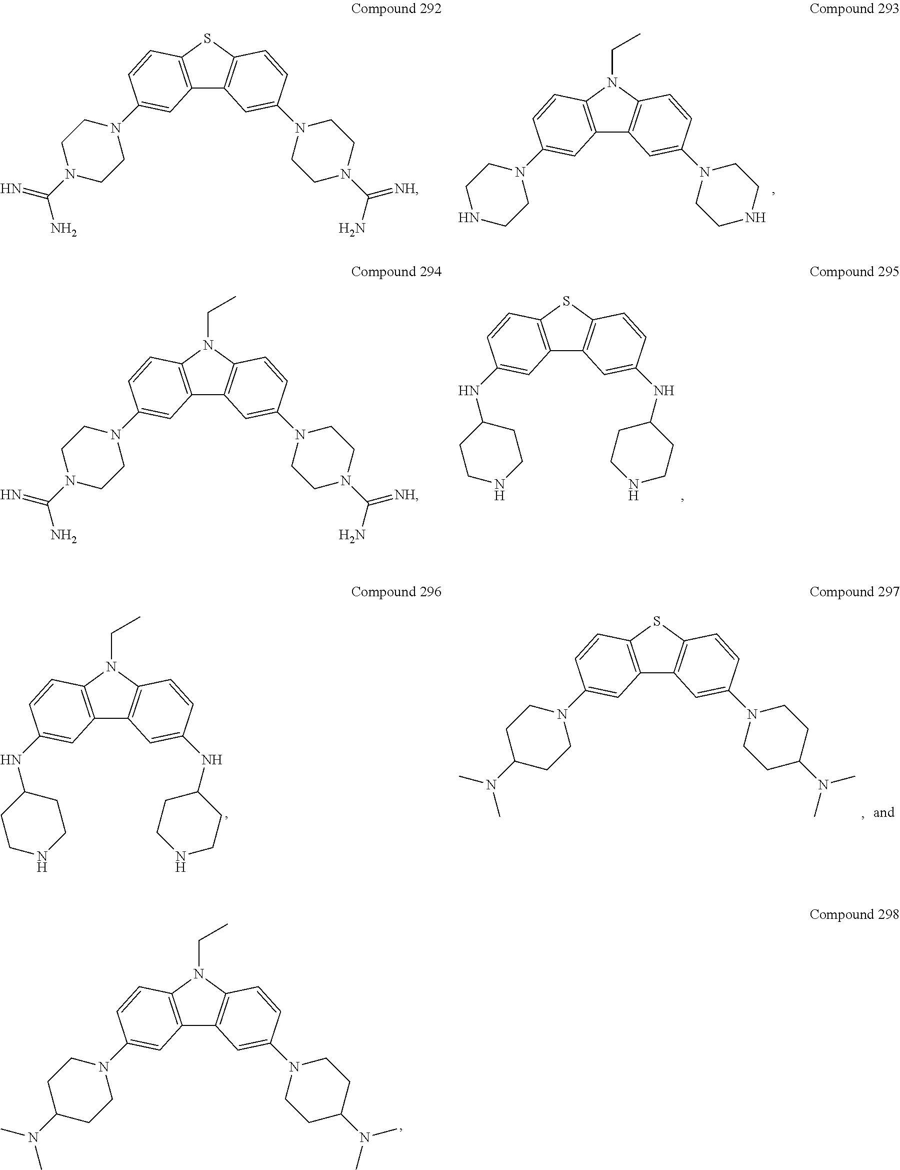

or a pharmaceutically acceptable salt thereof.

In some embodiments, the compound is not Compound 279. In some embodiments, the compound is not any one or more of Compounds 279 to 298.

The present disclosure also provides anti-malarial compounds of Formula VIII:

Q-X—Z—X-Q (VIII)

or a pharmaceutically acceptable salt thereof,
wherein:

Z is

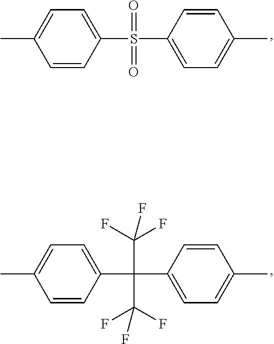

or phenyl;

each Q is

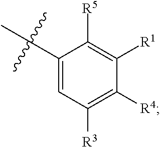

each X is, independently, O, S, or N;

each R¹ is, independently, H, CF₃, C(CH₃)₃, halo, or OH;

each R³ is, independently, H, —NHR², —(CH₂)₁₋₂NH₂, —NH₂, —NH(CH₂)₁₋₃NH₂,

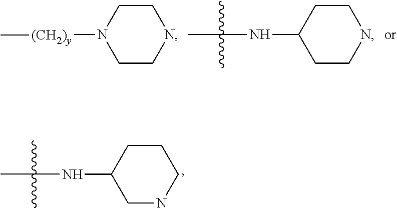

wherein each y is, independently, 1 or 2;

each R² is, independently, H, or the free base or salt form of —(CH₂)₁₋₄NH₂ or —(CH₂)₁₋₄NHC(═NH)NH₂;

each R⁴ is, independently, H, —CF₃, —NHC(═O)(CH₂)₁₋₆NHC(═NH)NH₂,

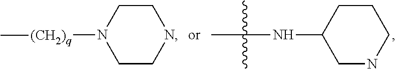

wherein each q is, independently, 1 or 2; and

each R⁵ is, independently, H, —(CH₂)₁₋₄NH₂, or CF₃;

wherein the compound comprises:

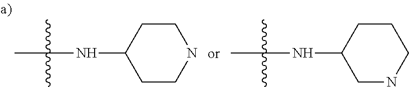 [SMILES: *NC1CCNCC1 ; "or" ; *NC1CCCNC1]

at at least one R³;

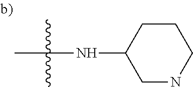 [SMILES: *NC1CCCNC1]

at at least one R⁴; or

c) —(CH₂)₁₋₄NH₂ at at least one R⁵.

In some embodiments, Z is

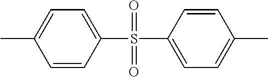

or phenyl.

In some embodiments, each X is O.

In some embodiments, each R¹ is, independently, H, CF₃, or halo.

In some embodiments, each R³ is, independently, H, —(CH₂)₁₋₂NH₂, —NH₂,

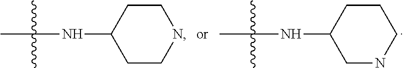

In some embodiments, each R⁴ is, independently, H, —CF₃, or

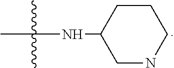 [SMILES: *NC1CCCNC1]

In some embodiments, each R⁵ is, independently, H, —(CH₂)₁₋₄NH₂, or CF₃.

In some embodiments: Z is

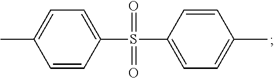

each Q is

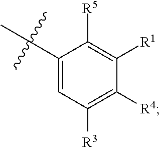

each X is O; each R¹ is, independently, H, CF₃, or halo; each R³ is, independently

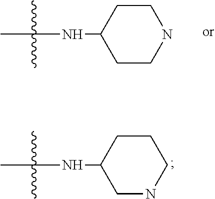 [SMILES: *NC1CCNCC1 ; *NC1CCCNC1]

each R⁴ is, independently, H, —CF₃ or

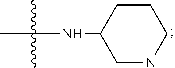 [SMILES: *NC1CCCNC1]

and each R⁵ is, independently, H, —(CH₂)₁₋₄NH₂, or CF₃

In some embodiments: Z is phenyl; each Q is

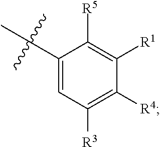

each X is O; each R¹ is, independently, H, CF₃, or halo; each R³ is, independently, H,

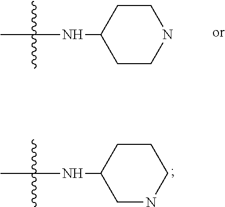 [SMILES: *NC1CCNCC1 ; *NC1CCCNC1]

each R⁴ is, independently, H, —CF₃, or

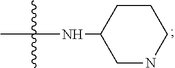 [SMILES: *NC1CCCNC1]

and each R⁵ is, independently, H, —CH₂NH₂, or CF₃.

In some embodiments, the compound of Formula VIII is:

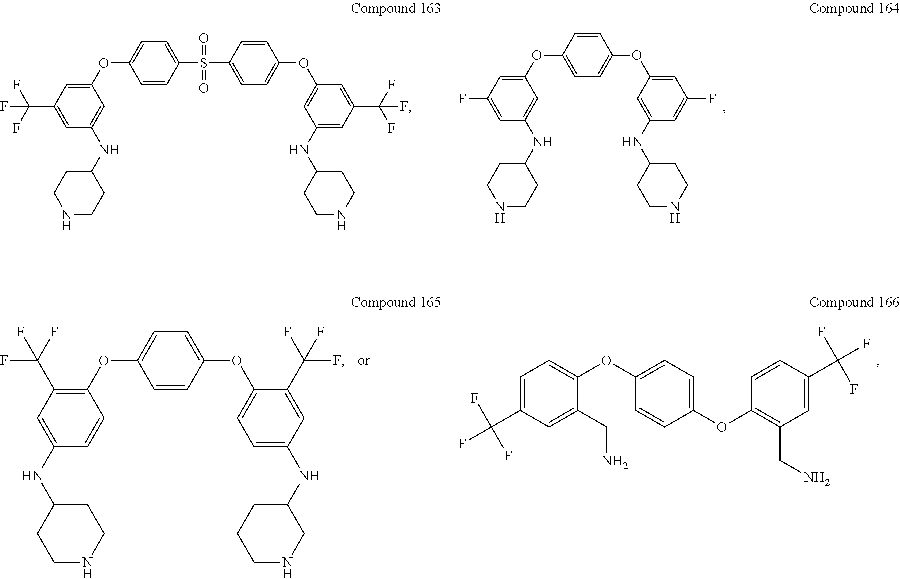 [SMILES: O=S(=O)(c1ccc(Oc2cc(NC3CCNCC3)cc(C(F)(F)F)c2)cc1)c1ccc(Oc2cc(NC3CCNCC3)cc(C(F)(F)F)c2)cc1 ; Fc1cc(NC2CCNCC2)cc(Oc2ccc(Oc3cc(F)cc(NC4CCNCC4)c3)cc2)c1 ; FC(F)(F)c1cc(NC2CCNCC2)ccc1Oc1ccc(Oc2ccc(NC3CCCNC3)cc2C(F)(F)F)cc1 ; NCc1cc(C(F)(F)F)ccc1Oc1ccc(Oc2ccc(C(F)(F)F)cc2CN)cc1]

or a pharmaceutically acceptable salt thereof.

In some embodiments, the compound is not any one or more of Compounds 163 to 166.

The present disclosure also provides anti-malarial compounds of Formula IX:

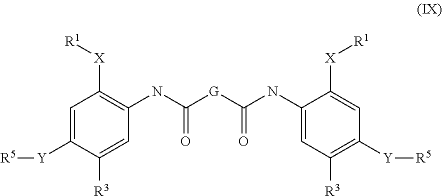

or a pharmaceutically acceptable salt thereof,
wherein:

G is

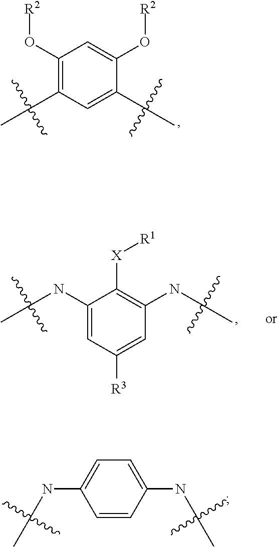

each X is, independently, O or S;

each Y is, independently, O or S;

each R¹ is, independently,

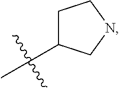

### —(CH₂)₁₋₄NH₂, or —(CH₂)₁₋₄NHC(═NH)NH₂;

each R² is, independently, H, C₁-C₈alkyl, —(CH₂)₁₋₄NH₂, or —(CH₂)₁₋₄NHC(═NH)NH₂;

each R³ is, independently, H, —CF₃, —C(CH₃)₃, halo, or OH; and

each R⁵ is, independently, —(CH₂)₁₋₄NH₂ or —(CH₂)₁₋₄NHC(═NH)NH₂.

In some embodiments, G is

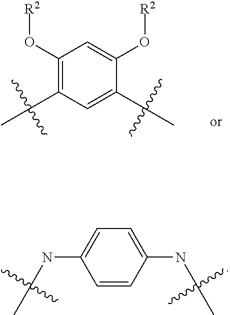

In some embodiments, each X is S. In some embodiments, each X is O.

In some embodiments, each Y is S. In some embodiments, each Y is O.

In some embodiments, each R¹ is, independently, —(CH₂)₁₋₄NH₂ or —(CH₂)₁₋₄NHC(═NH)NH₂. In some embodiments, each R¹ is, independently, —(CH₂)₂NH₂ or —(CH₂)₄NHC(═NH)NH₂.

In some embodiments, each R² is, independently, C₁-C₃alkyl or —(CH₂)₁₋₄NH₂. In some embodiments, each R² is, independently, C₁-C₃alkyl or —(CH₂)₁₋₂NH₂. In some embodiments, each R² is, independently, methyl or —(CH₂)₂NH₂. In some embodiments, each R² is methyl or —(CH₂)₂NH₂.

In some embodiments, each R³ is, independently, CF₃, —C(CH₃)₃, or halo. In some embodiments, each R³ is —CF₃.

In some embodiments, each R⁵ is, independently, —(CH₂)₁₋₄NHC(═NH)NH₂. In some embodiments, each R⁴ is —(CH₂)₄NH—C(═NH)NH₂.

In some embodiments, G is

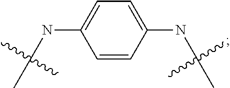 [SMILES: *Nc1ccc(N*)cc1]

each X is, independently, O or S; each Y is, independently, O or S; each R¹ is —(CH₂)₁₋₄NHC(═NH)NH₂; each R³ is, independently, H or CF₃; and each R⁵ is, independently, —(CH₂)₁₋₄NH₂ or —(CH₂)₁₋₄NHC(═NH)NH₂.

In some embodiments, G is

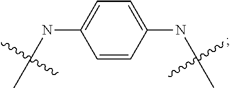 [SMILES: *Nc1ccc(N*)cc1]

each X is O; each Y is O; each R¹ is —(CH₂)₃₋₄NHC(═NH)NH₂; each R³ is, independently, H or CF₃; and each R⁵ is, independently, —(CH₂)₃₋₄NH₂ or —(CH₂)₁₋₄NHC(═NH)NH₂.

In some embodiments, the compound of Formula IX is:

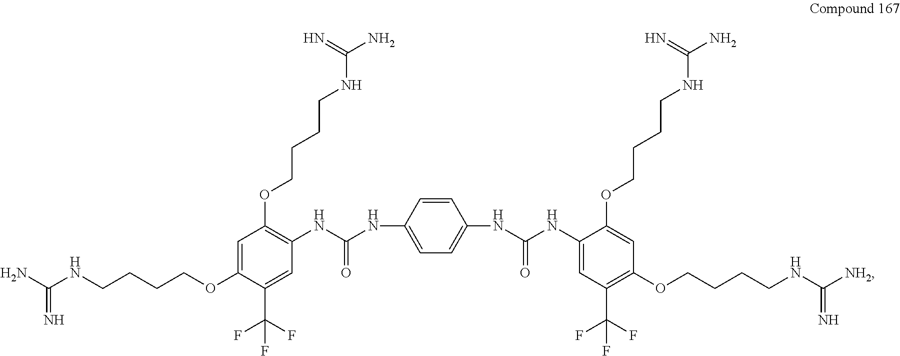 [SMILES: N=C(N)NCCCCOc1cc(OCCCCNC(=N)N)c(C(F)(F)F)cc1NC(=O)Nc1ccc(NC(=O)Nc2cc(C(F)(F)F)c(OCCCCNC(=N)N)cc2OCCCCNC(=N)N)cc1]

or a pharmaceutically acceptable salt thereof.

In some embodiments, the compound is not Compound 167.

The present disclosure also provides anti-malarial compounds of Formula Xa:

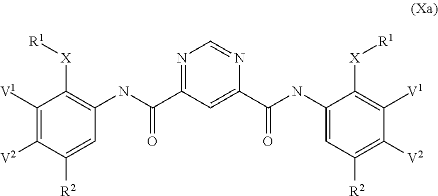

or a pharmaceutically acceptable salt thereof,
wherein:

each X is, independently, O, S, or S(═O)₂;

each R¹ is, independently, —(CH₂)₁₋₄C(═O)OH, —(CH₂)₁₋₄NH₂, —(CH₂)₁₋₄NHC(═O)C₁₋₄alkyl, —(CH₂)₁₋₄OH, or —(CH₂)₁₋₄C(═O)OC₁₋₄alkyl;

each R² is, independently, H, halo, —CF₃, or —C(CH₃)₃;

each V² is H; and

each V¹ is, independently, amino or —NC(═O)—R³, where each R³ is, independently, —C₁₋₄alkyl, —(CH₂)₁₋₄—NH₂, —(CH₂)₁₋₄—NH—C(═NH)NH₂, or aryl (optionally substituted with one or more, independently, halo, cyano, or —C₁₋₄alkoxy).

In some embodiments, each X is S.

In some embodiments, each R¹ is, independently, —(CH₂)₁₋₄C(═O)OH, —(CH₂)₁₋₄NHC(═O)C₁₋₄alkyl, —(CH₂)₁₋₄OH, or —(CH₂)₁₋₄C(═O)OC₁₋₄alkyl. In some embodiments, each R¹ is, independently, —(CH₂)₂C(═O)OH, —(CH₂)₂OH, —(CH₂)₂NHC(═O)C₁₋₄alkyl, or —(CH₂)₂C(═O)OC₁₋₄alkyl. In some embodiments, each R¹ is, independently, —(CH₂)₂C(═O)OH, —(CH₂)₂OH, —(CH₂)₂NHC(═O)CH₃, or —(CH₂)₂C(═O)OCH₃.

In some embodiments, each R² is, independently, halo or —CF₃. In some embodiments, each R² is, independently, Br or —CF₃.

In some embodiments, each V¹ is, independently, —NC(═O)—R³, where each R³ is, independently, —(CH₂)₁₋₄—NH₂ or —(CH₂)₁₋₄—NH—C(═NH)NH₂. In some embodiments, each V¹ is, independently, —NC(═O)—R³, wherein each R³ is, independently, —(CH₂)₃₋₄—NH₂ or —(CH₂)₃₋₄—NH—C(═NH)NH₂. In some embodiments, each V¹ is, independently, —NC(═O)—R³, wherein each R³ is, independently, —(CH₂)₃₋₄—NH—C(═NH)NH₂. In some embodiments, each V¹ is —NC(═O)—R³, wherein each R³ is —(CH₂)₄—NH—C(═NH)NH₂. In some embodiments, each V¹ is, independently, —NC(═O)—R³, where each R³ is, independently, phenyl, thienyl, or pyridyl (each, independently, optionally substituted with one or more, independently, halo, cyano, or —C₁₋₄alkoxy).

In some embodiments, each X is, independently, O or S; each R¹ is, independently, —(CH₂)₁₋₄NH₂; each R² is, independently, halo, —CF₃, or —C(CH₃)₃; each V² is H; and each V¹ is, independently, amino or —NC(═O)—R³, where each R³ is, independently, —C₁₋₄alkyl or aryl (optionally substituted with one or more, independently, halo, cyano, or —C₁₋₄alkoxy).

In some embodiments, each X is, independently, O or S; each R¹ is, independently, —(CH₂)₂₋₃NH₂; each R² is, independently, halo, —CF₃, or —C(CH₃)₃; each V² is H; and each V¹ is, independently, amino or —NC(═O)—R³, where each R³ is, independently, —C₁₋₄alkyl or phenyl, thienyl, or pyridyl (each, independently, optionally substituted with one or more, independently, halo, cyano, or —C₁₋₄alkoxy).

In some embodiments, each X is, independently, O, S, or S(═O)₂; each R¹ is, independently, —(CH₂)₁₋₄C(═O)OH, —(CH₂)₁₋₄NHC(═O)C₁₋₄alkyl, —(CH₂)₁₋₄OH, or —(CH₂)₁₋₄C(═O)OC₁₋₄alkyl; each R² is, independently, H, halo, —CF₃, or —C(CH₃)₃; each V² is H; and each V¹ is, independently, —NC(═O)—R³, where each R³ is, independently, —(CH₂)₁₋₄—NH₂ or —(CH₂)₁₋₄—NH—C(═NH)NH₂.

In some embodiments, each X is S; each R¹ is, independently, —(CH₂)₁₋₄C(═O)OH, —(CH₂)₁₋₄NHC(═O)C₁₋₄alkyl, —(CH₂)₁₋₄OH, or —(CH₂)₁₋₄C(═O)OC₁₋₄alkyl; each R² is, independently, halo or —CF₃; and each V¹ is, independently, —NC(═O)—R³, where each R³ is, independently, —(CH₂)₁₋₄—NH₂ or —(CH₂)₁₋₄—NH—C(═NH)NH₂.

In some embodiments, each X is S; each R¹ is, independently, —(CH₂)₂C(═O)OH, —(CH₂)₂NHC(═O)C₁₋₄alkyl, —(CH₂)₂OH, or —(CH₂)₂C(═O)OC₁₋₄alkyl; each R² is, independently, Br or —CF₃; and each V¹ is, independently, —NC(═O)—R³, where each R³ is, independently, —(CH₂)₃₋₄—NH₂ or —(CH₂)₃₋₄—NH—C(═NH)NH₂.

In some embodiments, each X is S; each R¹ is, independently, —(CH₂)₂C(═O)OH, —(CH₂)₂NHC(═O)CH₃, —(CH₂)₂OH, or —(CH₂)₂C(═O)OCH₃; each R² is, independently, Br or —CF₃; and each V¹ is, independently, —NC(═O)—R³, where each R³ is, independently, —(CH₂)₄—NH₂ or —(CH₂)₄—NH—C(═NH)NH₂.

In some embodiments, the compound of Formula Xa is chosen from:

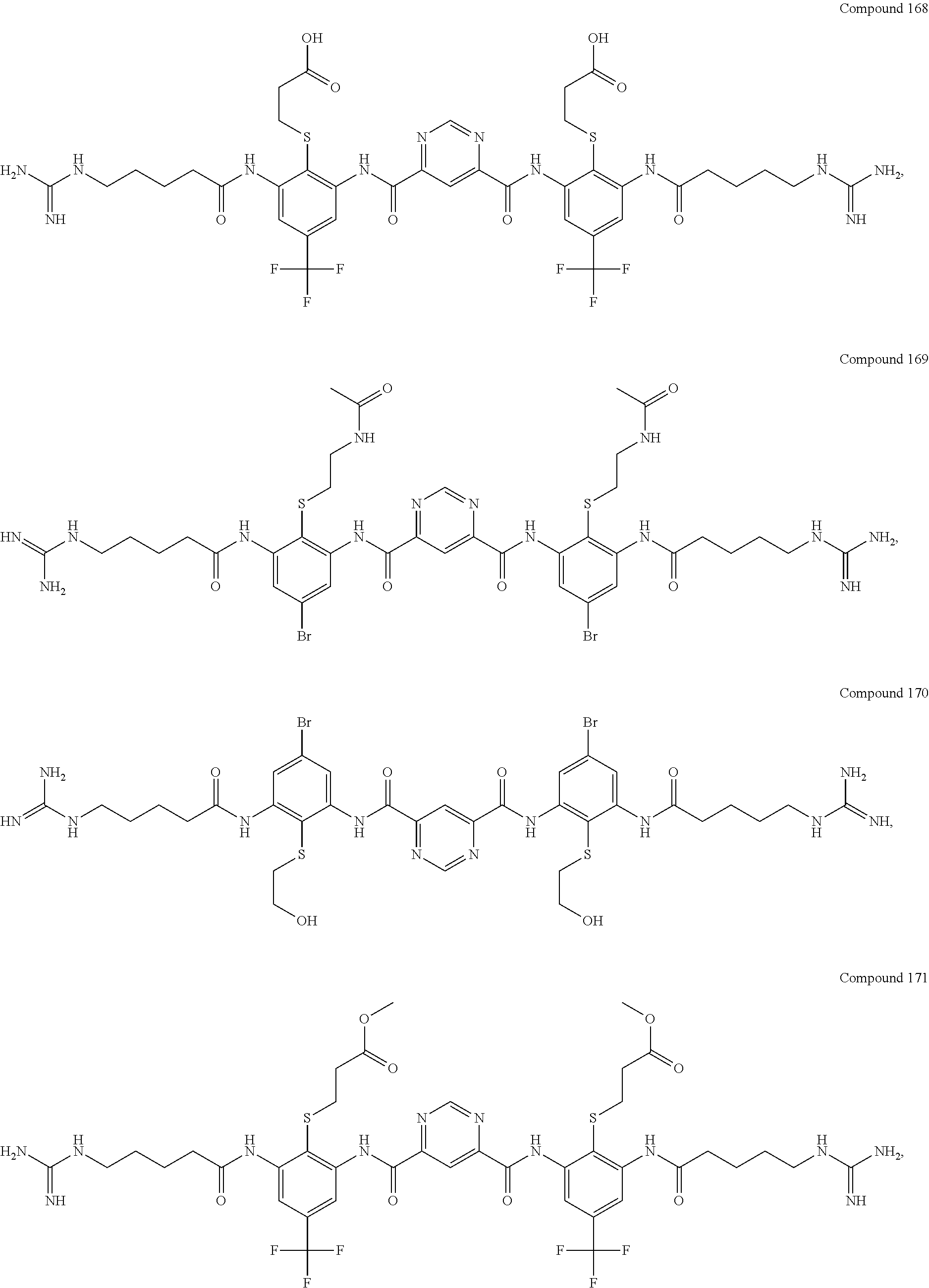 [SMILES: N=C(N)NCCCCC(=O)Nc1cc(C(F)(F)F)cc(NC(=O)c2cc(C(=O)Nc3cc(C(F)(F)F)cc(NC(=O)CCCCNC(=N)N)c3SCCC(=O)O)ncn2)c1SCCC(=O)O ; CC(=O)NCCSc1c(NC(=O)CCCCNC(=N)N)cc(Br)cc1NC(=O)c1cc(C(=O)Nc2cc(Br)cc(NC(=O)CCCCNC(=N)N)c2SCCNC(C)=O)ncn1 ; N=C(N)NCCCCC(=O)Nc1cc(Br)cc(NC(=O)c2cc(C(=O)Nc3cc(Br)cc(NC(=O)CCCCNC(=N)N)c3SCCO)ncn2)c1SCCO ; COC(=O)CCSc1c(NC(=O)CCCCNC(=N)N)cc(C(F)(F)F)cc1NC(=O)c1cc(C(=O)Nc2cc(C(F)(F)F)cc(NC(=O)CCCCNC(=N)N)c2SCCC(=O)OC)ncn1]

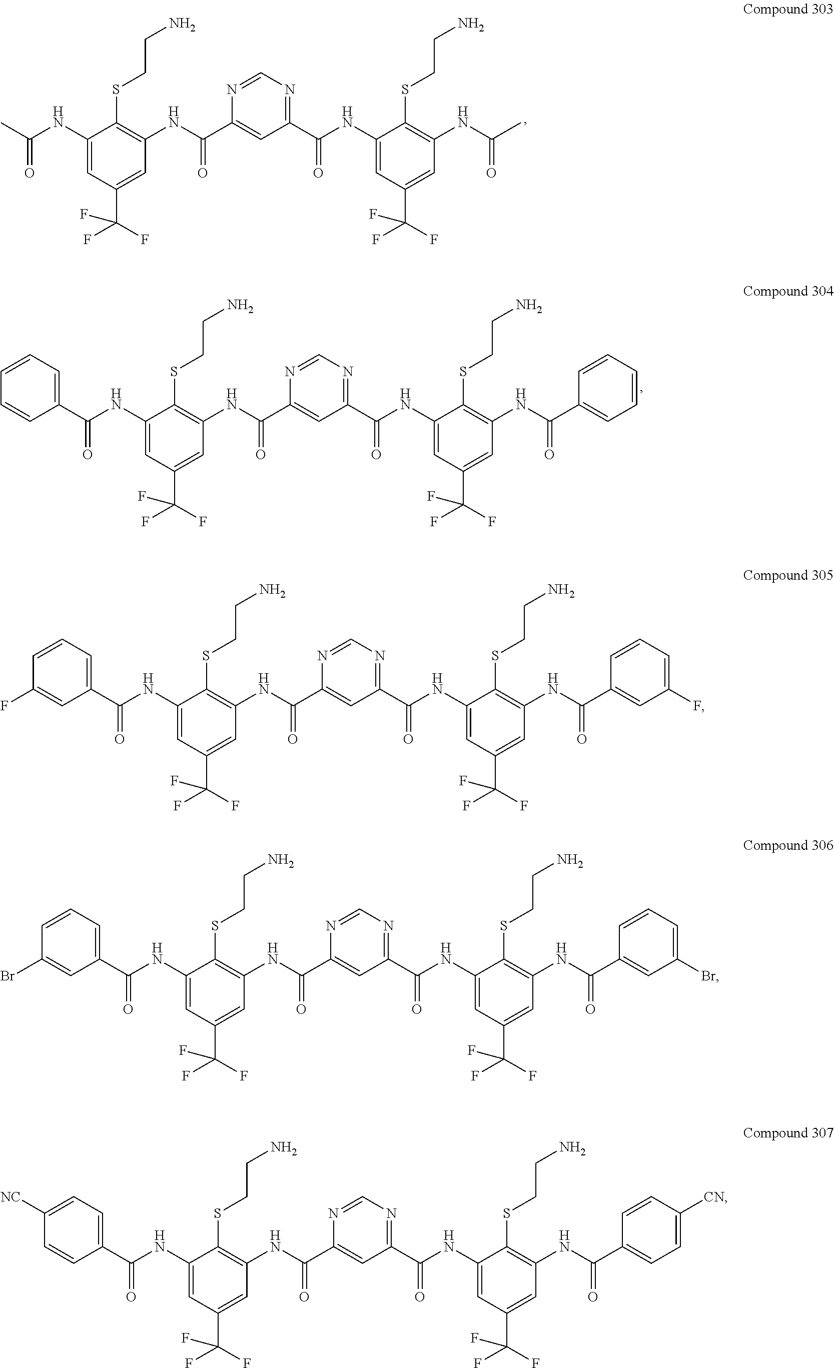

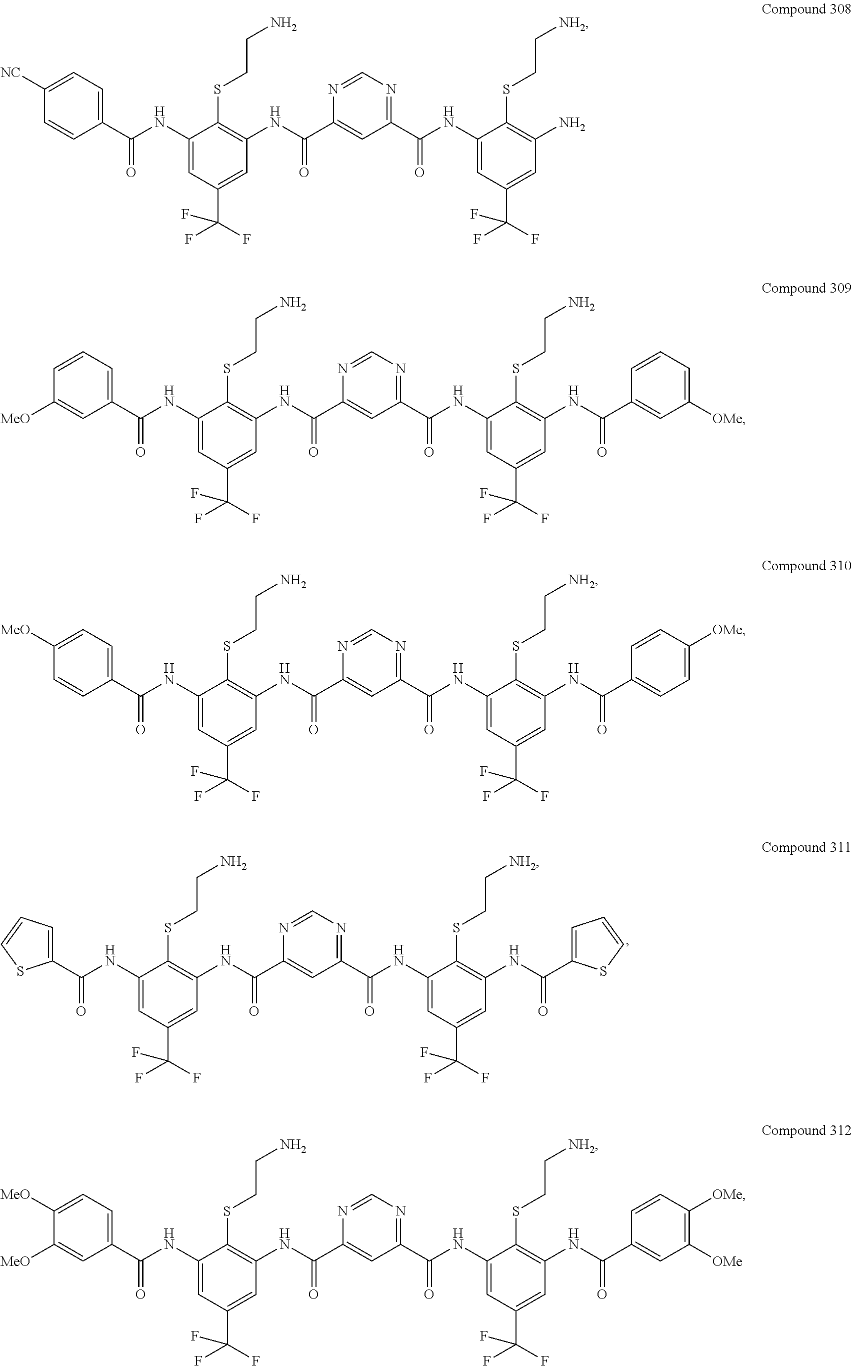

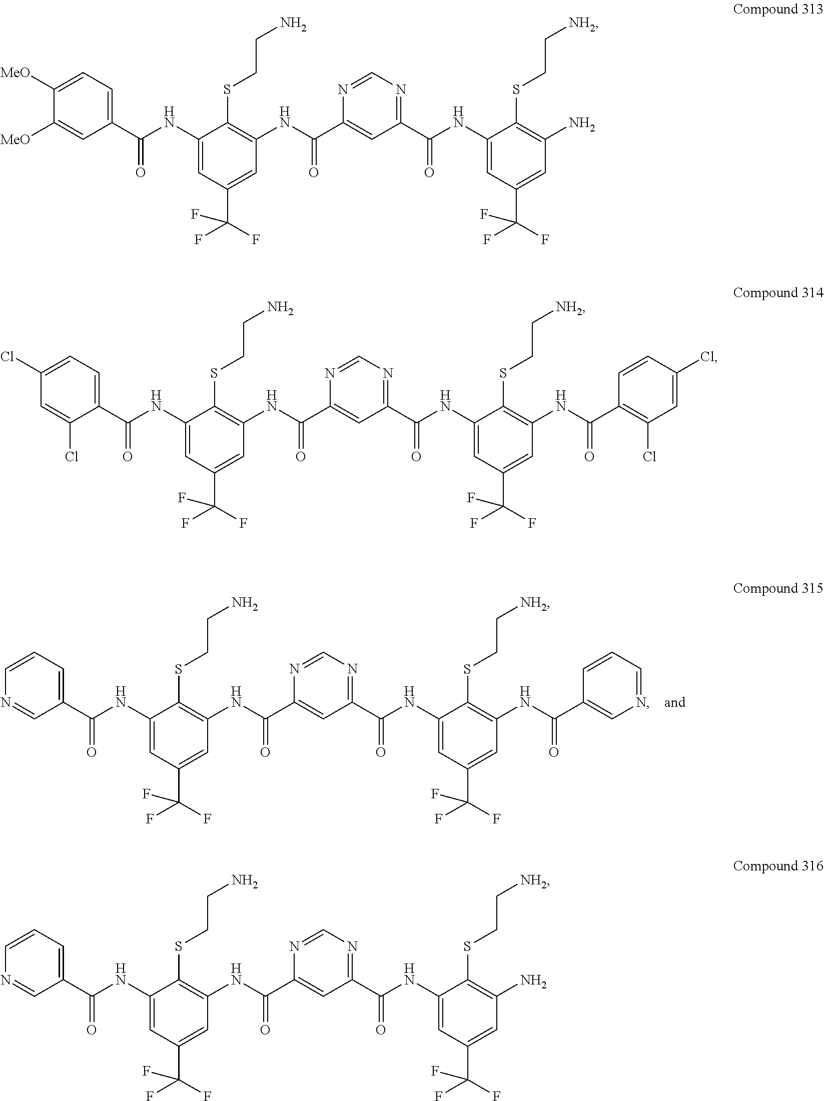 [SMILES: COc1ccc(C(=O)Nc2cc(C(F)(F)F)cc(NC(=O)c3cc(C(=O)Nc4cc(C(F)(F)F)cc(N)c4SCCN)ncn3)c2SCCN)cc1OC ; NCCSc1c(NC(=O)c2cc(C(=O)Nc3cc(C(F)(F)F)cc(NC(=O)c4ccc(Cl)cc4Cl)c3SCCN)ncn2)cc(C(F)(F)F)cc1NC(=O)c1ccc(Cl)cc1Cl ; NCCSc1c(NC(=O)c2cccnc2)cc(C(F)(F)F)cc1NC(=O)c1cc(C(=O)Nc2cc(C(F)(F)F)cc(NC(=O)c3cccnc3)c2SCCN)ncn1 ; "and" ; NCCSc1c(N)cc(C(F)(F)F)cc1NC(=O)c1cc(C(=O)Nc2cc(C(F)(F)F)cc(NC(=O)c3cccnc3)c2SCCN)ncn1]

or a pharmaceutically acceptable salt thereof.

In some embodiments, the compound is not any one or more of Compounds 301 to 316.

The present disclosure also provides anti-malarial compounds of Formula Xb:

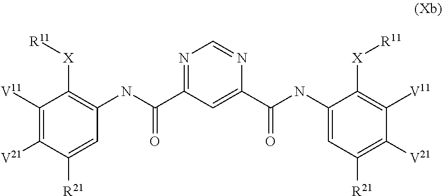

or a pharmaceutically acceptable salt thereof,
wherein:

each X is, independently, O or S;

each R¹¹ is, independently, —(CH₂)₁₋₄NH₂ or —C₁₋₄alkyl;

each R²¹ is, independently, H, halo, —CF₃, or —C(CH₃)₃;

each V¹¹ is H; and

each V² is, independently, —S—(CH₂)₁₋₄—NH₂ or —S—(CH₂)₁₋₄—NHC(═NH)NH₂.

In some embodiments, each R¹¹ is, independently, —C₁₋₄alkyl.

In some embodiments, each R²¹ is, independently, halo or —CF₃.

In some embodiments, each V² is, independently, —S—(CH₂)₂₋₃—NH₂ or —S—(CH₂)₂₋₃—NHC(═NH)NH₂.

In some embodiments, each X is, independently, O or S; each R¹¹ is, independently, —C₁₋₄alkyl; each R²¹ is, independently, halo or —CF₃; each V¹¹ is H; and each V² is, independently, —S—(CH₂)₂₋₃—NH₂ or —S—(CH₂)₂₋₃—NHC(═NH)NH₂.

In some embodiments, the compound of Formula Xb is:

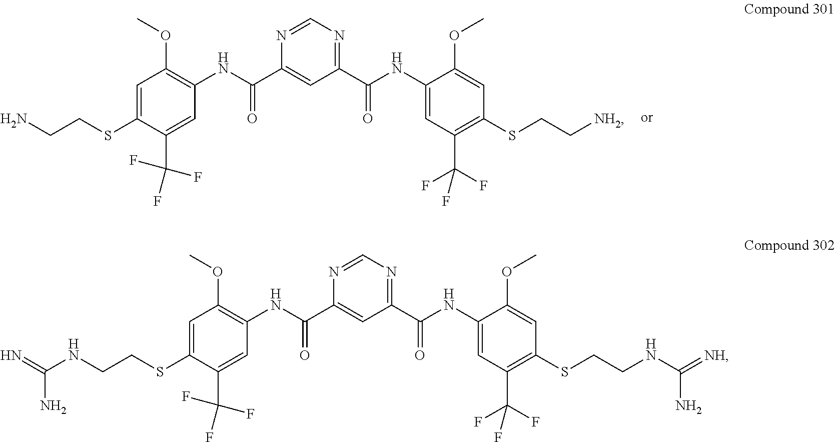 [SMILES: COc1cc(SCCN)c(C(F)(F)F)cc1NC(=O)c1cc(C(=O)Nc2cc(C(F)(F)F)c(SCCN)cc2OC)ncn1 ; "or" ; COc1cc(SCCNC(=N)N)c(C(F)(F)F)cc1NC(=O)c1cc(C(=O)Nc2cc(C(F)(F)F)c(SCCNC(=N)N)cc2OC)ncn1]

or a pharmaceutically acceptable salt thereof.

In some embodiments, the compound is not Compound 301 or 302.

The present disclosure also provides anti-malarial compounds of Formula XI:

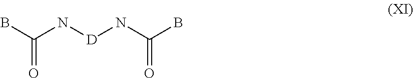

or a pharmaceutically acceptable salt thereof,
wherein:

D is

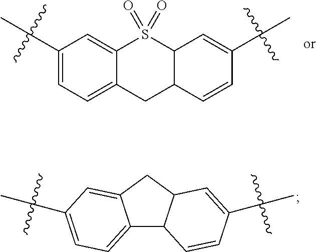 [SMILES: *C1=CC2C(C=C1)Cc1ccc(*)cc1S2(=O)=O ; *C1=CC2Cc3cc(*)ccc3C2C=C1]

each B is, independently

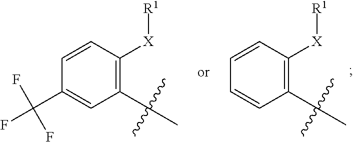

each X is, independently, O or S; and

each R¹ is, independently, —(CH₂)₁₋₄NHC(═NH)NH₂;

In some embodiments, D is

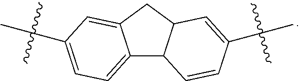 [SMILES: *C1=CC2Cc3cc(*)ccc3C2C=C1]

In some embodiments, each B is

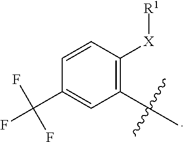

In some embodiments, each B is

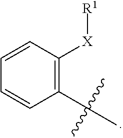

In some embodiments, each X is O. In some embodiments, each X is S.

In some embodiments, each R¹ is, independently, —(CH₂)₂₋₄NHC(═NH)NH₂. In some embodiments, each R¹ is, independently, —(CH₂)₂₋₃NHC(═NH)NH₂. In some embodiments, each R¹ is, independently, —(CH₂)₂NHC(═NH)NH₂.

In some embodiments, D is

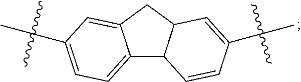 [SMILES: *C1=CC2Cc3cc(*)ccc3C2C=C1]

each B is

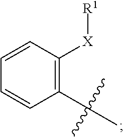

each X is O; and each R¹ is, independently, —(CH₂)₁₋₄NHC(═NH)NH₂.

In some embodiments, D is

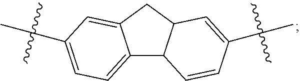 [SMILES: *C1=CC2Cc3cc(*)ccc3C2C=C1]

each B is

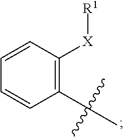

each X is O; and each R¹ is, independently, —(CH₂)₂₋₃NHC(═NH)NH₂.

In some embodiments, the compound of Formula XI is:

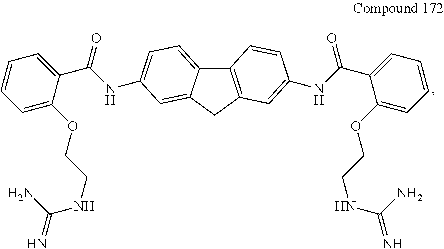 [SMILES: N=C(N)NCCOc1ccccc1C(=O)Nc1ccc2c(c1)Cc1cc(NC(=O)c3ccccc3OCCNC(=N)N)ccc1-2]

or a pharmaceutically acceptable salt thereof.

In some embodiments, the compound is not Compounds 172.

The present disclosure also provides anti-malarial compounds of Formula XII:

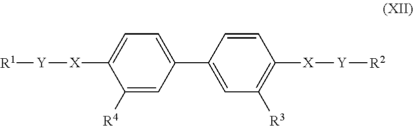

or a pharmaceutically acceptable salt thereof,
wherein:

each X is, independently, —NH—, O, S, or absent;

each Y is, independently, —C(═O), —C(═S), or absent;

R¹ is —(CH₂)ₙNHC(═NH)NH₂, —NH(CH₂)ₙNH₂, —NH(CH₂)ₙNC(═N)NH₂, —(CH₂)ₙNH₂, —O—(CH₂)ₙNH₂, —O—(CH₂)ₙNC(═N)NH₂, —CH═CH—CH₂NH₂, —CH═CH—CH₂NC(═N)NH₂, —CH═CH—(CH₂)₂NH₂, —CH═CH—(CH₂)₂NC(═N)NH₂, —C≡C—CH₂NH₂, —C≡C—(CH₂)₂NH₂, —CH≡CH—CH₂NC(═N)NH₂, or —C≡C—(CH₂)₂—NC(═N)NH₂, where n is 2, 3, or 4; or an aryl, heteroaryl, or heterocycle (each of which is optionally substituted with one or more W);

R² is —(CH₂)ₙNHC(═NH)NH₂, —NH(CH₂)ₙNH₂, —NH(CH₂)ₙNC(═N)NH₂, —(CH₂)ₙNH₂, —O—(CH₂)ₙNH₂, —O—(CH₂)ₙNC(═N)NH₂, —CH═CH—CH₂NH₂, —CH═CH—CH₂NC(═N)NH₂, —CH═CH—(CH₂)₂NH₂, —CH═CH—(CH₂)₂NC(═N)NH₂, —C≡C—CH₂NH₂, —C≡C—(CH₂)₂NH₂, —CH≡CH—CH₂NC(═N)NH₂, or —C≡C—(CH₂)₂—NC(═N)NH₂, where n is 2, 3, or 4; or an aryl, heteroaryl, or heterocycle (each of which is optionally substituted with one or more W);

R³ is H, —CF₃, —NH(CH₂)ₙNH₂, —NH(CH₂)ₙNC(═N)NH₂, —(CH₂)ₙNH₂, —(CH₂)ₙNC(═N)NH₂, —O—(CH₂)ₙNH₂, —O—(CH₂)ₙNC(═N)NH₂, —CH═CH—CH₂NH₂, —CH═CH—CH₂NC(═N)NH₂, —CH═CH—(CH₂)₂NH₂, —CH═CH—(CH₂)₂NC(═N)NH₂, —C≡C—CH₂NH₂, —C≡C—(CH₂)₂NH₂, —CH≡CH—CH₂NC(═N)NH₂, or —C≡C—(CH₂)₂—NC(═N)NH₂, where n is 2, 3, or 4;

R⁴ is H, —CF₃, —NH(CH₂)ₙNH₂, —NH(CH₂)ₙNC(═N)NH₂, —(CH₂)ₙNH₂, —(CH₂)ₙNC(═N)NH₂, —O—(CH₂)ₙNH₂, —O—(CH₂)ₙNC(═N)NH₂, —CH═CH—CH₂NH₂, —CH═CH—CH₂NC(═N)NH₂, —CH═CH—(CH₂)₂NH₂, —CH═CH—(CH₂)₂NC(═N)NH₂, —C≡C—CH₂NH₂, —C≡C—(CH₂)₂NH₂, —CH≡CH—CH₂NC(═N)NH₂, or —C≡C—(CH₂)₂—NC(═N)NH₂, where n is 2, 3, or 4; and

each W is, independently, halo, —CF₃, —S—(CH₂)₁₋₅NHC(═NH)NH₂, —S—(CH₂)₁₋₅NH₂, —S—(CH₂)₁₋₅N(CH₃)₂, —S—(CH₂)₁₋₅NHS(═O)₂—C₁₋₄alkyl, —C₁₋₄alkyl, —O—(CH₂)₁₋₅NHC(═NH)NH₂; or —O-heterocycle or heterocycle wherein the heterocycle is optionally substituted with one or more cyano, amino, guanyl, C₁₋₄alkyl, guanidino, hydroxyl, amidino, C₁₋₄alkoxy, CF₃, or halo.

In some embodiments, each X is, independently, —NH—, O, or S. In some embodiments, each X is —NH—.

In some embodiments, each Y is, independently, —C(═O), or —C(═S). In some embodiments, each Y is —C(═O).

In some embodiments, R¹ is —(CH₂)ₙNHC(═NH)NH₂, —NH(CH₂)ₙNH₂, —NH(CH₂)ₙNC(═N)NH₂, —(CH₂)ₙNH₂, —O—(CH₂)ₙNH₂, —O—(CH₂)ₙNC(═N)NH₂, —CH═CH—CH₂NH₂, —CH═CH—CH₂NC(═N)NH₂, —CH═CH—(CH₂)₂NH₂, —CH═CH—(CH₂)₂NC(═N)NH₂, —C≡C—CH₂NH₂, —C≡C—(CH₂)₂NH₂, —CH≡CH—CH₂NC(═N)NH₂, or —C≡C—(CH₂)₂—NC(═N)NH₂, where n is 2, 3, or 4, or an aryl optionally substituted with one or more W. In some embodiments, R¹ is —(CH₂)₂₋₄NHC(═NH)NH₂, —NH(CH₂)₂₋₄NH₂, —NH(CH₂)₂₋₄NC(═N)NH₂, —(CH₂)₂₋₄NH₂, —O—(CH₂)₂₋₄NH₂, or —O—(CH₂)₂₋₄NC(═N)NH₂, or an aryl optionally substituted with one or more W. In some embodiments, R¹ is —NH(CH₂)₂₋₄NH₂, —(CH₂)₂₋₄NHC(═NH)NH₂, —(CH₂)₂₋₄NH₂, —O—(CH₂)₂₋₄NH₂, or phenyl optionally substituted with one or more W. In some embodiments, R¹ is —(CH₂)₂₋₄NHC(═NH)NH₂, or phenyl substituted with one or more W. In some embodiments, R¹ is —(CH₂)₄NHC(═NH)NH₂ or phenyl substituted with one or two W.

In some embodiments, R² is —(CH₂)ₙNHC(═NH)NH₂, —NH(CH₂)ₙNH₂, —NH(CH₂)ₙNC(═N)NH₂, —(CH₂)ₙNH₂, —O—(CH₂)ₙNH₂, —O—(CH₂)ₙNC(═N)NH₂, —CH═CH—CH₂NH₂, —CH═CH—CH₂NC(═N)NH₂, —CH═CH—(CH₂)₂NH₂, —CH═CH—(CH₂)₂NC(═N)NH₂, —C≡C—CH₂NH₂, —C≡C—(CH₂)₂NH₂, —CH≡CH—CH₂NC(═N)NH₂, or —C≡C—(CH₂)₂—NC(═N)NH₂, where n is 2, 3, or 4, or an aryl optionally substituted with one or more W. In some embodiments, R² is —(CH₂)₂₋₄NHC(═NH)NH₂, —NH(CH₂)₂₋₄NH₂, —NH(CH₂)₂₋₄NC(═N)NH₂, —(CH₂)₂₋₄NH₂, —O—(CH₂)₂₋₄NH₂, or —O—(CH₂)₂₋₄NC(═N)NH₂, or an aryl optionally substituted with one or more W. In some embodiments, R² is —NH(CH₂)₂₋₄NH₂, —(CH₂)₂₋₄NHC(═NH)NH₂, —(CH₂)₂₋₄NH₂, —O—(CH₂)₂₋₄NH₂, or phenyl optionally substituted with one or more W. In some embodiments, R² is —(CH₂)₂₋₄NHC(═NH)NH₂, or phenyl substituted with one or more W. In some embodiments, R² is —(CH₂)₄NHC(═NH)NH₂ or phenyl substituted with one or two W.

In some embodiments, R³ is H or —CF₃.

In some embodiments, R⁴ is H or —CF₃.

In some embodiments, each W is, independently, halo, —CF₃, —S—(CH₂)₁₋₅NH₂, —S—(CH₂)₁₋₅NHC(═NH)NH₂, —S—(CH₂)₁₋₅N(CH₃)₂, —S—(CH₂)₁₋₅NHS(═O)₂—C₁₋₄alkyl, —C₁₋₄alkyl, —O—(CH₂)₁₋₅NHC(═NH)NH₂; or —O-heterocycle wherein the heterocycle is optionally substituted with one or more cyano, amino, guanyl, C₁₋₄alkyl, guanidino, hydroxyl, amidino, C₁₋₄alkoxy, CF₃, or halo. In some embodiments, each W is, independently, halo, —CF₃, —S—(CH₂)₁₋₅NH₂, —S—(CH₂)₁₋₅NHC(═NH)NH₂, —S—(CH₂)₁₋₅N(CH₃)₂, —C₁₋₄alkyl, amino, or C₁₋₄alkoxy. In some embodiments, each W is, independently, halo, —CF₃, —S—(CH₂)₁₋₅NH₂, —C₁₋₄alkyl, or amino. In some embodiments, each W is, independently, halo, —CF₃, or —S—(CH₂)₁₋₅NH₂.

In some embodiments, each X is, independently, —NH—, O, or S; each Y is, independently, —C(═O) or —C(═S); R¹ is —(CH₂)ₙNHC(═NH)NH₂, —NH(CH₂)ₙNH₂, —NH(CH₂)ₙNC(═N)NH₂, —(CH₂)ₙNH₂, —O—(CH₂)ₙNH₂, —O—(CH₂)ₙNC(═N)NH₂, —CH═CH—CH₂NH₂, —CH═CH—CH₂NC(═N)NH₂, —CH═CH—(CH₂)₂NH₂, —CH═CH—(CH₂)₂NC(═N)NH₂, —C≡C—CH₂NH₂, —C≡C—(CH₂)₂NH₂, —CH≡CH—CH₂NC(═N)NH₂, or —C≡C—(CH₂)₂—NC(═N)NH₂, where n is 2, 3, or 4, or an aryl optionally substituted with one or more W;

R² is —(CH₂)ₙNHC(═NH)NH₂, —NH(CH₂)ₙNH₂, —NH(CH₂)ₙNC(═N)NH₂, —(CH₂)ₙNH₂, —O—(CH₂)ₙNH₂, —O—(CH₂)ₙNC(═N)NH₂, —CH═CH—CH₂NH₂, —CH═CH—CH₂NC(═N)NH₂, —CH═CH—(CH₂)₂NH₂, —CH═CH—(CH₂)₂NC(═N)NH₂, —C≡C—CH₂NH₂, —C≡C—(CH₂)₂NH₂, —CH≡CH—CH₂NC(═N)NH₂, or —C≡C—(CH₂)₂—NC(═N)NH₂, where n is 2, 3, or 4, or an aryl optionally substituted with one or more W;

R³ is H, —CF₃, —NH(CH₂)ₙNH₂, —NH(CH₂)ₙNC(═N)NH₂, —(CH₂)ₙNH₂, —(CH₂)ₙNC(═N)NH₂, —O—(CH₂)ₙNH₂, —O—(CH₂)ₙNC(═N)NH₂, —CH═CH—CH₂NH₂, —CH═CH—CH₂NC(═N)NH₂, —CH═CH—(CH₂)₂NH₂, —CH═CH—(CH₂)₂NC(═N)NH₂, —C≡C—CH₂NH₂, —C≡C—(CH₂)₂NH₂, —CH≡CH—CH₂NC(═N)NH₂, or —C≡C—(CH₂)₂—NC(═N)NH₂, where n is 2, 3, or 4;

R⁴ is H, —CF₃, —NH(CH₂)ₙNH₂, —NH(CH₂)ₙNC(═N)NH₂, —(CH₂)ₙNH₂, —(CH₂)ₙNC(═N)NH₂, —O—(CH₂)ₙNH₂, —O—(CH₂)ₙNC(═N)NH₂, —CH═CH—CH₂NH₂, —CH═CH—CH₂NC(═N)NH₂, —CH═CH—(CH₂)₂NH₂, —CH═CH—(CH₂)₂NC(═N)NH₂, —C≡C—CH₂NH₂, —C≡C—(CH₂)₂NH₂, —CH≡CH—CH₂NC(═N)NH₂, or —C≡C—(CH₂)₂—NC(═N)NH₂, where n is 2, 3, or 4; and

each W is, independently, halo, —CF₃, —S—(CH₂)₁₋₅NHC(═NH)NH₂, —S—(CH₂)₁₋₅NH₂, —S—(CH₂)₁₋₅N(CH₃)₂, —S—(CH₂)₁₋₅NHS(═O)₂—C₁₋₄alkyl, —C₁₋₄alkyl, —O—(CH₂)₁₋₅NHC(═NH)NH₂, O-heterocycle wherein the heterocycle is optionally substituted with one or more cyano, amino, guanyl, C₁₋₄alkyl, guanidino, hydroxyl, amidino, C₁₋₄alkoxy, CF₃, or halo.

In some embodiments, each X is —NH—; each Y is —C(═O); R¹ is —(CH₂)₂₋₄NHC(═NH)NH₂, —NH(CH₂)₂₋₄NH₂, —NH(CH₂)₂₋₄NC(═N)NH₂, —(CH₂)₂₋₄NH₂, —O—(CH₂)₂₋₄NH₂, or —O—(CH₂)₂₋₄NC(═N)NH₂, or an aryl optionally substituted with one or more W; R² is —(CH₂)₂₋₄NHC(═NH)NH₂, —NH(CH₂)₂₋₄NH₂, —NH(CH₂)₂₋₄NC(═N)NH₂, —(CH₂)₂₋₄NH₂, —O—(CH₂)₂₋₄NH₂, or —O—(CH₂)₂₋₄NC(═N)NH₂, or an aryl optionally substituted with one or more W; R³ is H or —CF₃; R⁴ is H or —CF₃; and each W is, independently, halo, —CF₃, —S—(CH₂)₁₋₅NH₂, —S—(CH₂)₁₋₅NHC(═NH)NH₂, —S—(CH₂)₁₋₅N(CH₃)₂, —C₁₋₄alkyl, amino, or C₁₋₄alkoxy.

In some embodiments, each X is —NH—; each Y is —C(═O); R¹ is —NH(CH₂)₂₋₄NH₂, —(CH₂)₂₋₄NHC(═NH)NH₂, —(CH₂)₂₋₄NH₂, —O—(CH₂)₂₋₄NH₂, or phenyl optionally substituted with one or more W; R² is —NH(CH₂)₂₋₄NH₂, —(CH₂)₂₋₄NHC(═NH)NH₂, —(CH₂)₂₋₄NH₂, —O—(CH₂)₂₋₄NH₂, or phenyl optionally substituted with one or more W; R³ is H or —CF₃; R⁴ is H or —CF₃; and each W is, independently, halo, —CF₃, —S—(CH₂)₁₋₅NH₂, —C₁₋₄alkyl, or amino.

In some embodiments, each X is —NH—; each Y is —C(═O); R¹ is —NH(CH₂)₄NH₂ or phenyl substituted with one or two W; R² is —NH(CH₂)₄NH₂ or phenyl substituted with one or two W; R³ is H or —CF₃; R⁴ is H or —CF₃; and each W is, independently, halo, —CF₃, or —S—(CH₂)₁₋₅NH₂.

In some embodiments, the compound of Formula XII is chosen from:

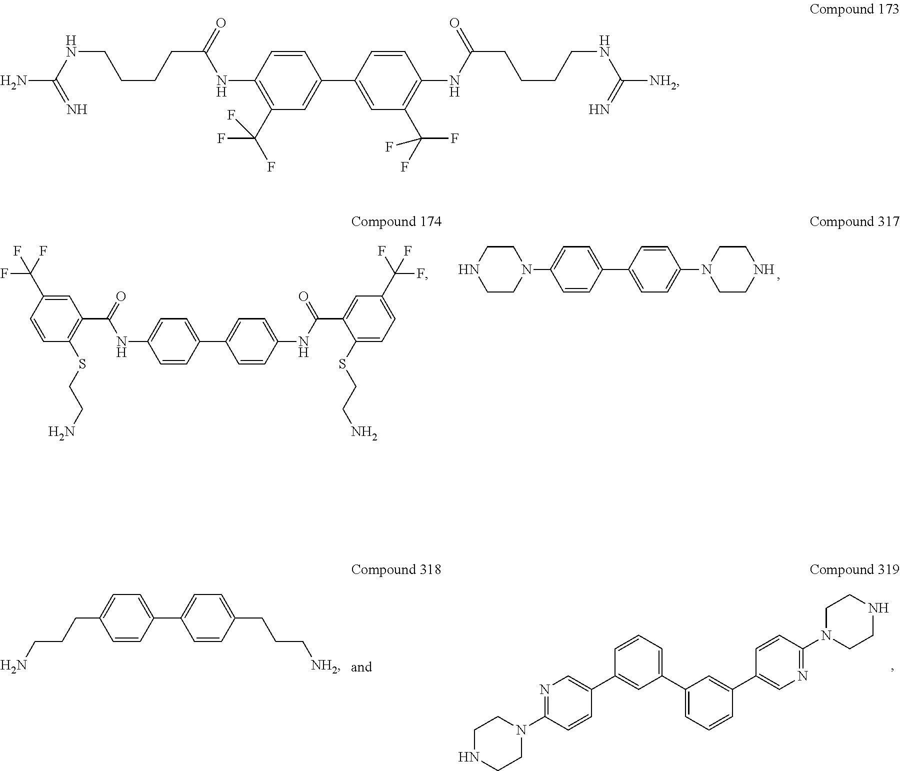 [SMILES: N=C(N)NCCCCC(=O)Nc1ccc(-c2ccc(NC(=O)CCCCNC(=N)N)c(C(F)(F)F)c2)cc1C(F)(F)F ; NCCSc1ccc(C(F)(F)F)cc1C(=O)Nc1ccc(-c2ccc(NC(=O)c3cc(C(F)(F)F)ccc3SCCN)cc2)cc1 ; c1cc(N2CCNCC2)ccc1-c1ccc(N2CCNCC2)cc1 ; NCCCc1ccc(-c2ccc(CCCN)cc2)cc1 ; c1cc(-c2ccc(N3CCNCC3)nc2)cc(-c2cccc(-c3ccc(N4CCNCC4)nc3)c2)c1]

or a pharmaceutically acceptable salt thereof.

In some embodiments, the compound is not any one or more of Compounds 317-319.

The present disclosure also provides anti-malarial compounds of Formula XIII:

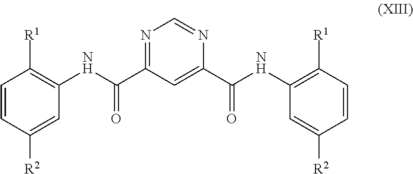

or pharmaceutically acceptable salt thereof,
wherein:

each R¹ is, independently, —S—(CH₂)₁₋₅NH₂, —S—(CH₂)₁₋₅NHC(═NH)NH₂, —C₁₋₄alkoxy, —S—(CH₂)₁₋₅N(C₁₋₄alkyl)₂, —O—(CH₂)₁₋₅NH₂, —O—(CH₂)₁₋₅NHC(═NH)NH₂, —(CH₂)₁₋₅NH₂, —O—(CH₂)₁₋₅N(C₁₋₄alkyl)₂, —C(═O)—(CH₂)₁₋₅NH₂, —C(═O)—(CH₂)₁₋₅NHC(═NH)NH₂, —C(═O)—(CH₂)₁₋₅N(C₁₋₄alkyl)₂, —(CH₂)₁₋₅NHC(═NH)NH₂, —(CH₂)₁₋₅N(C₁₋₄alkyl)₂, —NHC(═O)—(CH₂)₁₋₅NHC(═NH)NH₂, —NHC(═O)—C₁₋₄alkyl, or —NHC(═O)—(CH₂)₁₋₅NHS(═O)₂—C₁₋₄alkyl; and

each R² is, independently, —CF₃, —(CH₂)₁₋₅NH₂, —C₁₋₄alkyl, or halo.

In some embodiments, each R¹ is, independently, —S—(CH₂)₁₋₅NH₂, —C₁₋₄alkoxy, or —(CH₂)₁₋₅NHC(═NH)NH₂. In some embodiments, each R¹ is, independently, —S—(CH₂)₂₋₃NH₂, —C₁₋₃alkoxy, or —(CH₂)₂₋₃NHC(═NH)NH₂. In some embodiments, each R¹ is, independently, —S—(CH₂)₂NH₂, methoxy, or —(CH₂)₂NHC(═NH)NH₂.

In some embodiments, each R² is, independently, —CF₃, —(CH₂)₁₋₂NH₂, —C₁₋₃alkyl, or halo. In some embodiments, each R² is, independently, —CF₃, —CH₂NH₂, methyl, ethyl, F, Cl, or Br.

In some embodiments, each R¹ is, independently, —S—(CH₂)₁₋₅NH₂, —C₁₋₄alkoxy, or —(CH₂)₁₋₅NHC(═NH)NH₂; and each R² is, independently, —CF₃, —(CH₂)₁₋₂NH₂, —C₁₋₃alkyl, or halo.

In some embodiments, each R¹ is, independently, —S—(CH₂)₂₋₃NH₂, —C₁₋₃alkoxy, or —(CH₂)₂₋₃NHC(═NH)NH₂; and each R² is, independently, —CF₃, —CH₂NH₂, methyl, ethyl, F, Cl, or Br.

In some embodiments, each R¹ is, independently, —S—(CH₂)₂NH₂, methoxy, or —(CH₂)₂NHC(═NH)NH₂; and each R² is, independently, —CF₃ or —CH₂NH₂.

In some embodiments, the compound of Formula XIII is chosen from:

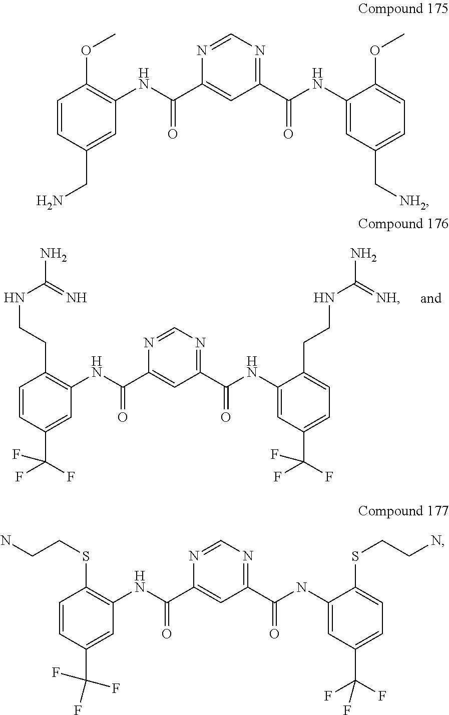

or a pharmaceutically acceptable salt thereof.

In some embodiments, the compound is not any one or more of Compounds 175 to 177.

The present disclosure also provides anti-malarial compounds of Formula XIV:

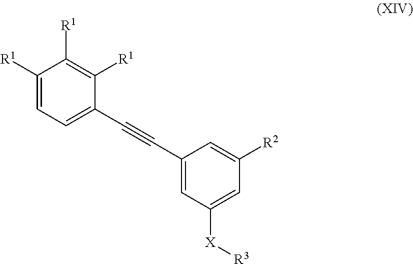

or pharmaceutically acceptable salt thereof,
wherein:

each R¹ is, independently, absent or halo, CF₃, cyano, amino, nitro, amidino, C₁₋₄alkyl, guanidino, hydroxyl, or C₁₋₄alkoxy;

each R² is halo, cyano, amino, nitro, amidino, C₁₋₄alkyl, guanidino, hydroxyl, CF₃, or C₁₋₄alkoxy; and

each R³ is, independently, —S—(CH₂)₁₋₅NH₂, —S—(CH₂)₁₋₅NHC(═NH)NH₂, —C₁₋₄alkoxy, —S—(CH₂)₁₋₅N(C₁₋₄alkyl)₂, —O—(CH₂)₁₋₅NH₂, —O—(CH₂)₁₋₅NHC(═NH)NH₂, —(CH₂)₁₋₅NH₂, —O—(CH₂)₁₋₅N(C₁₋₄alkyl)₂, —C(═O)—(CH₂)₁₋₅NH₂, —C(═O)—(CH₂)₁₋₅NHC(═NH)NH₂, —C(═O)—(CH₂)₁₋₅N(C₁₋₄alkyl)₂, —(CH₂)₁₋₅NHC(═NH)NH₂, —(CH₂)₁₋₅N(C₁₋₄alkyl)₂, —NHC(═O)—(CH₂)₁₋₅NHC(═NH)NH₂, —NHC(═O)—C₁₋₄alkyl, or —NHC(═O)—(CH₂)₁₋₅NHS(═O)₂—C₁₋₄alkyl.

In some embodiments, each R¹ is, independently, absent or halo, CF₃, cyano, amino, nitro, amidino, C₁₋₄alkyl, guanidino, hydroxyl, or C₁₋₄alkoxy. In some embodiments, each R¹ is, independently, absent or halo, CF₃, cyano, amino, nitro, C₁₋₃alkyl, hydroxyl, or C₁₋₃alkoxy. In some embodiments, each R¹ is, independently, absent or halo or CF₃. In some embodiments, each R¹ is, independently, absent or F or CF₃.

In some embodiments, each R² is halo, cyano, amino, nitro, amidino, C₁₋₄alkyl, guanidino, hydroxyl, CF₃, or C₁₋₄alkoxy. In some embodiments, each R² is halo, cyano, amino, nitro, C₁₋₄alkyl, hydroxyl, CF₃, or C₁₋₄alkoxy. In some embodiments, each R² is halo. In some embodiments, each R² is Br.

In some embodiments, each R³ is, independently, —S—(CH₂)₁₋₅NH₂, —O—(CH₂)₁₋₅NH₂, —S—(CH₂)₁₋₅NHC(═NH)NH₂, —S—(CH₂)₁₋₅N(C₁₋₄alkyl)₂, —O—(CH₂)₁₋₅NHC(═NH)NH₂, —O—(CH₂)₁₋₅N(C₁₋₄alkyl)₂, —(CH₂)₁₋₅NH₂, —(CH₂)₁₋₅NHC(═NH)NH₂, or —(CH₂)₁₋₅N(C₁₋₄alkyl)₂. In some embodiments, each R³ is, independently, —(CH₂)₁₋₅NH₂, —(CH₂)₁₋₅NHC(═NH)NH₂, or —(CH₂)₁₋₅N(C₁₋₄alkyl)₂. In some embodiments, each R³ is, independently, —(CH₂)₁₋₅NH₂. In some embodiments, each R³ is, independently, —(CH₂)₂₋₃NH₂.

In some embodiments, each R¹ is, independently, absent or halo, CF₃, cyano, amino, nitro, amidino, C₁₋₄alkyl, guanidino, hydroxyl, or C₁₋₄alkoxy; each R² is halo, cyano, amino, nitro, amidino, C₁₋₄alkyl, guanidino, hydroxyl, CF₃, or C₁₋₄alkoxy; and each R³ is, independently, —S—(CH₂)₁₋₅NH₂, —S—(CH₂)₁₋₅NHC(═NH)NH₂, —S—(CH₂)₁₋₅N(C₁₋₄alkyl)₂, —O—(CH₂)₁₋₅NH₂, —O—(CH₂)₁₋₅NHC(═NH)NH₂, —O—(CH₂)₁₋₅N(C₁₋₄alkyl)₂, —(CH₂)₁₋₅NH₂, —(CH₂)₁₋₅NHC(═NH)NH₂, or —(CH₂)₁₋₅N(C₁₋₄alkyl)₂.

In some embodiments, each R¹ is, independently, absent or halo, CF₃, cyano, amino, nitro, C₁₋₃alkyl, hydroxyl, or C₁₋₃alkoxy; each R² is halo, cyano, amino, nitro, C₁₋₄alkyl, hydroxyl, CF₃, or C₁₋₄alkoxy; and each R³ is, independently, —(CH₂)₁₋₅NH₂, —(CH₂)₁₋₅NHC(═NH)NH₂, or —(CH₂)₁₋₅N(C₁₋₄alkyl)₂.

In some embodiments, each R¹ is, independently, absent or halo or CF₃; each R² is halo; and each R³ is, independently, —(CH₂)₁₋₅NH₂.

In some embodiments, each R¹ is, independently, absent or F or CF₃; each R² is Br; and each R³ is, independently, —(CH₂)₂₋₃NH₂.

In some embodiments, the compound of Formula XIV is:

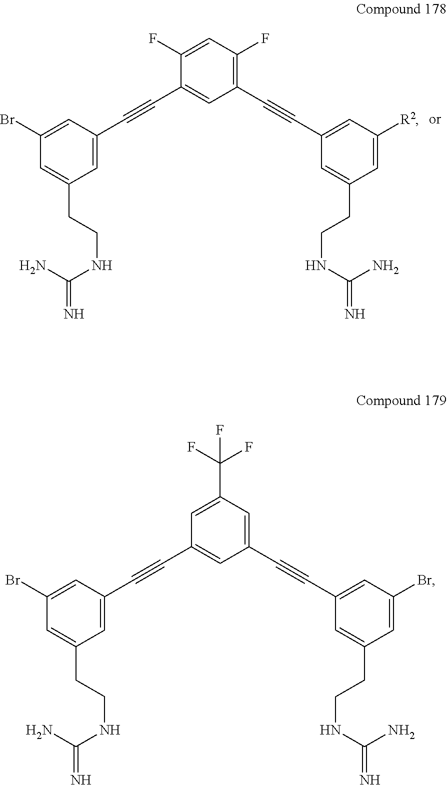

or a pharmaceutically acceptable salt thereof.

In some embodiments, the compound is Compound 178 or 179.

The present disclosure also provides anti-malarial compounds of Formula XV:

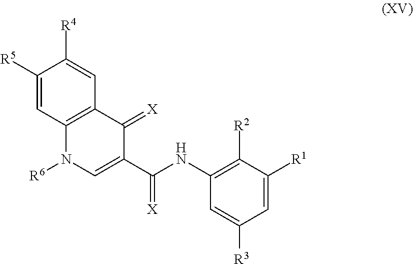

or a pharmaceutically acceptable salt thereof,
wherein:

each X is, independently, O or S;

R¹ is amino, cyano, C₁₋₄alkyl, guanidino, hydroxyl, amidino, C₁₋₄alkoxy, CF₃, or halo;

R² is —S—(CH₂)₁₋₅NH₂, —S—(CH₂)₁₋₅NHC(═NH)NH₂, —S—(CH₂)₁₋₅N(C₁₋₄alkyl)₂, —O—(CH₂)₁₋₅NH₂, —O—(CH₂)₁₋₅NHC(═NH)NH₂, —(CH₂)₁₋₅NH₂, —O—(CH₂)₁₋₅N(C₁₋₄alkyl)₂, —C(═O)—(CH₂)₁₋₅NH₂, —C(═O)—(CH₂)₁₋₅NHC(═NH)NH₂, —C(═O)—(CH₂)₁₋₅N(C₁₋₄alkyl)₂, —(CH₂)₁₋₅NHC(═NH)NH₂, —(CH₂)₁₋₅N(C₁₋₄alkyl)₂, —NHC(═O)—(CH₂)₁₋₅NHC(═NH)NH₂, —NHC(═O)—C₁₋₄alkyl, or —NHC(═O)—(CH₂)₁₋₅NHS(═O)₂—C₁₋₄alkyl;

R³ is CF₃, amino, cyano, C₁₋₄alkyl, guanidino, hydroxyl, amidino, C₁₋₄alkoxy, or halo;

R⁴ is halo, amino, cyano, C₁₋₄alkyl, guanidino, hydroxyl, amidino, C₁₋₄alkoxy, or CF₃;

R⁵ is a heterocycloalkyl; and

R⁶ is a cycloalkyl.

In some embodiments, each X is O. In some embodiments, each X is S.

In some embodiments, R¹ is amino, cyano, C₁₋₄alkyl, hydroxyl, C₁₋₄alkoxy, CF₃, or halo. In some embodiments, R¹ is amino, cyano, C₁₋₂alkyl, C₁₋₂alkoxy, CF₃, or halo. In some embodiments, R¹ is amino, CF₃, or halo. In some embodiments, R¹ is amino.

In some embodiments, R² is —S—(CH₂)₁₋₅NH₂, —S—(CH₂)₁₋₅NHC(═NH)NH₂, —O—(CH₂)₁₋₅NH₂, —O—(CH₂)₁₋₅NHC(═NH)NH₂, —(CH₂)₁₋₅NH₂, —C(═O)—(CH₂)₁₋₅NH₂, —C(═O)—(CH₂)₁₋₅NHC(═NH)NH₂, —(CH₂)₁₋₅NHC(═NH)NH₂, —NHC(═O)—C₁₋₄alkyl, or —NHC(═O)—(CH₂)₁₋₅NHC(═NH)NH₂. In some embodiments, R² is —S—(CH₂)₁₋₅NH₂, —S—(CH₂)₁₋₅NHC(═NH)NH₂, —O—(CH₂)₁₋₅NH₂, —O—(CH₂)₁₋₅NHC(═NH)NH₂, —(CH₂)₁₋₅NH₂, or —(CH₂)₁₋₅NHC(═NH)NH₂. In some embodiments, R² is —S—(CH₂)₁₋₅NH₂, —O—(CH₂)₁₋₅NH₂, or —(CH₂)₁₋₅NH₂. In some embodiments, R² is —S—(CH₂)₂NH₂, —O—(CH₂)₂NH₂, or —(CH₂)₂NH₂.

In some embodiments, R³ is CF₃, amino, cyano, C₁₋₄alkyl, guanidino, hydroxyl, amidino, C₁₋₄alkoxy, or halo. In some embodiments, R³ is CF₃, amino, cyano, C₁₋₄alkyl, hydroxyl, C₁₋₄alkoxy, or halo. In some embodiments, R³ is CF₃, amino, cyano, C₁₋₄alkyl, or halo. In some embodiments, R³ is CF₃, amino, or halo. In some embodiments, R³ is CF₃.

In some embodiments, R⁴ is halo, amino, cyano, C₁₋₄alkyl, hydroxyl, C₁₋₄alkoxy, or CF₃. In some embodiments, R⁴ is halo, amino, C₁₋₄alkyl, C₁₋₄alkoxy, or CF₃. In some embodiments, R⁴ is halo, amino, or CF₃. In some embodiments, R⁴ is halo.

In some embodiments, R⁵ is morpholino, thiomorpholino, piperazinyl, tetrahydrofuranyl, tetrahydrothienyl, 2,3-dihydrobenzofuryl, 1,3-benzodioxole, benzo-1,4-dioxane, piperidinyl, pyrrolidinyl, isoxazolidinyl, oxazolidinyl, isothiazolidinyl, pyrazolidinyl, thiazolidinyl, or imidazolidinyl. In some embodiments, R⁵ is piperazinyl, morpholino, tetrahydrofuranyl, piperidinyl, pyrrolidinyl, isoxazolidinyl, oxazolidinyl, pyrazolidinyl, or imidazolidinyl. In some embodiments, R⁵ is piperazinyl, morpholino, piperidinyl, or pyrazolidinyl. In some embodiments, R⁵ is piperazinyl.

In some embodiments, R⁶ is cyclopropyl, cyclobutyl, cyclopentyl, cyclohexyl, cycloheptyl, cyclooctyl, cyclononyl. In some embodiments, R⁶ is cyclopropyl, cyclobutyl, cyclopentyl, or cyclohexyl. In some embodiments, R⁶ is cyclopropyl, cyclobutyl, or cyclohexyl. In some embodiments, R⁶ is cyclopropyl.

In some embodiments, each X is O; R¹ is amino, cyano, C₁₋₄alkyl, hydroxyl, C₁₋₄alkoxy, CF₃, or halo; R² is —S—(CH₂)₁₋₅NH₂, —S—(CH₂)₁₋₅NHC(═NH)NH₂, —O—(CH₂)₁₋₅NH₂, —(CH₂)₁₋₅NH₂, —O—(CH₂)₁₋₅NHC(═NH)NH₂, —C(═O)—(CH₂)₁₋₅NH₂, —C(═O)—(CH₂)₁₋₅NHC(═NH)NH₂, —(CH₂)₁₋₅NHC(═NH)NH₂, —NHC(═O)—C₁₋₄alkyl, or —NHC(═O)—(CH₂)₁₋₅NHC(═NH)NH₂; R³ is CF₃, amino, cyano, C₁₋₄alkyl, guanidino, hydroxyl, amidino, C₁₋₄alkoxy, or halo; R⁴ is halo, amino, cyano, C₁₋₄alkyl, hydroxyl, C₁₋₄alkoxy, or CF₃; R⁵ is morpholino, thiomorpholino, piperazinyl, tetrahydrofuranyl, tetrahydrothienyl, 2,3-dihydrobenzofuryl, 1,3-benzodioxole, benzo-1,4-dioxane, piperidinyl, pyrrolidinyl, isoxazolidinyl, oxazolidinyl, isothiazolidinyl, pyrazolidinyl, thiazolidinyl, or imidazolidinyl; and R⁶ is cyclopropyl, cyclobutyl, cyclopentyl, cyclohexyl, cycloheptyl, cyclooctyl, cyclononyl.

In some embodiments, each X is O; R¹ is amino, cyano, C₁₋₂alkyl, C₁₋₂alkoxy, CF₃, or halo; R² is —S—(CH₂)₁₋₅NH₂, —S—(CH₂)₁₋₅NHC(═NH)NH₂, —O—(CH₂)₁₋₅NH₂, —(CH₂)₁₋₅NH₂, —O—(CH₂)₁₋₅NHC(═NH)NH₂, or —(CH₂)₁₋₅NHC(═NH)NH₂; R³ is CF₃, amino, cyano, C₁₋₄alkyl, hydroxyl, C₁₋₄alkoxy, or halo; R⁴ is halo, amino, C₁₋₄alkyl, C₁₋₄alkoxy, or CF₃; R⁵ is piperazinyl, morpholino, tetrahydrofuranyl, piperidinyl, pyrrolidinyl, isoxazolidinyl, oxazolidinyl, pyrazolidinyl, or imidazolidinyl; and R⁶ is cyclopropyl, cyclobutyl, cyclopentyl, or cyclohexyl.

In some embodiments, each X is O; R¹ is amino, CF₃, or halo; R² is —S—(CH₂)₁₋₅NH₂, —O—(CH₂)₁₋₅NH₂, or —(CH₂)₁₋₅NH₂; R³ is CF₃, amino, cyano, C₁₋₄alkyl, or halo; R⁴ is halo, amino, or CF₃; R⁵ is piperazinyl, morpholino, piperidinyl, or pyrazolidinyl; and R⁶ is cyclopropyl, cyclobutyl, or cyclohexyl.

In some embodiments, each X is O; R¹ is amino; R² is —S—(CH₂)₂NH₂, —O—(CH₂)₂NH₂, or —(CH₂)₂NH₂; R³ is CF₃, amino, or halo; R⁴ is halo; R⁵ is piperazinyl; and R⁶ is cyclopropyl, cyclobutyl, or cyclohexyl.

In some embodiments, the compound of Formula XV is:

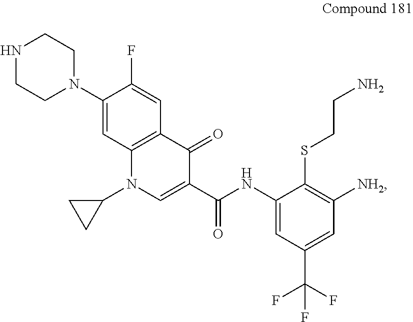 [SMILES: NCCSc1c(N)cc(C(F)(F)F)cc1NC(=O)c1cn(C2CC2)c2cc(N3CCNCC3)c(F)cc2c1=O]

or a pharmaceutically acceptable salt thereof.

The present disclosure also provides anti-malarial compounds of Formula XVI:

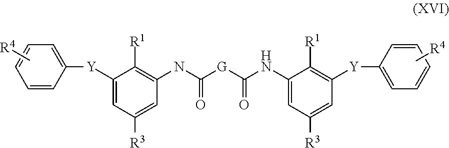

or a pharmaceutically acceptable salt thereof,
wherein:

G is

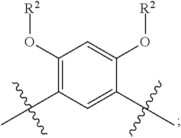

each Y is —NH—C(═O)—;

each R¹ is, independently, H or —S—(CH₂)₁₋₄NH₂, or —S—(CH₂)₁₋₄NHC(═NH)NH₂;

each R² is, independently, H, C₁-C₄alkyl, —(CH₂)₁₋₄NH₂, or —(CH₂)₁₋₄NHC(═NH)NH₂;

each R³ is, independently, H, —CF₃, —C(CH₃)₃, halo, or OH; and

each R⁴ is, independently, H, —CF₃, —C(CH₃)₃, halo, cyano, or OH.

In some embodiments, each R¹ is, independently, H or —S—(CH₂)₁₋₄NH₂. In some embodiments, each R¹ is, independently, H or —S—(CH₂)₂NH₂.

In some embodiments, each R² is, independently, C₁-C₄alkyl or —(CH₂)₁₋₄NH₂. In some embodiments, each R² is, independently, methyl or —(CH₂)₂NH₂.

In some embodiments, each R³ is, independently, —CF₃, —C(CH₃)₃, or halo. In some embodiments, each R³ is, independently, —CF₃ or halo.

In some embodiments, each R⁴ is, independently, —CF₃, halo, or cyano. In some embodiments, each R⁴ is, independently, halo or cyano.

In some embodiments, each R¹ is, independently, H or —S—(CH₂)₁₋₄NH₂; each R² is, independently, C₁-C₄alkyl or —(CH₂)₁₋₄NH₂; each R³ is, independently, —CF₃, —C(CH₃)₃, or halo; and each R⁴ is, independently, —CF₃, halo, or cyano.

In some embodiments, each R¹ is, independently, H or —S—(CH₂)₂NH₂; each R² is, independently, methyl or —(CH₂)₂NH₂; each R³ is, independently, —CF₃ or halo; and each R⁴ is, independently, halo or cyano.

In some embodiments, the compound of Formula XVI is:

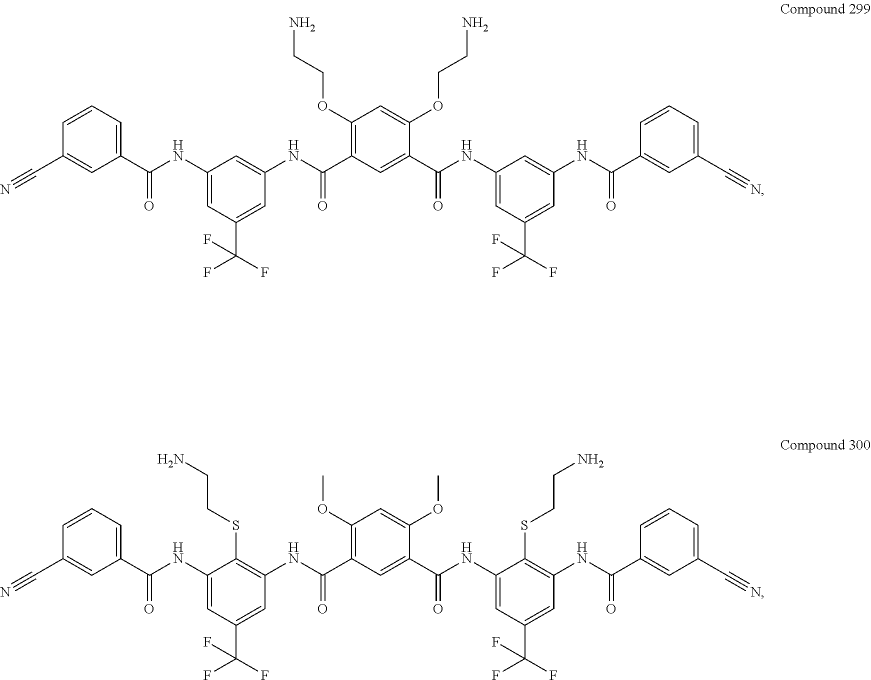 [SMILES: N#Cc1cccc(C(=O)Nc2cc(NC(=O)c3cc(C(=O)Nc4cc(NC(=O)c5cccc(C#N)c5)cc(C(F)(F)F)c4)c(OCCN)cc3OCCN)cc(C(F)(F)F)c2)c1 ; COc1cc(OC)c(C(=O)Nc2cc(C(F)(F)F)cc(NC(=O)c3cccc(C#N)c3)c2SCCN)cc1C(=O)Nc1cc(C(F)(F)F)cc(NC(=O)c2cccc(C#N)c2)c1SCCN]

or a pharmaceutically acceptable salt thereof.

In some embodiments, the compound is not Compound 299 or 300.

The present disclosure also provides pharmaceutical compositions comprising any one or more of the foregoing compounds, or a pharmaceutically acceptable salt thereof, and a pharmaceutically acceptable carrier.

The present disclosure also provides methods of treating malaria in a mammal comprising administering to the mammal, optionally in need thereof, a therapeutically effective amount of any one or more of the foregoing compounds, or a pharmaceutically acceptable salt thereof.

The present disclosure also provides methods of killing or inhibiting the growth of a Plasmodium species comprising contacting the species with an effective amount of any one or more of the foregoing compounds, or a pharmaceutically acceptable salt thereof.

The present disclosure also provides any one or more of the foregoing compounds for treating malaria in a mammal, or killing or inhibiting the growth of a Plasmodium species.

The present disclosure also provides any one or more of the foregoing compounds for use in the manufacture of a medicament for treating malaria in a mammal, or killing or inhibiting the growth of a Plasmodium species.

The present disclosure also provides uses of any one or more of the foregoing compounds for treating malaria in a mammal, or killing or inhibiting the growth of a Plasmodium species.

The present disclosure also provides uses of any one or more of the foregoing compounds in the manufacture of a medicament for treating malaria in a mammal, or killing or inhibiting the growth of a Plasmodium species.

The present disclosure also provides methods of treating malaria in a mammal or killing or inhibiting the growth of a Plasmodium species comprising administering to the mammal, optionally in need thereof, a therapeutically effective amount of, or contacting the species with an effective amount of any one or more of the following compounds:

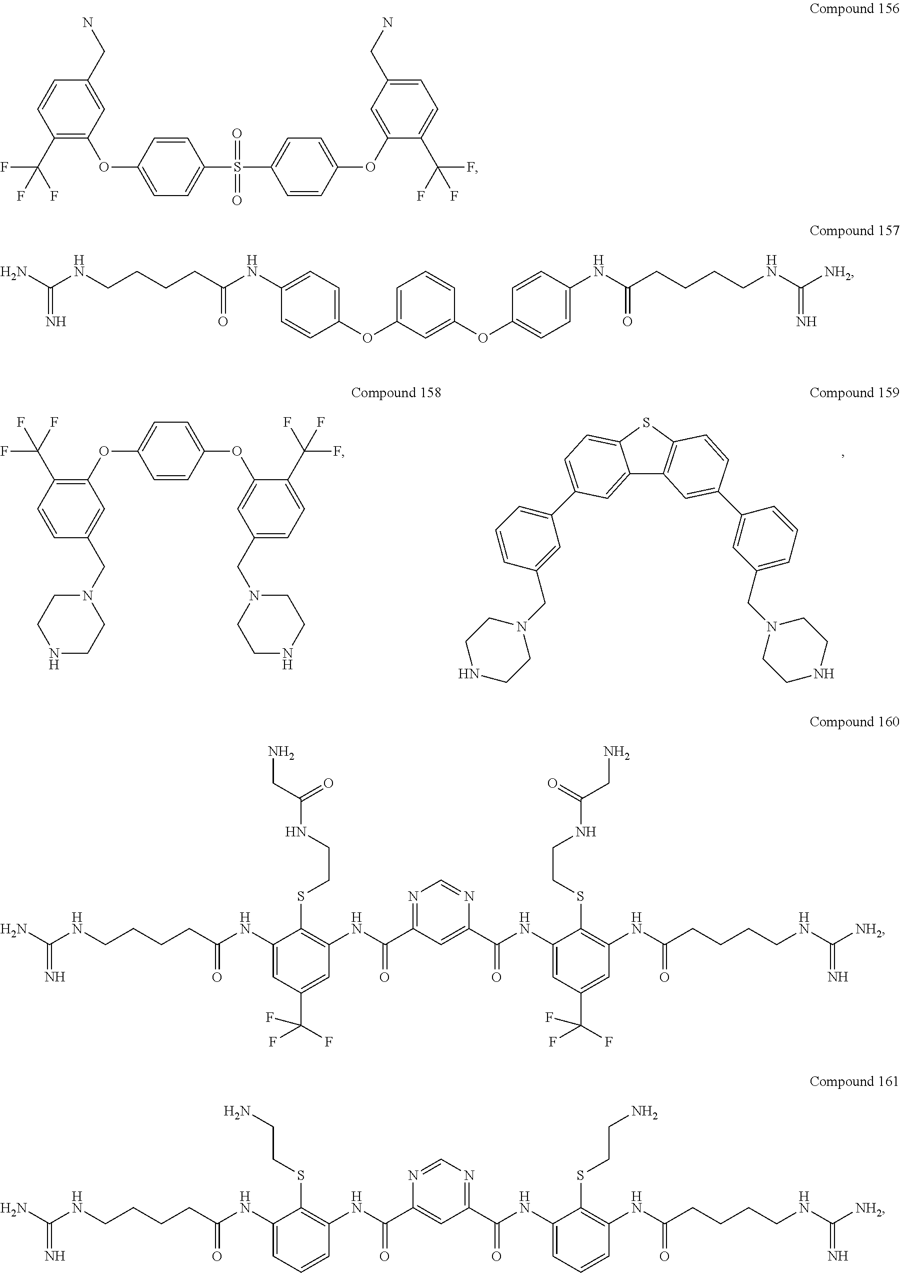 [SMILES: NCc1ccc(C(F)(F)F)c(Oc2ccc(S(=O)(=O)c3ccc(Oc4cc(CN)ccc4C(F)(F)F)cc3)cc2)c1 ; N=C(N)NCCCCC(=O)Nc1ccc(Oc2cccc(Oc3ccc(NC(=O)CCCCNC(=N)N)cc3)c2)cc1 ; FC(F)(F)c1ccc(CN2CCNCC2)cc1Oc1ccc(Oc2cc(CN3CCNCC3)ccc2C(F)(F)F)cc1 ; c1cc(CN2CCNCC2)cc(-c2ccc3sc4ccc(-c5cccc(CN6CCNCC6)c5)cc4c3c2)c1 ; N=C(N)NCCCCC(=O)Nc1cc(C(F)(F)F)cc(NC(=O)c2cc(C(=O)Nc3cc(C(F)(F)F)cc(NC(=O)CCCCNC(=N)N)c3SCCNC(=O)CN)ncn2)c1SCCNC(=O)CN ; N=C(N)NCCCCC(=O)Nc1cccc(NC(=O)c2cc(C(=O)Nc3cccc(NC(=O)CCCCNC(=N)N)c3SCCN)ncn2)c1SCCN]

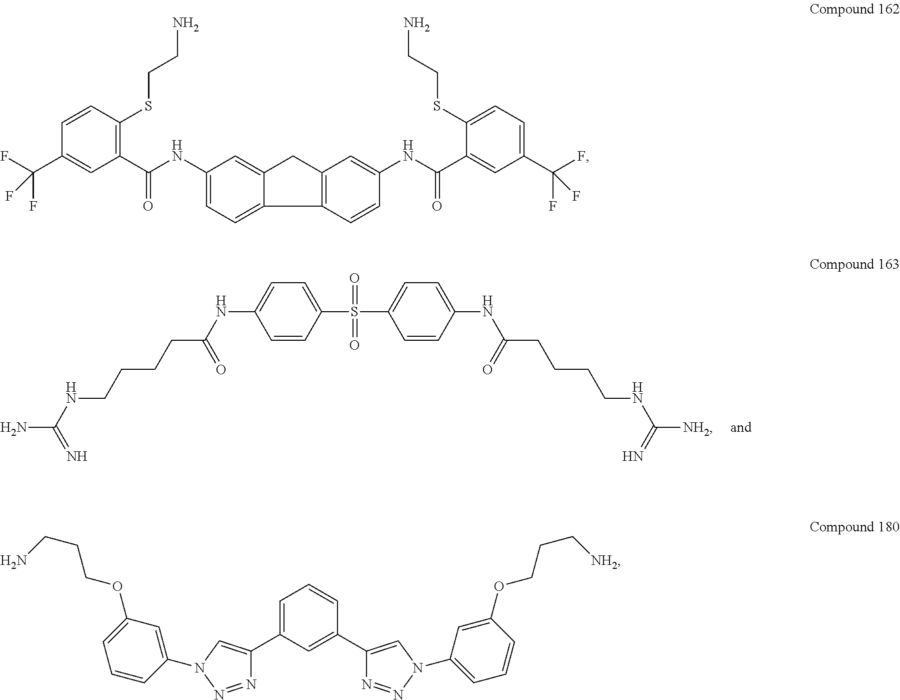 [SMILES: NCCSc1ccc(C(F)(F)F)cc1C(=O)Nc1ccc2c(c1)Cc1cc(NC(=O)c3cc(C(F)(F)F)ccc3SCCN)ccc1-2 ; N=C(N)NCCCCC(=O)Nc1ccc(S(=O)(=O)c2ccc(NC(=O)CCCCNC(=N)N)cc2)cc1 ; "and" ; NCCCOc1cccc(-n2cc(-c3cccc(-c4cn(-c5cccc(OCCCN)c5)nn4)c3)nn2)c1]

or a pharmaceutically acceptable salt thereof.

Polyamides and polyesters that are useful for the present disclosure can be prepared by typical condensation polymerization and addition polymerization processes (see, for example, G. Odian, Principles of Polymerization, John Wiley & Sons, Third Edition (1991), and M. Steven, Polymer Chemistry, Oxford University Press (1999)). Most commonly, the polyamides are prepared by a) thermal dehydration of amine salts of carboxylic acids, b) reaction of acid chlorides with amines, and c) aminolysis of esters. Methods a) and c) are of limited use in polymerizations of aniline derivatives which are generally prepared utilizing acid chlorides. The skilled chemist, however, will recognize that there are many alternative active acylating agents, for example phosphoryl anhydrides, active esters or azides, which may replace an acid chloride and which, depending of the particular polymer being prepared, may be superior to an acid chloride. The acid chloride route is probably the most versatile and has been used extensively for the synthesis of aromatic polyamides.

Homopolymers derived from substituted aminobenzoic acid derivatives can also prepared in a stepwise fashion. A stepwise process comprises coupling an N-protected amino acid to an amine (or hydroxy group) and subsequently removing the amine-protecting group and repeating the process. These techniques have been highly refined for synthesis of specific peptides, allow for the synthesis of specific sequences, and both solid-phase and solution techniques for peptide synthesis are directly applicable to the present disclosure. An alternative embodiment of the present disclosure is the corresponding polysulfonamides that can be prepared in analogous fashion by substituting sulfonyl chlorides for carboxylic acid chlorides.

The most common method for the preparation of polyureas is the reaction of diamines with diisocyanates (see, Yamaguchi et al., Polym. Bull., 2000, 44, 247). This exothermic reaction can be carried out by solution techniques or by interfacial techniques. One skilled in organic and polymer chemistry will appreciate that the diisocyanate can be replaced with a variety of other bis-acylating agents, such as phosgene or N,N′-(diimidazolyl)carbonyl, with similar results. Polyurethanes are prepared by comparable techniques using a diisocyanate and a dialcohol or by reaction of a diamine with a bis-chloroformate.

The syntheses of compounds described herein can be carried out by routine and/or known methods such as those disclosed in, for example, U.S. Patent Application Publication Nos. 2005-0287108, 2006-0041023, U.S. Pat. No. 7,173,102, International Publication Nos. WO 2005/123660, WO 2004/082643, and WO 2006/093813, and U.S. Application Publication No. 2010-0081665, each of which is incorporated herein by reference in its entirety. Numerous pathways are available to incorporate polar and nonpolar side chains. Phenolic groups on the monomer can be alkylated. Alkylation of the commercially available phenol will be accomplished with standard Williamson ether synthesis for the non-polar side chain with ethyl bromide as the alkylating agent. Polar sidechains can be introduced with bifunctional alkylating agents such as BOC—NH(CH₂)₂Br. Alternately, the phenol group can be alkylated to install the desired polar side chain function by employing the Mitsonobu reaction with BOC—NH(CH₂)₂—OH, triphenyl phosphine, and diethyl acetylenedicarboxylate. Standard conditions for reduction of the nitro groups and hydrolysis of the ester afford the amino acid. With the aniline and benzoic acid in hand, coupling can be effected under a variety of conditions. Alternatively, the hydroxy group of the (di)nitrophenol can be converted to a leaving group and a functionality introduced under nucleophilic aromatic substitution conditions. Other potential scaffolds that can be prepared with similar sequences are methyl 2-nitro-4-hydroxybenzoate and methyl 2-hydroxy-4-nitrobenzoate.

Compounds described herein can also be synthesized by solid-phase synthetic procedures well know to those of skill in the art (see, Tew et al., Proc. Natl. Acad. Sci. USA, 2002, 99, 5110-5114; Barany et al., Int. J. Pept. Prot. Res., 1987, 30, 705-739; Solid-phase Synthesis: A Practical Guide, Kates, S. A., and Albericio, F., eds., Marcel Dekker, New York (2000); and Döwald, F. Z., Organic Synthesis on Solid Phase: Supports, Linkers, Reactions, 2nd Ed., Wiley-VCH, Weinheim (2002)).

The compounds described herein can also be designed using computer-aided computational techniques, such as de novo design techniques, to embody the amphiphilic properties. In general, de novo design of compounds is performed by defining a three-dimensional framework of the backbone assembled from a repeating sequence of monomers using molecular dynamics and quantum force field calculations. Next, side groups are computationally grafted onto the backbone to maximize diversity and maintain drug-like properties. The best combinations of functional groups are then computationally selected to produce a cationic, amphiphilic structures. Representative compounds can be synthesized from this selected library to verify structures and test their biological activity. Novel molecular dynamic and coarse grain modeling programs have also been developed for this approach because existing force fields developed for biological molecules, such as peptides, were unreliable in these oligomer applications (see, Car et al., Phys. Rev. Lett., 1985, 55, 2471-2474; Siepmann et al., Mol. Phys., 1992, 75, 59-70; Martin et al., J. Phys. Chem., 1999, 103, 4508-4517; and Brooks et al., J. Comp. Chem., 1983, 4, 187-217). Several chemical structural series of compounds have been prepared. See, for example, International Publication No. WO 2002/100295, which is incorporated herein by reference in its entirety. The compounds described herein can be prepared in a similar manner. Molecular dynamic and coarse grain modeling programs can be used for a design approach. See, for example, U.S. Application Publication No. 2004-0107056, and U.S. Application Publication No. 2004-0102941, each of which is incorporated herein by reference in its entirety.

After verifying the suitability of the force field by comparing computed predictions of the structure and thermodynamic properties to molecules that have similar torsional patterns and for which experimental data are available, the fitted torsions can then be combined with bond stretching, bending, one-four, van der Waals, and electrostatic potentials borrowed from the CHARMM (see, Brooks et al., J. Comp. Chem., 1983, 4, 187-217) and TraPPE (Martin et al., J. Phys. Chem., 1999, 103, 4508-4517; and Wick et al., J. Phys. Chem., 2000, 104, 3093-3104) molecular dynamics force fields. To identify conformations that can adopt periodic folding patterns with polar groups and apolar groups lined up on the opposite sides, initial structures can be obtained with the Gaussian package (see, Frisch et al., Gaussian 98 (revision A.7) Gaussian Inc., Pittsburgh, Pa. 1998). Then, the parallelized plane-wave Car-Parrinello CP-MD (see, Car et al., Phys. Rev. Lett., 1985, 55, 2471-2474) program, (see, Röthlisberger et al., J. Chem. Phys., 1996, 3692-3700) can be used to obtain energies at the minimum and constrained geometries. The conformations of the compounds without side-chains can be investigated in the gas phase. Both MD and MC methods can be used to sample the conformations. The former is useful for global motions of the compound. With biasing techniques (see, Siepmann et al., Mol. Phys., 1992, 75, 59-70; Martin et al., J. Phys. Chem., 1999, 103, 4508-4517; and Vlugt et al., Mol. Phys., 1998, 94, 727-733), the latter allows efficient sampling for compounds with multiple local minimum configurations that are separated by relatively large barriers.

The potential conformations are examined for positions to attach pendant groups that will impart amphiphilic character to the secondary structure. Compounds selected from the gas phase studies with suitable backbone conformations and with side-chains at the optimal positions to introduce amphiphilicity can be further evaluated in a model interfacial system. n-hexane/water can be chosen because it is simple and cheap for calculations while it mimics well the lipid/water bilayer environment. Compound secondary structures that require inter-compound interactions can be identified by repeating the above-mentioned calculations using a periodically repeated series of unit cells of various symmetries (so called variable cell molecular dynamics or Monte Carlo technique) with or without solvent. The results of these calculations can guide the selection of candidates for synthesis.

The compounds described herein can be administered in any conventional manner by any route where they are active. Administration can be systemic, topical, or oral. For example, administration can be, but is not limited to, parenteral, subcutaneous, intravenous, intramuscular, intraperitoneal, transdermal, oral, buccal, sublingual, or ocular routes, or intravaginally, by inhalation, by depot injections, or by implants. The mode of administration can depend on the pathogen or microbe to be targeted. The selection of the specific route of administration can be selected or adjusted by the clinician according to methods known to the clinician to obtain the desired clinical response.

In some embodiments, it may be desirable to administer one or more compounds, or a pharmaceutically acceptable salt thereof, locally to an area in need of treatment. This may be achieved, for example, and not by way of limitation, by local infusion during surgery, topical application, e.g., in conjunction with a wound dressing after surgery, by injection, by means of a catheter, by means of a suppository, or by means of an implant, wherein the implant is of a porous, non-porous, or gelatinous material, including membranes, such as sialastic membranes, or fibers.

The compounds described herein can be administered either alone or in combination (concurrently or serially) with other pharmaceuticals. For example, the anti-malarial compounds can also be administered in combination with other anti-malarial compounds such as, for example, any one or more of artemisinin, quinine, artesunate, sulfadoxine-pyrimethamine, hydroxychloroquine, chloroquine, amodiaquine, pyrimethamine, sulphadoxine, proguanil, mefloquine, atovaquone, primaquine, halofantrine, doxycycline, clindamycin.

In some embodiments, the compounds described herein can also be administered in combination with (i.e., as a combined formulation or as separate formulations) with antibiotics, such as, for example: 1) protein synthesis inhibitors including, but not limited to, amikacin, anisomycin, apramycin, azithromycin, blasticidine S, brefeldin A, butirosin, chloramphenicol, chlortetracycline, clindamycin, clotrimazole, cycloheximide, demeclocycline, dibekacin, dihydrostreptomycin, doxycycline, duramycin, emetine, erythromycin, fusidic acid, G 418, gentamicin, helvolic acid, hygromycin B, josamycin, kanamycin, kirromycin, lincomycin, meclocycline, mepartricin, midecamycin, minocycline, neomycin, netilmicin, nitrofurantoin, nourseothricin, oleandomycin, oxytetracycline, paromomycin, puromycin, rapamycin, ribostamycin, rifampicin, rifamycin, rosamicin, sisomicin, spectinomycin, spiramycin, streptomycin, tetracycline, thiamphenicol, thiostrepton, tobramycin, tunicamycin, tylosin, viomycin, and virginiamycin; 2) DNA synthesis interfering agents including, but not limited to, camptothecin, 10-deacetylbaccatin III, azacytidine, 7-aminoactinomycin D, 8-quinolinol, 9-dihydro-13-acetylbaccatin III, aclarubicin, actinomycin D, actinomycin I, actinomycin V, bafilomycin A1, bleomycin, capreomycin, chromomycin, cinoxacin, ciprofloxacin, cis-diammineplatinum(II) dichloride, coumermycin A1, L(+)-lactic acid, cytochalasin B, cytochalasin D, dacarbazine, daunorubicin, distamycin A, doxorubicin, echinomycin, enrofloxacin, etoposide, flumequine, formycin, fumagillin, ganciclovir, gliotoxin, lomefloxacin, metronidazole, mithramycin A, mitomycin C, nalidixic acid, netropsin, nitrofurantoin, nogalamycin, nonactin, novobiocin, ofloxacin, oxolinic acid, paclitaxel, phenazine, phleomycin, pipemidic acid, rebeccamycin, sinefungin, streptonigrin, streptozocin, succinylsulfathiazole, sulfadiazine, sulfadimethoxine, sulfaguanidine purum, sulfamethazine, sulfamonomethoxine, sulfanilamide, sulfaquinoxaline, sulfasalazine, sulfathiazole, trimethoprim, tubercidin, 5-azacytidine, cordycepin, and formycin A; 3) cell wall synthesis interfering agents including, but not limited to, (+)-6-aminopenicillanic acid, 7-Aminodesacetoxycephalosporanic acid, amoxicillin, ampicillin, azlocillin, bacitracin, carbenicillin, cefaclor, cefamandole, cefazolin, cefmetazole, cefoperazone, cefotaxime, cefsulodin, ceftriaxone, cephalexin, cephalosporin C, cephalothin, cephradine, cloxacillin, D-cycloserine, dicloxacillin, D-penicillamine, econazole, ethambutol, lysostaphin, moxalactam, nafcillin, nikkomycin Z, nitrofurantoin, oxacillin, penicillic, penicillin G, phenethicillin, phenoxymethylpenicillinic acid, phosphomycin, pipemidic acid, piperacillin, ristomycin, and vancomycin; 4) cell membrane permeability interfering agents (ionophores) including, but not limited to, 2-mercaptopyridine, 4-bromocalcimycin A23187, alamethicin, amphotericin B, calcimycin A23187, chlorhexidine, clotrimazole, colistin, econazole, hydrocortisone, filipin, gliotoxin, gramicidin A, gramicidin C, ionomycin, lasalocid A, lonomycin A, monensin, narasin, nigericin, nisin, nonactin, nystatin, N-(6-aminohexyl)-5-chloro-1-naphthalenesulfonamide, phenazine, pimaricin, polymyxin B, DL-penicillamine, polymyxin B, praziquantel, salinomycin, surfactin, and valinomycin; 5) enzyme inhibitors including, but not limited to, (+)-usnic acid, (±)-miconazole, (S)-(+)-camptothecin, 1-deoxymannojirimycin, 2-heptyl-4-hydroxyquinoline N-oxide, cordycepin, 1,10-phenanthroline, 6-diazo-5-oxo-L-norleucine, 8-quinolinol, antimycin, antipain, ascomycin, azaserine, bafilomycin, cerulenin, chloroquine, cinoxacin, ciprofloxacin, mevastatin, concanamycin A, concanamycin C, coumermycin A1, L(+)-lactic acid, cyclosporin A, econazole, enrofloxacin, etoposide, flumequine, formycin A, furazolidone, fusaric acid, geldanamycin, gliotoxin, gramicidin A, gramicidin C, herbimycin A, indomethacin, irgasan, lomefloxacin, mycophenolic acid, myxothiazol, nalidixic acid, netropsin, niclosamide, N-(6-aminohexyl)-5-chloro-1-naphthalenesulfonamide, nikkomycin, nogalamycin, nonactin, N-methyl-1-deoxynojirimycin, novobiocin, ofloxacin, oleandomycin, oligomycin, oxolinic acid, piericidin A, pipemidic acid, radicicol, rapamycin, rebeccamycin, sinefungin, staurosporine, stigmatellin, succinylsulfathiazole, succinylsulfathiazole, sulfadiazine, sulfadimethoxine, sulfaguanidine, sulfamethazine, sulfamonomethoxine, sulfanilamide, sulfaquinoxaline, sulfasalazine, sulfathiazole, triacsin C, trimethoprim, and vineomycin A1; and 6) membrane modifiers including, but not limited to, paracelsin.

The means and methods for administration are known in the art and an artisan can refer to various pharmacologic references for guidance (see, for example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980)).

The amount of compound to be administered is that amount which is therapeutically effective. The dosage to be administered will depend on the characteristics of the subject being treated, e.g., the particular animal treated, age, weight, health, types of concurrent treatment, if any, and frequency of treatments, and can be easily determined by one of skill in the art (e.g., by the clinician). The standard dosing for protamine can be used and adjusted (i.e., increased or decreased) depending upon the factors described above. The selection of the specific dose regimen can be selected or adjusted or titrated by the clinician according to methods known to the clinician to obtain the desired clinical response.

The amount of a compound described herein that will be effective in the treatment and/or prevention of a particular disease, condition, or disorder will depend on the nature and extent of the disease, condition, or disorder, and can be determined by standard clinical techniques. In addition, in vitro or in vivo assays may optionally be employed to help identify optimal dosage ranges. The precise dose to be employed in the compositions will also depend on the route of administration, and the seriousness of the disorder, and should be decided according to the judgment of the practitioner and each patient's circumstances. However, a suitable dosage range for oral administration is, generally, from about 0.001 milligram to about 200 milligrams per kilogram body weight, from about 0.01 milligram to about 100 milligrams per kilogram body weight, from about 0.01 milligram to about 70 milligrams per kilogram body weight, from about 0.1 milligram to about 50 milligrams per kilogram body weight, from 0.5 milligram to about 20 milligrams per kilogram body weight, or from about 1 milligram to about 10 milligrams per kilogram body weight. In some embodiments, the oral dose is about 5 milligrams per kilogram body weight.

In some embodiments, suitable dosage ranges for intravenous (i.v.) administration are from about 0.01 mg to about 500 mg per kg body weight, from about 0.1 mg to about 100 mg per kg body weight, from about 1 mg to about 50 mg per kg body weight, or from about 10 mg to about 35 mg per kg body weight. Suitable dosage ranges for other modes of administration can be calculated based on the forgoing dosages as known by those skilled in the art. For example, recommended dosages for intradermal, intramuscular, intraperitoneal, subcutaneous, epidural, sublingual, intracerebral, intravaginal, transdermal administration or administration by inhalation are in the range of from about 0.001 mg to about 200 mg per kg of body weight, from about 0.01 mg to about 100 mg per kg of body weight, from about 0.1 mg to about 50 mg per kg of body weight, or from about 1 mg to about 20 mg per kg of body weight. Effective doses may be extrapolated from dose-response curves derived from in vitro or animal model test systems. Such animal models and systems are well known in the art.

The compounds described herein can be formulated for parenteral administration by injection, such as by bolus injection or continuous infusion. The compounds can be administered by continuous infusion subcutaneously over a period of about 15 minutes to about 24 hours. Formulations for injection can be presented in unit dosage form, such as in ampoules or in multi-dose containers, with an added preservative. The compositions can take such forms as suspensions, solutions or emulsions in oily or aqueous vehicles, and can contain formulatory agents such as suspending, stabilizing and/or dispersing agents. In some embodiments, the injectable is in the form of short-acting, depot, or implant and pellet forms injected subcutaneously or intramuscularly. In some embodiments, the parenteral dosage form is the form of a solution, suspension, emulsion, or dry powder.

For oral administration, the compounds described herein can be formulated by combining the compounds with pharmaceutically acceptable carriers well known in the art. Such carriers enable the compounds to be formulated as tablets, pills, dragees, capsules, emulsions, liquids, gels, syrups, caches, pellets, powders, granules, slurries, lozenges, aqueous or oily suspensions, and the like, for oral ingestion by a patient to be treated. Pharmaceutical preparations for oral use can be obtained by, for example, adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents can be added, such as, but not limited to, the cross-linked polyvinyl pyrrolidone, agar, or alginic acid or a salt thereof such as sodium alginate.

Orally administered compositions can contain one or more optional agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions may be coated to delay disintegration and absorption in the gastrointestinal tract thereby providing a sustained action over an extended period of time. Selectively permeable membranes surrounding an osmotically active driving compound are also suitable for orally administered compounds. Oral compositions can include standard vehicles such as mannitol, lactose, starch, magnesium stearate, sodium saccharine, cellulose, magnesium carbonate, etc. Such vehicles are suitably of pharmaceutical grade.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations which can be used orally include, but are not limited to, push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added.

For buccal administration, the compositions can take the form of, such as, tablets or lozenges formulated in a conventional manner.

For administration by inhalation, the compounds described herein can be delivered in the form of an aerosol spray presentation from pressurized packs or a nebulizer, with the use of a suitable propellant, such as dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, such as gelatin for use in an inhaler or insufflator can be formulated containing a powder mix of the compound and a suitable powder base such as lactose or starch.

The compounds described herein can also be formulated in rectal compositions such as suppositories or retention enemas, such as containing conventional suppository bases such as cocoa butter or other glycerides. The compounds described herein can also be formulated in vaginal compositions such as vaginal creams, suppositories, pessaries, vaginal rings, and intrauterine devices.

In transdermal administration, the compounds can be applied to a plaster, or can be applied by transdermal, therapeutic systems that are consequently supplied to the organism. In some embodiments, the compounds are present in creams, solutions, powders, fluid emulsions, fluid suspensions, semi-solids, ointments, pastes, gels, jellies, and foams, or in patches containing any of the same.

The compounds described herein can also be formulated as a depot preparation. Such long acting formulations can be administered by implantation (for example subcutaneously or intramuscularly) or by intramuscular injection. Depot injections can be administered at about 1 to about 6 months or longer intervals. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

In yet another embodiment, the compounds can be delivered in a controlled release system. In one embodiment, a pump may be used (see Langer, supra; Sefton, CRC Crit. Ref. Biomed. Eng., 1987, 14, 201; Buchwald et al., Surgery, 1980, 88, 507 Saudek et al., N. Engl. J. Med., 1989, 321, 574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger et al., J. Macromol. Sci. Rev. Macromol. Chem., 1983, 23, 61; see, also Levy et al., Science, 1985, 228, 190; During et al., Ann. Neurol., 1989, 25, 351; Howard et al., J. Neurosurg., 1989, 71, 105). In yet another embodiment, a controlled-release system can be placed in proximity of the target of the compounds described herein, such as the liver, thus requiring only a fraction of the systemic dose (see, e.g., Goodson, in Medical Applications of Controlled Release, supra, vol. 2, pp. 115-138 (1984)). Other controlled-release systems discussed in the review by Langer, Science, 1990, 249, 1527-1533) may be used.

It is also known in the art that the compounds can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The pharmaceutical compositions can also comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols. In some embodiments, the compounds described herein can be used with agents including, but not limited to, topical analgesics (e.g., lidocaine), barrier devices (e.g., GelClair), or rinses (e.g., Caphosol).

In some embodiments, the compounds described herein can be delivered in a vesicle, in particular a liposome (see, Langer, Science, 1990, 249, 1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

Suitable compositions include, but are not limited to, oral non-absorbed compositions. Suitable compositions also include, but are not limited to saline, water, cyclodextrin solutions, and buffered solutions of pH 3-9.

The compounds described herein, or pharmaceutically acceptable salts thereof, can be formulated with numerous excipients including, but not limited to, purified water, propylene glycol, PEG 400, glycerin, DMA, ethanol, benzyl alcohol, citric acid/sodium citrate (pH3), citric acid/sodium citrate (pH5), tris(hydroxymethyl)amino methane HCl (pH7.0), 0.9% saline, and 1.2% saline, and any combination thereof. In some embodiments, excipient is chosen from propylene glycol, purified water, and glycerin.

In some embodiments, the excipient is a multi-component system chosen from 20% w/v propylene glycol in saline, 30% w/v propylene glycol in saline, 40% w/v propylene glycol in saline, 50% w/v propylene glycol in saline, 15% w/v propylene glycol in purified water, 30% w/v propylene glycol in purified water, 50% w/v propylene glycol in purified water, 30% w/v propylene glycol and 5 w/v ethanol in purified water, 15% w/v glycerin in purified water, 30% w/v glycerin in purified water, 50% w/v glycerin in purified water, 20% w/v Kleptose in purified water, 40% w/v Kleptose in purified water, and 25% w/v Captisol in purified water. In some embodiments, the excipient is chosen from 50% w/v propylene glycol in purified water, 15% w/v glycerin in purified water, 20% w/v Kleptose in purified water, 40% w/v Kleptose in purified water, and 25% w/v Captisol in purified water. In some embodiments, the excipient is chosen from 20% w/v Kleptose in purified water, 20% w/v propylene glycol in purified water, and 15% w/v glycerin in purified water.

In some embodiments, the composition comprises 50 mg/mL of compound in 20% w/v Kleptose in purified water.

In some embodiments, the formulation can be lyophilized to a solid and reconstituted with, for example, water prior to use.

When administered to a mammal (e.g., to an animal for veterinary use or to a human for clinical use) the compounds can be administered in isolated form.

When administered to a human, the compounds can be sterile. Water is a suitable carrier when the compound of Formula I is administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The compositions described herein can take the form of a solution, suspension, emulsion, tablet, pill, pellet, capsule, capsule containing a liquid, powder, sustained-release formulation, suppository, aerosol, spray, or any other form suitable for use. Examples of suitable pharmaceutical carriers are described in Remington's Pharmaceutical Sciences, A. R. Gennaro (Editor) Mack Publishing Co.

In one embodiment, the compounds are formulated in accordance with routine procedures as a pharmaceutical composition adapted for administration to humans. Typically, compounds are solutions in sterile isotonic aqueous buffer. Where necessary, the compositions can also include a solubilizing agent. Compositions for intravenous administration may optionally include a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the compound is to be administered by infusion, it can be dispensed, for example, with an infusion bottle containing sterile pharmaceutical grade water or saline. Where the compound is administered by injection, an ampoule of sterile water for injection or saline can be provided so that the ingredients may be mixed prior to administration.

The pharmaceutical compositions can be in unit dosage form. In such form, the composition can be divided into unit doses containing appropriate quantities of the active component. The unit dosage form can be a packaged preparation, the package containing discrete quantities of the preparations, for example, packeted tablets, capsules, and powders in vials or ampules. The unit dosage form can also be a capsule, cachet, or tablet itself, or it can be the appropriate number of any of these packaged forms.

In some embodiments, a composition of the present disclosure is in the form of a liquid wherein the active agent (i.e., one of the facially amphiphilic polymers or oligomers disclosed herein) is present in solution, in suspension, as an emulsion, or as a solution/suspension. In some embodiments, the liquid composition is in the form of a gel. In other embodiments, the liquid composition is aqueous. In other embodiments, the composition is in the form of an ointment.

In yet other embodiments, the composition is in the form of a solid article. For example, in some embodiments, the ophthalmic composition is a solid article that can be inserted in a suitable location in the eye, such as between the eye and eyelid or in the conjunctival sac, where it releases the active agent as described, for example, U.S. Pat. No. 3,863,633; U.S. Pat. No. 3,867,519; U.S. Pat. No. 3,868,445; U.S. Pat. No. 3,960,150; U.S. Pat. No. 3,963,025; U.S. Pat. No. 4,186,184; U.S. Pat. No. 4,303,637; U.S. Pat. No. 5,443,505; and U.S. Pat. No. 5,869,079. Release from such an article is usually to the cornea, either via the lacrimal fluid that bathes the surface of the cornea, or directly to the cornea itself, with which the solid article is generally in intimate contact. Solid articles suitable for implantation in the eye in such fashion are generally composed primarily of polymers and can be bioerodible or non-bioerodible. Bioerodible polymers that can be used in the preparation of ocular implants carrying one or more of the anti-microbial, facially amphiphilic polymer or oligomer active agents in accordance with the present disclosure include, but are not limited to, aliphatic polyesters such as polymers and copolymers of poly(glycolide), poly(lactide), poly(epsilon-caprolactone), poly-(hydroxybutyrate) and poly(hydroxyvalerate), polyamino acids, polyorthoesters, polyanhydrides, aliphatic polycarbonates and polyether lactones. Suitable non-bioerodible polymers include silicone elastomers.

The ophthalmic and otic compositions are preferably sterile and have physical properties (e.g., osmolality and pH) that are specially suited for application to ophthalmic or otic tissues, including tissues that have been compromised as the result of preexisting disease, trauma, surgery or other physical conditions. For example, aqueous compositions of the disclosure typically have a pH in the range of from 4.5 to 8.0, from 6.0 to 8.0, from 6.5 to 8.0, or from 7.0 to 8.0.

Suitable ophthalmically acceptable compositions, formulations, and excipients are those that cause no substantial detrimental effect, even of a transient nature.

Suitable otically acceptable compositions, formulations, and excipients are those that cause no substantial detrimental effect, even of a transient nature.

Ophthalmically and otically acceptable excipients include, but are not limited to, viscosity-enhancing agents, preservatives, stabilizers, antioxidants, suspending agents, solubilizing agents, buffering agents, lubricating agents, ophthalmically or otically acceptable salts, and combinations thereof.

For example, aqueous ophthalmic compositions of the present disclosure, when in suspension or solution form, are suitably viscous or mucoadhesive, or both viscous or mucoadhesive, and thus comprise a viscosity-enhancing agent. Examples of suitable viscosity-enhancing agents include, but are not limited to, glycerin, polyvinyl alcohol, polyvinyl pyrrolidone, methylcellulose, hydroxypropylmethylcellulose, hydroxyethyl-cellulose, carboxymethylcellulose, hydroxypropylcellulose, and/or various gelling agents. For example, in some embodiments, the viscosity-enhancing agent is chosen from methylcellulose, hydroxypropyl-methylcellulose, polyvinyl alcohol, and glycerol. Such agents are generally employed in the compositions of the disclosure at a concentration of about 0.01% to about 3% by weight.

Thus, for ophthalmic compositions, in some embodiments, the ophthalmically acceptable excipient is a viscosity-enhancing agent or a promoter of mucoadhesion, such as carboxymethylcellulose. In such embodiments, the concentration of carboxymethylcellulose in the aqueous suspension or solution is 0.1% to 5% by weight or about 0.1% to about 2.5% by weight. The carboxymethylcellulose is preferably in the form of sodium carboxymethylcellulose substituted to a degree that the sodium content of the sodium carboxymethylcellulose is about 1% to about 20%.

In other embodiments, the ophthalmic composition is an in situ gellable aqueous composition such as an in situ gellable aqueous solution. Such a composition comprises a gelling agent in a concentration effective to promote gelling upon contact with the eye or with lacrimal fluid in the exterior of the eye, enabling the composition to remain in the eye for a prolonged period without loss by lacrimal drainage. Suitable gelling agents non-restrictively include thermosetting polymers such as tetra-substituted ethylene diamine block copolymers of ethylene oxide and propylene oxide (e.g., poloxamine 1307); polycarbophil; and polysaccharides such as gellan, carrageenan (e.g., kappa-carrageenan and iota-carrageenan), chitosan and alginate gums.

For example, in some embodiments of the present disclosure, the ophthalmic composition is an in situ gellable aqueous solution, suspension or solution/suspension, comprising from about 0.1% to about 6.5% or from about 0.5% to about 4.5% by weight, based on the total weight of the composition, of one or more compounds. A suitable gelling agent in this embodiment is polycarbophil. In other embodiments, the composition is an in situ gellable aqueous solution, suspension or solution/suspension, such as a solution, comprising about 0.1% to about 2% by weight of a polysaccharide that gels when it contacts an aqueous medium having the ionic strength of lacrimal fluid. A suitable polysaccharide is gellan gum, or a low acetyl clarified grade of gellan gum such as that sold under the trademark Gelrite®. Suitable partially deacylated gellan gums are disclosed in U.S. Pat. No. 5,190,927.

In yet other embodiments, the composition is an in situ gellable aqueous solution, suspension or solution/suspension, comprising about from 0.2% to about 3% or from about 0.5% to about 1% by weight of a gelling polysaccharide, chosen from gellan gum, alginate gum and chitosan, and about 1% to about 50% of a water-soluble film-forming polymer, preferably selected from alkylcelluloses (e.g., methylcellulose, ethylcellulose), hydroxyalkylcelluloses (e.g., hydroxyethylcellulose, hydroxypropyl methylcellulose), hyaluronic acid and salts thereof, chondroitin sulfate and salts thereof, polymers of acrylamide, acrylic acid and polycyanoacrylates, polymers of methyl methacrylate and 2-hydroxyethyl methacrylate, polydextrose, cyclodextrins, polydextrin, maltodextrin, dextran, polydextrose, gelatin, collagen, natural gums (e.g., xanthan, locust bean, acacia, tragacanth and carrageenan gums and agar), polygalacturonic acid derivatives (e.g., pectin), polyvinyl alcohol, polyvinylpyrrolidone and polyethylene glycol. The composition can optionally contain a gel-promoting counterion such as calcium in latent form, for example encapsulated in gelatin.

In yet other embodiments, the composition is an in situ gellable aqueous solution, suspension or solution/suspension comprising about 0.1% to about 5% of a carrageenan gum, e.g., a carrageenan gum having no more than 2 sulfate groups per repeating disaccharide unit, such as e.g., kappa-carrageenan, having 18-25% ester sulfate by weight, iota-carrageenan, having 25-34% ester sulfate by weight, and mixtures thereof.

In still other embodiments, the composition comprises a bioerodible polymer substantially as disclosed in U.S. Pat. No. 3,914,402.

In some embodiments, the composition comprises an ophthalmically acceptable mucoadhesive polymer, chosen from, for example, hydroxypropylmethylcellulose, carboxymethylcellulose, carbomer (acrylic acid polymer), poly(methylmethacrylate), polyacrylamide, polycarbophil, polyethylene oxide, acrylic acid/butyl acrylate copolymer, sodium alginate, and dextran.

Ophthalmic compositions of the disclosure can incorporate a means to inhibit microbial growth, for example through preparation and packaging under sterile conditions and/or through inclusion of an antimicrobially effective amount of an ophthalmically acceptable preservative.

Suitable preservatives include, but are not limited to, mercury-containing substances such as phenylmercuric salts (e.g., phenylmercuric acetate, borate and nitrate) and thimerosal; stabilized chlorine dioxide; quaternary ammonium compounds such as benzalkonium chloride, cetyltrimethylammonium bromide and cetylpyridinium chloride; imidazolidinyl urea; parabens such as methylparaben, ethylparaben, propylparaben and butylparaben, and salts thereof; phenoxyethanol; chlorophenoxyethanol; phenoxypropanol; chlorobutanol; chlorocresol; phenylethyl alcohol; disodium EDTA; and sorbic acid and salts thereof.

Several preservatives may precipitate in the presence of other excipients in the composition and/or in the presence of the polymers and oligomers in the ophthalmic compositions. For example, benzalkonium chloride can precipitate in a composition using iota-carrageenan as a gelling agent. Thus, in those embodiments of the disclosure in which a preservative is present, the preservative is one that does not precipitate but remains in solution in the composition.

In some embodiments, the ophthalmic composition further comprises an additional ophthalmically acceptable excipient. The additional ophthalmically acceptable excipient is selected from a buffering agent, a solubilizing agent, a surfactant, a lubricating agent, and an ophthalmically acceptable salt, or any combination thereof.

Optionally one or more stabilizers can be included in the compositions to enhance chemical stability where required. Suitable stabilizers include, but are not limited to, chelating agents or complexing agents, such as, for example, the calcium complexing agent ethylene diamine tetraacetic acid (EDTA). For example, an appropriate amount of EDTA or a salt thereof, e.g., the disodium salt, can be included in the composition to complex excess calcium ions and prevent gel formation during storage. EDTA or a salt thereof can suitably be included in an amount of about 0.01% to about 0.5%. In those embodiments containing a preservative other than EDTA, the EDTA or a salt thereof, more particularly disodium EDTA, can be present in an amount of about 0.025% to about 0.1% by weight.

One or more antioxidants can also be included in the ophthalmic compositions. Suitable antioxidants include, but are not limited to, ascorbic acid, sodium metabisulfite, sodium bisulfite, acetylcysteine, polyquatemium-1, benzalkonium chloride, thimerosal, chlorobutanol, methyl paraben, propyl paraben, phenylethyl alcohol, edetate disodium, sorbic acid, or other agents know to those of skill in the art. Such preservatives are typically employed at a level of from about 0.001% to about 1.0% by weight.

In some embodiments, the compounds are solubilized at least in part by an ophthalmically acceptable solubilizing agent. Certain ophthalmically acceptable nonionic surfactants, for example polysorbate 80, can be useful as solubilizing agents, as can ophthalmically acceptable glycols, polyglycols, e.g., polyethylene glycol 400 (PEG-400), and glycol ethers.

Suitable solubilizing agents for solution and solution/suspension compositions are cyclodextrins. Suitable cyclodextrins can be chosen from α-cyclodextrin, β-cyclodextrin, γ-cyclodextrin, alkylcyclodextrins (e.g., methyl-β-cyclodextrin, dimethyl-β-cyclodextrin, diethyl-β-cyclodextrin), hydroxyalkylcyclodextrins (e.g., hydroxyethyl-β-cyclodextrin, hydroxypropyl-β-cyclodextrin), carboxy-alkylcyclodextrins (e.g., carboxymethyl-β-cyclodextrin), sulfoalkylether cyclodextrins (e.g., sulfobutylether-β-cyclodextrin), and the like. Ophthalmic applications of cyclodextrins have been reviewed in Rajewski et al., Journal of Pharmaceutical Sciences, 1996, 85, 1155-1159.

An ophthalmically acceptable cyclodextrin can optionally be present in an ophthalmic composition at a concentration from about 1 to about 200 mg/ml, from about 5 to about 100 mg/ml, or from about 10 to about 50 mg/ml.

In some embodiments, the ophthalmic composition optionally contains a suspending agent. For example, in those embodiments in which the ophthalmic composition is an aqueous suspension or solution/suspension, the composition can contain one or more polymers as suspending agents. Useful polymers include, but are not limited to, water-soluble polymers such as cellulosic polymers, for example, hydroxypropyl methylcellulose, and water-insoluble polymers such as cross-linked carboxyl-containing polymers. However, in some embodiments, ophthalmic compositions do not contain substantial amounts of solid particulate matter, whether of the anti-microbial polymer or oligomer active agent, an excipient, or both, as solid particulate matter, if present, can cause discomfort and/or irritation of a treated eye.

One or more ophthalmically acceptable pH adjusting agents and/or buffering agents can be included in the ophthalmic compositions, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an ophthalmically acceptable range.

One or more ophthalmically acceptable salts can be included in the compositions of the disclosure in an amount required to bring osmolality of the composition into an ophthalmically acceptable range. Such salts include, but are not limited to, those having sodium, potassium or ammonium cations and chloride, citrate, ascorbate, borate, phosphate, bicarbonate, sulfate, thiosulfate or bisulfite anions. In some embodiments, salts include sodium chloride, potassium chloride, sodium thiosulfate, sodium bisulfite and ammonium sulfate. In some embodiments, the salt is sodium chloride.

Optionally an ophthalmically acceptable xanthine derivative such as caffeine, theobromine or theophylline can be included in the compositions, e.g., as disclosed in U.S. Pat. No. 4,559,343. Inclusion of the xanthine derivative can reduce ocular discomfort associated with administration of the composition.

Optionally one or more ophthalmically acceptable surfactants, preferably nonionic surfactants, or co-solvents can be included in the compositions to enhance solubility of the components of the compositions or to impart physical stability, or for other purposes. Suitable nonionic surfactants include, but are not limited to, polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40; polysorbate 20, 60 and 80; polyoxyethylene/polyoxypropylene surfactants (e.g., Pluronic® F-68, F84 and P-103); cyclodextrin; or other agents known to those of skill in the art. Typically, such co-solvents or surfactants are employed in the compositions at a level of from about 0.01% to about 2% by weight.

One or more ophthalmic lubricating agents can also be included optionally in the compositions to promote lacrimation or as a “dry eye” medication. Such agents include, but are not limited to, polyvinyl alcohol, methylcellulose, hydroxypropyl methylcellulose, polyvinylpyrrolidone, and the like. It will be understood that promotion of lacrimation is beneficial in the present disclosure only where lacrimation is naturally deficient, to restore a normal degree of secretion of lacrimal fluid. Where excessive lacrimation occurs, residence time of the composition in the eye can be reduced.

Ophthalmic compositions of the present disclosure typically include a combination of one or more of the optional excipients listed above. For example, in some embodiments, the ophthalmic composition can optionally further comprise glycerin in an amount from about 0.5% to about 5%, from about 1% to about 2.5%, or from about 1.5% to about 2% by weight. Glycerin can be useful to increase viscosity of the composition and for adjustment of osmolality. Independently of the presence of glycerin, the composition can also further comprise a cyclodextrin, such as hydroxypropyl-β-cyclodextrin, in an amount from about 0.5% to about 25% by weight, as a solubilizing agent, and an antimicrobially effective amount of a preservative, e.g., imidazolidinyl urea in an amount from about 0.03% to about 0.5%; methylparaben in an amount from about 0.015% to about 0.25%; propylparaben in an amount from about 0.005% to about 0.01%; phenoxyethanol in an amount from about 0.25% to about 1%; disodium EDTA in an amount from about 0.05% to about 0.2%; thimerosal in an amount from 0.001% to about 0.15%; chlorobutanol in an amount from about 0.1% to about 0.5%; and/or sorbic acid in an amount from about 0.05% to about 0.2%; all by weight.

The otic compositions also optionally comprise one or more otically acceptable excipients. Otically acceptable excipients include, but are not limited to, one or more of the preservatives, stabilizers, antioxidants, viscosity-enhancing agents, buffering agents, solubilizing agents, surfactants, lubricating agents, or acceptable salts described above, or combinations thereof, as described above for the ophthalmic compositions.

Thus, for example, in some embodiments, an otic composition optionally comprises one or more buffering agents, solubilizing agents, and antioxidants, typically in an aqueous solution. In some embodiments, the otic composition further comprises glycerin (e.g., anhydrous glycerin) or propylene glycol as a viscosity-enhancing agent. The otic composition may also comprise a surfactant in combination with the glycerin or propylene glycol to aid in the removal of cerum 30 (ear wax). Sodium bicarbonate may also be used if wax is to be removed from the ear.

Thus, e.g., in some embodiments, the otic composition is a sterile aqueous solution comprising one or more of the disclosed polymers or oligomers, glycerin, sodium bicarbonate, and, optionally, a preservative, in purified water.

The ophthalmic and otic compositions can be prepared by methods known in the art and described in patents and publications cited herein and incorporated herein by reference.

The compounds described herein can also be incorporated into compositions such as, for example, polishes, paints, sprays, or detergents formulated for application to a surface to inhibit the growth of a Mycobacterium species thereon. These surfaces include, but are not limited to, countertops, desks, chairs, laboratory benches, tables, floors, bed stands, tools, equipment, doorknobs, windows, and the like. The compounds described herein can also be incorporated into soaps and hand lotions. The present compositions, including the cleansers, polishes, paints, sprays, soaps, and detergents, can contain one or more of the compounds described herein. In addition, the compositions can optionally contain one or more of each of the following: solvents, carriers, thickeners, pigments, fragrances, deodorizers, emulsifiers, surfactants, wetting agents, waxes, and/or oils. For example, in some embodiments, the compounds can be incorporated into a formulation for external use as a pharmaceutically acceptable skin cleanser, particularly for the surfaces of human hands. Cleansers, polishes, paints, sprays, soaps, hand lotions, and detergents and the like containing the compounds described herein can be useful in homes and institutions, particularly but not exclusively, in hospital settings for the prevention of nosocomial infections.

The present disclosure also provides pharmaceutical packs or kits comprising one or more containers filled with one or more compounds described herein. Optionally associated with such container(s) can be a notice in the form prescribed by a governmental agency regulating the manufacture, use or sale of pharmaceuticals or biological products, which notice reflects approval by the agency of manufacture, use or sale for human administration for treating a condition, disease, or disorder described herein. In some embodiments, the kit contains more than one compound described herein. In some embodiments, the kit comprises a compound described herein in a single injectable dosage form, such as a single dose within an injectable device such as a syringe with a needle.

The present disclosure also provides methods of treating malaria in an animal comprising administering to the animal a therapeutically effective amount of a compound, or a pharmaceutically acceptable salt thereof. In any of the above embodiments, the malaria can be chloroquine-sensitive or chloroquine-resistant.

The present disclosure also provides methods of killing or inhibiting the growth of a Plasmodium species comprising contacting the species with an effective amount of a compound, or a pharmaceutically acceptable salt thereof. In any of the above embodiments, the malaria can be chloroquine-sensitive or chloroquine-resistant.

The present disclosure also provides methods of disrupting a food vacuole of a Plasmodium species comprising contacting the species with an effective amount of a compound, or a pharmaceutically acceptable salt thereof. In any of the above embodiments, the malaria can be chloroquine-sensitive or chloroquine-resistant.

The anti-malarial compounds can be useful as anti-malarial agents in a number of applications. For example, compounds can be used therapeutically to treat malaria in animals, including humans and non-human vertebrates such as wild, domestic and farm animals. The malarial infection in an animal can be treated by administering to the animal an effective amount of a compound, or a pharmaceutical composition comprising the same. The compound, or composition thereof, can be administered systemically or topically and can be administered to any body site or tissue.

The present disclosure also provides compounds, or a salt thereof, or compositions comprising the same, for use in treating a malarial infection in an animal. The present disclosure also provides compounds, or a salt thereof, or compositions comprising the same, for use in killing or inhibiting the growth of a Plasmodium species. The present disclosure also provides compounds, or a salt thereof, or compositions comprising the same, for use in preparation of a medicament for treating a malarial infection in an animal. The present disclosure also provides compounds, or a salt thereof, or compositions comprising the same, for use in preparation of a medicament for killing or inhibiting the growth of a Plasmodium species.

The compounds described herein can be combined with one, two, or three other anti-malarial compounds described herein to form a cocktail. This cocktail can also include other anti-malarial compounds. Other anti-malarial compounds include, but are not limited to, any one or more of artemisinin, quinine, artesunate, sulfadoxine-pyrimethamine, hydroxychloroquine, chloroquine, amodiaquine, pyrimethamine, sulphadoxine, proguanil, mefloquine, atovaquone, primaquine, halofantrine, doxycycline, and clindamycin.

One of skill in the art will recognize that the compounds can be tested for anti-malarial activity by methods well known to those of skill in the art. Any compound found to be active can be purified to homogeneity and re-tested to obtain an accurate IC₅₀.

Thus, the present disclosure provides methods of treating malaria in an animal comprising administering to the animal in need thereof an effective amount of a compound or a slat thereof. The present disclosure provides methods of treating malaria in an animal comprising administering to the animal in need thereof a composition comprising a compound, or a salt thereof. The present disclosure provides methods of killing or inhibiting the growth of a Plasmodium species comprising contacting the species with an effective amount of a compound, or salt thereof. The present disclosure provides methods of killing or inhibiting the growth of a Plasmodium species comprising contacting the species with a composition comprising a compound, or salt thereof. The present disclosure provides methods of killing or inhibiting the growth of a chloroquine-sensitive or chloroquine-resistant Plasmodium species comprising contacting the species with an effective amount of a compound, or salt thereof. The present disclosure provides methods of killing or inhibiting the growth of a chloroquine-sensitive or chloroquine-resistant Plasmodium species comprising contacting the species with a composition comprising a compound, or salt thereof. The present disclosure provides methods of disrupting a food vacuole of a Plasmodium species comprising contacting the species with an effective amount of a compound, or salt thereof. The present disclosure provides methods of disrupting a food vacuole of a Plasmodium species comprising contacting the species with a composition comprising a compound, or salt thereof.

In order that the disclosure disclosed herein may be more efficiently understood, examples are provided below. It should be understood that these examples are for illustrative purposes only and are not to be construed as limiting the disclosure in any manner. Throughout these examples, molecular cloning reactions, and other standard recombinant DNA techniques, were carried out according to methods described in Maniatis et al., Molecular Cloning—A Laboratory Manual, 2nd ed., Cold Spring Harbor Press (1989), using commercially available reagents, except where otherwise noted.

### EXAMPLES

### Example 1

### Anti-Malarial Activity

Numerous compounds were screened in cultures of the malaria parasite P. falciparum 3D7 (chloroquine-sensitive) and DD2 (chloroquine-resistant). The compounds were also screened for toxixcity against 3T3 cells, HepG2 cells, and in a hemolysis assay. In brief, P. falciparum parasites were cultured in RPMI 1640 (Invitrogen) supplemented with Albumax II (Invitrogen). For synchronization, schizont stage parasites were magnet purified using a SuperMACS™ II Cell Separation Unit (Miltenyi Biotech). For IC₅₀ determinations, synchronized ring-stage parasites were plated at 0.5% parasitemia and 8% hematocrit in 96-well plates at a total volume of 50 μL. Serial dilutions of 2× concentration of the respective compound, ranging from 2.5 μM to 20 nM, were added to the wells to bring the total volume up to 100 μL and 4% hematocrit. Compounds were assayed for a 72 hour period, after which the cultures were fixed with a solution of 4% paraformaldehyde and 0.08% glutaraldhyde in PBS. The cells were then permeabilized with 0.25% Triton X-100, and SYTOX Green Nucleic Acid Stain (Invitrogen) in PBS was added to a final concentration of 5 μM. DNA content, as an indicator of parasitemia, was analyzed on an Accuri C6 Flow Cytometer with C-Sampler. IC₅₀ curves were generated using GraphPad Prism (GraphPad Software). Data is presented in Table 1, below.

TABLE 1

| | P. falciparum | | Cytotoxicity | | |
|---|---|---|---|---|---|
| | 3D7 | DD2 | 3T3 | HepG2 | Hemolysis |
| | IC₅₀ | IC₅₀ | EC₅₀ | EC₅₀ | EC₅₀ |
| Compound | (nM) | (nM) | (μM) | (μM) | (μg/mL) |
| 100 | 35 | 135 | 769 | 830 | >1000 |
| 131 | 42 | 65 | 322 | 356 | 212 |
| 101 | 47 | 113 | 74.4 | 27 | 583 |
| 102 | 64 | 238 | <2 | 49.8 | 21 |
| 153 | 77.1 | — | 310.7 | 453 | 115.6 |
| 120 | 83 | 45.9 | 65.1 | 165 | 97.9 |
| 152 | 88.3 | — | 406 | 502 | 0.8 |
| 121 | 108 | 170 | >1000 | >1000 | 378 |
| 132 | 112.1 | — | 323 | 456 | >1000 |
| 133 | 116.3 | — | 295 | 551 | >1000 |
| 134 | 125.6 | — | 654 | 905 | >1000 |
| 163 | 127 | — | — | — | — |
| 135 | 130.9 | — | 623 | 697 | >1000 |
| 103 | 147 | 207 | 256 | 382 | 254 |
| 122 | 148 | 267 | 64 | 87 | >1000 |
| 123 | 157 | 331 | 284 | 433 | 822 |
| 136 | 178 | 421 | >1000 | >1000 | 33 |
| 137 | 187.4 | — | 272 | 422 | >1000 |
| 104 | 200 | 301 | 25 | 39 | 2 |
| 173 | 204 | 274 | >2000 | >2000 | >1000 |
| 105 | 225 | — | 44 | 81 | — |
| 138 | 225.8 | — | 78 | 85 | 60 |
| 168 | 227 | 98 | >100 | >100 | >1000 |
| 156 | 258 | — | 25 | 50 | 9 |
| 155 | 272 | 457 | 131 | 100 | 6 |
| 106 | 286 | — | 79 | 89 | — |
| 107 | 295 | 484 | >1000 | >1000 | >1000 |
| 108 | 306 | 419 | — | — | 564 |
| 146 | 308 | 331 | 373 | 373 | 123 |
| 169 | 317 | 150 | — | — | 210 |
| 124 | 320 | — | 20 | 24 | 82 |
| 157 | 322 | 244 | >100 | >100 | 299 |
| 163 | 357 | — | 60 | 98 | — |
| 125 | 358 | 268 | insol | insol | 1.8 |
| 175 | 423 | 564 | 302 | 362 | >1000 |
| 139 | 430 | — | 36 | 23 | — |
| 109 | 438 | 601 | — | — | 4 |
| 154 | 472.9 | — | 210 | 471 | 352 |
| 110 | 563 | 740 | — | — | 45 |
| 170 | 569 | 591 | 110 | 165 | 59 |
| 174 | 593 | 393 | <16 | <16 | 1 |
| 140 | 601.7 | — | >1449 | > 1449 | >1000 |
| 111 | 610 | 431 | 32 | 84 | 65 |
| 164 | 610 | 980 | 23 | 78 | 15 |
| 141 | 625 | 709 | 28 | 55 | 13 |
| 176 | 629 | — | 523 | 608 | 314 |
| 180 | 641.4 | — | 40 | 78 | 63 |
| 112 | 653 | 993 | — | — | 87 |
| 158 | 655 | 1095 | 14 | 29 | 17 |
| 178 | 656 | 555 | 29 | 26 | 119 |
| 126 | 660 | — | 140 | 562 | >1000 |
| 113 | 668 | 611 | — | — | 54 |
| 114 | 672 | 656 | 31 | 57 | 61 |
| 115 | 680 | 664 | 26 | 47 | 35 |
| 162 | 683 | 827 | — | — | 8.9 |
| 181 | 688 | 740 | 81 | 154 | >1000 |
| 147 | 696 | 829 | >100 | >100 | 560 |
| 116 | 706 | 398 | 76 | 95 | 39 |
| 167 | 724 | 951 | 37 | 60 | 7 |
| 117 | 732 | 803 | 5 | 9 | 12 |
| 127 | 738.2 | — | 132 | 413 | 239 |
| 118 | 740 | 1154 | — | — | 11 |
| 142 | 795 | — | 229 | 594 | 538 |
| 119 | 870 | 688 | — | — | >1000 |
| 159 | 870 | 1076 | 20 | 19 | 6 |
| 179 | 880 | 795 | insol | insol | 22 |
| 128 | 881 | 886 | 121 | 232 | 58 |
| 171 | 893 | 524 | — | — | — |
| 165 | 904 | 1183 | — | — | 16 |
| 160 | 925 | — | 66 | 224 | 250 |
| 161 | 939 | — | 697 | 676 | — |
| 166 | 991 | 1226 | 17 | 71 | 364 |
| 148 | — | 81 | >1000 | >1000 | >1000 |
| 143 | — | 102 | 74 | 119 | 94 |
| 129 | — | 240 | 20 | 41 | 103 |
| 144 | — | 247 | 27 | 90 | 3 |
| 177 | — | 255 | 24 | 22 | 16 |
| 172 | — | 297 | Insol. | Insol. | 22 |
| 149 | — | 493 | 362 | 391 | >1000 |
| 150 | — | 530 | 168 | 157 | 197 |
| 151 | — | 563 | 469 | 408 | >1000 |
| 145 | — | 658 | 14 | 28 | 6 |
| 130 | — | 976 | — | — | 170 |
| 220 | 1552 | — | >1000 | >1000 | >1000 |
| 221 | 80 | — | >1000 | >1000 | >1000 |
| 222 | 551 | — | 764 | >1000 | >1000 |
| 182 | 560 | — | 330 | 345 | 641 |
| 183 | 383 | — | 694 | 739 | 5 |
| 301 | 29 | — | 716 | 778 | <1 |
| 223 | 516 | — | 18 | 82 | 21 |
| 224 | >2500 | — | >1000 | >1000 | 6 |
| 225 | 461 | — | 35 | 179 | 14 |
| 268 | 87 | — | 74 | 447 | 113 |
| 226 | 581 | — | 51 | 211 | 9 |
| 227 | 650 | — | 54 | 117 | 3 |
| 184 | 129 | — | 478 | >1000 | 4 |
| 302 | 333 | — | 687 | >1000 | 4 |
| 185 | 692 | — | 396 | 804 | 516 |
| 279 | 773 | — | 436 | 885 | >1000 |
| 228 | 552 | — | 31 | 304 | >1000 |
| 229 | 628 | — | 3 | 262 | 234 |
| 230 | 370 | — | 42 | 57 | 83 |
| 231 | 369 | — | 998 | >1000 | 29 |
| 186 | 177 | — | 63 | >1000 | 107 |
| 269 | 320 | — | >1000 | >1000 | >1000 |
| 232 | 273 | — | 37 | 56 | 6 |
| 233 | 737 | — | 61 | 88 | 2 |
| 234 | 655 | — | >1000 | >1000 | <8 |
| 270 | 108 | — | 398 | 702 | 317 |
| 235 | >2000 | — | 14 | 156 | 24 |
| 236 | >2000 | — | <14 | 186 | 223 |
| 237 | >2000 | — | 2 | 222 | 148 |
| 238 | 109 | — | 108 | 310 | 147 |
| 271 | 29 | — | 172 | 347 | 134 |
| 272 | 38 | — | 65 | 214 | 180 |
| 273 | 1283 | — | 747 | >1000 | >1000 |
| 274 | >2000 | — | 72 | 164 | 131 |
| 239 | 102 | — | 149 | 288 | 232 |
| 240 | 121 | — | 461 | 904 | 871 |
| 275 | 1702 | — | 124 | 342 | 97 |
| 187 | 41 | — | 42 | 130 | 182 |
| 280 | >100 | — | 29 | 79 | 296 |
| 241 | >2000 | — | 34 | 38 | 89 |
| 242 | >2000 | — | 71 | 584 | >1000 |
| 188 | 137 | — | 1 | 9 | 4 |
| 243 | 58 | — | 523 | 723 | 697 |
| 244 | 149 | — | 20 | 19 | 15 |
| 189 | >2000 | — | >1000 | >1000 | >1000 |
| 190 | 26 | — | 33 | 41 | 4 |
| 276 | 1115 | — | 42 | 154 | 65 |
| 277 | 570 | — | 31 | 64 | 40 |
| 245 | 541 | — | >1000 | >1000 | >1000 |
| 246 | 5 | — | >1000 | >1000 | >1000 |
| 191 | 458 | — | >1000 | >1000 | >1000 |
| 192 | 182 | — | 953 | >1000 | 32 |
| 247 | 34 | — | <14 | 74 | 9 |
| 193 | 45 | — | >1000 | >1000 | >1000 |
| 194 | 187 | — | >1000 | >1000 | >1000 |
| 195 | 45 | — | >1000 | >1000 | >1000 |
| 196 | 30 | — | >1000 | 940 | 164 |
| 197 | 14 | — | 405 | 881 | 187 |
| 248 | 3 | — | 50 | 54 | 24 |
| 249 | 28 | — | 7 | 46 | 261 |
| 250 | 556 | — | >1000 | >1000 | >1000 |
| 198 | 5 | — | 7 | 30 | 2 |
| 303 | 34 | — | 887 | >1000 | 62 |
| 251 | 12 | — | 559 | 692 | 459 |
| 252 | 4 | — | >1000 | >1000 | 12 |
| 199 | 10 | — | 192 | 353 | 34 |
| 200 | 4 | — | 27 | 37 | 24 |
| 201 | <1 | — | 137 | 234 | 48 |
| 202 | 7 | — | 632 | 648 | 331 |
| 203 | 85 | — | 15 | 45 | 66 |
| 253 | 107 | — | 419 | 606 | 8 |
| 254 | 154 | — | 32 | 49 | 36 |
| 204 | 103 | — | 477 | 483 | 88 |
| 205 | 70 | — | 40 | 307 | 176 |
| 206 | 179 | — | >1000 | >1000 | >1000 |
| 255 | 152 | — | 29 | 45 | 28 |
| 207 | 182 | — | 478 | 819 | 29 |
| 208 | 139 | — | 122 | 183 | 12 |
| 319 | >100 | — | 57 | 83 | 52 |
| 256 | 59 | — | >1000 | >1000 | >1000 |
| 257 | >100 | — | 40 | 220 | >1000 |
| 209 | 66 | — | 431 | 516 | 30 |
| 258 | 58 | — | 165 | 305 | 545 |
| 259 | >100 | — | >1000 | >1000 | 287 |
| 317 | >100 | — | 553 | 500 | >1000 |
| 299 | >100 | — | 920 | 920 | 251 |
| 304 | 69 | — | 934 | 934 | 3 |
| 305 | 78 | — | 898 | 904 | 223 |
| 306 | >100 | — | 814 | 814 | 793 |
| 307 | 41 | — | 846 | 892 | 757 |
| 308 | >100 | — | 22 | 34 | 2 |
| 281 | >100 | — | 2 | 5 | 4 |
| 260 | >100 | — | >1000 | >1000 | 16 |
| 282 | >100 | — | 57 | 126 | >1000 |
| 283 | >100 | — | 153 | 284 | >1000 |
| 284 | >100 | — | 6 | 28 | 2 |
| 261 | 78 | — | >1000 | 932 | >1000 |
| 262 | >100 | — | >1000 | >1000 | >1000 |
| 210 | 29 | — | 22 | 31 | — |
| 211 | >100 | — | >1000 | >1000 | >1000 |
| 212 | >100 | — | >1000 | >1000 | — |
| 309 | 79 | — | >1000 | >1000 | — |
| 310 | 84 | — | >1000 | 884 | 81 |
| 311 | >100 | — | 923 | 923 | 15 |
| 285 | 86 | — | 587 | 815 | >1000 |
| 286 | 80 | — | 83 | 221 | 10 |
| 287 | >100 | — | 84 | 200 | 302 |
| 312 | — | — | >10000 | >1000 | 6 |
| 313 | — | — | 92 | 545 | 14 |
| 314 | — | — | >1000 | 827 | — |
| 263 | — | — | 6 | 1 | 4 |
| 315 | — | — | 21 | 34 | 2 |
| 316 | — | — | 33 | 41 | 22 |
| 264 | — | — | >1000 | >1000 | >1000 |
| 288 | — | — | >1000 | >1000 | >1000 |
| 318 | >100 | — | 624 | >1000 | >1000 |
| 213 | 35 | — | 244 | 51 | 129 |
| 214 | >100 | — | >1000 | >1000 | >1000 |
| 215 | >100 | — | >1000 | >1000 | >1000 |
| 265 | >100 | — | >1000 | >1000 | >1000 |
| 266 | 59 | — | 38 | 197 | 1 |
| 216 | >100 | — | >1000 | >1000 | >1000 |
| 217 | >100 | — | >1000 | >1000 | >1000 |
| 289 | >100 | — | 248 | 425 | — |
| 290 | >100 | — | >1000 | >1000 | — |
| 278 | >100 | — | >1000 | >1000 | — |
| 291 | >100 | — | 345 | 410 | — |
| 267 | 29 | — | 651 | 823 | — |
| 292 | 88 | — | 710 | >1000 | — |
| 293 | >100 | — | 469 | >1000 | — |
| 218 | 56 | — | 46 | 233 | — |
| 294 | >100 | — | >1000 | >1000 | — |
| 295 | — | — | >1000 | >1000 | — |
| 296 | — | — | 439 | >1000 | — |
| 297 | — | — | 518 | 675 | — |
| 298 | — | — | >1000 | >1000 | — |
| 219 | — | — | 919 | >1000 | — |
| 300 | — | — | 848 | 848 | — |

Various modifications of the disclosure, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

1-144. (canceled)

145. A compound of:
a) Formula I:
R¹-A₁-X—Y-A₂-Y—X-A₁-R² (I)
or a pharmaceutically acceptable salt thereof,
wherein:
each X is, independently, —NR⁸, —N(R⁸)N(R⁸)—, O, or S, wherein each R⁸ is, independently, hydrogen or alkyl;
each Y is, independently, C═O, C═S, or O═S═O;
each A₁ and A₂ is, independently, aryl optionally substituted with one or more W, or heteroaryl optionally substituted with one or more W;
each W is, independently, halo, —CF₃, cyano, C₁₋₄alkoxy, —S—(CH₂)₁₋₅NHC(═NH)NH₂, —S—(CH₂)₁₋₅NH₂, —S—(CH₂)₁₋₅N(CH₃)₂, —S—(CH₂)₁₋₅NHS(═O)₂—C₁₋₄alkyl, —C₁₋₄alkyl, —O—(CH₂)₁₋₅NHC(═NH)NH₂, or O-heterocycle (wherein the heterocycle is optionally substituted with one or more cyano, amino, C₁₋₄alkyl, guanidino, hydroxyl, amidino, C₁₋₄alkoxy, CF₃, or halo); and
R¹ and R² are, independently, —(CH₂)₁₋₄NH₂, —(CH₂)₁₋₅NHC(═NH)NH₂, —S—(CH₂)₁₋₅N(CH₃)₂, —O—(CH₂)₁₋₅NHC(═NH)NH₂, —S—(CH₂)₁₋₅NHC(═NH)NH₂, —O—(CH₂)₁₋₅N(CH₃)₂, amino, —O-heterocycle or —O-cycloalkyl (wherein the heterocycle and cycloalkyl are optionally substituted), —S-heterocycle (wherein the heterocycle is optionally substituted), —NHC(═O)—(CH₂)₁₋₅NHC(═NH)NH₂, —NHC(═O)—C₁₋₄alkyl, heterocycle (wherein the heterocycle is optionally substituted), —NHC(═O)—(CH₂)₁₋₅NHS(═O)₂—C₁₋₄alkyl, —NHC(═O)—(CH₂)₁₋₅-aryl (wherein either or both —(CH₂)₁₋₅ and aryl is optionally substituted), —NHC(═O)-aryl (wherein the aryl is optionally substituted), or —NHC(═O)—(CH₂)₁₋₅NH-aryl (wherein either or both —NH and aryl is optionally substituted), wherein the optional substituents are chosen from one or more cyano, amino, C₁₋₄alkyl, guanidino, hydroxyl, amidino, C₁₋₄alkoxy, CF₃, halo, and —N(CH₃)₂;
provided that the compound of Formula I is not one of the following: 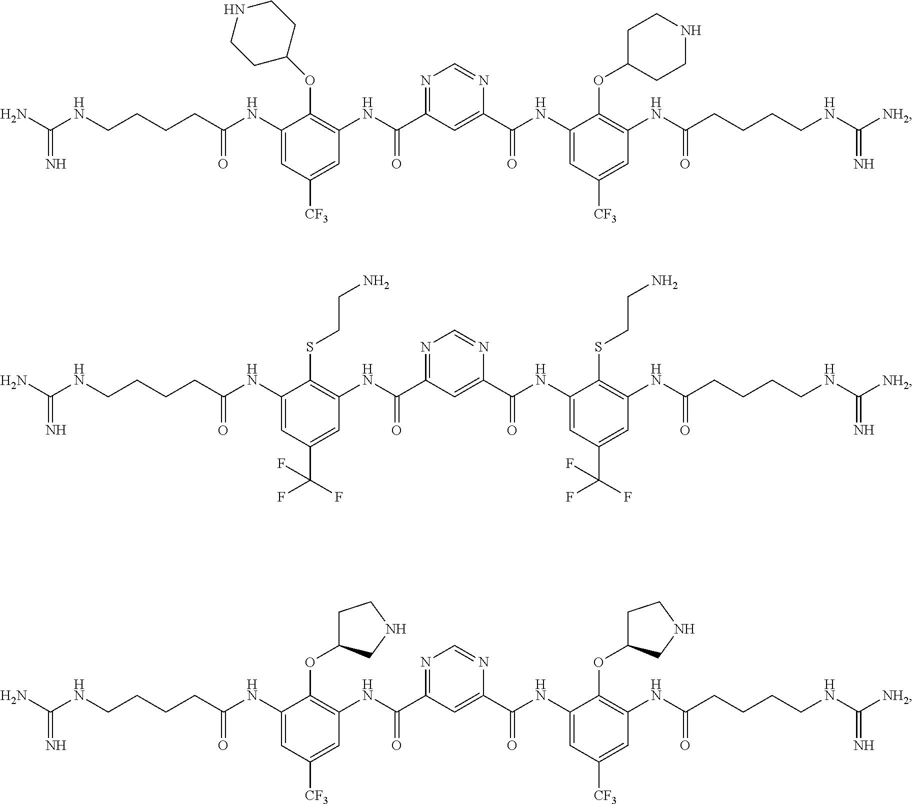 [SMILES: N=C(N)NCCCCC(=O)Nc1cc(C(F)(F)F)cc(NC(=O)c2cc(C(=O)Nc3cc(C(F)(F)F)cc(NC(=O)CCCCNC(=N)N)c3OC3CCNCC3)ncn2)c1OC1CCNCC1 ; N=C(N)NCCCCC(=O)Nc1cc(C(F)(F)F)cc(NC(=O)c2cc(C(=O)Nc3cc(C(F)(F)F)cc(NC(=O)CCCCNC(=N)N)c3SCCN)ncn2)c1SCCN ; N=C(N)NCCCCC(=O)Nc1cc(C(F)(F)F)cc(NC(=O)c2cc(C(=O)Nc3cc(C(F)(F)F)cc(NC(=O)CCCCNC(=N)N)c3O[C@H]3CCNC3)ncn2)c1O[C@H]1CCNC1]
or its enantiomer, 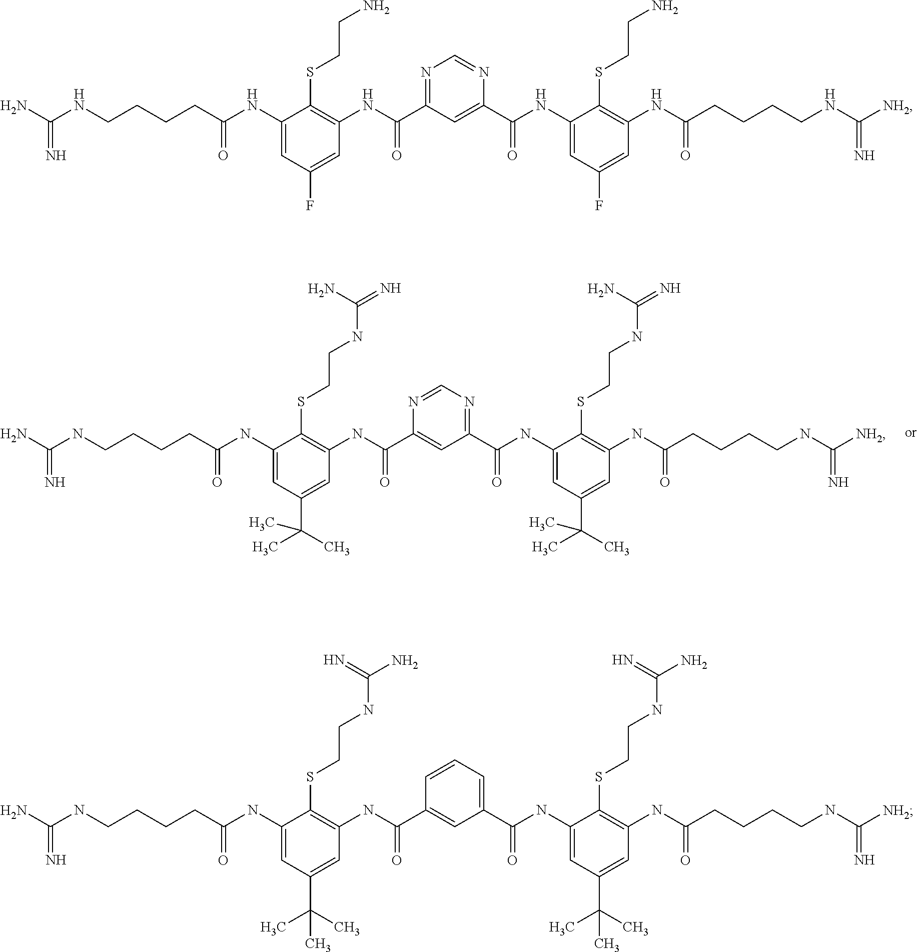 [SMILES: N=C(N)NCCCCC(=O)Nc1cc(F)cc(NC(=O)c2cc(C(=O)Nc3cc(F)cc(NC(=O)CCCCNC(=N)N)c3SCCN)ncn2)c1SCCN ; CC(C)(C)c1cc(NC(=O)CCCCNC(=N)N)c(SCCNC(=N)N)c(NC(=O)c2cc(C(=O)Nc3cc(C(C)(C)C)cc(NC(=O)CCCCNC(=N)N)c3SCCNC(=N)N)ncn2)c1 ; "or" ; CC(C)(C)c1cc(NC(=O)CCCCNC(=N)N)c(SCCNC(=N)N)c(NC(=O)c2cccc(C(=O)Nc3cc(C(C)(C)C)cc(NC(=O)CCCCNC(=N)N)c3SCCNC(=N)N)c2)c1]
b) Formula IIa: 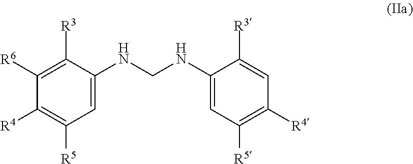
or a pharmaceutically acceptable salt thereof,
wherein:
R³ and R^{3′} are, independently, H or —O—C₁₋₄alkyl;
R⁶ is H, —(CH₂)₁₋₄NHC(═NH)NH₂, or —(CH₂)₁₋₄NH₂;
R⁵ and R^{5′} are, independently, H, —CF₃, —(CH₂)₁₋₄NHC(═NH)NH₂, or —(CH₂)₁₋₄NH₂; and
R⁴ and R^{4′} are, independently, H, —S—(CH₂)₁₋₄NHC(═NH)NH₂, —S—(CH₂)₁₋₄NH₂, 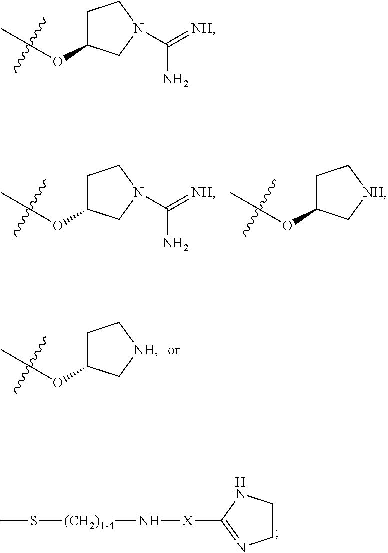
c) Formula III:
R¹—X-A₁-A₂-A₁-X—R² (III)
or a pharmaceutically acceptable salt thereof,
wherein:
A₂ is aryl optionally substituted with one or more W, or heteroaryl optionally substituted with one or more W;
each A₁ is, independently, aryl optionally substituted with one or more W, or heteroaryl optionally substituted with one or more W;
each X is, independently, absent, —NR⁸, —N(R⁸)N(R⁸)—, O, or S, wherein each R₈ is, independently, hydrogen or alkyl;
R¹ and R² are, independently, absent, —C(═O)—(CH₂)₁₋₅NHC(═NH)NH₂, hydrogen, —(CH₂)₁₋₅NHC(═NH)NH₂, —C(═O)—CH(NH₂)C₁₋₄alkyl, halo, —NO₂, —CF₃, —N⁺(═O)O⁻, amino, —S—(CH₂)₁₋₅NHC(═NH)NH₂, —O—(CH₂)₁₋₅NHC(═NH)NH₂, —(CH₂)₁₋₅NHC(═NH)NH₂, —S—(CH₂)₁₋₅N(CH₃)₂, —O—(CH₂)₁₋₅N(CH₃)₂, —NHC(═O)—(CH₂)₁₋₅NHC(═NH)NH₂, —NHC(═O)—C₁₋₄alkyl, or —NHC(═O)—(CH₂)₁₋₅NHS(═O)₂—C₁₋₄alkyl; and
each W is, independently, —O—(CH₂)₁₋₅NHC(═NH)NH₂, —O—(CH₂)₁₋₅NH₂, —CF₃, halo, —C₁₋₄alkyl, —N((CH₂)₁₋₅—NH₂)((CH₂)₁₋₅N(C₁₋₄alkyl)₂), —N((CH₂)₁₋₅N(C₁₋₄alkyl)₂)₂, —C₁₋₄alkylamino, —(CH₂)₁₋₅NHC(═NH)NH₂, —S—(CH₂)₁₋₅NHC(═NH)NH₂, —S—(CH₂)₁₋₅NH₂, —S—(CH₂)₁₋₅N(CH₃)₂, —S—(CH₂)₁₋₅NHS(═O)₂—C₁₋₄alkyl, —C≡C—(CH₂)₁₋₅—NHC(═NH)NH₂, —C≡C—(CH₂)₁₋₅—NH₂, or aryl or heterocycle each optionally substituted with one or more —CF₃, cyano, amino, C₁₋₄alkyl, C₁₋₄alkoxy, guanidino, hydroxyl, amidino, halo, —(CH₂)₁₋₄NH₂, —O—(CH₂)₁₋₄NH₂, —(CH₂)₁₋₄NHC(═NH)NH₂, or —O—(CH₂)₁₋₄NHC(═NH)NH₂;
d) Formula IV:
Het¹-X—Y-Het²-Y—X-Het¹ (IV)
or a pharmaceutically acceptable salt thereof,
wherein:
each X is, independently, —NR⁸, —N(R⁸)N(R⁸)—, O, or S, wherein each R⁸ is, independently, hydrogen or alkyl;
each Y is, independently, C═O, C═S, or O═S═O;
each Het¹ is, independently, a fused bicyclic ring chosen from naphthalene, isobenzofuran, indolizine, isoindole, indole, purine, isoquinoline, quinolone, phthalazine, naphthyridine, quinoxaline, quinazoline, pteridine, chroman, isochroman, indoline, isoindoline, and 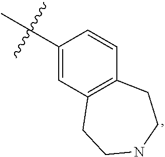 [SMILES: *c1ccc2c(c1)CCNCC2]
each of which is optionally substituted with one or more W;
Het² is an aryl or heteroaryl chosen from thiophene, furan, pyran, pyrrole, imidazole, pyrazole, isothiaole, isoxazole, pyridine, pyrazine, pyrimidine, and pyridazine; and
each W is, independently, —(CH₂)₁₋₅NH₂, —C₁₋₄alkyl, —(CH₂)₁₋₅NHC(═NH)NH₂, —O—(CH₂)₁₋₅NHC(═NH)NH₂, —O—(CH₂)₁₋₅NH₂, —CF₃, halo, C₁₋₄alkyl, —S—(CH₂)₁₋₅NH₂, —N((CH₂)₁₋₅N(C₁₋₄alkyl)₂)₂, —N((CH₂)₁₋₅—NH₂)((CH₂)₁₋₅N(C₁₋₄alkyl)₂), —S—(CH₂)₁₋₅N(CH₃)₂, —(CH₂)₁₋₅NHC(═NH)NH₂, —S—(CH₂)₁₋₅NHC(═NH)NH₂, —S—(CH₂)₁₋₅NHS(═O)₂—C₁₋₄alkyl, or cycloalkyl optionally substituted with one or more —CF₃, cyano, amino, nitro, C₁₋₄alkyl, C₁₋₄alkoxy, guanidino, hydroxyl, amidino, or halo;
e) Formula VII: 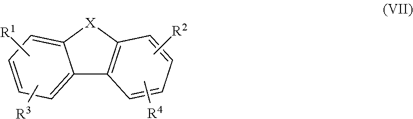
or a pharmaceutically acceptable salt thereof,
wherein:
X is C(R⁷)C(R⁸), C(R⁷)(R⁸), S, or —N(R⁹);
R⁷, R⁸, and R⁹ are, independently, C₁-C₄alkyl, —(CH₂)₀₋₄NH₂, or —(CH₂)₀₋₄NHC(═NH)NH₂;
R¹ and R² are, independently, H, C₁-C₈alkyl, C₁-C₈alkoxy, halo, OH, CN, CF₃, or haloC₁-C₈alkyl;
R³ and R⁴ are, independently, H, carbocycle(R⁵)(R⁶), —NH-carbocycle(R⁵)(R⁶);
each R⁵ and each R⁶ are, independently, H, C₁-C₈alkyl, C₁-C₈alkoxy, halo, amino, amidino, OH, CF₃, —N—(CH₃)₂, —O—(CH₂)₁₋₅—NH₂, —O—(CH₂)₁₋₅NHC(═NH)NH₂, —S—(CH₂)₁₋₅—NH₂, —N((CH₂)₁₋₅NH₂)₂, —S—(CH₂)₁₋₅NHC(═NH)NH₂, —C(═O)NH(CH₂)₁₋₅NH₂, —(CH₂)₁₋₈—NH₂, —(CH₂)₁₋₅N((CH₂)₁₋₅NH₂)₂, aromatic group, heterocycle, —(CH₂)₁₋₈—NH—(CH₂)₁₋₈—NH₂, or —(CH₂)₁₋₈—NH—C(═NH)NH₂;
f) Formula VIII:
Q-X—Z—X-Q (VIII)
or a pharmaceutically acceptable salt thereof,
wherein:
Z is 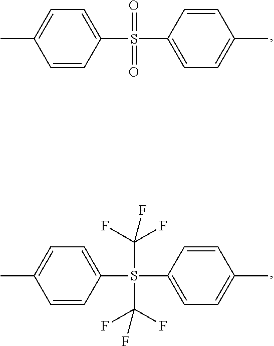
or phenyl;
each Q is 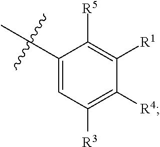
each X is, independently, O, S, or N;
each R¹ is, independently, H, CF₃, C(CH₃)₃, halo, or OH;
each R³ is, independently, H, —NHR², —(CH₂)₁₋₂NH₂, —NH₂, —NH(CH₂)₁₋₃NH₂, 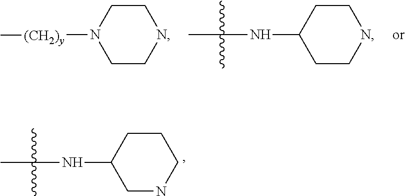
wherein each y is, independently, 1 or 2;
each R² is, independently, H, or the free base or salt form of —(CH₂)₁₋₄NH₂ or —(CH₂)₁₋₄NHC(═NH)NH₂;
each R⁴ is, independently, H, —CF₃, —NHC(═O)(CH₂)₁₋₆NHC(═NH)NH₂, 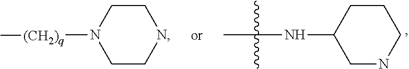
wherein each q is, independently, 1 or 2; and
each R⁵ is, independently, H, —(CH₂)₁₋₄NH₂, or CF₃;
wherein the compound comprises: 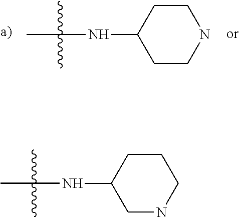 [SMILES: *NC1CCNCC1 ; *NC1CCCNC1]
and at least one R³, 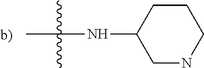 [SMILES: *NC1CCCNC1]
and at least one R⁴; or
c) —(CH₂)₁₋₄NH₂ and at least one R⁵;
g) Formula IX: 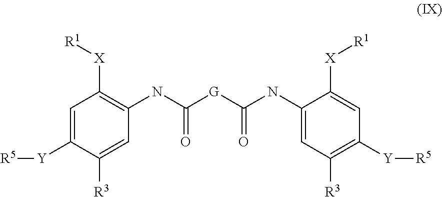
or a pharmaceutically acceptable salt thereof,
wherein:
G is 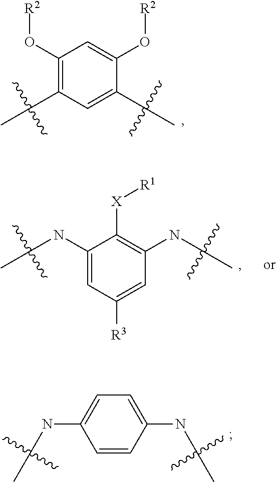
each X is, independently, O or S;
each Y is, independently, O or S;
each R¹ is, independently, 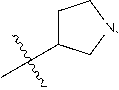
—(CH₂)₁₋₄NH₂, or —(CH₂)₁₋₄NHC(═NH)NH₂;
each R² is, independently, H, C₁-C₈alkyl, —(CH₂)₁₋₄NH₂, or —(CH₂)₁₋₄NHC(═NH)NH₂;
each R³ is, independently, H, —CF₃, —C(CH₃)₃, halo, or OH; and
each R⁵ is, independently, —(CH₂)₁₋₄NH₂ or —(CH₂)₁₋₄NHC(═NH)NH₂;
h) Formula Xa: 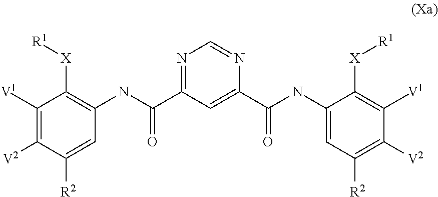
or a pharmaceutically acceptable salt thereof,
wherein:
each X is, independently, O, S, or S(═O)₂;
each R¹ is, independently, —(CH₂)₁₋₄C(═O)OH, —(CH₂)₁₋₄NH₂, —(CH₂)₁₋₄OH, —(CH₂)₁₋₄NHC(═O)C₁₋₄alkyl, or —(CH₂)₁₋₄C(═O)OC₁₋₄alkyl;
each R² is, independently, H, halo, —CF₃, or —C(CH₃)₃;
each V² is H; and
each V¹ is, independently, amino or —NC(═O)—R³, where each R³ is, independently, —C₁₋₄alkyl, —(CH₂)₁₋₄—NH₂, —(CH₂)₁₋₄—NH—C(═NH)NH₂, or aryl (optionally substituted with one or more, independently, halo, cyano, or —C₁₋₄alkoxy);
i) Formula Xb: 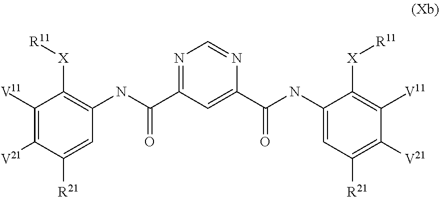
or a pharmaceutically acceptable salt thereof,
wherein:
each X is, independently, O or S;
each R¹¹ is, independently, —(CH₂)₁₋₄NH₂ or —C₁₋₄alkyl;
each R²¹ is, independently, H, halo, —CF₃, or —C(CH₃)₃;
each V¹¹ is H; and
each V² is, independently, —S—(CH₂)₁₋₄—NH₂ or —S—(CH₂)₁₋₄—NHC(═NH)NH₂;
j) Formula XI: 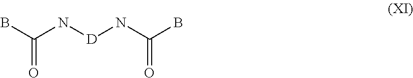
or a pharmaceutically acceptable salt thereof,
wherein:
D is 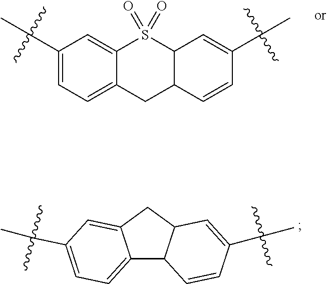 [SMILES: *C1=CC2C(C=C1)Cc1ccc(*)cc1S2(=O)=O ; *C1=CC2Cc3cc(*)ccc3C2C=C1]
each B is, independently, 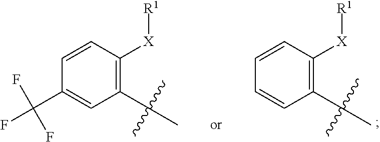
each X is, independently, O or S; and
each R¹ is, independently, —(CH₂)₁₋₄NHC(═NH)NH₂;
k) Formula XII: 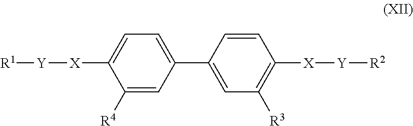
or a pharmaceutically acceptable salt thereof,
wherein:
each X is, independently, —NH—, O, S, or absent;
each Y is, independently, —C(═O), —C(═S), or absent;
R¹ is —(CH₂)ₙNHC(═NH)NH₂, —NH(CH₂)ₙNH₂, —NH(CH₂)ₙNC(═N)NH₂, —(CH₂)ₙNH₂, —O—(CH₂)ₙNH₂, —O—(CH₂)ₙNC(═N)NH₂, —CH═CH—CH₂NH₂, —CH═CH—CH₂NC(═N)NH₂, —CH═CH—(CH₂)₂NH₂, —CH═CH—(CH₂)₂NC(═N)NH₂, —C≡C—CH₂NH₂, —C≡C—(CH₂)₂NH₂, —CH≡CH—CH₂NC(═N)NH₂, or —C≡C—(CH₂)₂—NC(═N)NH₂, where n is 2, 3, or 4; or an aryl, heteroaryl, or heterocycle (each of which is optionally substituted with one or more W);
R² is —(CH₂)ₙNHC(═NH)NH₂, —NH(CH₂)ₙNH₂, —NH(CH₂)ₙNC(═N)NH₂, —(CH₂)ₙNH₂, —O—(CH₂)ₙNH₂, —O—(CH₂)ₙNC(═N)NH₂, —CH═CH—CH₂NH₂, —CH═CH—CH₂NC(═N)NH₂, —CH═CH—(CH₂)₂NH₂, —CH═CH—(CH₂)₂NC(═N)NH₂, —C≡C—CH₂NH₂, —C≡C—(CH₂)₂NH₂, —CH≡CH—CH₂NC(═N)NH₂, or —C≡C—(CH₂)₂—NC(═N)NH₂, where n is 2, 3, or 4; or an aryl, heteroaryl, or heterocycle (each of which is optionally substituted with one or more W);
R³ is H, —CF₃, —NH(CH₂)ₙNH₂, —NH(CH₂)ₙNC(═N)NH₂, —(CH₂)ₙNH₂, —(CH₂)ₙNC(═N)NH₂, —O—(CH₂)ₙNH₂, —O—(CH₂)ₙNC(═N)NH₂, —CH═CH—CH₂NH₂, —CH═CH—CH₂NC(═N)NH₂, —CH═CH—(CH₂)₂NH₂, —CH═CH—(CH₂)₂NC(═N)NH₂, —C≡C—CH₂NH₂, —C≡C—(CH₂)₂NH₂, —CH≡CH—CH₂NC(═N)NH₂, or —C≡C—(CH₂)₂—NC(═N)NH₂, where n is 2, 3, or 4;
R⁴ is H, —CF₃, —NH(CH₂)ₙNH₂, —NH(CH₂)ₙNC(═N)NH₂, —(CH₂)ₙNH₂, —(CH₂)ₙNC(═N)NH₂, —O—(CH₂)ₙNH₂, —O—(CH₂)ₙNC(═N)NH₂, —CH═CH—CH₂NH₂, —CH═CH—CH₂NC(═N)NH₂, —CH═CH—(CH₂)₂NH₂, —CH═CH—(CH₂)₂NC(═N)NH₂, —C≡C—CH₂NH₂, —C≡C—(CH₂)₂NH₂, —CH≡CH—CH₂NC(═N)NH₂, or —C≡C—(CH₂)₂—NC(═N)NH₂, where n is 2, 3, or 4; and
each W is, independently, halo, —CF₃, —S—(CH₂)₁₋₅NHC(═NH)NH₂, —S—(CH₂)₁₋₅NH₂, —S—(CH₂)₁₋₅N(CH₃)₂, —S—(CH₂)₁₋₅NHS(═O)₂—C₁₋₄alkyl, —C₁₋₄alkyl, —O—(CH₂)₁₋₅NHC(═NH)NH₂; or —O-heterocycle or heterocycle wherein the heterocycle is optionally substituted with one or more cyano, amino, guanyl, C₁₋₄alkyl, guanidino, hydroxyl, amidino, C₁₋₄alkoxy, CF₃, or halo;
l) Formula XIII: 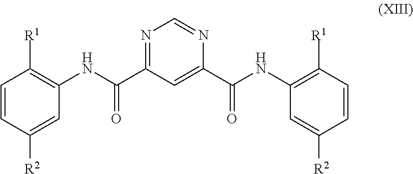
or pharmaceutically acceptable salt thereof,
wherein:
each R¹ is, independently, —S—(CH₂)₁₋₅NH₂, —S—(CH₂)₁₋₅NHC(═NH)NH₂, —C₁₋₄alkoxy, —S—(CH₂)₁₋₅N(C₁₋₄alkyl)₂, —O—(CH₂)₁₋₅NH₂, —O—(CH₂)₁₋₅NHC(═NH)NH₂, —(CH₂)₁₋₅NH₂, —O—(CH₂)₁₋₅N(C₁₋₄alkyl)₂, —C(═O)—(CH₂)₁₋₅NH₂, —C(═O)—(CH₂)₁₋₅NHC(═NH)NH₂, —C(═O)—(CH₂)₁₋₅N(C₁₋₄alkyl)₂, —(CH₂)₁₋₅NHC(═NH)NH₂, —(CH₂)₁₋₅N(C₁₋₄alkyl)₂, —NHC(═O)—(CH₂)₁₋₅NHC(═NH)NH₂, —NHC(═O)—C₁₋₄alkyl, or —NHC(═O)—(CH₂)₁₋₅NHS(═O)₂—C₁₋₄alkyl; and
each R² is, independently, —CF₃, —(CH₂)₁₋₅NH₂, —C₁₋₄alkyl, or halo;
m) Formula XIV: 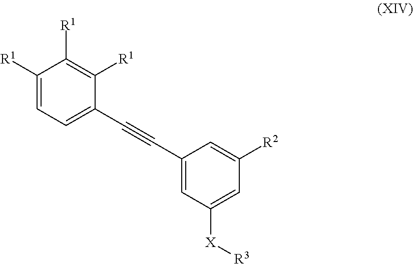
or pharmaceutically acceptable salt thereof,
wherein:
each R¹ is, independently, absent or halo, CF₃, cyano, amino, nitro, amidino, C₁₋₄alkyl, guanidino, hydroxyl, or C₁₋₄alkoxy;
each R² is halo, cyano, amino, nitro, amidino, C₁₋₄alkyl, guanidino, hydroxyl, CF₃, or C₁₋₄alkoxy; and
each R³ is, independently, —S—(CH₂)₁₋₅NH₂, —S—(CH₂)₁₋₅NHC(═NH)NH₂, —C₁₋₄alkoxy, —S—(CH₂)₁₋₅N(C₁₋₄alkyl)₂, —O—(CH₂)₁₋₅NH₂, —O—(CH₂)₁₋₅NHC(═NH)NH₂, —(CH₂)₁₋₅NH₂, —O—(CH₂)₁₋₅N(C₁₋₄alkyl)₂, —C(═O)—(CH₂)₁₋₅NH₂, —C(═O)—(CH₂)₁₋₅NHC(═NH)NH₂, —C(═O)—(CH₂)₁₋₅N(C₁₋₄alkyl)₂, —(CH₂)₁₋₅NHC(═NH)NH₂, —(CH₂)₁₋₅N(C₁₋₄alkyl)₂, —NHC(═O)—(CH₂)₁₋₅NHC(═NH)NH₂, —NHC(═O)—C₁₋₄alkyl, or —NHC(═O)—(CH₂)₁₋₅NHS(═O)₂—C₁₋₄alkyl;
n) Formula XV: 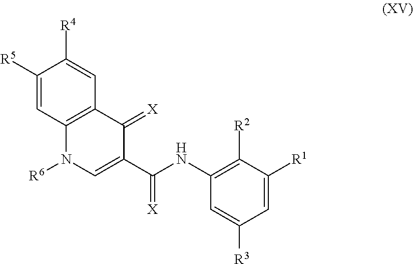
or a pharmaceutically acceptable salt thereof,
wherein:
each X is, independently, O or S;
R¹ is amino, cyano, C₁₋₄alkyl, guanidino, hydroxyl, amidino, C₁₋₄alkoxy, CF₃, or halo;
R² is —S—(CH₂)₁₋₅NH₂, —S—(CH₂)₁₋₅NHC(═NH)NH₂, —S—(CH₂)₁₋₅N(C₁₋₄alkyl)₂, —O—(CH₂)₁₋₅NH₂, —O—(CH₂)₁₋₅NHC(═NH)NH₂, —(CH₂)₁₋₅NH₂, —O—(CH₂)₁₋₅N(C₁₋₄alkyl)₂, —C(═O)—(CH₂)₁₋₅NH₂, —C(═O)—(CH₂)₁₋₅NHC(═NH)NH₂, —C(═O)—(CH₂)₁₋₅N(C₁₋₄alkyl)₂, —(CH₂)₁₋₅NHC(═NH)NH₂, —(CH₂)₁₋₅N(C₁₋₄alkyl)₂, —NHC(═O)—(CH₂)₁₋₅NHC(═NH)NH₂, —NHC(═O)—C₁₋₄alkyl, or —NHC(═O)—(CH₂)₁₋₅NHS(═O)₂—C₁₋₄alkyl;
R³ is CF₃, amino, cyano, C₁₋₄alkyl, guanidino, hydroxyl, amidino, C₁₋₄alkoxy, or halo;
R⁴ is halo, amino, cyano, C₁₋₄alkyl, guanidino, hydroxyl, amidino, C₁₋₄alkoxy, or CF₃;
R⁵ is a heterocycloalkyl; and
R⁶ is a cycloalkyl; or
o) Formula XVI: 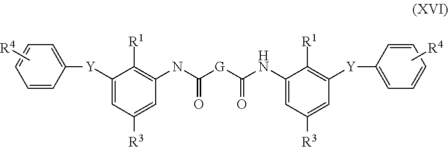
or a pharmaceutically acceptable salt thereof,
wherein:
G is 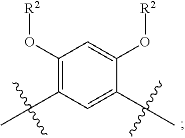
each Y is —NH—C(═O)—;
each R¹ is, independently, H or —S—(CH₂)₁₋₄NH₂, or —S—(CH₂)₁₋₄NHC(═NH)NH₂;
each R² is, independently, H, C₁-C₄alkyl, —(CH₂)₁₋₄NH₂, or —(CH₂)₁₋₄NHC(═NH)NH₂;
each R³ is, independently, H, —CF₃, —C(CH₃)₃, halo, or OH; and
each R⁴ is, independently, H, —CF₃, —C(CH₃)₃, halo, cyano, or OH.

146. A pharmaceutical composition comprising a compound of claim 145, or a pharmaceutically acceptable salt thereof, and a pharmaceutically acceptable carrier.

147. A method of treating malaria in a mammal, or killing or inhibiting the growth of a Plasmodium species, comprising administering to the mammal, optionally in need thereof, or contacting the species with, an effective amount of a compound:
a) of Formula I:
R¹-A₁-X—Y-A₂-Y—X-A₁-R² (I)
or a pharmaceutically acceptable salt thereof,
wherein:
each X is, independently, —NR⁸, —N(R⁸)N(R⁸)—, O, or S, wherein each R⁸ is, independently, hydrogen or alkyl;
each Y is, independently, C═O, C═S, or O═S═O;
each A₁ and A₂ is, independently, aryl optionally substituted with one or more W, or heteroaryl optionally substituted with one or more W;
each W is, independently, halo, —CF₃, cyano, C₁₋₄alkoxy, —S—(CH₂)₁₋₅NHC(═NH)NH₂, —S—(CH₂)₁₋₅NH₂, —S—(CH₂)₁₋₅N(CH₃)₂, —S—(CH₂)₁₋₅NHS(═O)₂—C₁₋₄alkyl, —C₁₋₄alkyl, —O—(CH₂)₁₋₅NHC(═NH)NH₂, or O-heterocycle (wherein the heterocycle is optionally substituted with one or more cyano, amino, C₁₋₄alkyl, guanidino, hydroxyl, amidino, C₁₋₄alkoxy, CF₃, or halo); and
R¹ and R² are, independently, —(CH₂)₁₋₄NH₂, —(CH₂)₁₋₅NHC(═NH)NH₂, —S—(CH₂)₁₋₅N(CH₃)₂, —O—(CH₂)₁₋₅NHC(═NH)NH₂, —S—(CH₂)₁₋₅NHC(═NH)NH₂, —O—(CH₂)₁₋₅N(CH₃)₂, amino, —O-heterocycle or —O-cycloalkyl (wherein the heterocycle and cycloalkyl are optionally substituted), —S-heterocycle (wherein the heterocycle is optionally substituted), —NHC(═O)—(CH₂)₁₋₅NHC(═NH)NH₂, —NHC(═O)—C₁₋₄alkyl, heterocycle (wherein the heterocycle is optionally substituted), —NHC(═O)—(CH₂)₁₋₅NHS(═O)₂—C₁₋₄alkyl, —NHC(═O)—(CH₂)₁₋₅-aryl (wherein either or both —(CH₂)₁₋₅ and aryl is optionally substituted), —NHC(═O)-aryl (wherein the aryl is optionally substituted), or —NHC(═O)—(CH₂)₁₋₅NH-aryl (wherein either or both —NH and aryl is optionally substituted), wherein the optional substituents are chosen from one or more cyano, amino, C₁₋₄alkyl, guanidino, hydroxyl, amidino, C₁₋₄alkoxy, CF₃, halo, and —N(CH₃)₂;
provided that the compound of Formula I is not one of the following: 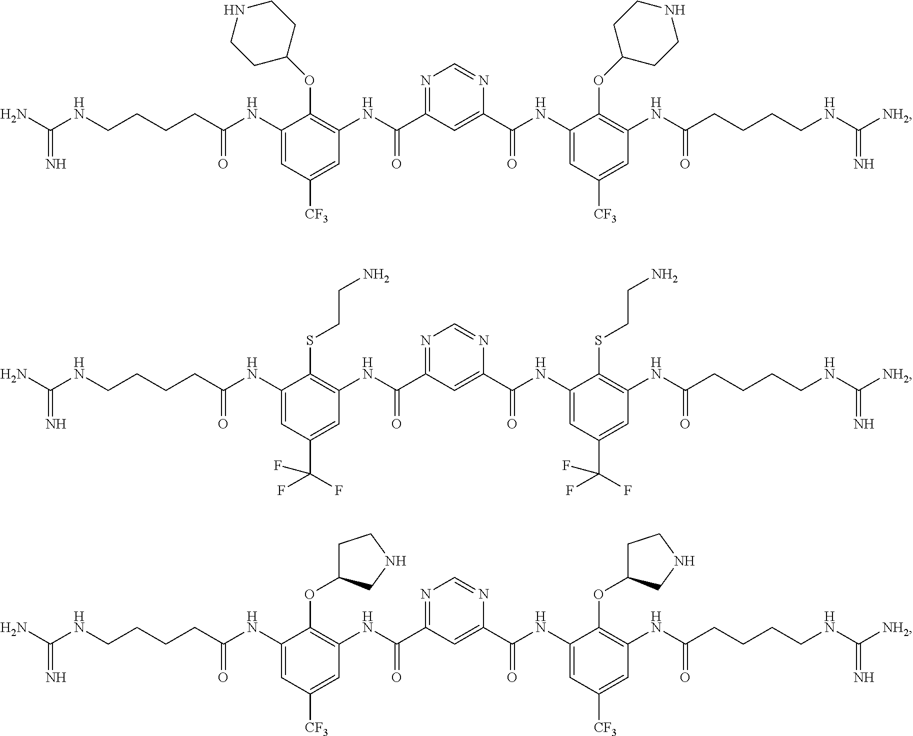 [SMILES: N=C(N)NCCCCC(=O)Nc1cc(C(F)(F)F)cc(NC(=O)c2cc(C(=O)Nc3cc(C(F)(F)F)cc(NC(=O)CCCCNC(=N)N)c3OC3CCNCC3)ncn2)c1OC1CCNCC1 ; N=C(N)NCCCCC(=O)Nc1cc(C(F)(F)F)cc(NC(=O)c2cc(C(=O)Nc3cc(C(F)(F)F)cc(NC(=O)CCCCNC(=N)N)c3SCCN)ncn2)c1SCCN ; N=C(N)NCCCCC(=O)Nc1cc(C(F)(F)F)cc(NC(=O)c2cc(C(=O)Nc3cc(C(F)(F)F)cc(NC(=O)CCCCNC(=N)N)c3O[C@H]3CCNC3)ncn2)c1O[C@H]1CCNC1]
or its enantiomer, 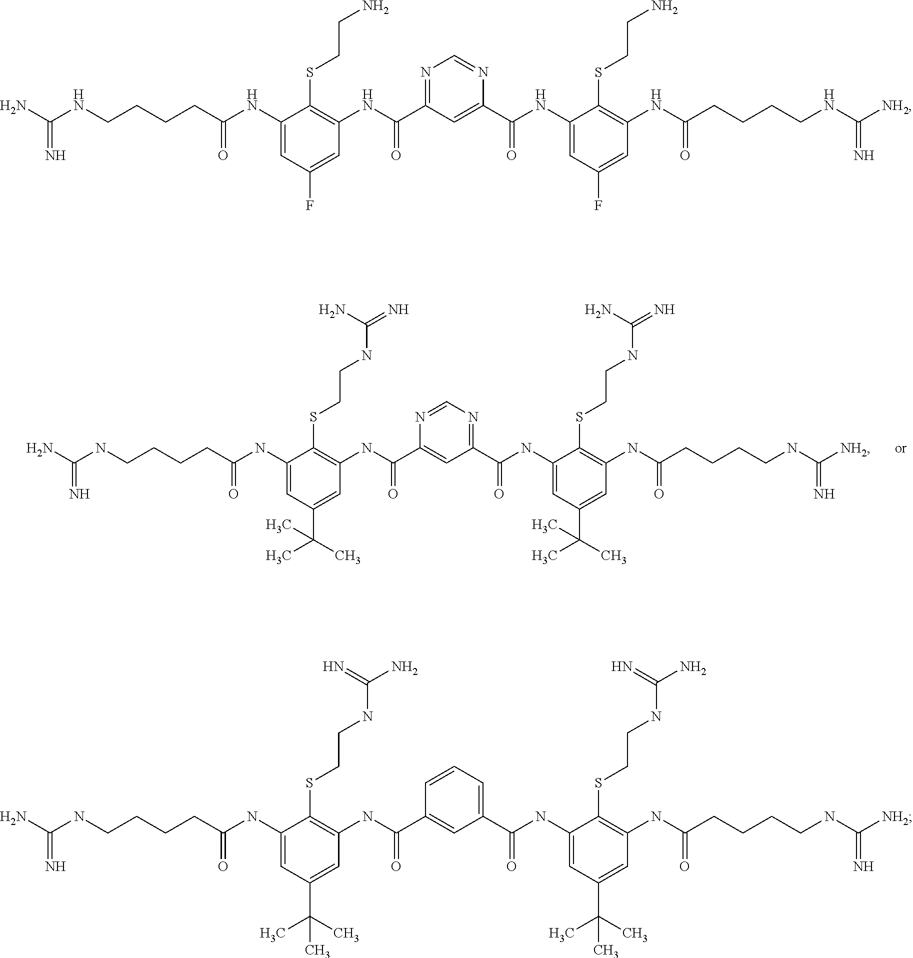 [SMILES: N=C(N)NCCCCC(=O)Nc1cc(F)cc(NC(=O)c2cc(C(=O)Nc3cc(F)cc(NC(=O)CCCCNC(=N)N)c3SCCN)ncn2)c1SCCN ; CC(C)(C)c1cc(NC(=O)CCCCNC(=N)N)c(SCCNC(=N)N)c(NC(=O)c2cc(C(=O)Nc3cc(C(C)(C)C)cc(NC(=O)CCCCNC(=N)N)c3SCCNC(=N)N)ncn2)c1 ; CC(C)(C)c1cc(NC(=O)CCCCNC(=N)N)c(SCCNC(=N)N)c(NC(=O)c2cccc(C(=O)Nc3cc(C(C)(C)C)cc(NC(=O)CCCCNC(=N)N)c3SCCNC(=N)N)c2)c1]
b) of Formula IIa: 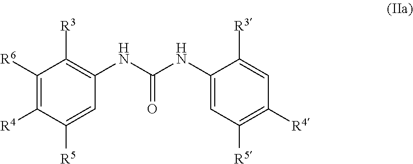
or a pharmaceutically acceptable salt thereof,
wherein:
R³ and R^{3′}are, independently, H or —O—C₁₋₄alkyl;
R⁶ is H, —(CH₂)₁₋₄NHC(═NH)NH₂, or —(CH₂)₁₋₄NH₂;
R⁵ and R^{5′} are, independently, H, —CF₃, —(CH₂)₁₋₄NHC(═NH)NH₂, or —(CH₂)₁₋₄NH₂; and
R⁴ and R^{4′}are, independently, H, —S—(CH₂)₁₋₄NHC(═NH)NH₂, —S—(CH₂)₁₋₄NH₂, 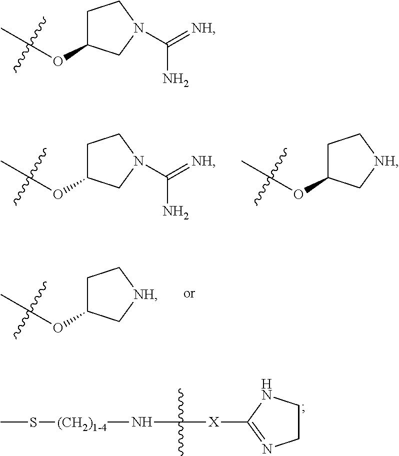
c) of Formula III:
R¹—X-A₁-A₂-A₁-X—R² (III)
or a pharmaceutically acceptable salt thereof,
wherein:
A₂ is aryl optionally substituted with one or more W, or heteroaryl optionally substituted with one or more W;
each A₁ is, independently, aryl optionally substituted with one or more W, or heteroaryl optionally substituted with one or more W;
each X is, independently, absent, —NR⁸, —N(R⁸)N(R⁹)—, O, or S, wherein each R⁸ is, independently, hydrogen or alkyl;
R¹ and R² are, independently, absent, —C(═O)—(CH₂)₁₋₅NHC(═NH)NH₂, hydrogen, —(CH₂)₁₋₅NHC(═NH)NH₂, —C(═O)—CH(NH₂)C₁₋₄alkyl, halo, —NO₂, —CF₃, —N⁺(═O)O⁻, amino, —S—(CH₂)₁₋₅NHC(═NH)NH₂, —O—(CH₂)₁₋₅NHC(═NH)NH₂, —(CH₂)₁₋₅NHC(═NH)NH₂, —S—(CH₂)₁₋₅N(CH₃)₂, —O—(CH₂)₁₋₅N(CH₃)₂, —NHC(═O)—(CH₂)₁₋₅NHC(═NH)NH₂, —NHC(═O)—C₁₋₄alkyl, or —NHC(═O)—(CH₂)₁₋₅NHS(═O)₂—C₁₋₄alkyl; and
each W is, independently, —O—(CH₂)₁₋₅NHC(═NH)NH₂, —O—(CH₂)₁₋₅NH₂, —CF₃, halo, —C₁₋₄alkyl, —N((CH₂)₁₋₅—NH₂)((CH₂)₁₋₅N(C₁₋₄alkyl)₂), —N((CH₂)₁₋₅N(C₁₋₄alkyl)₂)₂, —C₁₋₄alkylamino, —(CH₂)₁₋₅NHC(═NH)NH₂, —S—(CH₂)₁₋₅NHC(═NH)NH₂, —S—(CH₂)₁₋₅NH₂, —S—(CH₂)₁₋₅N(CH₃)₂, —S—(CH₂)₁₋₅NHS(═O)₂—C₁₋₄alkyl, —C≡C—(CH₂)₁₋₅—NHC(═NH)NH₂, —C≡C—(CH₂)₁₋₅—NH₂, or aryl or heterocycle each optionally substituted with one or more —CF₃, cyano, amino, C₁₋₄alkyl, C₁₋₄alkoxy, guanidino, hydroxyl, amidino, halo, —(CH₂)₁₋₄NH₂, —O—(CH₂)₁₋₄NH₂, —(CH₂)₁₋₄NHC(═NH)NH₂, or —O—(CH₂)₁₋₄NHC(═NH)NH₂;
d) of Formula IV:
Het¹-X—Y-Het²-Y—X-Het¹ (IV)
or a pharmaceutically acceptable salt thereof,
wherein:
each X is, independently, —NR⁸, —N(R⁸)N(R⁸)—, O, or S, wherein each R⁸ is, independently, hydrogen or alkyl;
each Y is, independently, C═O, C═S, or O═S═O;
each Het¹ is, independently, a fused bicyclic ring chosen from naphthalene, isobenzofuran, indolizine, isoindole, indole, purine, isoquinoline, quinolone, phthalazine, naphthyridine, quinoxaline, quinazoline, pteridine, chroman, isochroman, indoline, isoindoline, and 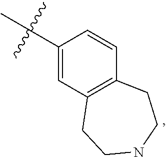 [SMILES: *c1ccc2c(c1)CCNCC2]
each of which is optionally substituted with one or more W;
Het² is an aryl or heteroaryl chosen from thiophene, furan, pyran, pyrrole, imidazole, pyrazole, isothiaole, isoxazole, pyridine, pyrazine, pyrimidine, and pyridazine; and
each W is, independently, —(CH₂)₁₋₅NH₂, —C₁₋₄alkyl, —(CH₂)₁₋₅NHC(═NH)NH₂, —O—(CH₂)₁₋₅NHC(═NH)NH₂, —O—(CH₂)₁₋₅NH₂, —CF₃, halo, C₁₋₄alkyl, —S—(CH₂)₁₋₅NH₂, —N((CH₂)₁₋₅N(C₁₋₄alkyl)₂)₂, —N((CH₂)₁₋₅—NH₂)((CH₂)₁₋₅N(C₁₋₄alkyl)₂), —S—(CH₂)₁₋₅N(CH₃)₂, —(CH₂)₁₋₅NHC(═NH)NH₂, —S—(CH₂)₁₋₅NHC(═NH)NH₂, —S—(CH₂)₁₋₅NHS(═O)₂—C₁₋₄alkyl, or cycloalkyl optionally substituted with one or more —CF₃, cyano, amino, nitro, C₁₋₄alkyl, C₁₋₄alkoxy, guanidino, hydroxyl, amidino, or halo;
e) of Formula VII: 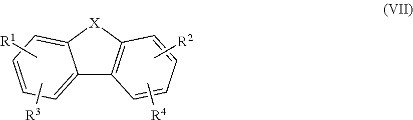
or a pharmaceutically acceptable salt thereof,
wherein:
X is C(R⁷)C(R⁸), C(R⁷)(R⁸), S, or —N(R⁹);
R⁷, R⁸, and R⁹ are, independently, C₁-C₄alkyl, —(CH₂)₀₋₄NH₂, or —(CH₂)₀₋₄NHC(═NH)NH₂;
R¹ and R² are, independently, H, C₁-C₈alkyl, C₁-C₈alkoxy, halo, OH, CN, CF₃, or haloC₁-C₈alkyl;
R³ and R⁴ are, independently, H, carbocycle(R⁵)(R⁶), —NH-carbocycle(R⁵)(R⁶);
each R⁵ and each R⁶ are, independently, H, C₁-C₈alkyl, C₁-C₈alkoxy, halo, amino, amidino, OH, CF₃, —N—(CH₃)₂, —O—(CH₂)₁₋₅—NH₂, —O—(CH₂)₁₋₅NHC(═NH)NH₂, —S—(CH₂)₁₋₅—NH₂, —N((CH₂)₁₋₅NH₂)₂, —S—(CH₂)₁₋₅NHC(═NH)NH₂, —C(═O)NH(CH₂)₁₋₅NH₂, —(CH₂)₁₋₈—NH₂, —(CH₂)₁₋₅N((CH₂)₁₋₅NH₂)₂, aromatic group, heterocycle, —(CH₂)₁₋₈—NH—(CH₂)₁₋₈—NH₂, or —(CH₂)₁₋₈—NH—C(═NH)NH₂;
f) of Formula VIII:
Q-X—Z—X-Q (VIII)
or a pharmaceutically acceptable salt thereof,
wherein:
Z is 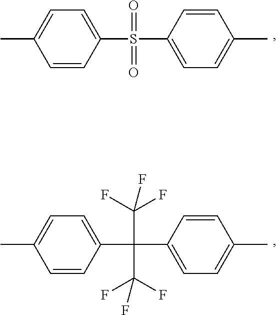
or phenyl;
each Q is 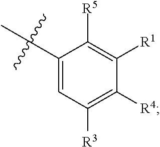
each X is, independently, O, S, or N;
each R¹ is, independently, H, CF₃, C(CH₃)₃, halo, or OH;
each R³ is, independently, H, —NHR², —(CH₂)₁₋₂NH₂, —NH₂, —NH(CH₂)₁₋₃NH₂, 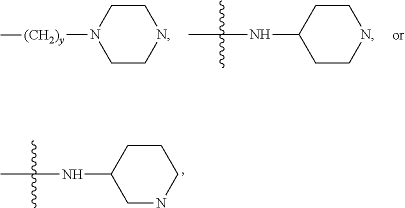
wherein each y is, independently, 1 or 2;
each R² is, independently, H, or the free base or salt form of —(CH₂)₁₋₄NH₂ or —(CH₂)₁₋₄NHC(═NH)NH₂;
each R⁴ is, independently, H, —CF₃, —NHC(═O)(CH₂)₁₋₆NHC(═NH)NH₂, 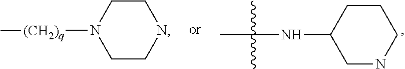
wherein each q is, independently, 1 or 2; and
each R⁵ is, independently, H, —(CH₂)₁₋₄NH₂, or CF₃;
wherein the compound comprises: 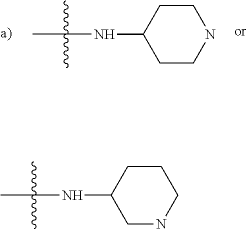 [SMILES: *NC1CCNCC1 ; *NC1CCCNC1]
and at least one R³; 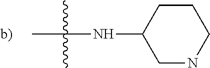 [SMILES: *NC1CCCNC1]
at least one R⁴; or
c) —(CH₂)₁₋₄NH₂ and at least one R⁵;
g) of Formula IX: 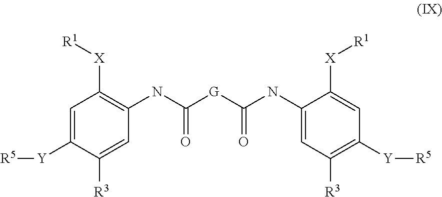
or a pharmaceutically acceptable salt thereof,
wherein:
G is 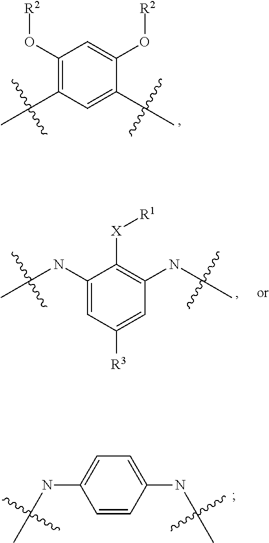
each X is, independently, O or S;
each Y is, independently, O or S;
each R¹ is, independently, 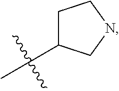
—(CH₂)₁₋₄NH₂, or —(CH₂)₁₋₄NHC(═NH)NH₂;
each R² is, independently, H, C₁-C₈alkyl, —(CH₂)₁₋₄NH₂, or —(CH₂)₁₋₄NHC(═NH)NH₂;
each R³ is, independently, H, —CF₃, —C(CH₃)₃, halo, or OH; and
each R⁵ is, independently, —(CH₂)₁₋₄NH₂ or —(CH₂)₁₋₄NHC(═NH)NH₂;
h) of Formula Xa: 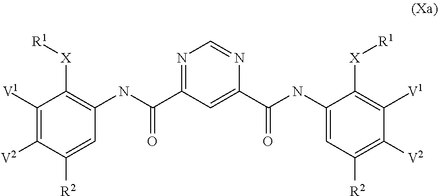
or a pharmaceutically acceptable salt thereof,
wherein:
each X is, independently, O, S, or S(═O)₂;
each R¹ is, independently, —(CH₂)₁₋₄C(═O)OH, —(CH₂)₁₋₄NH₂, —(CH₂)₁₋₄OH, —(CH₂)₁₋₄NHC(═O)C₁₋₄alkyl, or —(CH₂)₁₋₄C(═O)OC₁₋₄alkyl;
each R² is, independently, H, halo, —CF₃, or —C(CH₃)₃;
each V² is H; and
each V¹ is, independently, amino or —NC(═O)—R³, where each R³ is, independently, —C₁₋₄alkyl, —(CH₂)₁₋₄—NH₂, —(CH₂)₁₋₄—NH—C(═NH)NH₂, or aryl (optionally substituted with one or more, independently, halo, cyano, or —C₁₋₄alkoxy);
i) of Formula Xb: 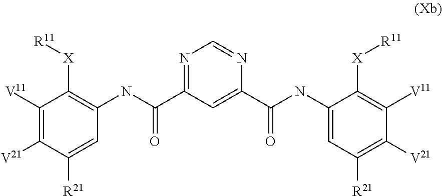
or a pharmaceutically acceptable salt thereof,
wherein:
each X is, independently, O or S;
each R¹¹ is, independently, —(CH₂)₁₋₄NH₂ or —C₁₋₄alkyl;
each R²¹ is, independently, H, halo, —CF₃, or —C(CH₃)₃;
each V¹¹ is H; and
each V² is, independently, —S—(CH₂)₁₋₄—NH₂ or —S—(CH₂)₁₋₄—NHC(═NH)NH₂;
j) of Formula XI: 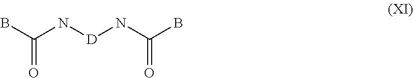
or a pharmaceutically acceptable salt thereof,
wherein:
D is 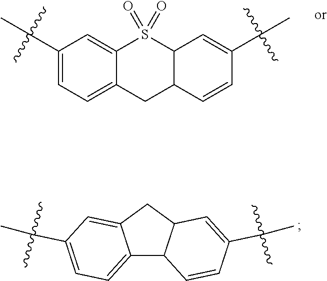 [SMILES: *C1=CC2C(C=C1)Cc1ccc(*)cc1S2(=O)=O ; *C1=CC2Cc3cc(*)ccc3C2C=C1]
each B is, independently, 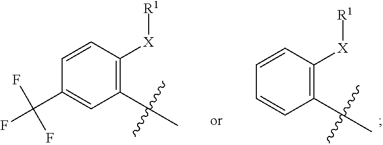
each X is, independently, O or S; and
each R¹ is, independently, —(CH₂)₁₋₄NHC(═NH)NH₂;
k) of Formula XII: 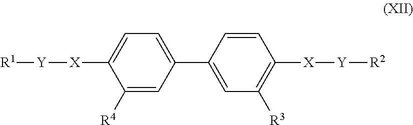
or a pharmaceutically acceptable salt thereof,
wherein:
each X is, independently, —NH—, O, S, or absent;
each Y is, independently, —C(═O), —C(═S), or absent;
R¹ is —(CH₂)ₙNHC(═NH)NH₂, —NH(CH₂)ₙNH₂, —NH(CH₂)ₙNC(═N)NH₂, —(CH₂)ₙNH₂, —O—(CH₂)ₙNH₂, —O—(CH₂)ₙNC(═N)NH₂, —CH═CH—CH₂NH₂, —CH═CH—CH₂NC(═N)NH₂, —CH═CH—(CH₂)₂NH₂, —CH═CH—(CH₂)₂NC(═N)NH₂, —C≡C—CH₂NH₂, —C≡C—(CH₂)₂NH₂, —CH≡CH—CH₂NC(═N)NH₂, or —C≡C—(CH₂)₂—NC(═N)NH₂, where n is 2, 3, or 4; or an aryl, heteroaryl, or heterocycle (each of which is optionally substituted with one or more W);
R² is —(CH₂)ₙNHC(═NH)NH₂, —NH(CH₂)ₙNH₂, —NH(CH₂)ₙNC(═N)NH₂, —(CH₂)ₙNH₂, —O—(CH₂)ₙNH₂, —O—(CH₂)ₙNC(═N)NH₂, —CH═CH—CH₂NH₂, —CH≡CH—CH₂NC(═N)NH₂, —CH═CH—(CH₂)₂NH₂, —CH═CH—(CH₂)₂NC(═N)NH₂, —C≡C—CH₂NH₂, —C≡C—(CH₂)₂NH₂, —CH≡CH—CH₂NC(═N)NH₂, or —C≡C—(CH₂)₂—NC(═N)NH₂, where n is 2, 3, or 4; or an aryl, heteroaryl, or heterocycle (each of which is optionally substituted with one or more W);
R³ is H, —CF₃, —NH(CH₂)ₙNH₂, —NH(CH₂)ₙNC(═N)NH₂, —(CH₂)ₙNH₂, —(CH₂)ₙNC(═N)NH₂, —O—(CH₂)ₙNH₂, —O—(CH₂)ₙNC(═N)NH₂, —CH═CH—CH₂NH₂, —CH═CH—CH₂NC(═N)NH₂, —CH═CH—(CH₂)₂NH₂, —CH═CH—(CH₂)₂NC(═N)NH₂, —C≡C—CH₂NH₂, —C≡C—(CH₂)₂NH₂, —CH≡CH—CH₂NC(═N)NH₂, or —C≡C—(CH₂)₂—NC(═N)NH₂, where n is 2, 3, or 4;
R⁴ is H, —CF₃, —NH(CH₂)ₙNH₂, —NH(CH₂)ₙNC(═N)NH₂, —(CH₂)ₙNH₂, —(CH₂)ₙNC(═N)NH₂, —O—(CH₂)ₙNH₂, —O—(CH₂)ₙNC(═N)NH₂, —CH═CH—CH₂NH₂, —CH═CH—CH₂NC(═N)NH₂, —CH═CH—(CH₂)₂NH₂, —CH═CH—(CH₂)₂NC(═N)NH₂, —C≡C—CH₂NH₂, —C≡C—(CH₂)₂NH₂, —CH≡CH—CH₂NC(═N)NH₂, or —C≡C—(CH₂)₂—NC(═N)NH₂, where n is 2, 3, or 4; and
each W is, independently, halo, —CF₃, —S—(CH₂)₁₋₅NHC(═NH)NH₂, —S—(CH₂)₁₋₅NH₂, —S—(CH₂)₁₋₅N(CH₃)₂, —S—(CH₂)₁₋₅NHS(═O)₂—C₁₋₄alkyl, —C₁₋₄alkyl, —O—(CH₂)₁₋₅NHC(═NH)NH₂; or —O-heterocycle or heterocycle wherein the heterocycle is optionally substituted with one or more cyano, amino, guanyl, C₁₋₄alkyl, guanidino, hydroxyl, amidino, C₁₋₄alkoxy, CF₃, or halo;
l) of Formula XIII: 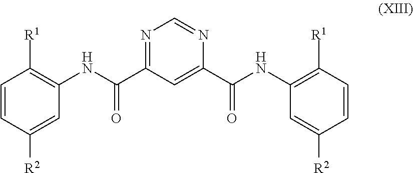
or pharmaceutically acceptable salt thereof,
wherein:
each R¹ is, independently, —S—(CH₂)₁₋₅NH₂, —S—(CH₂)₁₋₅NHC(═NH)NH₂, —C₁₋₄alkoxy, —S—(CH₂)₁₋₅N(C₁₋₄alkyl)₂, —O—(CH₂)₁₋₅NH₂, —O—(CH₂)₁₋₅NHC(═NH)NH₂, —(CH₂)₁₋₅NH₂, —O—(CH₂)₁₋₅N(C₁₋₄alkyl)₂, —C(═O)—(CH₂)₁₋₅NH₂, —C(═O)—(CH₂)₁₋₅NHC(═NH)NH₂, —C(═O)—(CH₂)₁₋₅N(C₁₋₄alkyl)₂, —(CH₂)₁₋₅NHC(═NH)NH₂, —(CH₂)₁₋₅N(C₁₋₄alkyl)₂, —NHC(═O)—(CH₂)₁₋₅NHC(═NH)NH₂, —NHC(═O)—C₁₋₄alkyl, or —NHC(═O)—(CH₂)₁₋₅NHS(═O)₂—C₁₋₄alkyl; and
each R² is, independently, —CF₃, —(CH₂)₁₋₅NH₂, —C₁₋₄alkyl, or halo;
m) of Formula XIV: 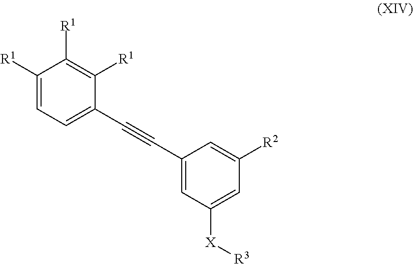
or pharmaceutically acceptable salt thereof,
wherein:
each R¹ is, independently, absent or halo, CF₃, cyano, amino, nitro, amidino, C₁₋₄alkyl, guanidino, hydroxyl, or C₁₋₄alkoxy;
each R² is halo, cyano, amino, nitro, amidino, C₁₋₄alkyl, guanidino, hydroxyl, CF₃, or C₁₋₄alkoxy; and
each R³ is, independently, —S—(CH₂)₁₋₅NH₂, —S—(CH₂)₁₋₅NHC(═NH)NH₂, —C₁₋₄alkoxy, —S—(CH₂)₁₋₅N(C₁₋₄alkyl)₂, —O—(CH₂)₁₋₅NH₂, —O—(CH₂)₁₋₅NHC(═NH)NH₂, —(CH₂)₁₋₅NH₂, —O—(CH₂)₁₋₅N(C₁₋₄alkyl)₂, —C(═O)—(CH₂)₁₋₅NH₂, —C(═O)—(CH₂)₁₋₅NHC(═NH)NH₂, —C(═O)—(CH₂)₁₋₅N(C₁₋₄alkyl)₂, —(CH₂)₁₋₅NHC(═NH)NH₂, —(CH₂)₁₋₅N(C₁₋₄alkyl)₂, —NHC(═O)—(CH₂)₁₋₅NHC(═NH)NH₂, —NHC(═O)—C₁₋₄alkyl, or —NHC(═O)—(CH₂)₁₋₅NHS(═O)₂—C₁₋₄alkyl;
n) of Formula XV: 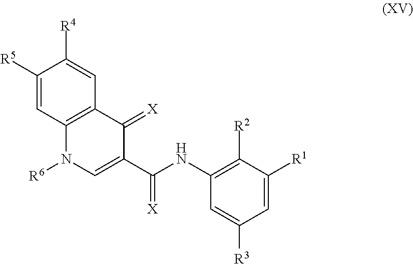
or a pharmaceutically acceptable salt thereof,
wherein:
each X is, independently, O or S;
R¹ is amino, cyano, C₁₋₄alkyl, guanidino, hydroxyl, amidino, C₁₋₄alkoxy, CF₃, or halo;
R² is —S—(CH₂)₁₋₅NH₂, —S—(CH₂)₁₋₅NHC(═NH)NH₂, —S—(CH₂)₁₋₅N(C₁₋₄alkyl)₂, —O—(CH₂)₁₋₅NH₂, —O—(CH₂)₁₋₅NHC(═NH)NH₂, —(CH₂)₁₋₅NH₂, —O—(CH₂)₁₋₅N(C₁₋₄alkyl)₂, —C(═O)—(CH₂)₁₋₅NH₂, —C(═O)—(CH₂)₁₋₅NHC(═NH)NH₂, —C(═O)—(CH₂)₁₋₅N(C₁₋₄alkyl)₂, —(CH₂)₁₋₅NHC(═NH)NH₂, —(CH₂)₁₋₅N(C₁₋₄alkyl)₂, —NHC(═O)—(CH₂)₁₋₅NHC(═NH)NH₂, —NHC(═O)—C₁₋₄alkyl, or —NHC(═O)—(CH₂)₁₋₅NHS(═O)₂—C₁₋₄alkyl;
R³ is CF₃, amino, cyano, C₁₋₄alkyl, guanidino, hydroxyl, amidino, C₁₋₄alkoxy, or halo;
R⁴ is halo, amino, cyano, C₁₋₄alkyl, guanidino, hydroxyl, amidino, C₁₋₄alkoxy, or CF₃;
R⁵ is a heterocycloalkyl; and
R⁶ is a cycloalkyl;
o) of Formula XVI: 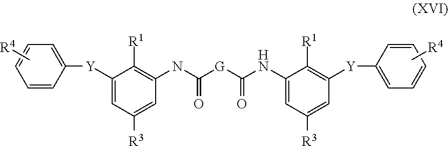
or a pharmaceutically acceptable salt thereof,
wherein:
G is 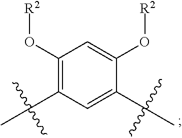
each Y is —NH—C(═O)—;
each R¹ is, independently, H or —S—(CH₂)₁₋₄NH₂, or —S—(CH₂)₁₋₄NHC(═NH)NH₂;
each R² is, independently, H, C₁-C₄alkyl, —(CH₂)₁₋₄NH₂, or —(CH₂)₁₋₄NHC(═NH)NH₂;
each R³ is, independently, H, —CF₃, —C(CH₃)₃, halo, or OH; and
each R⁴ is, independently, H, —CF₃, —C(CH₃)₃, halo, cyano, or OH;
p) of Formula II:
R₁-A₁-X—Z—X-A₁-R² (II)
or a pharmaceutically acceptable salt thereof,
wherein:
each X is, independently, —NR⁸, —N(R⁸)N(R⁸)—, O, or S, wherein each R⁸ is, independently, hydrogen or alkyl;
each Z is, independently, C═O, C═S, or O═S═O;
each A₁ is, independently, aryl optionally substituted with one or more W, or heteroaryl optionally substituted with one or more W;
each W is, independently, —CF₃, halo, C₁₋₄alkyl, —O—(CH₂)₁₋₅NHC(═NH)NH₂, —N((CH₂)₁₋₅—NH₂)((CH₂)₁₋₅N(C₁₋₄alkyl)₂), —N((CH₂)₁₋₅N(C₁₋₄alkyl)₂)₂, heterocycle, —S—(CH₂)₁₋₅NHC(═NH)NH₂, —S—(CH₂)₁₋₅NH₂, —S—(CH₂)₁₋₅N(CH₃)₂, —C₁₋₄alkyl, —S—(CH₂)₁₋₅NHS(═O)₂—C₁₋₄alkyl, O-heterocycle (wherein the heterocycle is optionally substituted with one or more cyano, amino, C₁₋₄alkyl, C₁₋₄alkoxy, guanidino, hydroxyl, amidino, or halo); and
R¹ and R² are, independently, hydrogen, halo, —NO₂, —S—(CH₂)₁₋₅NHC(═NH)NH₂, —O—(CH₂)₁₋₅NHC(═NH)NH₂, —CF₃, —N⁺(═O)O⁻, —(CH₂)₁₋₅NHC(═NH)NH₂, amino, —S—(CH₂)₁₋₅N(CH₃)₂, —O—(CH₂)₁₋₅N(CH₃)₂, —O-heterocycle (wherein the heterocycle is optionally substituted), —S-heterocycle (wherein the heterocycle is optionally substituted), —NHC(═O)—C₁₋₄alkyl, —NHC(═O)—(CH₂)₁₋₅NHC(═NH)NH₂, —NHC(═O)—(CH₂)₁₋₅-aryl (wherein either or both the —(CH₂)₁₋₅ or phenyl is optionally substituted), —NHC(═O)—(CH₂)₁₋₅NH-aryl (wherein either or both —NH and/or aryl is optionally substituted), —NHC(═O)-aryl (wherein the aryl is optionally substituted), or —NHC(═O)—(CH₂)₁₋₅NHS(═O)₂—C₁₋₄alkyl, wherein the optional substituents are chosen from one or more cyano, amino, C₁₋₄alkyl, guanidino, hydroxyl, amidino, C₁₋₄alkoxy, CF₃, and halo;
q) of Formula V: 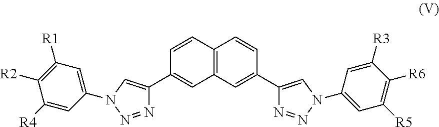
or a pharmaceutically acceptable salt thereof,
wherein:
R¹ is H, —NH(CH₂)ₙNH₂, —NH(CH₂)ₙNC(═N)NH₂, —(CH₂)ₙNH₂, —O—(CH₂)ₙNH₂, —(CH₂)ₙNC(═N)NH₂, —O—(CH₂)ₙNC(═N)NH₂, —CH═CH—CH₂NH₂, —CH═CH—CH₂NC(═N)NH₂, —CH═CH—(CH₂)₂NH₂, —CH═CH—(CH₂)₂NC(═N)NH₂, —C≡C—CH₂NH₂, —C≡C—(CH₂)₂NH₂, —CH≡CH—CH₂NC(═N)NH₂, or —C≡C—(CH₂)₂—NC(═N)NH₂, where n is 2, 3, or 4;
R² is H, —NH(CH₂)ₙNH₂, —NH(CH₂)ₙNC(═N)NH₂, —(CH₂)ₙNH₂, —O—(CH₂)ₙNH₂, —(CH₂)ₙNC(═N)NH₂, —O—(CH₂)ₙNC(═N)NH₂, —CH═CH—CH₂NH₂, —CH═CH—CH₂NC(═N)NH₂, —CH═CH—(CH₂)₂NH₂, —CH═CH—(CH₂)₂NC(═N)NH₂, —C≡C—CH₂NH₂, —C≡C—(CH₂)₂NH₂, —CH≡CH—CH₂NC(═N)NH₂, or —C≡C—(CH₂)₂—NC(═N)NH₂, where n is 2, 3, or 4;
R³ is H, —NH(CH₂)ₙNH₂, —NH(CH₂)ₙNC(═N)NH₂, —(CH₂)ₙNH₂, —O—(CH₂)ₙNH₂, —(CH₂)ₙNC(═N)NH₂, —O—(CH₂)ₙNC(═N)NH₂, —CH═CH—CH₂NH₂, —CH═CH—CH₂NC(═N)NH₂, —CH═CH—(CH₂)₂NH₂, —CH═CH—(CH₂)₂NC(═N)NH₂, —C≡C—CH₂NH₂, —C≡C—(CH₂)₂NH₂, —CH≡CH—CH₂NC(═N)NH₂, or —C≡C—(CH₂)₂—NC(═N)NH₂, where n is 2, 3, or 4;
R⁴ is H, —NH(CH₂)ₙNH₂, —NH(CH₂)ₙNC(═N)NH₂, —(CH₂)ₙNH₂, —O—(CH₂)ₙNH₂, —(CH₂)ₙNC(═N)NH₂, —O—(CH₂)ₙNC(═N)NH₂, —CH═CH—CH₂NH₂, —CH═CH—CH₂NC(═N)NH₂, —CH═CH—(CH₂)₂NH₂, —CH═CH—(CH₂)₂NC(═N)NH₂, —C≡C—CH₂NH₂, —C≡C—(CH₂)₂NH₂, —CH≡CH—CH₂NC(═N)NH₂, or —C≡C—(CH₂)₂—NC(═N)NH₂, where n is 2, 3, or 4;
R⁵ is H, —NH(CH₂)ₙNH₂, —NH(CH₂)ₙNC(═N)NH₂, —(CH₂)ₙNH₂, —O—(CH₂)ₙNH₂, —(CH₂)ₙNC(═N)NH₂, —O—(CH₂)ₙNC(═N)NH₂, —CH═CH—CH₂NH₂, —CH═CH—CH₂NC(═N)NH₂, —CH═CH—(CH₂)₂NH₂, —CH═CH—(CH₂)₂NC(═N)NH₂, —C≡C—CH₂NH₂, —C≡C—(CH₂)₂NH₂, —CH≡CH—CH₂NC(═N)NH₂, or —C≡C—(CH₂)₂—NC(═N)NH₂, where n is 2, 3, or 4; and
R⁶ is H, —NH(CH₂)ₙNH₂, —NH(CH₂)ₙNC(═N)NH₂, —(CH₂)ₙNH₂, —O—(CH₂)ₙNH₂, —(CH₂)ₙNC(═N)NH₂, —O—(CH₂)ₙNC(═N)NH₂, —CH═CH—CH₂NH₂, —CH═CH—CH₂NC(═N)NH₂, —CH═CH—(CH₂)₂NH₂, —CH═CH—(CH₂)₂NC(═N)NH₂, —C≡C—CH₂NH₂, —C≡C—(CH₂)₂NH₂, —CH≡CH—CH₂NC(═N)NH₂, or —C≡C—(CH₂)₂—NC(═N)NH₂, where n is 2, 3, or 4;
or a pharmaceutically acceptable salt thereof;
r) of Formula VI: 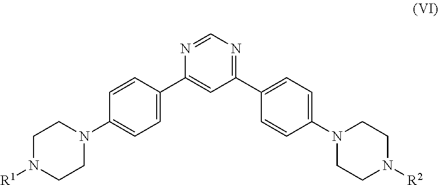
or a pharmaceutically acceptable salt thereof, wherein R¹ and R² are, independently, hydrogen, —C₁₋₄alkyl, —C(═NH)NH₂, —(CH₂)ₙNH₂, or —(CH₂)ₙNC(═NH)NH₂, where n is 2, 3, or 4; or
s) chosen from: 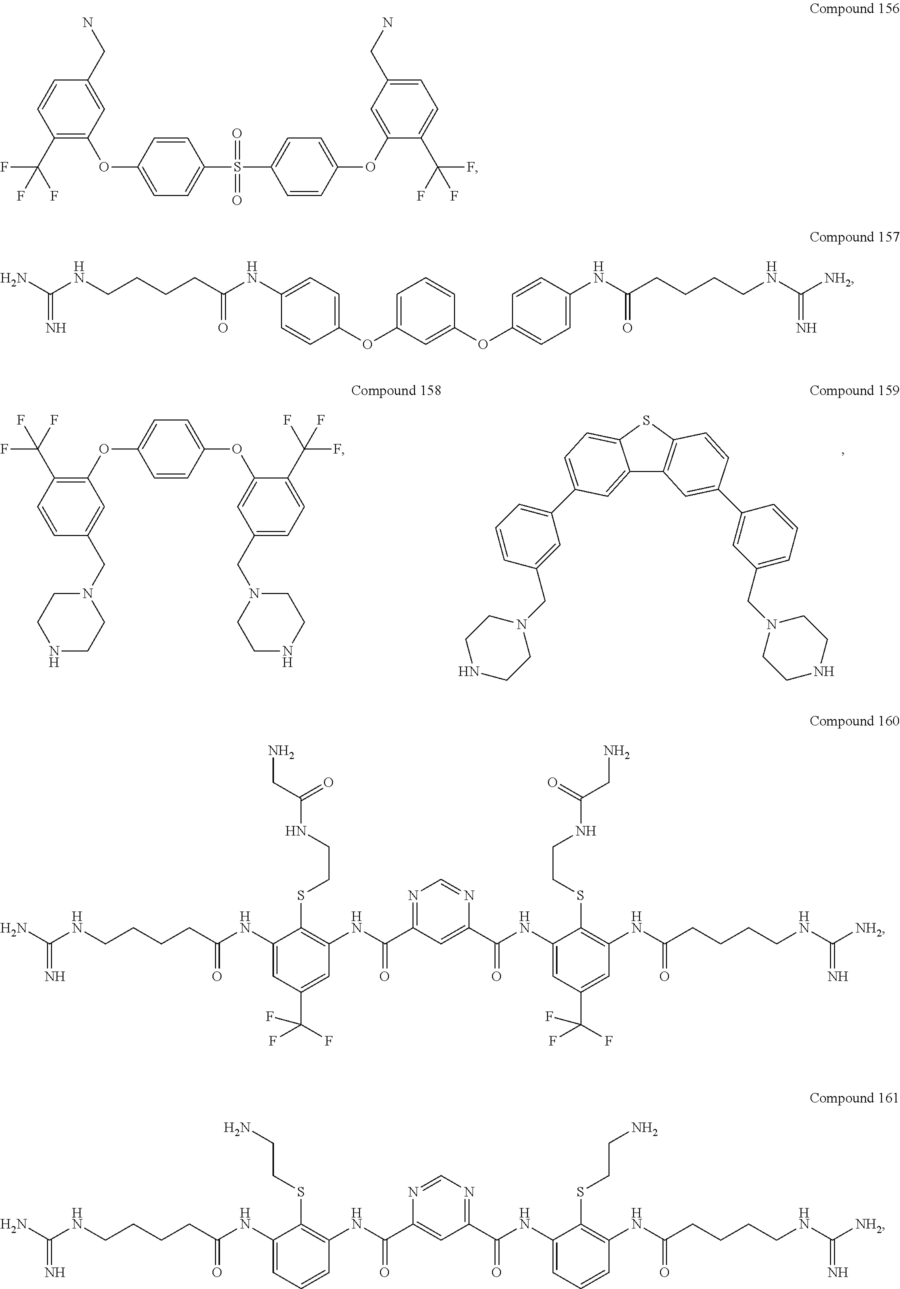 [SMILES: NCc1ccc(C(F)(F)F)c(Oc2ccc(S(=O)(=O)c3ccc(Oc4cc(CN)ccc4C(F)(F)F)cc3)cc2)c1 ; N=C(N)NCCCCC(=O)Nc1ccc(Oc2cccc(Oc3ccc(NC(=O)CCCCNC(=N)N)cc3)c2)cc1 ; FC(F)(F)c1ccc(CN2CCNCC2)cc1Oc1ccc(Oc2cc(CN3CCNCC3)ccc2C(F)(F)F)cc1 ; c1cc(CN2CCNCC2)cc(-c2ccc3sc4ccc(-c5cccc(CN6CCNCC6)c5)cc4c3c2)c1 ; N=C(N)NCCCCC(=O)Nc1cc(C(F)(F)F)cc(NC(=O)c2cc(C(=O)Nc3cc(C(F)(F)F)cc(NC(=O)CCCCNC(=N)N)c3SCCNC(=O)CN)ncn2)c1SCCNC(=O)CN ; N=C(N)NCCCCC(=O)Nc1cccc(NC(=O)c2cc(C(=O)Nc3cccc(NC(=O)CCCCNC(=N)N)c3SCCN)ncn2)c1SCCN] 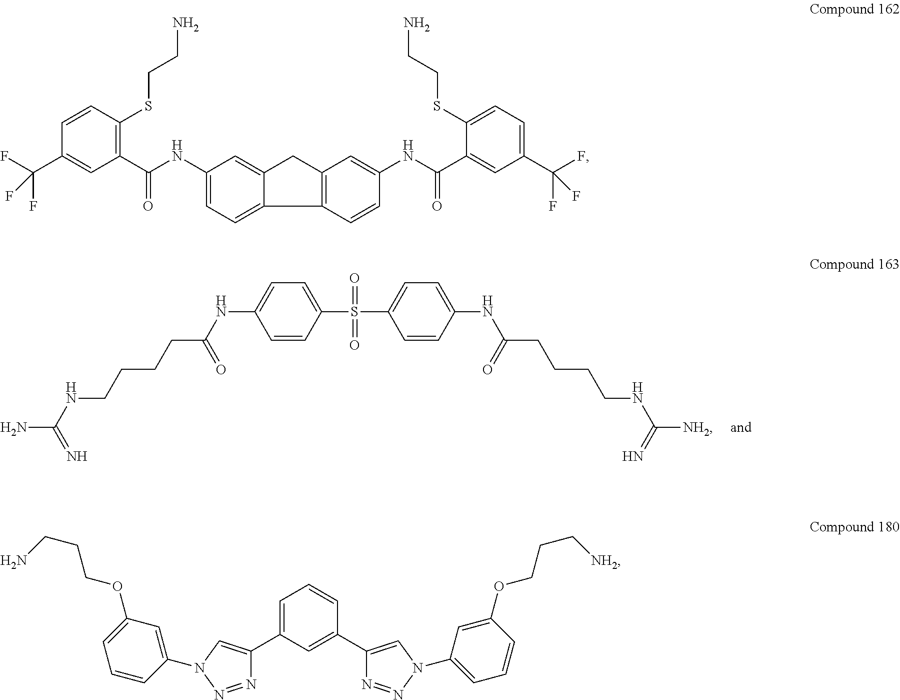 [SMILES: NCCSc1ccc(C(F)(F)F)cc1C(=O)Nc1ccc2c(c1)Cc1cc(NC(=O)c3cc(C(F)(F)F)ccc3SCCN)ccc1-2 ; N=C(N)NCCCCC(=O)Nc1ccc(S(=O)(=O)c2ccc(NC(=O)CCCCNC(=N)N)cc2)cc1 ; "and" ; NCCCOc1cccc(-n2cc(-c3cccc(-c4cn(-c5cccc(OCCCN)c5)nn4)c3)nn2)c1]
or a pharmaceutically acceptable salt thereof.

148. The method of claim 147 wherein the malaria is chloroquine-sensitive or chloroquine-resistant.
